(19) United States
(12) Patent Application Publication
(10) Pub. No.: US 2024/0358710 A1
(43) Pub. Date: Oct. 31, 2024

(54) METHODS, COMPOSITIONS AND DEVICES FOR TREATING CANCER WITH ILLUDOFULVENES

(71) Applicant: AF Chemicals, LLC

(72) Inventor: MICHAEL KELNER, San Diego, CA (US)

(73) Assignee: AF Chemicals, LLC, (US)

(21) Appl. No.: 18/755,609

(22) Filed: Jun. 26, 2024

Related U.S. Application Data
(63) Continuation of application No. 17/508,729, filed on Oct. 22, 2021, now Pat. No. 12,023,339.
Application No. 17/508,729 is a continuation of application No. 16/708,005, filed on Dec. 9, 2019, now Pat. No. 11,160,807.
(60) Provisional application No. 62/777,774, filed on Dec. 11, 2018.
(60) Provisional application No. 62/796,409, filed on Jan. 24, 2019.
(60) Provisional application No. 62/940,081, filed on Nov. 25, 2019.

(30) Foreign Application Priority Data
Dec. 9, 2019 (EP) EP19214360

(51) Int. Cl. A61K 31/5375; A61K 31/047; A61K 31/121; A61K 31/122; A61K 31/135; A61K 31/15; A61K 31/16; A61K 31/18; A61K 31/192; A61K 31/198; A61K 31/351; A61K 31/357; A61K 31/401; C12Q 1/6886

(52) U.S. Cl. CPC A61K 31/5375; A61K 31/047; A61K 31/121; A61K 31/122; A61K 31/135; A61K 31/15; A61K 31/16; A61K 31/18; A61K 31/192; A61K 31/198; A61K 31/351; A61K 31/357; A61K 31/401; C12Q 1/6886; C12Q 2600/158; C12Q 2600/16

(57) ABSTRACT

In an embodiment of the invention, a method of treating cancer includes screening to select a patient population that will respond to an Illudofulvene Analog composition based on the presence of one or more Transcription Coupled Repair (TCR) mutations, deletions or other events interfering with TCR biomarker expression in combination with upregulation of Myc and/or PTGR biomarker expression and treating the patient population with the Illudofulvene Analog composition.

### PRIORITY CLAIM

This application claims priority to (i) U.S. Utility application Ser. No. 17/508,729 entitled “METHODS, COMPOSITIONS AND DEVICES FOR TREATING CANCER WITH ILLUDOFULVENES”, inventor: Michael Kelner filed Oct. 22, 2021 which claims priority to (ii) U.S. Utility application Ser. No. 16/708,005 entitled “METHODS, COMPOSITIONS AND DEVICES FOR TREATING CANCER WITH ILLUDOFULVENES”, inventor: Michael Kelner filed Dec. 9, 2019, which issued Nov. 2, 2021 as U.S. Pat. No. 11,160,807, which claims priority to (iii) U.S. Provisional Application No. 62/777,774 entitled “METHODS, COMPOSITIONS AND DEVICES FOR TREATING CANCER WITH ILLUDOFULVENES”, inventor: Michael Kelner filed Dec. 11, 2018, (iv) U.S. Provisional Application No. 62/796,409 entitled “METHODS, COMPOSITIONS AND DEVICES FOR TREATING CANCER WITH ILLUDOFULVENES”, inventor: Michael Kelner filed Jan. 24, 2019, and (v) U.S. Provisional Application No. 62/796,409 entitled “METHODS, COMPOSITIONS AND DEVICES FOR TREATING CANCER WITH ILLUDOFULVENES”, inventor: Michael Kelner filed Nov. 25, 2019 which applications (i)-(v) are herein expressly incorporated by reference in their entireties and for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file MKEL-01048US5_ST26.TXT, created on Jun. 26, 2024, 1,301,742 bytes, machine format IBM-PC, MS-Windows operating system, is hereby incorporated by reference.

### TECHNICAL FIELD

The present invention relates to compositions, methods and devices for treating cancer with illudofulvenes including methods to detect the expression levels of genes encoding biomarkers in cancer patients and to predict the responsiveness of cancer patients to illudofulvenes.

### BACKGROUND ART

DNA microarrays have been used to measure gene expression in samples from patients and to facilitate diagnosis of disease. Gene expression can reveal the presence of cancer in a patient in addition to the type, stage, and origin of the cancer. Gene expression may even have a role in predicting the efficacy of cancer therapies. In recent decades, the National Cancer Institute (NCI) has tested cancer therapeutics for their effect in limiting the growth of 60 human cancer cell lines. The NCI has also measured gene expression in those 60 cancer cell lines using DNA microarrays. Various studies have explored the relationship between gene expression and therapeutic effect using the NCI datasets.

Illudofulvenes have been proposed as medicants for Affinity Drug Conjugates in U.S. Pat. No. 10,285,955 issued May 14, 2019 to M. J. Kelner entitled Affinity Medicant Conjugates, which is incorporated by reference herein in its entirety and for all purposes.

### SUMMARY OF INVENTION

There exists a continuing need for delivery of chemotherapeutic agents for which tumors do not have a medicant resistant phenotype and which inhibit tumor growth, especially solid tumor growth, and which have an adequate therapeutic index to be effective for in vivo treatment. The illudofulvenes of the present invention have shown efficacy in humans and animals for a wide range of therapeutic applications including: cancer, adenocarcinoma, carcinoma, breast cancer, prostate cancer, ovarian cancer, endometrial cancer, and neuroendocrine tumors. For example, veterinary and agricultural applications can include treatment of cancer, adenocarcinoma, carcinoma, ovarian cancer, endometrial cancer, and neuroendocrine tumors in farmyard and/or companion animals.

The invention features methods for detecting expression of a biomarker (e.g., one, two, three, four, five, ten, or all of the biomarkers shown in Tables 1-8, such as Excision Repair Cross-Complementation (ERCC) e.g., ERCC2 (SEQ ID NO:21)) in a patient having cancer, such as a human patient having cancer that is resistant to one or more cancer therapies other than an Illudofulvene Analog (e.g., a patient with prostate cancer, ovarian cancer, or hepatocellular carcinoma (HCC) that is resistant to one or more cancer therapies including radiation therapy and/or chemotherapy based on Other Drugs as set forth herein), and for determining responsiveness of a cancer patient (e.g., a patient with prostate cancer, ovarian cancer, or HCC) to treatment with an Illudofulvene Analog. The invention also features methods of treating cancer in a patient in need thereof (e.g., a patient with prostate cancer, ovarian cancer, or HCC or a treatment resistant form thereof) that include administering an Illudofulvene Analog to the patient, in which the patient is or has been determined to be responsive to an Illudofulvene Analog according to the diagnostic methods described herein. Without wishing to be bound by any theory, it is believed that sensitivity to Illudofulvene Analog treatment in patients can be caused by a deficiency in a component of the transcription-coupled repair (TCR) pathway or the post-replication repair pathway (e.g. ERCC or RAD18 E3 Ubiquitin Protein Ligase (RAD18)), or alternatively sensitivity to Illudofulvene Analogs can be caused by an upregulation or increase in a gene product (i.e., protein, e.g., MYC Proto-Oncogene (MYC), and/or Prostaglandin Reductase (PTGR), e.g., PTGR1, and/or PTGR2). A Southern blot, a northern blot, mRNA/RNA microarray, protein array, gene sequencing, immunohistochemical staining requiring additional tissue sampling and a readout by a competent pathologist can be utilized to identify the patient population.

The methods of this invention include predicting the sensitivity of illudofulvenes, preferably in the form of a pharmaceutical composition, to an animal in need thereof. In a further embodiment, pharmaceutical compositions are disclosed containing illudofulvenes of the present invention in combination with a pharmaceutically acceptable carrier.

In an embodiment of the present invention, an illudofulvenes can target cancer cells with reduced toxicity to normal cells. In an embodiment of the present invention, these compounds are useful in treatment of tumors in which a marker is expressed. In various embodiments of the present invention, pharmaceutical compositions comprising the illudofulvene compounds are used in the treatment of tumors in which the marker is expressed. In various embodiments of the present invention, methods of using the pharmaceutical compositions comprise these compounds to treat tumors in which the marker is expressed. In various embodiments of the present invention, pharmaceutical compositions comprising these compounds are used in the treatment of one or more of the following diseases: cancer, adenocarcinoma, carcinoma, breast cancer, prostate cancer, ovarian cancer, endometrial cancer.

### BRIEF DESCRIPTION OF THE DRAWINGS

This invention is described with respect to specific embodiments thereof. Additional features can be appreciated from the Figures in which:

FIG. 1 shows the tumor regression produced by administration of an Illudofulvene Analog 002, according to an embodiment of the invention;

FIG. 2 shows that expression of MYC causes cancer cells to become sensitive to the illudofulvene (Analog 002), according to an embodiment of the invention;

FIG. 3 shows a comparison of Cell Count (%) as a function of the Illudofulvene Analog 002 concentration for two cell types (NIH3T3 MYC+ and NIH3T3 MYC-null), according to an embodiment of the invention;

FIG. 4A shows that MYC expression causes cells to become sensitive to Illudofulvene Analog 002, according to an embodiment of the invention;

FIG. 4B shows that MYC expression is not sensitive to Mithramycin;

FIG. 4C shows that MYC expression is not sensitive to Mitomycin C;

FIG. 4D shows that MYC expression is not sensitive to Topotecan; and

FIG. 5 shows a scheme relating the responsiveness of the cancer patient to treatment with Illudofulvenes based on the relative expression of the biomarkers.

### DETAILED DESCRIPTION OF THE INVENTION

The term ‘upregulated’ means that the messenger RNA and/or the gene product (protein) is increased above basal level of a housekeeping gene by at least approximately one point nine (1.9) standard deviations which corresponds with ninety (90) percent confidence assuming a normal distribution. The phrase ‘increased expression’ means that the messenger RNA and/or the gene product (protein) is increased above a basal level of a housekeeping gene by at least approximately one point nine (1.9) standard deviations which corresponds with ninety (90) percent confidence assuming a normal distribution. The term ‘downregulated’ means that the messenger RNA and/or the gene product (protein) is decreased below basal levels of a housekeeping gene by at least approximately one point nine (1.9) standard deviations which corresponds with ninety (90) percent confidence assuming a normal distribution. The phrase ‘decreased expression’ means that the messenger RNA and/or the gene product (protein) is decreased below a basal level of a housekeeping gene by at least approximately one point nine (1.9) standard deviations which corresponds with ninety (90) percent confidence assuming a normal distribution. In relation to upregulation and/or downregulation, approximately means plus or minus ten (10) percent.

The phrase ‘not upregulated’ means that the messenger RNA and/or the gene product (protein) are within the normal range of the basal level of a housekeeping gene. A basal expression level is the expression level of an mRNA or a protein under normal circumstances. The phrase ‘determining if a cancerous tumor is sensitive to an illudofulvene analog’ means that the indicated gene is either upregulated or downregulated as specified. The phrase ‘using a selected technique’ means a technique selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

The phrase ‘determining the regulation of expression of a PTGR gene’ means probing the level of expression of the PTGR gene in the (tissue) sample using a selected technique. The phrase ‘determining the regulation of expression of a MYC gene’ means probing the level of expression of the MYC gene in the (tissue) sample using a selected technique. The phrase ‘determining the regulation of expression of a TCR gene’ means probing the level of expression of the TCR gene in the (tissue) sample using a selected technique. The phrase ‘determining the regulation of expression of a Housekeeping gene’ means probing the level of expression of the Housekeeping gene in the (tissue) sample using a selected technique. The phrase ‘determining a relative ratio of expression of a gene is high’ means calculating that the determined regulation of the gene is 1.9 standard deviations above the determined regulation of a housekeeping gene. The phrase ‘determining a relative ratio of expression of a gene is deficient’ means calculating that the determined regulation of the gene is 1.9 standard deviations below the determined regulation of a housekeeping gene.

The phrase ‘Other Drugs’ means docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, 5-fluorouracil (5-FU), a histone deacetylase (HDAC) inhibitor, ipilimumab, bortezomib, carfilzomib, thalidomide, lenalidomide, pomalidomide, dexamethasone, cyclophosphamide, vincristine, melphalan, tegafur, irinotecan, cetuximab, leucovorin, SN-38, everolimus, temsirolimus, bleomycin, lomustine, depsipeptide, erlotinib, cisplatin, busulfan, epirubicin, arsenic trioxide, bendamustine, fulvestrant, teniposide, adriamycin, decitabine, estramustine, azaguanine, aclarubicin, mitomycin, paclitaxel, taxotere, APO010, ara-c, methylprednisolone, methotrexate, methyl-gag, belinostat, idarubicin, IL4-PR38, valproic acid, all-trans retinoic acid (ATRA), cytoxan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, celecoxib, iressa, anastrozole, and rituximab.

‘Acylfulvene’ means an Illudofulvene subgroup with the following structural molecular formula:

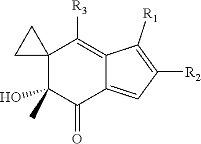

where R₁, R₂, R₃, R₈, R₉, R₁₀, R₁₁, are as set forth in Formula P1, as given herein.

‘Formula P1’ means R₁ represents —H, —CR₉R₈OH, —CHR₉CHR₈OH, —CHR₁₀CHR₉CHR₈OH, —CH₂CHR₁₀CHR₉CHR₈OH, —CR₉R₈OH, —CHR₉CHR₈OH, —CHR₁₀CHR₉CHR₈OH, —CH₂CHR₁₀CHR₉CHR₈OH, —C(═O)H, —CH₂(C═O)H, —CH₂CH₂(C═O)H, —CH₂CH₂CH₂(C═O)H, —CH₂CH₂CH₂CH₂(C═O)H, —CR₉R₈(C═O)H, —CHR₉CHR₈(C═O)H, —CH₂CHR₉CHR₈(C═O)H, —CR₉R₈(C═O)R₁₀, —CHR₉CHR₈(C═O)R₁₀, —CH₂CHR₉CHR₈(C═O)R₁₀, —CH₂CH₂CHR₉CHR₈(C═O)R₁₀, —CO₂H, —CHR₉CO₂H, —CHR₈CHR₉CO₂H, —CHR₁₀CHR₈CHR₉CO₂H, —CO₂R₁₀, —CHR₉CO₂R₁₀, —CHR₈CHR₉CO₂R₁₀, —CH₂CHR₈CHR₉CO₂R₁₀, —CHR₉CH₂CH₂CHR₈CO₂H, —CHR₉CH₂CH₂CHR₈CO₂R₁₀, —CR₈═CH₂, —CHR₈CH═CH₂, —CH₂CHR₈CH═CH₂, —CH₂CH₂CHR₈CH═CH₂, —CR₈═CHR₉, —CHR₈CR═CH₂, —CH₂CHR₈CR₉═CH₂, —CH₂CH₂CHR₈CR₉CH₂, —CR₈═CR₉R₁₀, —CHR₈CH═CR₉R₁₀, —CH₂CHR₈CH═CHR₉R₁₀, —CH₂CH₂CHR₈CH═CHR₉R₁₀, —Cl, —Br, —I, —F, —NO₂, —NR₈R₉, —CH₂Cl, —CH₂Br, —CH₂I, —CH₂F, —CR₈Cl₂, —CR₈Br₂, —CR₈I₂, —CR₈F₂, —CCl₃, —CBr₃, —CI₃, —CF₃, —CHR₈Cl, —CR₈R₉Cl, —CHR₈CHR₉Cl, —CHR₁₀CHR₈CHR₉Cl, —CH₂CHR₁₀CHR₈CHR₉Cl, —CHR₈Br, —CR₈R₉Br, —CHR₈CHR₉Br, —CHR₁₀CHR₈CHR₉Br, —CH₂CHR₁₀CHR₈CHR₉Br, —CHR₈I, —CR₈R₉I, —CHR₈CHR₉Br, —CHR₁₀CHR₈CHR₉I, —CH₂CHR₁₀CHR₈CHR₉I, —CR₈R₉NH₂, —CHR₈CHR₉NH₂, —CHR₁₀CHR₈CHR₉NH₂, —CH₂CH R₁₀CHR₈CHR₉NH₂—CR₉R₁₀NHR₈, —CHR₉CHR₁₀NHR₈, —CH₂CHR₉CHR₁₀NHR₈, —CH₂CH₂CHR₉CHR₁₀NHR₈, —CHR₉NHR₈R₁₀, —CHR₉CH₂NHR₈R₁₀, —CH₂CH₂CHR₉NR₁₀R₈, —CH₂CH₂CH₂CH₂NHR₈, —CR₉R₈OR₁₀, —CH₂CR₈R₉OR₁₀, —CH₂CH₂CR₈R₉OR₁₀, —CH₂CH₂CH₂R₈R₉OR₁₀, —CH₂CH₂CH₂CH₂R₈R₉OR₁₀, —CHR₈OC(═O)CHR₉R₁₀, —CHR8OC(═O)CHR₉R₁₀, —CH₂CH₂OC(═O)CHR₉R₁₀, —CH₂CH₂CHR₈OC(═O)CHR₉R₁₀, —CH₂CH₂CH₂CH₂OC(═O)CHR₉R₁₀, —CHR₈(═O)OCHR₉R₁₀, —CHR₈(═O)OCHR₉R₁₀, —CH₂CHR₈(═O)OCHR₉R₁₀, —CH₂CH₂CHR₈(═O)OCHR₉R₁₀, —CH₂CH₂CH₂CHR₈(═O)OCHR₉R₁₀, —R₁₂C═CR₉CH₂OH, —R₁₂C═CR₉C(═O)H, —R₁₂C═CR₉CH₂OR₁₀, —R₁₂C═CR₉C(═O)R₁₀, —CH₃, —CH₂CH₂, —CHR₈CH₂, —CHR₈CH₂CH₂, —CHR₈CHR₉CH₃, —OCH₃, —OCR₈R₉R₁₀, —OCH₂CR₈R₉R₁₀, —OCR₉R₈CHR₁₀, —OCHR₈CH₂CH₃, —OCHR₈CHR₉CH₃, —NR₈CH₃, —NR₈CH₂CH₃, —NR₉CHR₈CH₃, —NR₉CHR₈CH₂CH₃, —NR₁₀CHR₈CHR₉CH₃, —OCH₂OR₈, —OCHR₈OR₉, —OCHR₈CH₂OR₉, —OCHR₈CHR₉OR₁₀, —OC(═O)OR₈, —OCH₂C(═O)OR₈, —OCHR₉C(═O)OR₈, —CR₈(═N)H, —CH₂CR₈(═N)H, —CH₂CR₈(═N)H, —CH₂CH₂CR₈(═N)H, —CH₂CH₂CH₂CR₈(═N)H, —CH₂CH₂CH₂CH₂CR₈(═N)H, —CR₈(═N)OH, —CH₂CR₈(═N)OH, —CH₂CR₈(═N)OH, —CH₂CH₂CR₈(═N)OH, —CH₂CH₂CH₂CR₈(═N)OH, —CH₂CH₂CH₂CH₂CR₈(═N)OH, —CR₈(═N)R₉, —CH₂CR₈(═N)R₉, —CH₂CR₈(═N)R₉, —CH₂CH₂CR₈(═N)R₉, —CH₂CH₂CH₂CR₈(═N)R₉, —CH₂CH₂CH₂CH₂CR₈(═N)R₉, —CR₈(═N)OR₉, —CH₂CR₈(═N)OR₉, —CH₂CR₈(═N)OR₉, —CH₂CH₂CR₈(═N)OR₉, —CH₂CH₂CH₂CR₈(═N)OR₉, —CH₂CH₂CH₂CH₂CR₈(═N)OR₉, —CR₈(═N)NR₉, —CH₂CR₈(═N)NR₉, —CH₂CR₈(═N)NR₉, —CH₂CH₂CR₈(═N)NR₉, —CH₂CH₂CH₂—CR₈(═N)NR₉, —CH₂CH₂CH₂CH₂CR₈(═N)NR₉, —CR₈(═N)NR₉S(═O)₂R₁₀, —CH₂C(R₈)(═N)NR₉S(═O)₂R₁₀, —CH₂CH₂C(R₈)(═N)NR₉S(═O)₂R₁₀, —CH₂CH₂CH₂C(R₈)(═N)NR₉S(═O)₂R₁₀, CH₂CH₂CH₂CH₂C(R₈)(═N)NR₉S(═O)₂R₁₀, —R₁₂N(R₈)C(═O)NR₉R₁₀, —R₁₂N(R₈)C(═S)NR₉R₁₀, —R₁₂N(OR₈)C(═O)NR₉R₁₀, —R₁₂N(OR₈)C(═S)NR₉R₁₀, —R₁₂OS(O₂)NH₂, —R₁₂NHS(O₂)NH₂, —R₁₂OS(O₂)NR₈R₉, —R₁₂NHS(O₂)NR₈R₉, —CH₂N(R₈)S(O₂)NR₉R₁₀, —CH₂CH₂N(R₈)S(O₂)NR₉R₁₀, —CH₂CH₂CH₂N(R₈)S(O₂)NR₉R₁₀, —CH₂N(R₈)S(O₂)CR₉R₁₀R₁₁, —CH₂CH₂N(R₈)S(O₂) CR₉R₁₀R₁₁, —CH₂CH₂CH₂N(R₈)S(O₂) CR₉R₁₀R₁₁, —N(R₈)C(═O)R₉, —CH₂N(R₈)C(═O)R₉, —CH₂CH₂N(R₈)C(═O)R₉, —CH₂CH₂CH₂N(R₈)C(═O)R₉, —CH₂N(R₈)(C═O)NR₉R₁₀, —CH₂CH₂N(R₈)(C═O)NR₉R₁₀, —CH₂CH₂N(R₈)(C═O)NR₉R₁₀, —CH₂N(R₈)(C═O)CR₉R₁₀R₁₁, —CH₂CH₂N(R₈)(C═O)CR₉R₁₀R₁₁, —CH₂CH₂N(R₈)(C═O) CR₉R₁₀R₁₁, —R₁₂N(OH)C(═O)NHOH, —R₁₂N(OH)C(═S)NHOH, —R₁₂N(OR₈)C(═O)NHOR₉, —R₁₂N(OR₈)C(═S)NHOR₉, —R₁₂OS(O₂)NHOH, —R₁₂NHS(O₂)NHOH, —R₁₂OS(O₂)NHOR₉, —R₁₂OS(O₂)N(R₈)OR₉, —R₁₂NR₈S(O₂)NHOR₉, —CR₉(═N)OR₈, —NH(OR₈), —C(C═O)NHR₈, —C(C═O)NR₉R₈, —NR₁₀(OR₈)C(═O)R₉, —N(OR₈)C(═O)NR₉, —NR₈(R₉)S—, —N(R₈)S(═O)R₉, —NR(R₈)S(═O)₂R₉, —OC(═O)NR₈, —N(OR₈)C(═O)OR₉, —N(R₈)C(═S)R₉, —O(S(═O)₂R₈, —R₁₂O(S(═O)₂R₈, —O(S(═O)₂NR₈, —R₁₂O(S(═O)₂NR₈, —S(═O)R₈, —R₁₂S(═O)R₈, —S(═O)₂R₈, —R₁₂S(═O)₂R₈, —NR₁₀(R₉)S(═O)₂NHR₈, —NR₉(C═O)R₈, —NR₉(C═O)OR₈, —NR₉O(C═O)OR₈, —NR₉(C═O)NR₈R₁₀, —R₁₂N(R₉)S(═O)₂NHR₈, —R₁₂N(R₉)(C═O)R₈, —R₁₂N(R₉)(C═O)OR₈, —R₁₂N(R₉)(C═O)NR₈, —N(═NR₁₀)R₈, —R₁₂—N(═NR₁₀)R₈, —C(R₁₀)(═N—N═)CR₈R₉; —N₃, —CH₂N₃, —CH₂CH₂N₃, —CH₂CH₂CH₂N₃, —CH₂CH₂CH₂CH₂N₃, —CHR₈N₃, —CR₉R₈N₃, —CHR₉CHR₈N₃, —CH₂CHR₉CHR₈N₃, —CH₂CH₂CH₂CHR₈N₃, —C(R₈)═N—R₉, —CH₂C(R₈)═N—R₉, —CH₂CH₂C(R₈)═N—R₉, —CH₂CH₂CH₂C(R₈)═N—R₉, —N₃, —CH₂CH₂N₃, —CH₂CH₂CH₂N₃, —CH₂CH₂CH₂CH₂N₃; and R2 and R3 each independently represent —H, —OH, —CH₃, —OCH₃, —C(═O)CH₃, —OC(═O)CH₃, —C(═O)OCH₃, —C(═O)CH₂CH₃, —OC(═O)CH₂CH₃, —C(═O)OCH₂CH₃, —CH₂CH₃, —CH(CH₃)₂, —C(CH₃)₃, —CH₂CH₂CH₃, —CH₂CH(CH₃)₂, —CHC(CH₃)₃, —CH₂OH, —NH₂, —CH₂NH₂, —N₃, and (C1-C4)alkyl; where R₈, R₉, R₁₀, R₁₁ each independently represent —H, —OH, —CH₃, —CH₂CH₃, —CH₂═CH₂, —CH₂CH₂═CH₂, —CH₂CH₂CH₂═CH₂, —C(H)═O, —CH₂C(H)═O, —CH₂CH₂C(H)═O, —CH(CH₃)CH₃, —CH₂CH₂CH₃, —CH₂CH(CH₃)CH₃, —CHC(CH₃)₂CH₃, —C(═O)CH₃, —C(═O)CH₂CH₃, —C(═O)NH₂, —CH₂OH, —CH₂CH₂OH, —C(H)(OH)C(H₂)(OH), —OCH₃, —OC(CH₃)₃, —OCH₂CH₃, —OCH(CH₃)₂, —CO₂H, —C(═O)OCH₃, —C(═O)OCH₂CH₃, —OC(═O)CH₃, —OC(═O)CH₂CH₃, —OC(═O)OCH₃. —OC(═O)OCH₂CH₃, —C(CH₃)₃, —CH₂CH₂(CH₃)₂, —CHC(CH₃)₃, —CH₂CH₂OH, CH₂CH₂CH₂OH, —Cl, —Br, —I, —F, —CH₂Cl, —CH₂Br, —CH₂I, —CH₂F, —CHCl₂, —CHBr₂, —CHI₂, —CHF₂, —CCl₃, —CBr₃, —CI₃, —CF₃, —NH₂, —CH₂NH₂, —NH(OH), —CH₂N(OH), —NH(OCH₃), —N(OCH₂CH₃), —CH₂NH(OCH₃), —CH₂N(OCH₂CH₃), —N₃, —CH₂N₃, —CH₂CH₂N₃, —CH═NH, —CH═NOH, —CH═NOCH₃, —SH, —SCH₃, —SCH₂CH₃, —NO₂, —CN, cyclopropane ring, saturated or unsaturated cyclobutane ring, saturated or unsaturated cyclopentane ring, saturated or unsaturated cyclohexane ring, benzene ring, phenolic ring, xylene ring, an amino acid(s), (C₁-C₄)alkyl, and R₁₂ represents —CH₂—, —CH₂CH₂—, —CH(CH₃)CH₂—, —C(CH₃)CH₂—, —CH═CH—, —O—, —S—, —O(C═O)—, —(C═O)O—, —NH—, —N(R₈)—, —N(OH)—, —CH₂—O—, —O—CH₂—, —CH₂CH₂—O—, —O—CH₂CH₂—, —S(═O)—, —(S═O)₂—, —NH(S═O)₂—, —N(OH)S(═O)₂—, —S(═O)₂NH—, —S(═O)₂N(OH)—, —NHS(═O)₂—, —N(OH)S(═O)₂—, —CH₂NH—, —CH₂N(R₈)—, —CH₂N(OH)—, —NHCH₂—, —N(R₈)CH₂—, —N(OH)CH₂—, —OC(C═O)O—, —OC(═O)NR₈—, —NR₈C(═O)O—.

An ‘Illudin’, which includes illudin M (where R₁, and R₃ are CH₃, and R₂ is OH), illudin S (where R₁ is CH₂OH, R₂ is OH, and R₃ is CH₃) and syn-illudins (which include an illudin ring derivative (where R₂ is the site of derivatization) or an illudin alkyl derivative (where R₁ is the site of derivatization) means an Illudofulvene subgroup with the following structural molecular formula:

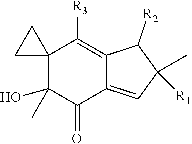

where R₁, R₂, R₃, are as set forth in Formula P2, as given herein.

‘Formula P2’ means where R₁, R₂, and R₃ each independently represent —H, —OH, —CH₃, —OCH₃, —C(═O)CH₃, —OC(═O)CH₃, —C(═O)OCH₃, —C(═O)CH₂CH₃, —OC(═O)CH₂CH₃, —C(═O)OCH₂CH₃, —CH₂CH₃, —CH(CH₃)₂, —C(CH₃)₃, —CH₂CH₂CH₃, —CH₂CH(CH₃)₂, —CHC(CH₃)₃, —CH₂OH, —NH₂, —NHOH, —CH₂NH₂, —CH₂NHOH, —N₃, and (C1-C4)alkyl.

Syn-Illudins are semi-synthetic derivatives of Illudins. Acylfulvenes are also semi-synthetic derivatives of Illudins. Syn-Illudins and Acylfulvenes have each been chemically modified at select sites to allow their use as medicants. The modifications in the Syn-Illudins do not alter any of the cyclic rings (cyclopropane, cyclopentane, cyclohexane) of the basic Illudin chemical structure. The modifications of Acylfulvenes differ from Syn-Illudins in that an additional double bond (an unsaturated bond) has been created in the 5 membered (cyclopentane) ring. Table 14 is a listing of IUPAC names of the Illudofulvene Analog according to various embodiments of the present invention.

During cancer treatment, critical time is often lost due to a trial and error approach to finding an effective therapy. In addition, cancer cells often develop resistance to a previously effective therapy. In such situations, patient outcome can be greatly improved by early detection of such resistance. Thus, there exists a need in the art for methods and devices that can predict the responsiveness of cancer patients to a medical treatment.

Exemplary types of cancer that can be diagnosed or treated with the methods include, e.g., prostate cancer, ovarian cancer (e.g., ovarian adenocarcinoma or embryonal carcinoma), liver cancer (e.g., HCC or hepatoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma, and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, or neoplasms of the central nervous system. For example, the cancer may be a solid tumor or a hematological cancer.

A first aspect of the invention features a method for detecting expression of a biomarker (e.g., one, two, three, four, five, ten, or all of the biomarkers shown in Tables 1-8, such as ERCC2 (SEQ ID NO:21)) in a patient having cancer (e.g., prostate cancer, ovarian cancer, or HCC), such as a patient having cancer that is resistant to one or more cancer therapies other than an illudofulvene (e.g., a patient with prostate cancer, ovarian cancer, or HCC that is resistant to one or more cancer therapies other than an illudofulvene). The method includes (a) contacting a sample (e.g., a tumor sample) from the patient including one or more nucleic acid molecules with a device (e.g., a microarray) including: i) one or more single-stranded nucleic acid molecules capable of specifically hybridizing with the nucleotides of one or more biomarkers of sensitivity selected from the biomarkers of Tables 1-8 (e.g., ERCC2 (SEQ ID NO:21)); and (b) detecting a level of expression of one or more of the biomarker(s) of sensitivity by performing microarray analysis or quantitative reverse transcriptase polymerase chain reaction (qRT-PCR). Expression of the biomarker(s) may be detected by determining the level of a messenger RNA (mRNA) transcribed from a nucleic acid molecule corresponding to a gene of the biomarker (e.g., a mRNA expressed from the ERCC2 (SEQ ID NO:21)) or a complementary DNA (cDNA) thereof.

A second aspect of the invention features a method of determining responsiveness of a patient having cancer (e.g., one of the cancers noted above, such as prostate cancer, ovarian cancer, or HCC) to an illudofulvene. In particular, the patient may have a cancer that is resistant to one or more cancer therapies other than an illudofulvene, such as prostate cancer, ovarian cancer, or HCC that is resistant to one or more cancer therapies other than an illudofulvene. The method includes a) contacting a sample (e.g., a tumor sample) from the patient including one or more nucleic acid molecules with a device (e.g., a microarray or a device for performing a qRT-PCR reaction) including: i) one or more single-stranded nucleic acid molecules capable of specifically hybridizing with the nucleotides of one or more biomarkers of sensitivity selected from the biomarkers of Tables 1-8 (e.g., ERCC2 (SEQ ID NO:21)); and b) measuring hybridization, or an amplification product resulting from hybridization, between the one or more nucleic acid molecules from the patient and the single-stranded nucleic acid molecules of the device to detect a level of expression of one or more of the biomarkers of sensitivity. The patient is determined to be responsive to an illudofulvene if: i) the level of expression of the biomarkers of sensitivity is substantially similar to the level of expression of the biomarkers of sensitivity (e.g., ERCC2 (SEQ ID NO:21)) in a cell (e.g., a cancer cell) or tissue (e.g., a tumor tissue) known to be sensitive to an illudofulvene.

The method of the second aspect can further include administering an illudofulvene to the patient if: i) the level of expression of the biomarkers of sensitivity (e.g., ERCC2 (SEQ ID NO:21)) is substantially similar to the level of expression of the biomarkers of sensitivity in a cell (e.g., a cancer cell) or tissue (e.g., a tumor tissue) known to be sensitive to an illudofulvene. The method can further include administering one or more cancer therapies other than an illudofulvene to the patient if: i) the level of expression of the biomarkers of sensitivity (e.g., ERCC2 (SEQ ID NO:21)) is substantially dissimilar to the level of expression of the biomarkers of sensitivity in a cell (e.g., a cancer cell) or tissue (e.g., a tumor tissue) known to be sensitive to an illudofulvene.

In particular, the one or more of the cancer therapies includes surgery, radiation, or a therapeutic agent, such as Other Drugs as set forth herein.

The invention also features a method of treating cancer in a patient in need thereof (e.g., one of the cancers noted above, such as prostate cancer, ovarian cancer, or HCC) that includes administering an illudofulvene to the patient, in which the patient has been determined to be responsive to an illudofulvene according to the method of the first or second aspect of the invention. In particular, the patient may have a cancer that is resistant to one or more cancer therapies other than an Illudofulvene Analog selected from Table 14 (e.g., a patient with prostate cancer, ovarian cancer, or HCC that is resistant to one or more cancer therapies other than an illudofulvene).

A third aspect of the invention features a method of treating a patient having cancer (e.g., one of the cancers noted above, such as prostate cancer, ovarian cancer, or HCC). In particular, the patient may have a cancer that is resistant to one or more cancer therapies which use other than an Illudofulvene Analog selected from Table 14 (e.g., a patient with prostate cancer, ovarian cancer, or HCC that is resistant to one or more cancer therapies other than an illudofulvene). The method includes a) contacting a sample (e.g., a tumor sample) from the patient including one or more nucleic acid molecules with a device (e.g., a microarray or a device for performing a qRT-PCR reaction) including: i) one or more single-stranded nucleic acid molecules capable of specifically hybridizing with the nucleotides of one or more biomarkers of sensitivity selected from the biomarkers of Tables 1-8 (e.g., ERCC2 (SEQ ID NO:21)); and b) measuring hybridization, or an amplification product resulting from hybridization, between the one or more nucleic acid molecules from the patient and the single-stranded nucleic acid molecules of the device to detect a level of expression of one or more of the biomarkers of sensitivity and c) administering an Illudofulvene Analog selected from Table 14 to the patient if: i) the level of expression of the biomarkers of sensitivity is substantially similar to the level of expression of the biomarkers of sensitivity in a cell (e.g., a cancer cell) or tissue (e.g., a tumor tissue) known to be sensitive to an illudofulvene.

The method of the third aspect of the invention may further include administering one or more additional therapies (e.g., surgery, radiation, or a therapeutic agent) to the patient prior to, concurrently with, or after administration of an Illudofulvene Analog selected from Table 14. In particular, the therapeutic agent may be selected from the group consisting of Other Drugs as set forth herein. The therapeutic agent can be administered parenterally (e.g. intravenously, intramuscularly, transdermally, intradermally, intra-arterially, intracranially, subcutaneously, intraorbitally, intraventricularly, intraspinally, intraperitoneally, or intranasally), enterally, or topically.

In the second or third aspect of the invention, an illudofulvene may be administered parenterally (e.g. intravenously, intramuscularly, transdermally, intradermally, intra-arterially, intracranially, subcutaneously, intraorbitally, intraventricularly, intraspinally, intraperitoneally, or intranasally), enterally, or topically. Preferably, an illudofulvene is administered by intravenous injection. An illudofulvene analog selected from Table 14 may be administered to the patient two or more times, such as one or more times daily, weekly, every two weeks, every three weeks, or monthly (e.g., one or more times per week for two weeks or more). Additionally, a second dose of an illudofulvene analog selected from Table 14 may be administered to the patient two weeks, three weeks, four weeks, or five weeks after administration of a prior dose of an illudofulvene. In particular, an illudofulvene analog selected from Table 14 is administered in a 3 week treatment regimen in which the illudofulvene analog selected from Table 14 is administered on day 1 an day 8. The treatment regimen may be repeated two to twenty times or more, as needed.

In particular, an illudofulvene analog selected from Table 14 may be administered to the patient at a dose of about 0.05 mg/kg to 6 mg/kg, such as at a dose of about 0.1 mg/kg to 1 mg/kg. For example, an illudofulvene may be administered at a dose of about 0.15 mg/kg, 0.20 mg/kg, 0.25 mg/kg, 0.30 mg/kg, 0.35 mg/kg, 0.40 mg/kg, 0.45 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, or 0.95 mg/kg. An illudofulvene may be administered to the patient in a treatment regimen at least once per week for at least two weeks and/or on day 1 and day 8 of a 3 week treatment regimen, in which the treatment regimen occurs one or more times (e.g., the treatment regimen is repeated two to twenty times). In particular, an illudofulvene is administered to the patient at a dose of about 0.1 mg/kg to 1 mg/kg, such as a dose of about 0.2 mg/kg to about 0.6 mg/kg (e.g., a dose of about 0.45 mg/kg). This dosage of an illudofulvene can be administered in a 3 week treatment regimen, in which an illudofulvene is administered on day 1 and day 8.

In the second or third aspect of the invention, the contacting step (a) and the measuring step (b) may occur prior to, concurrent with, or after administration of an illudofulvene analog selected from Table 14 to the patient. Additionally, the contacting step (a) and the measuring step (b) may occur two or more times, e.g., during treatment with an illudofulvene. For example, the contacting step (a) and the measuring step (b) may occur two or more times to assess the continued sensitivity of the patient to the Illudofulvene Analog selected from Table 14.

In any of the above aspects of the invention, the device can include at least two, at least three, at least four, at least five, at least six, at least seven, at least eight, at least nine, at least ten, or more single-stranded nucleic acid molecules capable of specifically hybridizing with the nucleotides of one or more biomarkers of sensitivity selected from the biomarkers of Tables 1-8 (e.g., ERCC2 (SEQ ID NO:21)). Any of these aspects can be used individually or in any combination with other aspects. In particular, one or more of the single-stranded nucleic acid molecules of the device have a length in the range of 10 to 100 nucleotides in length (e.g., a length in the range of 20 to 60 nucleotides).

In any of the above aspects of the invention, the method may include converting the level of expression of one or more of the biomarkers of sensitivity (e.g., one, two, three, four, five, ten, or all of the biomarkers shown in Tables 1-8, such as ERCC2 (SEQ ID NO:21)) into a mean score, in which the mean score indicates the responsiveness of the patient to an Illudofulvene Analog selected from Table 14. In particular, the mean score and/or the difference score above a cutoff value indicates that the patient is responsive to an illudofulvene, such as if the cutoff value is above a specific numeric value.

In any of the above aspects of the invention, the biomarker of sensitivity may be selected from one or more of the ‘Select Sensitive Genes’, where the Select Sensitive Genes means ‘ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26), XPA (SEQ ID NOS:27-28), RAD18 (SEQ ID NOS:29-30), ERCC6L (SEQ ID NOS:31-32), ERCC6L2 (SEQ ID NOS:33-34), MYC (SEQ ID NOS:222-223), ERCC1 (SEQ ID NOS:236-237), PTGR1 (SEQ ID NOS:249-250), and/or PTGR2 (SEQ ID NOS:262-263)’.

For example, the biomarkers of sensitivity may include ERCC8 (SEQ ID NOS:1-2) and ERCC6 (SEQ ID NOS:3-4). For example, the biomarkers of sensitivity may include ERCC8 (SEQ ID NOS:1-2), ERCC6 (SEQ ID NOS:34), and GTF2H1 (SEQ ID NOS:5-6). For example, the biomarkers of sensitivity may include ERCC8 (SEQ ID NOS:1-2), ERCC6 (SEQ ID NOS:3-4), GTF2H1 (SEQ ID NOS:5-6), and GTF2H2 (SEQ ID NOS:7-8). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8) and GTF2H3 (SEQ ID NOS:9-10). For example, the biomarkers of sensitivity may include ERCC8 (SEQ ID NOS:1-2), ERCC6 (SEQ ID NOS:34), GTF2H1 (SEQ ID NOS:5-6), GTF2H2 (SEQ ID NOS:7-8), GTF2H3 (SEQ ID NOS:9-10) and GTF2H4 (SEQ ID NOS:11-12). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS: 11-12) and GTF2H5 (SEQ ID NOS:13-14). For example, the biomarkers of sensitivity may include ERCC8 (SEQ ID NOS:1-2), ERCC6 (SEQ ID NOS:34), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), and USP7 (SEQ ID NOS:15-16). For example, the biomarkers of sensitivity may include ERCC8 (SEQ ID NOS:1-2), ERCC6 (SEQ ID NOS:34), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), USP7 (SEQ ID NOS:15-16), and UVSSA (SEQ ID NOS:17-18). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), USP7 (SEQ ID NOS:15-16), UVSSA (SEQ ID NOS:17-18) and ERCC3 (SEQ ID NOS:19-20). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NO:13 or SEQ ID NO:14 or SEQ ID NO:101-111), USP7 (SEQ ID NOS:15-16), UVSSA (SEQ ID NOS:17-18), ERCC3 (SEQ ID NOS:19-20), and ERCC2 (SEQ ID NOS:21-22). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS: 11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), and ERCC4 (SEQ ID NOS:23-24). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS: 11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24) and ERCC5 (SEQ ID NOS:25-26). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS: 11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26) and XPA (SEQ ID NOS:27-28). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS: 11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26), XPA (SEQ ID NOS:27-28) and RAD18 (SEQ ID NOS:29-30). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26), XPA (SEQ ID NOS:27-28), RAD18 (SEQ ID NOS:29-30) and ERCC6L (SEQ ID NOS:31-32). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26), XPA (SEQ ID NOS:27-28), RAD18 (SEQ ID NOS:29-30), ERCC6L (SEQ ID NOS:31-32) and ERCC6L2 (SEQ ID NOS:33-34). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26), XPA (SEQ ID NOS:27-28), RAD18 (SEQ ID NOS:29-30), ERCC6L (SEQ ID NOS:31-32), ERCC6L2 (SEQ ID NOS:33-34) and/or MYC (SEQ ID NOS:222-223). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS: 11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26), XPA (SEQ ID NOS:27-28), RAD18 (SEQ ID NOS:29-30), ERCC6L (SEQ ID NOS:31-32), ERCC6L2 (SEQ ID NOS:33-34), MYC (SEQ ID NOS:222-223), and/or ERCC1 (SEQ ID NOS:236-237). For example, the biomarkers of sensitivity may include ERCC8 (SEQ ID NOS:1-2), ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26), XPA (SEQ ID NOS:27-28), RAD18 (SEQ ID NOS:29-30), ERCC6L (SEQ ID NOS:31-32), ERCC6L2 (SEQ ID NOS:33-34), MYC (SEQ ID NOS:222-223), ERCC1 (SEQ ID NOS:236-237) and/or PTGR1 (SEQ ID NOS:249-250). For example, the biomarkers of sensitivity may include the Select Sensitive Genes.

In any of the above aspects of the invention, the device can be a microarray, such as a deoxyribonucleic acid (DNA)-based platform. Alternatively, the device can be for performing a qRT-PCR reaction (e.g., the device is used with a system for detecting the amplification product, for example, by fluorescence or by another method). The methods may also utilize both a microarray and a qRT-PCR. Thus, the expression level of the biomarkers of sensitivity (e.g., one, two, three, four, five, ten, or all of the biomarkers shown in Tables 1-8, such as ERCC2 (SEQ ID NO:21)) can be measured using qRT-PCR (Table 4). In particular, the level of expression of one or more of the biomarkers of sensitivity is determined by detecting the level of mRNA transcribed from one or more genes encoding one or more of the biomarkers of Tables 1-8.

In any of the above aspects of the invention, the cancer is selected from a solid tumor cancer and a hematological cancer. For example, the cancer is prostate cancer, ovarian cancer, HCC, multiple myeloma, breast cancer, acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), Hodgkin's lymphoma, cervical cancer, renal cell carcinoma (RCC), esophageal cancer, melanoma, glioma, pancreatic cancer, gastrointestinal stromal tumors (GIST), sarcoma, estrogen receptor-positive (ERpos) breast cancer, non-small cell lung carcinoma (NSCLC), colon cancer, bladder cancer, or squamous cell carcinoma of the head and neck (SCCHN). In particular, the cancer is prostate cancer, such as prostate cancer that is resistant to one or more cancer therapies other than an illudofulvene. The cancer may also be ovarian cancer, such as ovarian cancer that is resistant to one or more cancer therapies other than an illudofulvene. Alternatively, the cancer may be HCC, such as HCC that is resistant to one or more cancer therapies other than an illudofulvene.

In any of the above aspects of the invention, the patient may exhibit cancer relapse (e.g., relapse of prostate cancer, ovarian cancer, or HCC), such as relapse after treatment with a therapeutic agent other than an illudofulvene. In particular, the patient may exhibit cancer relapse prior to treatment with an illudofulvene. Alternatively, the patient may have not been administered a treatment for cancer.

### Described Methods

The expression levels of the biomarkers shown in Tables 1-8 in a patient having cancer are useful for predicting the responsiveness of the patient to an illudofulvene. These patients may already be determined to be resistance to a therapy other than an illudofulvene, such as Other Drugs as set forth herein.

A device, such as a microarray, with one or more single-stranded oligonucleotide probes that have substantial identity (e.g., at least 85%, 90%, 95%, 99%, or 100% sequence identity) to a sequence that is complementary or identical to the nucleic acid sequence of one or more biomarkers shown in Tables 1-8 can be used according to the methods described herein to assess the responsiveness of a cancer patient to treatment with an illudofulvene. For example, the probes can be used to detect one or more (e.g., two, three, four, five, ten, or all of the biomarkers shown in Tables 1-8, such as ERCC2 (SEQ ID NO:21) or ERCC3 (SEQ ID NO:19) in a sample (e.g., a tumor sample) from a patient having cancer (e.g., prostate cancer, ovarian cancer, or hepatocellular carcinoma (HCC)).

Accordingly, the invention features individual biomarkers (e.g. ERCC2 (SEQ ID NO:21) or ERCC3 (SEQ ID NO:19)) and sets of biomarkers shown in Tables 1-8 that can be used to determine the responsiveness of a cancer patient to an illudofulvene at various stages of disease progression (e.g., patients diagnosed with cancer or patients after cancer recurrence) and at different times during the treatment process (e.g., prior to administration of any cancer treatment, after administration of one or more cancer treatments other than an illudofulvene, prior to administration of an illudofulvene, or during administration of an illudofulvene). Additionally, the methods can be used to determine the an illudofulvene responsiveness of a patient with cancer that is resistant to one or more cancer therapies other than an illudofulvene, such as Other Drugs as set forth herein.

The invention provides methods of determination for assessing whether a patient with a cancer may be responsive to treatment with illudofulvenes by detecting the expression magnitude of either mRNA, or the corresponding protein produced from this mRNA, of one or more of the biomarkers shown in Table 6 (e.g. ERCC2 (SEQ ID NO:21) in a biological sample (e.g. a direct tumor biopsy or alternatively collection of free circulating tumor cells in peripheral blood) obtained from the patient using a device (e.g. a cDNA/mRNA microarray or protein array). Table 6 also contains components of the TCR post-replication repair pathway, PTGR1, PTGR2, and Myc. The expression level of one or more of these biomarkers can be compared to the expression level of the same biomarker(s) in a cell or tissue known to be sensitive to illudofulvenes based on a known defect in the TCR-NER pathway. The patient may be responsive to illudofulvene treatment if the expression level of one or more of the biomarkers (e.g. the Select Sensitive Genes) are similar to the expression levels of the corresponding biomarker in a cell or tissue known to be sensitive to an Illudofulvene Analog.

The invention features methods for identifying biomarkers (e.g., one or more of the biomarkers of Tables 1-8) for determining the responsiveness of a cancer patient to a cancer treatment, such as an Illudofulvene Analog selected from Table 14. Such methods can involve, for example, an algorithm based on growth inhibition (GI50), tumor growth inhibition (TGI), and lethal concentration (LD50) values derived from an In vitro Cancer Growth Inhibition Screen that was performed using a tumor obtained from a patient that was subjected to treatment with an Illudofulvene. In addition, or alternatively, the expression of genes associated with the TCR-NER pathway in this tumor can be measured (e.g., using a microarray (e.g., an Affymetrix HG-U133A or HG-U133_Plus_2 array)) and their expression compared to expression of a control or housekeeping gene (e.g. beta actin (ACTB), transferrin receptor (TFRC), glyceraldehyde-3-phosphate dehydrogenase (GAPD), or ubiquitin) to determine the responsiveness of a cancer patient to a cancer treatment, such as an Illudofulvene.

The invention also features methods of treating a patient having cancer that is resistant to one or more cancer therapies other than treatment including an illudofulvene, by detecting the expression levels of one or more of the biomarkers shown in Table 6 (e.g. ERCC2 (SEQ ID NO:21) in a sample (e.g. a direct tumor biopsy or alternatively collection of free circulating tumor cells in peripheral blood) obtained from the patient and then administering an illudofulvene based on the expression levels of these biomarkers. In summary, a patient having cancer may be administered an illudofulvene if the expression level of one or more of the biomarkers of sensitivity are similar to the expression levels of the biomarker of sensitivity in a cell or tissue known to be sensitive to illudofulvenes. Thus, the methods described can be used to identify cancer patients who can be predicted to respond to illudofulvenes, such as patients having, e.g., prostate cancer, ovarian cancer, hepatocellular carcinoma (HCC), cervical cancer, renal cell carcinoma (RCC), esophageal cancer, melanoma, glioma, pancreatic cancer, gastrointestinal stromal tumors (GIST), sarcoma, estrogen receptor-positive (ERpos) breast cancer, non-small cell lung carcinoma (NSCLC), colon cancer, bladder cancer, squamous cell carcinoma of the head and neck (SCCHN), acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), and Hodgkin's lymphoma. Alternatively, a patient having cancer would not be administered an illudofulvene if the expression levels of sensitivity of all biomarkers were substantially dissimilar to the expression level of biomarkers of sensitivity in a cell or tissue known to be sensitive to illudofulvenes.

Methods are described herein for identifying biomarkers of drug responsiveness, detecting this biomarker gene expression in samples from cancer patients, determining the responsiveness of a cancer patient to treatment with an illudofulvene, and treating cancer patients with an illudofulvene.

### Methods of Determining the Responsiveness of a Patient to an Illudofulvene.

The invention features diagnostic methods for the detection and screening of cancer patients (e.g., a patient having cancer that is resistant to one or more cancer therapies other than an illudofulvene) that may be responsive to an illudofulvene analog selected from Table 14 using one or more of the biomarkers shown in Tables 1-8 (e.g. ERCC2 (SEQ ID NO:21) or ERCC3 (SEQ ID NO:19)). The methods of the invention may be used for predicting a patient's responsiveness to an illudofulvene, and optionally, treating the cancer patient throughout the progression of cancer and/or in cases of recurrence (e.g., after a first line treatment, a second line treatment, and/or a third line treatment).

The invention provides individual biomarkers (e.g. ERCC2 (SEQ ID NO:21) and sets of biomarkers (e.g., two or more of the biomarkers listed in Tables 1-8), the expression levels of which, as detected in a biological sample (e.g., a tumor sample, such as a biopsy) obtained from a cancer patient (e.g., a human with cancer), are indicative of responsiveness to an Illudofulvene Analog. The biomarkers were identified using methods similar to those previously described in, e.g., W Yang, et al. Genomics of drug sensitivity in cancer (GDSC): a resource for therapeutic biomarker discovery in cancer cells. Nucleic Acids Research, 41(D1): D955, 2013, which is incorporated by reference herein in its entirety for all purposes.

In particular, an algorithm based on the obtained in vitro cell culture values (i.e. growth inhibition values (GI50), or the total growth inhibition (TGI), or the lethal concentration (LC50)) of cell lines (e.g., NCI60 cells) subjected to treatment with an illudofulvene, and gene expression is determined in these same cell lines (e.g., by microarray analysis, reverse transcriptase polymerase chain reaction (RT-PCR), quantitative real-time PCR (qPCR), or next generation sequencing). After normalization, genes with, e.g., a Pearson correlation coefficient greater than about 0.5 or below about −0.5 to one of the obtained in vitro cell culture values, can be classified as biomarkers of sensitivity or resistance, respectively. A probability or significance value can also be calculated for the correlation between the obtained in vitro cell culture values (i.e. growth inhibition values (GI50), or the total growth inhibition (TGI), or the lethal concentration (LC50)) and the normalized expression of a specific gene.

In particular, a probability value of <0.05 is a statistically significant cut-off value as known in the art for establishing whether the expression level of A GENE, e.g., the genes shown in Tables 1-8, correlate with the likelihood of cancer treatment sensitivity, such as sensitivity to an illudofulvene. Thus, a probability value of <0.05 can be used to estimate the statistical significance of the expression level of the genes of Tables 1-8 for predicting patient responsiveness to treatment with an illudofulvene according to the methods described herein.

### Biomarkers of Sensitivity.

The expression levels of one or more biomarkers of Tables 1-8 can be used to determine cancer patient responsiveness to treatment with an illudofulvene. Once determined to be responsive, the patient can be treated with an illudofulvene. In particular, the biomarker ERCC8 (SEQ ID NO:1) may be used to assess a cancer patient's (e.g., a patient having cancer that is resistant to one or more cancer therapies other than an illudofulvene) responsiveness to an illudofulvene. The expression level of the biomarker ERCC8 (SEQ ID NO:1) may be assessed using nucleic acid amplification methods (e.g., PCR) or a device (e.g., a microarray). As is described above, the expression level of ERCC8 (SEQ ID NO:1) in the patient sample may then be compared, e.g., to the expression level of ERCC8 (SEQ ID NO:1) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC8 (SEQ ID NO:1) may be used alone to predict cancer patient responsiveness to treatment with an illudofulvene or in combination with one or more additional biomarkers (e.g., one, two, three, four, five, ten, or all of the biomarkers shown in Tables 1-8), such as ERCC6 (SEQ ID NOS:3-4), GTF2H1 (SEQ ID NOS:5-6), GTF2H2 (SEQ ID NOS:7-8), GTF2H3 (SEQ ID NOS:9-10), GTF2H4 (SEQ ID NOS:11-12), GTF2H5 (SEQ ID NOS:13-14), USP7 (SEQ ID NOS:15-16), UVSSA (SEQ ID NOS:17-18), ERCC3 (SEQ ID NOS:19-20), ERCC2 (SEQ ID NOS:21-22), ERCC4 (SEQ ID NOS:23-24), ERCC5 (SEQ ID NOS:25-26), XPA (SEQ ID NOS:27-28), RAD18 (SEQ ID NOS:29-30), ERCC6L (SEQ ID NOS:31-32), ERCC6L2 (SEQ ID NOS:33-34), MYC (SEQ ID NOS:222-223), ERCC1 (SEQ ID NOS:236-237), PTGR1 (SEQ ID NOS:249-50), and/or PTGR2 (SEQ ID NOS:262-263). The expression level of the biomarker(s) may be determined using, e.g., a microarray, PCR, or other techniques described herein, for example, using a nucleic acid probe sequence based on the target sequences shown in Tables 1-8.

The expression level of the biomarker ERCC6 (SEQ ID NO:3) may be assessed using nucleic acid amplification methods (e.g., PCR) or a device (e.g., a microarray). As is described above, the expression level of ERCC6 (SEQ ID NO:3) in the patient sample may then be compared, e.g., to the expression level of ERCC6 (SEQ ID NO:3) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC6 (SEQ ID NO:3) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker GTF2H1 (SEQ ID NO:5) may be used to assess a cancer patient's (e.g., a patient having cancer that is resistant to one or more cancer therapies other than an illudofulvene) responsiveness to an illudofulvene. The expression level of the biomarker GTF2H1 (SEQ ID NO:5) may be assessed using nucleic acid amplification methods (e.g., PCR) or a device (e.g., a microarray). As is described above, the expression level of GTF2H1 (SEQ ID NO:5) in the patient sample may then be compared, e.g., to the expression level of GTF2H1 (SEQ ID NO:5) in a cell (e.g., a cancer cell) or tissue (e.g., a tumor tissue) known to be sensitive or resistant to treatment with an illudofulvene and used to determine the cancer patient's responsiveness to an illudofulvene. The biomarker GTF2H1 (SEQ ID NO:5) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker GTF2H2 (SEQ ID NO:7) may be used to assess a cancer patient's (e.g., a patient having cancer that is resistant to one or more cancer therapies other than an illudofulvene) responsiveness to an illudofulvene. The expression level of the biomarker GTF2H2 (SEQ ID NO:7) may be assessed using nucleic acid amplification methods (e.g., PCR) or a device (e.g., a microarray). As is described above, the expression level of GTF2H2 (SEQ ID NO:7) in the patient sample may then be compared, e.g., to the expression level of GTF2H2 (SEQ ID NO:7) in a cell (e.g., a cancer cell) or tissue (e.g., a tumor tissue) known to be sensitive or resistant to treatment with an illudofulvene and used to determine the cancer patient's responsiveness to an illudofulvene. The biomarker GTF2H2 (SEQ ID NO:7) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker GTF2H3 (SEQ ID NO:9) may be used to assess a cancer patient's (e.g., a patient having cancer that is resistant to one or more cancer therapies other than an illudofulvene) responsiveness to an illudofulvene. The expression level of the biomarker GTF2H3 (SEQ ID NO:9) may be assessed using nucleic acid amplification methods (e.g., PCR) or a device (e.g., a microarray). As is described above, the expression level of GTF2H3 (SEQ ID NO:9) in the patient sample may then be compared, e.g., to the expression level of GTF2H3 (SEQ ID NO:9) in a cell (e.g., a cancer cell) or tissue (e.g., a tumor tissue) known to be sensitive or resistant to treatment with an illudofulvene and used to determine the cancer patient's responsiveness to an illudofulvene. The biomarker GTF2H3 (SEQ ID NO:9) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker GTF2H4 (SEQ ID NO:11) may be used to assess a cancer patient's (e.g., a patient having cancer that is resistant to one or more cancer therapies other than an illudofulvene) responsiveness to an illudofulvene. The expression level of the biomarker GTF2H4 (SEQ ID NO:11) may be assessed using nucleic acid amplification methods (e.g., PCR) or a device (e.g., a microarray). As is described above, the expression level of GTF2H4 (SEQ ID NO:11) in the patient sample may then be compared, e.g., to the expression level of GTF2H4 (SEQ ID NO:11) in a cell (e.g., a cancer cell) or tissue (e.g., a tumor tissue) known to be sensitive or resistant to treatment with an illudofulvene and used to determine the cancer patient's responsiveness to an illudofulvene. The biomarker GTF2H4 (SEQ ID NO:11) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker GTF2H5 (SEQ ID NO:13) may be used to assess a cancer patient's (e.g., a patient having cancer that is resistant to one or more cancer therapies other than an illudofulvene) responsiveness to an illudofulvene. The expression level of the biomarker GTF2H5 (SEQ ID NO:13) may be assessed using nucleic acid amplification methods (e.g., PCR) or a device (e.g., a microarray). As is described above, the expression level of GTF2H5 (SEQ ID NO:13) in the patient sample may then be compared, e.g., to the expression level of GTF2H5 (SEQ ID NO:13) in a cell (e.g., a cancer cell) or tissue (e.g., a tumor tissue) known to be sensitive or resistant to treatment with an illudofulvene and used to determine the cancer patient's responsiveness to an illudofulvene. The biomarker GTF2H5 (SEQ ID NO:13) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker USP7 (SEQ ID NO:15) may be used to assess a cancer patient's (e.g., a patient having cancer that is resistant to one or more cancer therapies other than an illudofulvene) responsiveness to an illudofulvene. The expression level of the biomarker USP7 (SEQ ID NO:15) may be assessed using nucleic acid amplification methods (e.g., PCR) or a device (e.g., a microarray). As is described above, the expression level of USP7 (SEQ ID NO:15) in the patient sample may then be compared, e.g., to the expression level of USP7 (SEQ ID NO:15) in a cell (e.g., a cancer cell) or tissue (e.g., a tumor tissue) known to be sensitive or resistant to treatment with an illudofulvene and used to determine the cancer patient's responsiveness to an illudofulvene. The biomarker USP7 (SEQ ID NO:15) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker UVSSA (SEQ ID NO:17) may be used to assess a cancer patient's (e.g., a patient having cancer that is resistant to one or more cancer therapies other than an illudofulvene) responsiveness to an illudofulvene. The expression level of the biomarker UVSSA (SEQ ID NO:17) may be assessed using nucleic acid amplification methods (e.g., PCR) or a device (e.g., a microarray). As is described above, the expression level of UVSSA (SEQ ID NO:17) in the patient sample may then be compared, e.g., to the expression level of UVSSA (SEQ ID NO:17) in a cell (e.g., a cancer cell) or tissue (e.g., a tumor tissue) known to be sensitive or resistant to treatment with an illudofulvene and used to determine the cancer patient's responsiveness to an illudofulvene. The biomarker UVSSA (SEQ ID NO:17) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker ERCC3 (SEQ ID NO:19) may be used to assess a cancer patient's (e.g., a patient having cancer that is resistant to one or more cancer therapies other than an illudofulvene) responsiveness to an illudofulvene. The expression level of the biomarker ERCC3 (SEQ ID NO:19) may be assessed using nucleic acid amplification methods (e.g., PCR) or a device (e.g., a microarray). As is described above, the expression level of ERCC3 (SEQ ID NO:19) in the patient sample may then be compared, e.g., to the expression level of ERCC3 (SEQ ID NO:19) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC3 (SEQ ID NO:19) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker ERCC2 (SEQ ID NO:21) may be used to assess a cancer patient's (e.g., a patient having cancer that is resistant to one or more cancer therapies other than an illudofulvene) responsiveness to an illudofulvene. The expression level of the biomarker ERCC2 (SEQ ID NO:21) may be assessed using nucleic acid amplification methods (e.g., PCR) or a device (e.g., a microarray). As is described above, the expression level of ERCC2 (SEQ ID NO:21) in the patient sample may then be compared, e.g., to the expression level of ERCC2 (SEQ ID NO:21) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC2 (SEQ ID NO:21) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker ERCC4 (SEQ ID NO:23) may be used to assess a cancer patient's (e.g., a patient having cancer that is resistant to one or more cancer therapies other than an illudofulvene) responsiveness to an illudofulvene. The expression level of the biomarker ERCC4 (SEQ ID NO:23) may be assessed using nucleic acid amplification methods (e.g., PCR) or a device (e.g., a microarray). As is described above, the expression level of ERCC4 (SEQ ID NO:23) in the patient sample may then be compared, e.g., to the expression level of ERCC4 (SEQ ID NO:23) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC4 (SEQ ID NO:23) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker ERCC5 (SEQ ID NO:25) may be used to assess a cancer patient's (e.g., a patient having cancer that is resistant to one or more cancer therapies other than an illudofulvene) responsiveness to an illudofulvene. The expression level of the biomarker ERCC5 (SEQ ID NO:25) may be assessed using nucleic acid amplification methods (e.g., PCR) or a device (e.g., a microarray). As is described above, the expression level of ERCC5 (SEQ ID NO:25) in the patient sample may then be compared, e.g., to the expression level of ERCC5 (SEQ ID NO:25) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC5 (SEQ ID NO:25) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker XPA (SEQ ID NO:27) may be used to assess a cancer patient's (e.g., a patient having cancer that is resistant to one or more cancer therapies other than an illudofulvene) responsiveness to an illudofulvene. The expression level of the biomarker XPA (SEQ ID NO:27) may be assessed using nucleic acid amplification methods (e.g., PCR) or a device (e.g., a microarray). As is described above, the expression level of XPA (SEQ ID NO:27) in the patient sample may then be compared, e.g., to the expression level of XPA (SEQ ID NO:27) in a cell (e.g., a cancer cell) or tissue (e.g., a tumor tissue) known to be sensitive or resistant to treatment with an illudofulvene and used to determine the cancer patient's responsiveness to an illudofulvene. The biomarker XPA (SEQ ID NO:27) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker RAD18 (SEQ ID NO:29) may be used to assess a cancer patient's (e.g., a patient having cancer that is resistant to one or more cancer therapies other than an illudofulvene) responsiveness to an illudofulvene. The expression level of the biomarker RAD18 (SEQ ID NO:29) may be assessed using nucleic acid amplification methods (e.g., PCR) or a device (e.g., a microarray). As is described above, the expression level of RAD18 (SEQ ID NO:29) in the patient sample may then be compared, e.g., to the expression level of RAD18 (SEQ ID NO:29) in a cell (e.g., a cancer cell) or tissue (e.g., a tumor tissue) known to be sensitive or resistant to treatment with an illudofulvene and used to determine the cancer patient's responsiveness to an illudofulvene. The biomarker RAD18 (SEQ ID NO:29) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker ERCC6/L (SEQ ID NO:31) may be used to assess a cancer patient's (e.g., a patient having cancer that is resistant to one or more cancer therapies other than an illudofulvene) responsiveness to an illudofulvene. The expression level of the biomarker ERCC6/L (SEQ ID NO:31) may be assessed using nucleic acid amplification methods (e.g., PCR) or a device (e.g., a microarray). As is described above, the expression level of ERCC6/L (SEQ ID NO:31) in the patient sample may then be compared, e.g., to the expression level of ERCC6/L (SEQ ID NO:31) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC6/L (SEQ ID NO:31) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker ERCC6/L2 (SEQ ID NO:33) may be used to assess a cancer patient's (e.g., a patient having cancer that is resistant to one or more cancer therapies other than an illudofulvene) responsiveness to an illudofulvene. The expression level of the biomarker ERCC6/L2 (SEQ ID NO:33) may be assessed using nucleic acid amplification methods (e.g., PCR) or a device (e.g., a microarray). As is described above, the expression level of ERCC6/L2 (SEQ ID NO:33) in the patient sample may then be compared, e.g., to the expression level of ERCC6/L2 (SEQ ID NO:33) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC6/L2 (SEQ ID NO:33) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker MYC (SEQ ID NO:222) may be used to assess a cancer patient's (e.g., a patient having cancer that is resistant to one or more cancer therapies other than an illudofulvene) responsiveness to an illudofulvene. The expression level of the biomarker MYC (SEQ ID NO:222) may be assessed using nucleic acid amplification methods (e.g., PCR) or a device (e.g., a microarray). As is described above, the expression level of MYC (SEQ ID NO:22) in the patient sample may then be compared, e.g., to the expression level of MYC (SEQ ID NO:222) in a cell (e.g., a cancer cell) or tissue (e.g., a tumor tissue) known to be sensitive or resistant to treatment with an illudofulvene and used to determine the cancer patient's responsiveness to an illudofulvene. The biomarker MYC (SEQ ID NO:222) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker ERCC1 (SEQ ID NO:236) may be used to assess a cancer patient's (e.g., a patient having cancer that is resistant to one or more cancer therapies other than an illudofulvene) responsiveness to an illudofulvene. The expression level of the biomarker ERCC1 (SEQ ID NO:236) may be assessed using nucleic acid amplification methods (e.g., PCR) or a device (e.g., a microarray). As is described above, the expression level of ERCC1 (SEQ ID NO:236) in the patient sample may then be compared, e.g., to the expression level of ERCC1 (SEQ ID NO:236) in a cell (e.g., a cancer cell) or tissue (e.g., a tumor tissue) known to be sensitive or resistant to treatment with an illudofulvene and used to determine the cancer patient's responsiveness to an illudofulvene. The biomarker ERCC1 (SEQ ID NO:236) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker PTGR1 (SEQ ID NO:249) may be used to assess a cancer patient's (e.g., a patient having cancer that is resistant to one or more cancer therapies other than an illudofulvene) responsiveness to an illudofulvene. The expression level of the biomarker PTGR1 (SEQ ID NO:249) may be assessed using nucleic acid amplification methods (e.g., PCR) or a device (e.g., a microarray). As is described above, the expression level of PTGR1 (SEQ ID NO:249) in the patient sample may then be compared, e.g., to the expression level of PTGR1 (SEQ ID NO:249) in a cell (e.g., a cancer cell) or tissue (e.g., a tumor tissue) known to be sensitive or resistant to treatment with an illudofulvene and used to determine the cancer patient's responsiveness to an illudofulvene. The biomarker PTGR1 (SEQ ID NO:249) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The biomarker PTGR2 (SEQ ID NO:262) may be used to assess a cancer patient's (e.g., a patient having cancer that is resistant to one or more cancer therapies other than an illudofulvene) responsiveness to an illudofulvene. The expression level of the biomarker PTGR2 (SEQ ID NO:262) may be assessed using nucleic acid amplification methods (e.g., PCR) or a device (e.g., a microarray). As is described above, the expression level of PTGR2 (SEQ ID NO:262) in the patient sample may then be compared, e.g., to the expression level of PTGR2 (SEQ ID NO:262) in a cell (e.g., a cancer cell) or tissue (e.g., a tumor tissue) known to be sensitive or resistant to treatment with an illudofulvene and used to determine the cancer patient's responsiveness to an illudofulvene. The biomarker MYC (SEQ ID NO:222) may be used alone to predict cancer patient responsiveness to treatment with an illudofulvene or in combination with one or more additional biomarkers (e.g., one, two, three, four, five, ten, or all of the biomarkers shown in Tables 1-8), such as the Select Sensitive Genes. The expression level of the biomarker(s) may be determined using, e.g., a microarray, PCR, or other techniques described herein, for example, using a nucleic acid probe sequence based on the target sequences shown in Tables 1-8.

The following Examples 1-4, are intended to illustrate, rather than limit, the invention.

### Example 1

Identification of Biomarkers of Sensitivity to an Illudofulvene using cell lines deficient in a specific DNA repair gene. Cell growth inhibition assays were performed using Illudofulvene against a bank of cell lines which are deficient in DNA repair based on lack of expression of a specific DNA repair gene (Table 21).

TABLE 21
Sensitivity of DNA repair Deficient Cells to Illudofulvene Analog

| | | GI50 | |
|---|---|---|---|
| | DNA- | value (nM) | Corresponding |
| | repair Gene | Mean +/ SD | GENE |
| Cell line | Deficient | (N = 3) | SEQ ID NOS: |
| AA8 | None (wild type) | 1440 ± 70 | None |
| UV20 | ERCC1 | 144 ± 4 | 236-237 |
| UV5 | ERCC2 | 60 ± 7 | 21, 22, 145-155 |
| UV24 | ERCC3 | 127 ± 13 | 19, 20, 134-144 |
| UV41 | ERCC4 | 167 ± 10 | 23, 24, 156-166 |
| UV135 | ERCC5 | 174 ± 22 | 25, 26, 167-177 |
| UV61 | ERCC6 | 366 ± 18 | 3, 4, 46-56 |
| EM9 | XRCC1 | 1197 ± 44 | None |

Studies clearly identified that a cell which is deficient in Transcription-coupled repair (TCR) (e.g. ERCC2 or ERCC3) is sensitive to Illudofulvene as compared to its parental line (AA8) whereas cells deficient in other DNA repair components, such as homologous recombination (e.g. XRCC1). These results are consistent with the finding that Illudofulvene Analogs produce a damage that is repaired only by the TCR genes and that other DNA-repair pathways (e.g. homologous recombination, Base-excision, etc.) are not involved (Kelner, et al. Cancer Research, 55:4936-40, 1995; and Jaspers, et al. DNA Repair 1:1027-38, 2002, both of which are herein expressly incorporated by reference in their entireties and for all purposes). TCR genes means Excision Repair Cross-Complementation (ERCC) 1 (ERCC1), ERCC2, ERCC3, ERCC4, ERCC5, ERCC6, ERCC6L, ERCC6L2, DNA Damage Recognition and Repair Factor (XPA), Ubiquitin Specific Peptidase7 (USP7), UV Stimulated Scaffold Protein A (UVSSA), E3 ubiquitin-protein ligase RAD18 (RAD18), General Transcription Factor IIH (GTF2H) Subunit 1 (GTF2H1), GTF2H2, GTF2H2, GTF2H3, GTF2H4, GTF2H5 genes.

### Example 2

Identification of Biomarkers of Sensitivity to An Illudofulvene Using Gene Expression data derived by DNA chip analysis of the 60 cancer cell lines of the NCI60 data set.

The expression of thousands of genes in each of the cell lines composing the NCI DTP 60 cell line has been analyzed using a variety of different DNA chips (or microarrays) and is publicly available at website dtp.cancer.gov/compare-web-public_compare/SearchAndDisplay/SearchAndDisplay.

Data has been generated using a variety of Affymetrix DNA chips including HG-U133A array (GENELOGIC_133 data set), HG-U133 Plus 2.0 array (GENELOGIC_133PLUS2 data set), the HG-95 (GENELOGIC_U95 data set), as well as others. Results from DNA microchips produced by other companies, e.g. Novartis, are also available. The data from these array experiments has been log it normalized to allow a direct comparison of a genes expression between different cell lines. For each array, the logit transformation was performed followed by a Z-transformation to mean zero and SD.

One can determine the growth inhibition (GI50), total growth inhibition (TGI), or lethal concentration (LC50) for a drug for each of the cell lines in the NCI DTP 60 cell line panel. One can use these 3 values to search for correlations against all gene expressions in all cell lines using the NCI's publicly available CELL MINER Software. Alternatively, one can down load for a specific gene its relative expression in each of the 60 cell lines using the NCI's publicly available COMPARE Software, and performs the expression for a specific gene.

A gene's expression in each cell line can then be correlated to the ability of an illudofulvene to hinder cell growth (GI50, TGI, or LC50 value) and a correlation coefficient determined to identify genes positively and negatively correlated to sensitivity to an Illudofulvene Analog. Positively correlated genes can be those in which as the expression of the gene increase, the tumor cells become sensitive to the drug. Negatively correlated genes can be those that as the expression of the gene decreases, the tumor cells become sensitive to the drug.

Analysis of the cell growth inhibition values for the Illudofulvene Analog 002, to specific gene expression values retrieved by COMPARE from data generated by the Affymetrix HG-U133 Plus 2.0 array again confirms that a deficiency in expression of components of TCR-pathway confers sensitivity to Illudofulvene Analog 002 (Table 16) including ERCC2, ERCC8, GTF2H1, USP7, and XPC. However, there was no correlation with components of other DNA repair pathways such as double strand repair (RAD50), NER (XPC), X-ray repair (XRCC1), etc.

Similar results are obtained with illudofulvene Analogs 142, 176, 184, (see Tables 17-20).

### Example 3

Identification of Biomarkers whose expression causing sensitivity to Illudofulvene Analog 002 followed by confirmation using Gene Expression data derived by DNA chip analysis of the 60 cancer cell lines of the NCI60 data set (see, Example 2 above) and also confirmed using cell lines deficient in a specific GENE (see, Example 1 above).

To further explore the mechanism(s) of action of Illudofulvene Analog 002, we used phage display technology to identify peptides capable of binding to the drug and utilized this information to identify potential target proteins for Illudofulvene Analog 002 binding. The proto-oncogene c-myc (Gene Symbol MYC) was identified as a putative target, and its role in sensitizing tumor cells to an Illudofulvene Analog was confirmed using Gene Expression data derived by DNA chip analysis of the 60 cancer cell lines of the NCI60 data set (see, Example 2 above) and then confirmed in vitro using cell lines deficient in a specific GENE (see, Example 1 above).

Three different ER2537-derived phage libraries (New England Biolabs, Beverly, MA) were used for biopanning studies; 7-mer, 12-mer, and C7C. The latter consisted of a 7-mer library flanked by two cysteines to allow disulfide formation. The aldehyde derivative of Illudofulvene Analog 002 was prepared by reacting Illudofulvene Analog 001 with acrolein, and then linked to biotin using Biotin-LC-hydrazide (Pierce). A 96 well avidin coated plate was coated with 75 μg of the target molecule (Illudofulvene Analog 001-biotin conjugate) in 150 μl of PBS, and rocked gently overnight at 4° C. The desired phage library was added to the wells and the plate gently rocked for 60 minutes at room temperature. Wells were rinsed ten times with TBST (50 mM Tris-HCl, pH 7.5, 150 mM NaCl, 0.1% v/v tween-20). Bound phage was eluted by applying excess target molecule (Illudofulvene Analog 001) and increasing salt concentration. One μl of the eluate was used for titering and the remaining eluate was amplified using standard techniques.

Using phage display panning we isolated 157 nonredundant sequences capable of binding to Illudofulvene Analog 002. Two algorithms were used to predict a sequence and 3D structure of a protein capable of binding to Illudofulvene Analog 002; “peptide elongation without substitution” and “peptide elongation with substitution”. Each of the 157 sequences was initially aligned using a FASTA program to proteins in the PIR database followed by the desired peptide elongation process. A complete description of the process has been published (Tsigelny I, Sharikov Y, Kelner M J, and Ten Eyck L F., Protein structure prediction on the basis of combinatorial peptide libraries screening. In: Protein Structure Prediction: A Bioinformatics Approach. Ed: I Tsigelny. International University Line Publishing, 2001, which is herein expressly incorporated by reference in its entirety for all purposes). The algorithm of elongation without substitution identified c-myc as a potential target of Illudofulvene Analog 002 and docking studies revealed a potential binding site for Illudofulvene Analog 002 that matched the peptides recovered by phage panning.

To determine if the MYC gene had clinical significance in regards to the therapeutic activity of Illudofulvene Analog 002, we inputted the in vitro cell culture values (i.e. growth inhibition values (GI50), or the total growth inhibition (TGI), or the lethal concentration (LC50) obtained for Illudofulvene Analog 002 from cell lines contained in the NCI 60 cell line panel and used CELLMINER to screen for correlation against the normalized expression of genes in cell lines in the NCI 60 cell line panel.

Using CELLMINER, after normalization, genes with, e.g., a Pearson correlation coefficient greater than about 0.2 or below about −0.2 can be classified as biomarkers of sensitivity or resistance, respectively. In particular, a correlation coefficient of about 0.2 or greater is a statistically significant cut-off known in the art for establishing whether the expression level of A GENE, e.g., the genes shown in Tables 1-8, correlate with the likelihood of cancer treatment sensitivity, such as sensitivity to Illudofulvene Analog 002. Thus, a correlation coefficient of about 0.2 or greater or about −0.2 or lower can be used to estimate the statistical significance of the expression level of the genes of Tables 1-8 for predicting patient responsiveness to treatment with an Illudofulvene Analog according to the methods described herein.

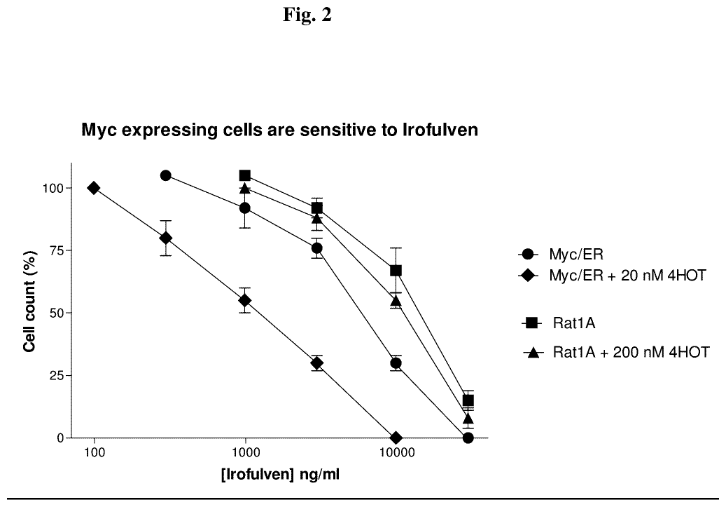

Using CELLMINER the correlation of MYC expression to Illudofulvene Analog 002 activity was identified at 0.42, and a p value of 0.000941 (highly significant) indicating MYC expression in a patient's tumor can be predictive of response to treatment with Illudofulvene Analog 002. A similar significant correlation between MYC expression and GI50 values for CAP-0142 (P<0.001) and CAP-0241 (P<0.001). To confirm the role of c-myc in Illudofulvene Analog 002 cytotoxicity, studies were conducted using the c-myc negative Rat 1A line, and c-myc positive daughter line Myc/Rat1A. The expression of c-myc in this cell line is under the control of a modified receptor that responds to 4-hydroxytamoxifen (4HOT). FIG. 2 shows that the presence of 4HOT expresses c-myc and induces sensitivity to expression of MYC causing cancer cells to become sensitive to illudofulvene (Analog 002) in the Myc/ER daughter, but not the Rat 1A parental cell line.

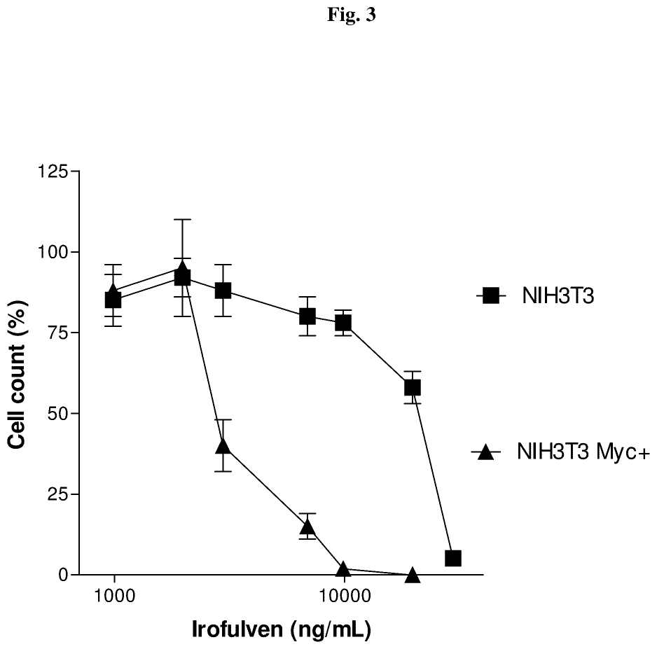

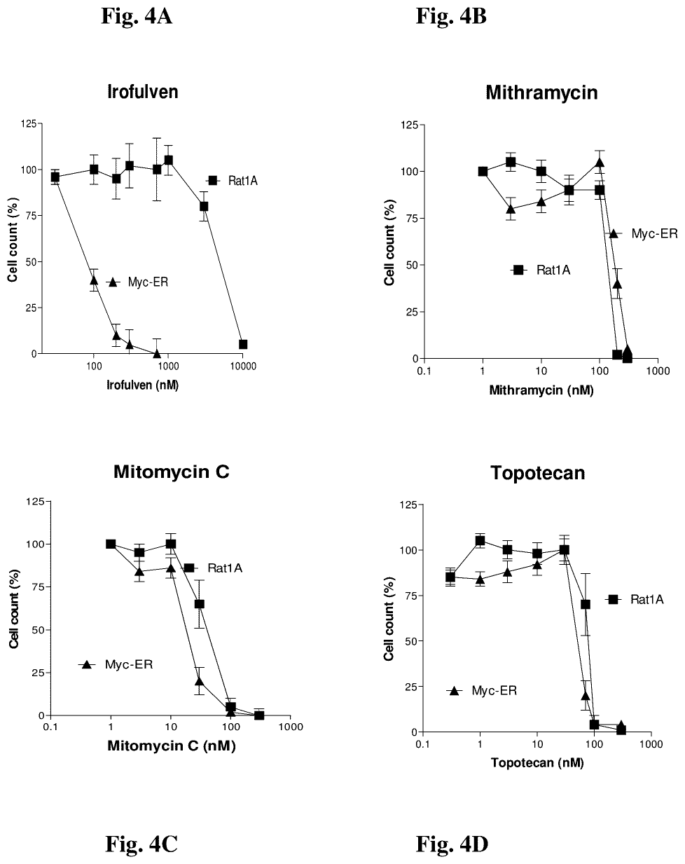

FIG. 3 shows the influence of c-myc expression on Illudofulvene Analog 002 cytotoxicity in a comparison of Cell Count (%) as a function of Analog 002 concentration for a paired c-myc expressing (NIH3T3 MYC+) cell line and a c-myc nonexpressing (NIH3T3) or null cell line. Expression of cellular c-myc conferred a greater than 10 fold increase in sensitivity to illudofulvene Analog 002 to the daughter NIH3T3 MYC+ cell line as compared to the parental c-myc null or nonexpressing NIH3T3 cell line, based on the decrease in cell count. The ability of c-myc expression to alter cellular sensitivity to other agents was also studied. The expression of c-myc, however, did not alter cellular sensitivity towards a variety of other anticancer agents (including mithramycin, mitomycin C, topotecan (as shown in FIG. 4B-FIG. 4D) or cisplatin (data not shown) as compared to Illudofulvene Analog 002 (see FIG. 4A).

### Example 4

Most relevant to clinical applications, Illudofulvene Analog 002 activity is independent of common resistance mechanisms such as the multi-medicant resistance phenotype, anti-apoptotic B-cell lymphoma 2 (Bcl-2) over expression, as well as tumor protein 53 (p53) and cyclin dependent kinase inhibitor 1 (p21/WAF1) mutations (see Tables 7 and 11).

Growth factors, including peptides and proteins are critical mediators of a wide range of cell-cell communication. They are important endocrine, paracrine and autocrine messengers. Growth factors function as neurotransmitters and neuromodulators, regulate chemotaxis, immune function, development, cell growth, and can influence tumor cells. The receptors that recognize growth factors are highly selective and define specific cell populations. As a result, growth factor receptors are a large and important class of medicant (including drug) targets. In addition to physiologic noncancerous cell populations, these receptors can also be expressed in various cancer cell populations.

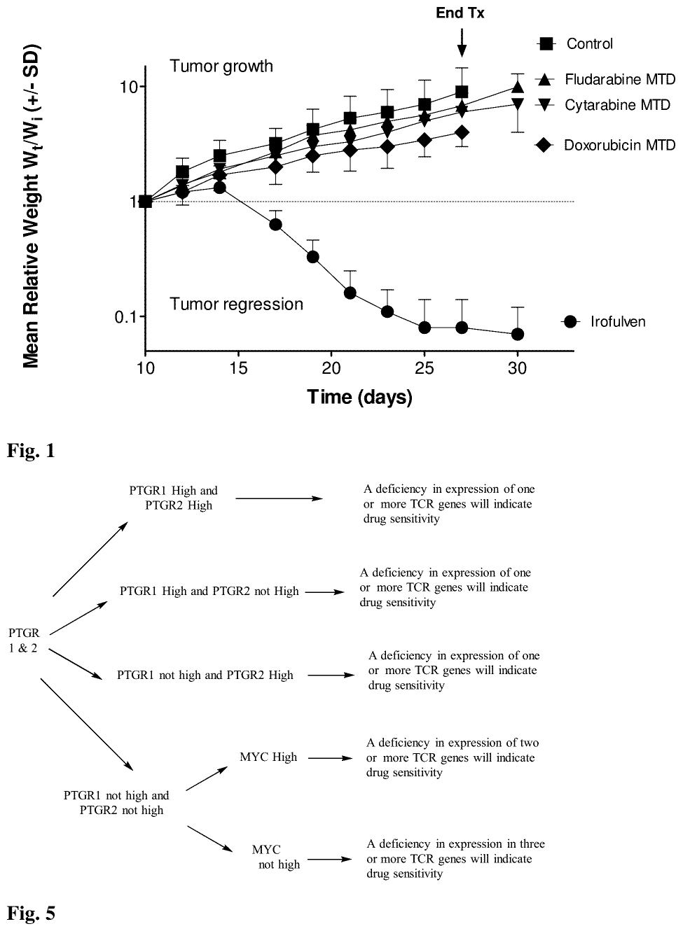

FIG. 1 shows the ability of the illudofulvene Analog 002 to induce tumor regression in the MV522 cell line (a lung-derived adenocarcinoma cell line) compared with other alternative compounds (i.e., Fludarabine, Cytarabine and Doxorubicin). In various embodiments of the invention, the MV522 cell line represents a “target” cell line. That is an illudofulvene analog that exhibits toxicity against this solid tumor cell line shows a desirable result. The 8392B cell line represents a hematopoietic cell line. In various embodiments of the invention, the 8392B cell line is considered a “nontarget” cell line. The two hour toxicity data represents the concentration of a given analog for which a two hour exposure will inhibit 50% of the DNA synthesis activity in a given cell line. The 48 hour exposure data represents the concentration at which a given analog with a 48 hour exposure will inhibit the growth or viability in a given cell line as defined by the standard Trypan Blue Exclusion assay. As an example, Illudofulvene Analog 002 will inhibit the target MV522 cell line at 110 nM with only a 2 hour exposure but has no inhibitory effect on the nontarget 8392B cell line at 26,000 nM (26 μM), see Table 13. Illudofulvene Analog 002 with a prolonged exposure period (e.g. 48 hours) can eventually inhibit the 8392B nontarget cell line, see Table 13. In contrast, Illudofulvene Analog 201 will inhibit the target MV522 cell line with only a 2 hour exposure (IC50=360 nM) but has minimal effect on the 8392B cell nontarget line with even a 48 hour exposure (IC50=26.000 nM) indicating superior anticancer activity as a monotherapeutic agent, see Table 13. In contrast to these two analogs. Illudofulvene Analog 224 displayed minimal toxicity as well as no differential toxicity between the target and nontarget cell line indicating it can have minimal properties as a monotherapeutic anticancer agent, see Table 13. Table 10 indicates that Illudofulvene Analog 001 or Illudofulvene Analog 002 have a novel mechanism of action versus chemotherapeutic agents including Other Drugs as set forth herein.

### Compositions and Methods of Administration.

In other embodiments, described is a pharmaceutical composition including an effective amount of an illudofulvene and a pharmaceutically acceptable carrier or vehicle. The compositions are suitable for veterinary or human administration.

The present pharmaceutical compositions can be in any form that allows for the composition to be administered to a patient. For example, the composition can be in the form of a solid or liquid. Typical routes of administration include, without limitation, parenteral, ocular and intra-tumor. Parenteral administration includes subcutaneous injections, intravenous, intramuscular or intrasternal injection or infusion techniques. In one aspect, the compositions are administered parenterally. In a specific embodiment, the compositions are administered intravenously.

Pharmaceutical compositions can be formulated so as to allow an illudofulvene to be bioavailable upon administration of the composition to a patient. Compositions can take the form of one or more dosage units, where for example, a tablet can be a single dosage unit, and a container of an illudofulvene in liquid form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the illudofulvene, the manner of administration, and the composition employed.

The pharmaceutically acceptable carrier or vehicle can be solid or particulate, so that the compositions are, for example, in tablet or powder form. The carrier(s) can be liquid. In addition, the carrier(s) can be particulate.

The composition can be in the form of a liquid, e.g., a solution, emulsion or suspension. In a composition for administration by injection, one or more of a surfactant, preservative, wetting agent, dispersing agent, suspending agent, buffer, stabilizer and isotonic agent can also be included.

The liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediamine tetraacetic acid; buffers such as acetates, citrates, phosphates or amino acids and agents for the adjustment of tonicity such as sodium chloride or dextrose. A parenteral composition can be enclosed in ampoule, a disposable syringe or a multiple-dose vial made of glass, plastic or other material. Physiological saline is an exemplary adjuvant. An injectable composition is preferably sterile.

The amount of the illudofulvene that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and can be decided according to the judgment of the practitioner and each patient's circumstances.

The compositions comprise an effective amount of an illudofulvene such that a suitable dosage will be obtained. Typically, this amount can be at least about 0.01% of an illudofulvene by weight of the composition. In an exemplary embodiment, pharmaceutical compositions are prepared so that a parenteral dosage unit (actual powder) dissolved and used to inject the patient was 98% drug by weight, although once diluted in an IV the dose contains from about 0.01% to about 2% by weight).

For intravenous administration, the composition can comprise from about 0.01 to about 100 mg of an illudofulvene per kg of the patient's body weight. In one aspect, the composition can include from about 1 to about 100 mg of an illudofulvene per kg of the patient's body weight. In another aspect, the amount administered will be in the range from about 0.1 to about 25 mg/kg of body weight of the illudofulvene.

Generally, the dosage of an illudofulvene administered to a patient is typically about 0.01 mg/kg to about 20 mg/kg of the patient's body weight. In one aspect, the dosage administered to a patient is between about 0.01 mg/kg to about 10 mg/kg of the patient's body weight. In another aspect, the dosage administered to a patient is between about 0.1 mg/kg and about 10 mg/kg of the patient's body weight. In yet another aspect, the dosage administered to a patient is between about 0.1 mg/kg and about 5 mg/kg of the patient's body weight. In yet another aspect the dosage administered is between about 0.1 mg/kg to about 3 mg/kg of the patient's body weight. In yet another aspect, the dosage administered is between about 1 mg/kg to about 3 mg/kg of the patient's body weight.

The illudofulvene can be administered by any convenient route, for example by infusion or bolus injection. Administration can be systemic or local. Various delivery systems are known, e.g., encapsulation in liposomes, micro-particles, microcapsules, capsules, etc., and can be used to administer an illudofulvene. In certain embodiments, more than one illudofulvene can be administered to a patient.

In specific embodiments, it can be desirable to administer one or more illudofulvene locally to the area in need of treatment. This can be achieved, for example, and not by way of limitation, by local infusion during surgery; topical application, e.g., in conjunction with a wound dressing after surgery; by injection; by means of a catheter; or by means of an implant, the implant being of a porous, non-porous, or gelatinous material, including membranes, such as sialastic membranes, or fibers. In one embodiment, administration can be by direct injection at the site (or former site) of a cancer, tumor or neoplastic or pre-neoplastic tissue. In another embodiment, administration can be by direct injection at the site (or former site) of a manifestation of an autoimmune disease.

In yet another embodiment, the illudofulvene can be delivered in a controlled release system, such as but not limited to, a pump or various polymeric materials can be used. In yet another embodiment, a controlled-release system can be placed in proximity of the target of the illudofulvene, e.g., the liver, thus requiring only a fraction of the systemic dose.

The present compositions can take the form of solutions, pellets, powders, sustained-release formulations, or any other form suitable for use.

In an embodiment, the illudofulvenes are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where an illudofulvene is to be administered by infusion, it can be dispensed, for example, with an infusion bottle containing sterile pharmaceutical grade water or saline. Where the illudofulvene can be administered by injection, an ampoule of sterile water for injection or saline can be provided so that the ingredients can be mixed prior to administration.

The composition can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

Whether in solid or liquid form, the present compositions can include a pharmacological agent used in the treatment of cancer, an autoimmune disease or an infectious disease.

The illudofulvene can also be used in an in vitro or ex vivo fashion, such as for the treatment of certain cancers, including, but not limited to leukemia and lymphomas, such treatment involving autologous stem cell transplants. This can involve a multi-step process in which the animal's autologous hematopoietic stem cells are harvested and purged of all cancer cells, the animal's remaining bone-marrow cell population can then eradicated via the administration of a high dose of an illudofulvenes with or without accompanying high dose radiation therapy, and the stem cell graft can be infused back into the animal. Supportive care can then be provided while bone marrow function is restored and the patient recovers.

A method of ablating cancer cells comprising the steps of selecting an illudofulvene which is cytotoxic to the cancer cell and treating the cancer cells with the illudofulvene.

In an embodiment of the present invention, a tumor sample from a patient can be harvested and processed to identify the relative expression of genes encoding the biomarkers described in this application that predict the responsiveness of the patient to Illudofulvenes. Once the relative expression of the biomarkers has been determined, the responsiveness of the cancer patient to treatment with Illudofulvenes, can be determined as outlined in the FIG. 5.

In an embodiment of the present invention, a cancer patient who has an upregulated expression of either PTGR1 or PTGR2 will display responsiveness to treatment with an Illudofulvene if there is a deficiency in any one (or more) of the TCR genes. In an embodiment of the present invention, a cancer patient who has an upregulated expression of either PTGR1 or PTGR2 or an upregulated expression of Myc will display responsiveness to treatment with an Illudofulvene if there is a deficiency in any one (or more) of the TCR genes.

In an embodiment of the present invention, a cancer patient who has an upregulated expression of Myc and does not have an upregulated expression either PTGR1 or PTGR2 will display responsiveness to treatment with an Illudofulvene if there is a deficiency in any two (or more) of the TCR genes. In an embodiment of the present invention, a cancer patient who does not have upregulated expression of PTGR1 and PTGR2 but has an upregulated expression of Myc will display responsiveness to treatment with an Illudofulvene if there is a deficiency in any two (or more) of the TCR genes.

In an embodiment of the present invention, a cancer patient who does not have an upregulated expression of PTGR1 and PTGR2 and does not have an upregulated expression of Myc will display responsiveness to treatment with an Illudofulvene if there is a deficiency in any three (or more) of the TCR genes. In an embodiment of the present invention, a cancer patient who does not have an upregulated expression of Myc and does not have an upregulated expression of PTGR1 and PTGR2 will display responsiveness to treatment with an Illudofulvene if there is a deficiency in any three (or more) of the TCR genes. In an embodiment of the present invention, a cancer patient who does not have an upregulated expression of PTGR1, PTGR2, and Myc will display responsiveness to treatment with an Illudofulvene if there is a deficiency in any three (or more) of the TCR genes.

Quantification of Gene mRNA Expression by Probe Analysis.

Tumor tissue can be harvested and total RNA can be purified using Trizol Reagent (Invitrogen Life Technology, 15596-018, Waltham. MA) and the RNeasy Mini Kit (Qiagen, 74104, Germantown, MD). The mRNA can then be purified (from the total RNA) using an Oliogotex mRNA kit (Qiagen, 70022, Germantown, MD). The isolated tissue mRNA can then be fixed to a nylon membrane (ThermoFisher, AM10102, Waltham. MA), by UV crosslinking (Stratagene, UC Crosslinker 2400, La Jolla, CA), for subsequent analysis of the relative quantity expressed by a specific gene. Probes can then be prepared by PCR labeling. Probes can be labeled with a radioactive isotope, e.g. P32 or P33, but the preferred approach is use of a nonradioactive label such as Digoxigen-11-dUTP (Sigma Aldrich, 1157315290, St Louis, MO). Other potential nonradioactive probe labeling methods include the use of Biotin-16-UTP, Biotin-11-CTP or Fluoroscein-12-UTP. In PCR labeling, a thermostable polymerase (Agilent, 600195, Santa Clara, CA) incorporates the Digoxigen-11-dUTP as it amplifies a specific region of template DNA that is often contained in a plasmid. The specificity of the probe (or gene under study) can be determined by the two PCR primers utilized. The result can be a highly labeled, highly specific and sensitive hybridization probe for quantifying the amount of corresponding mRNA in a tissue sample. The hybridization of the probe to the membrane can be performed normally in roller bottles in a hybridization oven, followed by high stringency washes at elevated temperatures up to 70° C., to remove nonspecific binding and background signal. The digoxigen labeled probes are detected using labeled anti-digoxigen antibody (Abcam product number 420) or Fab fragments (Sigma Aldrich, 11207733910, St Louis, MO). If another label is utilized for probe labeling, such as biotin, then an anti-biotin labeled antibody can be used. Visualization and quantification of signal can be performed using a phosphoimager (Storm, 860 imager, Ramsey, MN) or a fluoroimager, depending upon the type of label used in the detection antibody.

In an embodiment of the invention, the tumor tissue is harvested and mRNA purified from the tumor. The mRNA is processed, subjected to electrophoresis and then analyzed with the desired probe for individual gene products, as well as for the three “housekeeping” genes (ACTB, TFRC, and GAPD). The required probes specific for detecting a gene product are prepared using PCR with the stated primers specific for that gene (Table 1) and labeled with a detection product as described in this application. The raw expression of each gene product is then determined using a densitometer appropriate for the label used (fluorescent, visible, photometer, radioisotope, etc.) Next a “relative ratio of expression” is determined for each of the gene products under investigation versus the individual “housekeeping genes” as described above in the Microarray section. These ratios are then compared to ratios obtained for a cell known to be sensitive to an Illudofulvene as well as to ratios for a cell know to be resistant to an Illudofulvene. One can then determine whether (i) a PTGR gene is upregulated, and/or (ii) the MYC gene is upregulated and (iii) there is a deficiency in a specific TCR gene. The tumor can then be classified as either sensitive, or resistant, to Illudofulvenes based on this analysis.

TABLE 1
Primers for use with gene mRNA expression,
according to embodiments of the invention.

| | Upper | Lower | | Primer |
|---|---|---|---|---|
| | Primer | Primer | | Annealing |
| | SEQ ID | SEQ ID | Probe | Temp |
| GENE | NOS: | NOS: | (bp) | (° C.) |
| ERCC1 | 275 | 276 | 467 | 59 |
| ERCC2 | 277 | 278 | 601 | 60 |
| ERCC3 | 279 | 280 | 415 | 59 |
| ERCC4 (set 1) | 281 | 282 | 1217 | 56 |
| ERCC4 (set 2) | 283 | 284 | 500 | 58 |
| ERRC5 | 285 | 286 | 872 | 56 |
| ERCC6 | 287 | 288 | 876 | 55 |
| ERCC6L | 289 | 290 | 629 | 62 |
| ERCC6L2 | 291 | 292 | 594 | 63 |
| ERCC8 | 293 | 294 | 534 | 50 |
| GTF2H1 | 295 | 296 | 1139 | 66 |
| GTF2H2 | 297 | 298 | 802 | 64 |
| GTF2H3 | 299 | 300 | 344 | 62 |
| GTF2H4 | 301 | 302 | 560 | 62 |
| GTF2H5 | 303 | 304 | 606 | 63 |
| MYC | 305 | 306 | 630 | 63 |
| PTGR1 | 307 | 308 | 621 | 61 |
| PTGR2 | 309 | 310 | 626 | 63 |
| RAD18 | 311 | 312 | 601 | 58 |
| USP7 | 313 | 314 | 600 | 62 |
| UVSSA | 315 | 316 | 595 | 62 |
| XPA | 317 | 318 | 459 | 64 |
| XPC | 319 | 320 | 855 | 62 |
| XPE | 321 | 322 | 629 | 62 |
| Controls | | | | |
| ACTB β-Actin | 323 | 324 | 566 | 59 |
| TFRC | 325 | 326 | 1096 | 53 |
| GAPD | 327 | 328 | 547 | 60 |

The cDNA genes under investigation were isolated internally (by applicant) from a lambda gt11 expression vector using primers above, then cloned into a plasmid. All sequences were confirmed by nucleotide sequencing. The genes at the time were not available from commercial sources.

### Microarray Analysis.

Tumor tissue can be harvested and total RNA can be purified using Trizol Reagent (Invitrogen Life Technology, 15596-018, Waltham. MA) and the RNeasy Mini Kit (Qiagen, 74104, Germantown, MD). The mRNA can then be purified (from the total RNA) using an Oliogotex mRNA kit (Qiagen, 70022, Germantown, MD). The Oliogotex mRNA kit (Qiagen, 70022, Germantown, MD) is the preferred method for preparing mRNA for Genechip® analysis such as the Affymetrix® platform. Prior to use in a Microarray analysis, the purified mRNA (˜1 to 2 μg) can be first reverse transcribed using a T7-Oligo(dT) Promoter Primer (Affymetrix GeneChip T7-Oligo(dT) Promoter Primer Kit 900375, Waltham. MA) in the first-strand cDNA synthesis reaction. Alternatively, total RNA (10 μg to 15 μg) can be used if of high quality.

Next an RNase H-mediated second-strand cDNA synthesis can be performed, the double-stranded cDNA can be purified, and this double-strand cDNA serves as the template in a subsequent in vitro transcription (IVT) reaction (Affymetrix GeneChip IVT kit 900688, Waltham. MA). The IVT reaction can be carried out in the presence of provided T7 RNA Polymerase and a biotinylated nucleotide analog/ribonucleotide mix for complementary RNA (cRNA) amplification and biotin labeling. The biotinylated cRNA targets are then cleaned up and fragmented using the buffer provided in the kit.

Next the cRNA under study and a control oligonucleotide are prepared in a hybridization solution (Affymetrix, 900454, Waltham. MA) and added to the Affymetrix HG-U133 Plus 2 array cartridge, then placed in the hybridization oven at 45 C.° with rotation at 60 RPM, for 16 hours. The HG-U133 cartridge can be then washed and stained using the appropriate Fluidics station per manufacturer's protocol. The array can then be scanned using an appropriate device such as the Affymetrix GeneChip scanner or a generic scanner such as the Agilent GeneArray scanner. Normalization and analysis of data can be accomplished using the NetAffx Software (Thermo Fisher, Waltham. MA), the Gene Spring GX 9 software (Agilent, Santa Clara, CA) or open source academic software such as R/Bioconductor.

The specific probe sequences and corresponding Affymetrix IDs, for the biomarkers involved, for the Human Genome U133 Plus 2.0 array (HG-U133 Plus 2) are provided in Tables 2 and 3.

In an embodiment of the invention, the tumor tissue is harvested and the relative expression of the individual gene products, as well as the mRNA expression for the three “housekeeping” genes (ACTB, TFRC, GAPD), is quantified by a microarray apparatus using the specific probes for each gene product (see Tables 2 and 3). Next a “relative ratio of expression” is determined for each of the gene products under s investigation versus the individual “housekeeping genes” (e.g. ERCC6 expression/ACTB expression). This is accomplished by dividing the raw value of expression for each gene product under study by the raw value of expression for the desired “housekeeping gene”. As an example the ERCC6/ACTB, ERCC6/TFRC, and the ERRC6/GAPD expression ratios for the tumor under study are calculated. Next an analysis is performed by comparing each relative ratio for each gene using each housekeeping gene, and comparing the ratio to the same ratio for a cell known to be sensitive to an Illudofulvene as well as a cell know to be resistant to an Illudofulvene. As an example the ERCC6/ACTB, ERCC6/TFRC, and the ERRC6/GAPD expression ratios for the tumor under study are compared to the same ratios in a cell known to be sensitive to an Illudofulvene as well as a cell know to be resistant to an Illudofulvene. Note that only one of the many gene ratios could be similar to a ratio derived from a cell known to be sensitive to an Illudofulvene in order to classify the tumor as TCR deficient. If any single component of the TCR pathway is deficient then that single deficiency is sufficient to render a tumor sensitive to Illudofulvenes (see Table 10). Similarly, the ratios for the individual expression of PTGR1, PTGR2, and MYC to the three “housekeeping” genes can be derived and each ratio can be compared to the same ratio in a cell known to be sensitive to an Illudofulvene as well as a cell know to be resistant to an Illudofulvene. This allows a classification as to whether PTGR1, PTGR2, or MYC are either upregulated or not upregulated and which specific gene(s) are upregulated for that individual tumor. The classification also allows determination of the specific TCR genes that are deficient.

TABLE 2
Probe Sequences to analyze not upregulated gene
products that confer sensitivity to Illudofulvenes

| Gene | Affymetrix ID | SEQ ID NOS: |
|---|---|---|
| ERCC8 | 201562_at | 35-45 |
| ERCC6 | 207347_at | 46-56 |
| GTF2H1 | 202451_at | 57-67 |
| GTF2H2 | 230177_at | 68-78 |
| GTF2H3 | 239346_at | 79-89 |
| GTF2H4 | 203577_at | 90-100 |
| GTF2H5 | 213357_at | 101-111 |
| USP7 | 2014498_at | 112-122 |
| UVSSA | 241201_at | 123-133 |
| ERCC3 | 202176_at | 134-144 |
| ERCC2 | 213468_at | 145-155 |
| ERCC4 | 210158_at | 156-166 |
| ERCC5 | 202414_at | 167-177 |
| XPA | 205672_at | 178-188 |
| RAD18 | 223417_at | 189-199 |
| ERCC6/L | 219650_at | 200-210 |
| ERCC6/L2 | 228211_at | 211-221 |
| ERCC1 | 203719_t | 238-248 |

Affymetrix IDs refer to the array type Human Genome U133 Plus 2.0 (HG-U133 Plus 2)

TABLE 3
Probe Sequences to analyze upregulated gene products
that confer sensitivity to Illudofulvenes

| Gene | Affymetrix ID | SEQ ID NOS: |
|---|---|---|
| MYC | 20243_s_at | 224-235 |
| PTGR1 | 231897_at | 251-261 |
| PTGR2 | 230774_at | 264-274 |

Affymetrix IDs refer to the array type Human Genome U133 Plus 2.0 (HG-U133 Plus 2)

### RT-PCR Analysis.

In an embodiment of the invention, the RT-PCR Analysis can be carried out using the Hydrolysis Taqman Fluorescent probe approach. Tumor tissue can be harvested and total RNA can be purified using Trizol Reagent (Invitrogen Life Technology, 15596-018, Waltham. MA) and the RNeasy Mini Kit (Qiagen, 74104, Germantown, MD). The mRNA can then be purified (from the total RNA) using an Oliogotex mRNA kit (Qiagen, 70022, Germantown, MD). Then reverse transcription can be performed using oligo(dT) and random hexamers and SuperScript® II (Thermofisher, #18064014, Waltham, MA) to produce cDNA. Each reaction mixture contains approximately 50 ng of tumor-derived cDNA.

The desired gene primers, and associated hydrolysis oligonucleotide probe (see Table 4) with a 5′ fluorescent reporter and a 3′ quencher, are obtained from Integrated DNA Technologies. RT-PCR reaction conditions are an initial denaturation for 2 minutes at 95° C. and 45 cycles of 94° C. for 15 seconds, annealing at 56° C. for 15 seconds and 72° C. for 30 seconds, with final extension at 72° C. for 10 minutes. The hydrolysis probe for the gene under study can be labeled at the 5′ end with the fluorophore FAM (fluorescein). A control gene can be amplified as an internal control to check the specificity of the reaction. The hydrolysis probe can be labeled at the 5′ end with the fluorophore HEX (555) to allow for differentiation of fluorescence arising from the biomarker hydrolysis probe and the internal control probe. All RT-PCR assays are run in triplicate using TaqMan® Gene Expression Master Mix (ThermoFisher. #4369016, Waltham. MA).

Quantitative results are obtained by monitoring the increase in fluorescence (which occurs upon release of the fluorescent reporter molecule from the hydrolysis probe and the associated quencher) and can be proportional to the amount of amplified product produced, using a Roche Light cycle Model 2.0 (or later version).

In an embodiment of the invention, the tumor tissue is harvested and the relative expression of the individual gene products, as well as the mRNA expression for the three “housekeeping” genes (ACTB, TFRC, GAPD), is quantified by RT-PCR using the desired primers (Table 4) specific for each gene product or “housekeeping gene”. Next a “relative ratio of expression” is determined for each of the gene products under s investigation versus the individual “housekeeping genes” as described above in the Microarray Analysis section. These ratios are then compared to ratios obtained for a cell known to be sensitive to an Illudofulvene as well as to ratios for a cell know to be resistant to an Illudofulvene. It can then be determined whether (i) a PTGR gene is upregulated, or (ii) the MYC gene is upregulated and (iii) there is a deficiency in at least one specific TCR gene. The tumor can then be classified as either sensitive, or resistant, to Illudofulvenes based on this analysis.

TABLE 4
Required RT-PCR primers and hydrolysis probe for each Biomarker.

| | Upper | Hydrolysis | Lower | | |
|---|---|---|---|---|---|
| | Primer | Probe | Primer | | |
| | SEQ ID | SEQ ID | SEQ ID | Probe | Temp |
| GENE | NOS: | NOS: | NOS: | (bp) | (° C.) |
| ERCC1 | 852 | 853 | 854 | 139 | 61 |
| ERCC2 | 855 | 856 | 857 | 141 | 60 |
| ERCC3 | 858 | 859 | 860 | 143 | 60 |
| ERCC4 | 861 | 862 | 863 | 137 | 60 |
| ERRC5 | 864 | 865 | 866 | 143 | 60 |
| ERCC6 | 867 | 868 | 869 | 148 | 60 |
| ERCC6L | 870 | 871 | 872 | 137 | 61 |
| ERCC6L2 | 873 | 874 | 875 | 148 | 60 |
| ERCC8 | 876 | 877 | 878 | 142 | 60 |
| GTF2H1 | 879 | 880 | 881 | 89 | 61 |
| GTF2H2 | 882 | 883 | 884 | 146 | 60 |
| GTF2H3 | 885 | 886 | 887 | 141 | 61 |
| GTF2H4 | 888 | 889 | 890 | 150 | 61 |
| GTF2H5 | 891 | 892 | 893 | 149 | 61 |
| MYC | 894 | 895 | 896 | 108 | 60 |
| PTGR1 | 897 | 898 | 899 | 150 | 60 |
| PTGR2 | 900 | 901 | 902 | 148 | 60 |
| RAD18 | 903 | 904 | 905 | 137 | 60 |
| USP7 | 906 | 907 | 908 | 135 | 60 |
| UVSSA | 909 | 910 | 911 | 114 | 61 |
| XPA | 912 | 913 | 914 | 140 | 60 |
| XPC | 915 | 916 | 917 | 144 | 61 |
| ACTB β-Actin | 918 | 919 | 920 | 148 | 61 |
| (control) | | | | | |
| TFRC (control) | 921 | 922 | 923 | 127 | 60 |
| GAPD (control) | 924 | 925 | 926 | 143 | 60 |

### TCR Tumor Mutation Panel (22 Genes) Analysis.

The TCR Tumor Mutation Panel targets a total of 22 clinically relevant genes. Specifically, the panel interrogates all exons of 22 genes known to be associated with sensitivity to Illudofulvenes.

The TCR Tumor Mutation Panel (see Table 5) is a highly multiplexed targeted re-sequencing assay for detecting somatic variants across key genes in cancer genomes [see (i) Illumina HiSeq® 2500 System User Guide. Illumina Proprietary Part #15035786 Rev. D, November 2014; (ii) HiSeq Rapid SBS Kit v2 Reagent Prep Guide. Illumina Proprietary Part #15058772 Rev. A, November 2014; (iii) HiSeq Rapid Cluster Kit v2 Reagent Prep Guide. Illumina Proprietary Part #15059131 Rev. A, November 2014; (iv) Sequencing Analysis Viewer Software User Guide. Illumina Proprietary Part #15020619 Rev. F, October 2014; of which (i)-(iv) are herein expressly incorporated by reference in their entireties for all purposes]. The analysis entails hybridization-based capture of 513 probes (custom mix synthesis from Integrative DNA Technologies) covering 47,848 base pairs that encompass the 281 exons and regulatory regions of these 22 genes. The generation of a library of multiplexed, enriched genomic regions can be followed by a sequencing-by-synthesis assay using massively parallel 100 base pair sequence reads. Once data are generated, a series of bioinformatics tools are used to align the reads to the genome, call variants, and annotate variants with respect to somatic status and functional predictions. This analysis can detect single nucleotide variants, short insertion/deletions, and splice variants predicted to be deleterious. In addition, the analysis can detect a set of targeted fusions or rearrangement.

Genomic DNA extracted from tumor tissue can be sheared, end repaired, A-tailed, adapter ligated, amplified, hybridized to the capture (bait) library primers, then re-amplified per the HiSeq Reagent Pre Guides (Illumina #15058772 and #15059131). The subsequent sample pool can then be sequenced to a depth of 300× on an Illumina HiSeq 2500 workstation per the user guide (Illumina #15035786). The primary data analysis can be accomplished by the on-board software (Real Time Analysis). Secondary analysis can be accomplished by uploading base call files (*.bcl) to a genomics server designed to generate reads and read alignments, followed by comparison to the DNA sequence for the 22 genes as listed in the latest version of the Genomic Reference Consortium Human (current build 37). This allows identification of tumors that are deficient in TCR capacity, on the basis of a deficiency in one (or more) of the components of the TCR pathways, and which can be responsive to treatment with an Illudofulvene. In addition, tumors that are deficient in PTGR metabolic activity (either PTGR1 or PTGR2) can also be identified, and such tumors excluded from treatment based on the expected lack of response to therapy.

In an embodiment of the invention, a tumor sample is obtained from a patient, prepared, and the genomic DNA isolated and processed as described in this application. The TCR 22 gene mutation panel (Table 5) is then obtained by sequencing in entirety these genes, and results analyzed versus the latest version of the Genomic Reference Consortium Human (current build 37, Table 6), as described in this application. Tumors deficient in TCR capacity, on the basis of a deficiency in one (or more) of the components of the TCR pathways due to a mutation, and which can be responsive to treatment with an Illudofulvene, can then be identified. Tumors that are deficient in PTGR metabolic activity (either PTGR1 or PTGR2) can also be identified, and such tumors excluded from treatment based on the expected lack of response to therapy, subject to the flow chart of FIG. 5. It can also be determined whether MYC is upregulated or downregulated.

TABLE 5
The twenty two (22) Gene
TCR Tumor Mutation Panel

| GENE | SEQ ID NOS: |
|---|---|
| ERCC1 | 329-343 |
| ERCC2 | 344-374 |
| ERCC3 | 375-402 |
| ERCC4 | 403-429 |
| ERCC5 | 430-440 and 451-475 |
| ERCC6 | 476-539 |
| ERCC6L | 540-571 |
| ERCC6L2 | 572-596 |
| GTF2H1 | 597-615 |
| GTF2H2 | 616-631 |
| GTF2H3 | 632-646 |
| GTF2H4 | 647-665 |
| GTF2H5 | 666-668 |
| MYC | 669-681 |
| PTGR1 | 682-696 |
| RAD18 | 697-714 |
| PTGR2 | 715-727 |
| XPA | 728-736 |
| XPC | 737-777 |
| USP7 | 768-809 |
| UVSSA | 810-835 |
| ERCC8 | 836-851 |

TABLE 6
A list of genes comprising components of the TCR-NER pathway, post-
replication repair pathway, or bio activation of Illudofulvenes of which an
alteration of one or more of the components will confer sensitivity to
illudofulvenes, according to various embodiments of the invention.

| Gene (HGNC | Gene | Representative | SEQ ID | Representative | SEQ ID |
|---|---|---|---|---|---|
| Symbol) | Aliases | mRNA Sequence | NO | Protein Sequence | NO |
| ERCC8 | CSA, | NM_001290285 | 1 | NP_000073 | 2 |
| | CKN1, | | | | |
| | UVSS2 | | | | |
| ERCC6 | CSB, | NM_001277058.1 | 3 | NP_000115.1 | 4 |
| | ARMD5, | | | | |
| | CKN2, | | | | |
| | COFS, | | | | |
| | COFS1, | | | | |
| | RAD26 | | | | |
| | POF11, | | | | |
| | UVSS1 | | | | |
| GTF2141 | P62, | NM_001142307.1 | 5 | NP_005307.1 | 6 |
| | BTF2P62, | | | | |
| | TUB1, | | | | |
| | TFIIH | | | | |
| | SUBUNIT 1 | | | | |
| GTF2H2 | BTF2P44. P44 | NM_001515.3 | 7 | NP_001506.1 | 8 |
| GTF2H3 | P34, TFB4 | NM_001516.4 | 9 | NP_001507.2 | 10 |
| GTF2H4 | P52, TFB2 | NM_001517.4 | 11 | NP_001508.1 | 12 |
| GTF2H5 | TTDA, | NM_207118.2 | 13 | NP_997001.1 | 14 |
| | TFB5, | | | | |
| | TGF2H5, | | | | |
| | TTD3, | | | | |
| | C6orf175, | | | | |
| | bA120J8.2 | | | | |
| USP7 | HAUSP, | NM_003470.3 | 15 | NP_0013461.2 | 16 |
| | TEF1 | | | | |
| UVSSA | KIAA1530, | NM_020894.3 | 17 | NP_001304863.1 | 18 |
| | UVSS3 | | | | |
| ERCC3 | XPB, | NM_001303418.1 | 19 | NP_001290347.1 | 20 |
| | RAD25, | | | | |
| | TTD2, | | | | |
| | BFT2 | | | | |
| ERCC2 | XPD, | NM_000400.3 | 21 | NP_000391.1 | 22 |
| | COFS2, | | | | |
| | EM9, | | | | |
| | TTD1 | | | | |
| ERCC4 | XPF, | NM_005236.2 | 23 | NP_005227.1 | 24 |
| | ERCC11, | | | | |
| | FANCQ, | | | | |
| | RAD1, | | | | |
| | XFEPS | | | | |
| ERCC5 | XPG, | NM_000123.3 | 25 | NP_000114.2 | 26 |
| | COFS3, | | | | |
| | ERCM2, | | | | |
| | UVDR, | | | | |
| | XPGC | | | | |
| XPA | XP1, | NM_001354975.1 | 27 | NP_000371.1 | 29 |
| | XPAC | | | | |
| RAD18 | RNF73 | NM_020165.3 | 29 | NP_064550.3 | 30 |
| ERCC6L | PICH, | NM_017669.3 | 31 | NP_060139.2 | 32 |
| | RAD26L | | | | |
| ERCC6/L2 | BMFS2, | NM_020207.4 | 33 | NP_064592.2 | 34 |
| | C9orf102, | | | | |
| | RAD26L, | | | | |
| | SR278 | | | | |
| MYC | v-myc, | NM_002467.5 | 222 | NP_002458 | 223 |
| | c-myc, | | | | |
| | bHLHe39, | | | | |
| | MYCC | | | | |
| ERCC1 | COFS4, | NM_202001.2 | 236 | NP_973730.1 | 237 |
| | UV20, | | | | |
| | RAD10 | | | | |
| PTGR1 | PGR1, | NM_001146109.1 | 249 | NP_001139581 | 250 |
| | PRG-1, | | | | |
| | LTBD4H, | | | | |
| | ZADH3 | | | | |
| PTGR2 | PGR2, | NM_001146154.1 | 262 | NP_001139626.1 | 263 |
| | ZADH1, | | | | |
| | HEL-S-298 | | | | |

TABLE 7
A list of biomarkers that when downregulated (expression
decreased) confer sensitivity to illudofulvenes, according
to various embodiments of the invention.

| Gene | Affymetrix ID | SEQ ID NO |
|---|---|---|
| ERCC8 | 201562_at | 35-45 |
| ERCC6 | 207347_at | 46-56 |
| GTF2H1 | 202451_at | 57-67 |
| GTF2H2 | 230177_at | 68-78 |
| GTF2H3 | 239346_at | 79-89 |
| GTF2H4 | 203577_at | 90-100 |
| GTF2H5 | 213357_at | 101-111 |
| USP7 | 2014498_at | 112-122 |
| UVSSA | 241201_at | 123-133 |
| ERCC3 | 202176_at | 134-144 |
| ERCC2 | 213468_at | 145-155 |
| ERCC4 | 210158_at | 156-166 |
| ERCC5 | 202414_at | 167-177 |
| XPA | 205672_at | 178-188 |
| RAD18 | 223417_at | 189-199 |
| ERCC6/L | 219650_at | 200-210 |
| ERCC6/L2 | 228211_at | 211-221 |
| ERCC1 | 203719_t | 238-248 |

Affymetrix IDs refer to the array type Human Genome U133 Plus 2.0 (HG-U133 Plus 2)

TABLE 8
A list of biomarkers that when upregulated (expression
increased) confer sensitivity to illudofulvenes,
according to various embodiments of the invention.

| Gene | Affymetrix ID | SEQ ID NO |
|---|---|---|
| MYC | 20243_s_at | 224-235 |
| PTGR1 | 231897_at | 251-261 |
| PTGR2 | 230774_at | 264-274 |

Affymetrix IDs refer to the array type Human Genome U133 Plus 2.0 (HG-U133 Plus 2).

TABLE 9
The toxicity of Illudofulven Analog 001/Analog 002
to either 2 hour or 48 hour incubation, according
to various embodiments of the invention.

| | Cytotoxicity? | |
|---|---|---|
| Tumor cell line | 2 hr incubation | 48 hr incubation |
| Myeloid leukemias | Yes | Yes |
| Breast carcinomas | Yes | Yes |
| Epidermoid | Yes | Yes |
| Ovarian | Yes | Yes |
| Lung carcinomas | Yes | Yes |
| Prostate carcinomas | Yes | Yes |
| B cell leukemias | No | Yes |
| T cell leukemias | No | Yes |
| Fibroblasts (normal) | No | Yes |

TABLE 10
A comparison of the DNA damage profile of Illudofulven
Analog 001/Analog 002 with Other Drugs and UV
irradiation, according to embodiments of the invention.

| | Other | | Illudofulvene |
|---|---|---|---|
| Gene | Drugs* | UV | Analog 001/002 |
| XP-A | + | + | + |
| XP-B | 0 | + | + |
| XP-C | + | + | 0 |
| XP-D | 0 | + | + |
| XP-E | + | + | 0 |
| XP-F | + | + | + |
| CS-A | +/− | + | ++ |
| CS-B | +/− | + | ++ |
| ERCC1 | + | + | + |
| ERCC5 | + | + | + |

*as defined herein.

TABLE 11
The multidrug resistance of Illudofulvene
Analog 001/Analog 002, according to
various embodiment of the invention.

| | Resistance to |
|---|---|
| Mechanism | Illudofulvene |
| of Multi-drug | Analog 001/ |
| Resistance | Analog 002 |
| Gp170/MDR1 | No |
| Gp180/MRP | No |
| Topoisomerase I | No |
| Topoisomerase II | No |
| MVP/LRP (vault) | No |
| Thiol content/GST pi | No |
| DNA repair | No |
| Myc expression | No |
| Bcl-2 expression | No |
| BRCA status | No |
| P53 status | No |
| P21 status | No |
| MGMT expression | No |
| Microtubulin alteration | No |

TABLE 12
The cytotoxic IC₅₀ values (micromolar, 2 hour exposure, N = 3, mean ± SD)
for illudin M, llludofulvene Analog 108 and Illudolulvene Analog 110
for cells expressing the estrogen receptor (ER) (MCF7)and cells not
expressing the ER (HT29), according to embodiments of the invention.

| Analog | HT29 (ER Negative) | MCF7 (ER positive) |
|---|---|---|
| Illudin M | 0.52 ± 0.10 | 0.48 ± 0.13 |
| 108 | >55 | 14.1 ± 2.8 |
| 110 | >19 | 2.0 ± 0.1 |

TABLE 13
The ability of Illudofulvene analogs to inhibit tumor cell growth, according
to various embodiments of the invention.

| | Mean IC50 value (nM) ± SD, N = 3 unless otherwise indicated | | | |
|---|---|---|---|---|
| Illudofulvene | MV522 Target Cell Line | | 8392B Nontarget Cell Line | |
| Analog | 2 hr exposure | 48 hr exposure | 2 hr exposure | 48 hr exposure |
| 001 | 2200 ± 100 | 350 ± 20 | | 830 ± 100 |
| 002 | 110 ± 40 | 70 ± 10 | 26000 ± 4500 | 800 ± 100 |
| 004 | 4200 | 600 | | |
| 008 | 870 ± 90 | 630 ± 80 | 12200 ± 700 | 15100 ± 2200 |
| 009 | 500 ± 30 | 850 ± 180 | 47100 ± 11000 | 43200 ± 2300 |
| 010 | 8900 ± 1500 | 170 ± 60 | 29400 ± 1600 | 14500 ± 1700 |
| 011 | 4900 ± 900 | 1200 (N = 2) | >100000 | 40400 ± 6700 |
| 012 | 5150 ± 1350 | 320 ± 90 | 42200 ± 5000 | 18800 ± 2800 |
| 013 | 5100 ± 700 | 270 ± 130 | 11900 ± 1300 | 4200 ± 400 |
| 014 | 115 ± 30 | 460 ± 120 | 9650 ± 200 | 1100 ± 300 |
| 015 | 1800 ± 200 | 480 ± 110 | 810 ± 260 | 1300 ± 150 |
| 016 | 490 ± 130 | 440 ± 90 | >100000 | 870 ± 60 |
| 017 | 2400 ± 360 | 320 ± 60 | 14700 ± 900 | |
| 018 | 8800 ± 2900 | | 4200 ± 1300 | |
| 019 | 470 ± 60 | 660 ± 80 | >75000 | |
| 020 | 530 ± 140 | 230 ± 10 | 25000 ± 3100 | |
| 021 | 2400 ± 1000 | 930 ± 250 | 34400 ± 9400 | |
| 022 | 700 ± 200 | 680 ± 180 | 31700 ± 1400 | |
| 023 | 2900 ± 1140 | 2750 ± 500 | >138000 | |
| 024 | 1800 ± 200 | 1200 ± 300 | 12800 ± 2100 | |
| 025 | 1300 ± 310 | 1200 ± 100 | >25000 | |
| 030 | | >3000 | | |
| 031 | | >3000 | | |
| 032 | 600 ± 190 | 210 ± 30 | >30000 | |
| 033 | 10000 ± 1100 | 4600 ± 200 | 29900 ± 3300 | |
| 034 | 1400 ± 170 | 490 ± 40 | >100000 | 4400 ± 200 |
| 035 | 5600 ± 600 | | >150000 | |
| 037 | 26000 ± 5000 | 29200 ± 2300 | >85000 | |
| 038 | 750 ± 60 | | 24900 ± 8000 | |
| 039 | 1500 ± 240 | 600 ± 40 | 24600 ± 2400 | 820 ± 250 |
| 040 | 3400 ± 360 | 700 ± 90 | 24000 ± 3300 | 5200 ± 470 |
| 060 | 19400 ± 1800 | | 27600 ± 3000 | |
| 062 | 2600 ± 300 | 660 ± 200 | 37100 ± 2300 | |
| 063 | 43000 ± 5700 | 580 ± 250 | | |
| 064 | 28000 ± 4600 | 1200 ± 300 | | |
| 065 | 6200 ± 1100 | 2500 ± 1200 | | |
| 075 | 19600 ± 9700 | | 62000 ± 3600 | |
| 076 | 24000 ± 6100 | | 39500 ± 7200 | |
| 077 | 9200 ± 1200 | | | |
| 078 | 20400 ± 6300 | | >100000 | |
| 079 | 7700 ± 3500 | | >100000 | |
| 080 | 8800 ± 2400 | | >100000 | |
| 081 | >80000 | | >80000 | |
| 082 | 50600 ± 7100 | | >100000 | |
| 083 | | 37200 ± 2900 | >42000 | |
| 084 | | 28200 ± 1400 | >42000 | |
| 085 | >40000 | | >40000 | |
| 087 | >40000 | 24700 ± 3900 | >40000 | |
| 089 | 19300 ± 5700 | 15500 ± 2800 | >60000 | |
| 090 | 2500 ± 400 | 2900 ± 400 | 1600 ± 200 | 3800 ± 300 |
| 094 | 800 ± 100 | 210 ± 20 | 9000 ± 1700 | 110 ± 10 |
| 096 | 2700 ± 400 | 6200 ± 600 | >88000 | >3000 |
| 097 | 2900 ± 100 | | >82000 | |
| 098 | 18800 ± 2500 | 4600 ± 250 | >65000 | 11700 ± 1800 |
| 099 | 8400 ± 1100 | 1800 ± 200 | 4000 ± 400 | 300 ± 20 |
| 100 | >10000 | 1700 ± 500 | | |
| 101 | >8000 | >7500 | | |
| 102 | >13000 | 1300 ± 100 | | |
| 103 | 31800 ± 4900 | 5900 ± 400 | 12100 ± 2000 | 2300 ± 200 |
| 104 | 6300 ± 400 | 6000 ± 500 | 36400 ± 6500 | 2700 ± 600 |
| 105 | 7300 ± 1200 | 2100 ± 400 | >100000 | |
| 106 | 5200 ± 1000 | | >83000 | |
| 107 | >50000 | 1600 ± 100 | >50000 | |
| 108 | 12300 ± 2300 | 520 ± 50 | >55000 | 6000 ± 1600 |
| 109 | >50000 | | >50000 | |
| 110 | >55000 | 1400 ± 100 | >55000 | 25300 ± 2100 |
| 111 | 16700 ± 2100 | 11900 ± 2800 | 34600 ± 2100 | 10200 ± 1000 |
| 112 | 10000 ± 2000 | 6700 ± 1200 | 14900 ± 100 | 5200 ± 300 |
| 113 | 85000 ± 700 | 14100 ± 3000 | >93000 | 7800 ± 1000 |
| 114 | 1500 ± 100 | 260 ± 70 | 25100 ± 1000 | 700 ± 100 |
| 115 | 1500 ± 100 | 70 ± 5 | 1600 ± 700 | 630 ± 60 |
| 116 | 400 ± 100 | 1000 ± 50 | 7000 ± 400 | 170 ± 30 |
| 117 | 1100 ± 100 | 100 ± 30 | 7900 ± 1600 | 10 ± 2 |
| 118 | 14000 ± 2000 | 740 ± 120 | 24500 ± 4500 | 2000 ± 400 |
| 119 | 1100 ± 70 | 270 ± 40 | >33000 | >10000 |
| 120 | 2800 ± 900 | 600 ± 100 | 19100 ± 4600 | 510 ± 110 |
| 121 | 300 ± 10 | 90 ± 10 | 15200 ± 6000 | 1300 ± 500 |
| 122 | 6400 ± 300 | 2400 ± 300 | 14500 ± 1200 | 1100 ± 300 |
| 123 | 1900 ± 400 | 600 ± 60 | 450 ± 30 | 2400 ± 500 |
| 124 | 2800 ± 700 | 870 ± 350 | >30000 | 2400 ± 550 |
| 125 | 3700 ± 600 | 1200 ± 200 | 15500 ± 1400 | 600 ± 100 |
| 126 | 2100 ± 500 | 900 ± 100 | >30000 | 330 ± 80 |
| 127 | 870 ± 30 | 340 ± 90 | >30000 | 100 ± 40 |
| 128 | 840 ± 230 | 370 ± 50 | >35000 | 800 ± 70 |
| 129 | >136000 | 19700 ± 1900 | >136000 | 39400 ± 9200 |
| 130 | 700 ± 100 | 130 ± 40 | 27,000 ± 7000 | 4400 ± 500 |
| 133 | 58800 ± 6600 | 15800 ± 2600 | 12200 ± 2300 | 2700 ± 400 |
| 134 | 50000 ± 6000 | 28000 ± 4000 | 43900 ± 5100 | 8500 ± 2000 |
| 135 | 1600 ± 300 | 22 ± 4 | 70 ± 20 | 22 ± 2 |
| 136 | 430 ± 10 | 130 ± 10 | >6200 | 25 ± 2 |
| 137 | 850 ± 110 | 1200 ± 100 | 8500 ± 1200 | 710 ± 60 |
| 138 | 2100 ± 200 | 1000 ± 200 | 5400 ± 200 | 820 ± 230 |
| 139 | 6400 ± 900 | 3400 ± 500 | 11600 ± 900 | 2600 ± 1000 |
| 140 | 17100 ± 5100 | >14000 | 12700 ± 300 | >14000 |
| 141 | 11400 ± 1000 | 3700 ± 800 | 13700 ± 1900 | 1100 ± 140 |
| 142 | 90 ± 10 | 24 ± 7 | 6400 ± 1100 | 80 ± 6 |
| 143 | 43500 ± 11300 | 11400 ± 1800 | 56500 ± 20000 | 3600 ± 700 |
| 146 | 2500 ± 400 | 740 ± 280 | 13,000 ± 1200 | |
| 147 | >76000 | 26100 ± 12900 | >76000 | 43800 ± 3000 |
| 148 | 17100 ± 1100 | 6800 ± 1100 | 61000 ± 11600 | 6700 ± 1600 |
| 149 | 2900 ± 1000 | 1500 500 | 44600 ± 1400 | 4100 ± 900 |
| 150 | 9500 ± 1600 | 1400 ± 400 | 59000 ± 5500 | 10600 ± 800 |
| 151 | 7900 ± 400 | 4200 ± 1600 | 25500 ± 1200 | 6600 ± 2300 |
| 152 | | 6400 ± 1200 | 49000 ± 7700 | 9100 ± 100 |
| 153 | 8700 ± 2700 | 10900 ± 3400 | >90000 | 15800 ± 9600 |
| 154 | >70000 | 61300 ± 10000 | >70000 | 46,700 ± 13100 |
| 155 | 8200 ± 1200 | 3600 ± 400 | 17,000 ± 4000 | 9100 ± 1100 |
| 156 | 7200 ± 500 | 3100 ± 100 | 32,300 ± 9,400 | 5500 ± 1200 |
| 157 | >400,000 | >123,000 | >350,000 | 13100 ± 1600 |
| 158 | >175,000 | >175,000 | >200,000 | 61,000 ± 9,000 |
| 159 | 2700 ± 400 | 120 ± 10 | 13,700 ± 4,200 | <10 nM |
| 160 | 1900 ± 200 | 500 ± 200 | 52,400 ± 17,800 | 3200 ± 1100 |
| 161 | 2800 ± 500 | 3300 ± 700 | 13,800 ± 3,400 | >10,000 |
| 163 | 3500 ± 800 | 820 ± 40 | 18600 ± 800 | 910 ± 100 |
| 164 | | 70 ± 10 | 3500 ± 1600 | 130 ± 40 |
| 165 | 7700 ± 1100 | 290 ± 40 | 11000 ± 3300 | 11000 ± 1000 |
| 166 | 6500 ± 600 | 7200 ± 1900 | 6500 ± 2100 | 6000 ± 1500 |
| 167 | 14800 ± 2200 | | 18500 ± 2300 | |
| 169 | 7100 ± 600 | | 2300 ± 600 | |
| 177 | 7500 ± 800 | 1900 ± 800 | 73000 ± 5000 | 4100 ± 1300 |
| 178 | 21000 ± 4000 | 1000 ± 100 | 32000 ± 9000 | >8000 |
| 180 | 19900 ± 300 | >4000 | 5200 ± 1800 | 660 ± 50 |
| 182 | 99000 ± 12000 | 38000 ± 8200 | 39000 ± 7000 | 18700 ± 2700 |
| 183 | >120,000 | >275,000 | >120,000 | >235,000 |
| 184 | 800 ± 300 | 210 ± 20 | >100,000 | >10000 |
| 185 | 1700 ± 600 | 1900 ± 100 | | |
| 186 | 144000 ± 32000 | 70000 ± 16000 | 79000 ± 24000 | 48000 ± 2000 |
| 187 | 1300 ± 400 | 900 ± 200 | 3200 ± 800 | 3200 ± 700 |
| 189 | 8900 ± 2500 | 6100 ± 2600 | 41,000 ± 3700 | |
| 190 | 19,000 ± 4000 | >9,000 | 56,000 ± 2000 | >9,000 |
| 191 | >140,000 | 49,000 ± 13000 | >140,000 | 15000 ± 4000 |
| 192 | 1,600 ± 200 | 700 ± 100 | 8700 ± 1700 | 200 ± 30 |
| 193 | 1400 ± 400 | 2500 ± 600 | 48,000 ± 7000 | >11,000 |
| 195 | 1400 ± 200 | 390 ± 120 | 21,000 ± 6000 | 4300 ± 1200 |
| 196 | 840 ± 100 | 450 ± 120 | 80,000 ± 5000 | >9,200 |
| 197 | 950 ± 70 | 500 ± 100 | 9500 ± 400 | [1,300 ± 100 |
| 198 | 700 ± 100 | 2800 ± 600 | >8,200 | >82,000 |
| 199 | 4700 ± 600 | 2500 ± 1100 | >93,000 | >9,300 |
| 201 | 360 ± 110 | 260 ± 70 | 13,000 ± 1700 | 26,000 ± 7000 |
| 202 | 1200 ± 100 | 650 ± 100 | >62,000 | >6200 |
| 203 | 760 ± 170 | 940 ± 330 | 48,000 ± 6000 | >5500 |
| 204 | 220 ± 40 | 1600 ± 300 | 4100 ± 800 | 8600 ± 800 |
| 205 | 8400 ± 2200 | 1200 ± 400 | >185,000 | >2,600 |
| 206 | 610 ± 40 | 230 ± 20 | 20,000 ± 1000 | 8200 ± 200 |
| 207 | 570 ± 60 | 410 ± 60 | | |
| 208 | 1200 ± 100 | 930 ± 160 | 25,000 ± 3000 | |
| 209 | 3900 ± 1100 | 610 ± 100 | >90,000 | |
| 210 | 40,000 ± 4000 | 5500 ± 600 | | |
| 211 | 470 ± 120 | 430 ± 100 | 59,000 ± 9000 | |
| 212 | 80 ± 10 | 55 ± 5 | | |
| 213 | 2300 ± 700 | 1700 ± 700 | | |
| 214 | 2900 ± 800 | 360 ± 30 | | |
| 215 | 26,000 ± 3000 | 490 ± 120 | | |
| 216 | 460 ± 60 | 150 ± 40 | | |
| 217 | 2,200 ± 100 | 2,200 ± 100 | 43,000 ± 4,000 | >7,000 |
| 218 | 10,000 ± 3,000 | 600 ± 200 | 15,000 ± 6,000 | 600 ± 100 |
| 219 | >52,000 | >52,00 | >52,000 | >52,000 |
| 220 | 90 ± 10 | 130 ± 10 | 101,000 ± 18,000 | 40,000 ± 3,000 |
| 221 | >21,000 | 2,500 ± 200 | >21,000 | >21,000 |
| 222 | 5,000 ± 100 | 1,100 ± 100 | 9,300 ± 200 | 330 ± 60 |
| 223 | 20,000 ± 3,700 | 2,700 ± 300 | >185,000 | >55,000 |
| 224 | >200,000 | >130,000 | >200,000 | >130,000 |
| 225 | 47,000 ± 4,000 | 55,000 ± 11,000 | >350,000 | 33,000 ± 13,000 |
| 226 | >59,000 | >59,000 | >59,000 | >59,000 |
| 227 | >57,000 | 4,400 ± 700 | >57,000 | 16,000 ± 4,000 |
| 228 | >38,000 | >38,000 | 24,000 ± 3,000 | >38,000 |
| 229 | >56,000 | >2,000 | >56,000 | >2,000 |
| 230 | 620 ± 80 | 100 ± 10 | 38,000 ± 5,000 | 1,000 ± 200 |
| 231 | 1,500 ± 100 | 280 ± 10 | 14,000 ± 4,000 | |
| 232 | 700 ± 100 | 460 ± 60 | 42,000 ± 6,000 | 3,300 ± 600 |
| 233 | 3,200 ± 300 | 350 ± 80 | >150,000 | 2,400 ± 700 |
| 234 | 3,000 ± 300 | 1,100 ± 400 | 24,000 ± 6,000 | 9,000 ± 1,000 |
| 235 | 3,500 ± 400 | 2,200 ± 400 | 49,000 ± 6,000 | 6,500 ± 1,600 |
| 236 | 49,000 ± 11,000 | 29,000 ± 5,000 | 48,000 ± 10,000 | |
| 237 | 1,200 ± 300 | 730 ± 140 | 22,000 ± 1,000 | 6,600 ± 900 |
| 238 | 780 ± 190 | 57 ± 8 | 23,000 ± 2,000 | 4,700 ± 1,200 |
| 239 | 420 ± 60 | 70 ± 20 | 39,000 ± 3,000 | 28,000 ± 4,000 |
| 240 | 2,900 ± 100 | 1,300 ± 200 | >24,000 | 1,300 ± 100 |
| 241 | 560 ± 90 | 110 ± 20 | >28,000 | 18,000 ± 4,000 |
| 242 | 2,400 ± 400 | 580 ± 150 | 18,000 ± 2,000 | 2,900 ± 600 |
| 243 | 2,200 ± 500 | 670 ± 240 | 64,000 ± 10,000 | 26,000 ± 6,000 |
| 244 | 1,600 ± 400 | 150 ± 10 | 87,000 ± 11,000 | 35,000 ± 7,000 |
| 245 | 3,400 ± 1000 | 440 ± 90 | 79,000 ± 7,000 | 14,000 ± 1,700 |
| 246 | 2,800 ± 260 | 1,900 ± 450 | 14,000 ± 2,000 | 6,200 ± 1,300 |
| 247 | 6,100 ± 2,000 | 1,200 ± 250 | 10,000 ± 1,400 | 7,100 ± 1,700 |
| 248 | 830 ± 100 | 200 ± 25 | 23,000 ± 1,000 | 610 ± 120 |
| 249 | 4,100 ± 820 | 420 ± 100 | 18,000 ± 3,500 | 19,000 ± 3,800 |
| 250 | 99,000 ± 21,000 | 137,000 ± 14,000 | >275,000 | 137,000 ± 10,000 |
| 251 | 128,000 ± 4,000 | 51,000 ± 1,000 | >275,000 | 82,000 ± 8,000 |
| 252 | >380,000 | 33,000 ± 3,000 | >380,000 | >380,000 |
| 253 | >380,000 | >38,000 | >380,000 | >380,000 |
| 254 | 2,700 ± 800 | 1,100 ± 100 | 43,000 ± 6,000 | >65,000 |
| 255 | 2,900 ± 500 | 55 ± 2 | 119,000 ± 15,000 | 99,000 ± 4,000 |
| 256 | 1,500 ± 200 | 880 ± 200 | 7,500 ± 800 | 7,100 ± 300 |
| 257 | 2,800 ± 600 | 320 ± 30 | 25,000 ± 2,000 | 26,000 ± 3,000 |
| 258 | >45,000 | >45,000 | >45,000 | >45,000 |
| 259 | 16000 ± 3000 | 2400 ± 200 | >85,000 | 4700 ± 400 |
| 260 | 1600 ± 500 | 150 ± 20 | >64,000 | 19000 ± 4500 |
| 261 | 6300 ± 1100 | 1000 ± 150 | 64000 ± 2000 | 38000 ± 2100 |
| 262 | 8700 ± 1300 | 3900 ± 570 | 287000 ± 14000 | 73000 ± 17000 |
| 263 | 2000 ± 300 | 1400 ± 200 | 124000 ± 18000 | 39000 ± 7000 |
| 264 | 1400 ± 100 | 76 ± 17 | >85,000 | 54000 ± 20000 |
| 265 | 810 ± 20 | 8 ± 1 | 1100 ± 200 | 250 ± 80 |
| 266 | 140 ± 20 | 70 ± 18 | 56000 ± 15000 | 32000 ± 7000 |
| 267 | 900 ± 160 | 160 ± 20 | >90,000 | 28000 ± 8000 |
| 268 | 2100 ± 200 | 330 ± 90 | 54,000 ± 16000 | >8,000 |
| 269 | 11000 ± 3000 | 850 ± 320 | 52000 ± 4000 | >7,000 |
| 270 | 8000 ± 1500 | 1300 ± 100 | >84,000 | 7100 ± 700 |
| 271 | 1700 ± 200 | 200 ± 90 | >93,000 | >9,300 |
| 272 | >46,000 | >4,700 | >47,000 | >4,700 |
| 273 | 30000 ± 5000 | >1,500 | >45,000 | >4,500 |
| 274 | 39000 ± 3000 | 1200 ± 300 | >46,000 | >4,500 |
| 275 | 1500 ± 300 | 370 ± 40 | >62,000 | >6,200 |
| 276 | 1500 ± 200 | 760 ± 100 | >61,000 | >6,100 |
| 277 | 760 ± 70 | 190 ± 20 | 31,000 ± 6000 | 9,800 ± 1000 |
| 278 | 1000 ± 100 | 270 ± 10 | >94000 | >9,400 |
| 279 | 1700 ± 400 | 190 ± 20 | >90000 | >9,000 |
| 280 | 2400 ± 800 | <80 | >83000 | >2,800 |
| 281 | 1800 ± 700 | 170 ± 10 | 27000 ± 2000 | 5000 ± 700 |
| 282 | 680 ± 60 | 110 ± 10 | >85000 | >8,500 |
| 283 | 2900 ± 1200 | 300 ± 20 | 40000 ± 4000 | >9,300 |
| 284 | 13,600 (N = 2) | 340 ± 20 | | >8,800 |
| 285 | 3800 ± 1100 | 310 ± 20 | 84000 ± 9000 | 2000 ± 100 |
| 286 | 48000 ± 10000 | 6300 ± 200 | 51000 ± 1700 | >8,800 |
| 287 | 455000 ± 22000 | 1100 ± 100 | 567000 ± 17000 | 4700 ± 400 |
| 288 | 1800 ± 600 | 150 ± 20 | 11000 ± 3200 | −9,000 |
| 289 | 51 ± 4 | 530 ± 150 | >290000 | >8,800 |
| 294 | 960 ± 170 | | | |
| 295 | 200 ± 44 | | | |
| 296 | 250 (N = 2) | | | |
| 297 | 2200 (N = 1) | | | |
| 298 | >7000 | | | |

TALE 14
The IUPAC name of Illudofulvene Analogs.

| | Illudofulvene | |
|---|---|---|
| Entry # | Analog | IUPAC Name of Illudofulvene Analog |
| 1 | 001 | (R)-6′-hydroxy-2′,4′,6′-trimethylspiro[cyclopropane-1,5′-inden]- |
| | | 7′(6′H)-one |
| 2 | 002 | (6′R)-6′-hydroxy-3′-(hydroxymethyl)-2′,4′,6′-trimethylspiro |
| | | [cyclopropane-1,5′-inden]-7′(6′H)-one |
| 3 | 003 | (6′R,6′′′R)-3′,3′′′-methylenebis(6′-hydroxy-2′,4′,6′- |
| | | trimethylspiro[cyclopropane-1,5′-inden]-7′(6′H)-one) |
| 4 | 004 | (R)-3′-bromo-6′-hydroxy-2′,4′,6′-trimethylspiro[cyclopropane-1,5′- |
| | | inden]-7′(6′H)-one |
| 5 | 005 | (R)-6′-hydroxy-3′-iodo-2′,4′,6′-trimethylspiro[cyclopropane-1,5′- |
| | | inden]-7′(6′H)-one |
| 6 | 006 | (R)-6′-hydroxy-3′-(4-hydroxybenzyl)-2′,4′,6′- |
| | | trimethylspiro[cyclopropane-1,5′-inden]-7′(6′H)-one |
| 7 | 007 | (R)-6′-hydroxy-3′-(4-methoxybenzyl)-2′,4′,6′- |
| | | trimethylspiro[cyclopropane-1,5′-inden]-7′(6′H)-one |
| 8 | 008 | (R)-((6′-hydroxy-2′,4′,6′-trimethyl-7′-oxo-6′,7′-dihydrospiro |
| | | [cyclopropane-1,5′-inden]-3′-yl)methoxy)methyl acetate |
| 9 | 009 | (R)-6′-hydroxy-3′-(3-hydroxypropyl)-2′,4′,6′- |
| | | trimethylspiro[cyclopropane-1,5′-inden]-7′(6′H)-one |
| 10 | 010 | (R)-3-(6′-hydroxy-2′,4′,6′-trimethyl-7′-oxo-6′,7′- |
| | | dihydrospiro[cyclopropane-1,5′-inden]-3′-yl)propanal |
| 11 | 011 | (R)-6′-hydroxy-2′,4′,6′-trimethyl-7′-oxo-6′,7′- |
| | | dihydrospiro[cyclopropane-1,5′-indene]-3′-carbaldehyde |
| 12 | 012 | (R)-6′-hydroxy-2′,4′,6′-trimethyl-3′-nitrospiro[cyclopropane-1,5′- |
| | | inden]-7′(6′H)-one |
| 13 | 013 | 4-hydroxy-5-(((R)-6′-hydroxy-2′,4′,6′-trimethyl-7′-oxo-6′,7′- |
| | | dihydrospiro[cyclopropane-1,5′-inden]-3′-yl)methyl)cyclohexane- |
| | | 1,3-dicarbaldehyde |
| 14 | 014 | (4a′S,7′R,9b′S)-7′-hydroxy-4a′,7′,9′-trimethyl-4a′,9b′-dihydro-4′H- |
| | | spiro[cyclopropane-1,8′-indeno[1,2-d][1,3]dioxin]-6′(7′H)-one |
| 15 | 015 | (R)-3′-(hydroxymethyl)-2′,4′,6′-trimethyl-7′-oxo-6′,7′- |
| | | dihydrospiro[cyclopropane-1,5′-inden]-6′-yl acetate |
| 16 | 016 | (R)-3′-(ethoxymethyl)-6′-hydroxy-2′,4′,6′- |
| | | trimethylspiro[cyclopropane-1,5′-inden]-7′(6′H)-one |
| 17 | 017 | (6′R,6′′′R)-3′,3′′′-(oxybis(methylene))bis(6′-hydroxy-2′,4′,6′- |
| | | trimethylspiro[cyclopropane-1,5′-inden]-7′(6′H)-one) |
| 18 | 018 | (R)-6′-hydroxy-2′,4′,6′-trimethyl-3′-((((2R,3S,4R,5R,6S)-3,4,5- |
| | | trihydroxy-6-(hydroxymethyl)tetrahydro-2H-pyran-2- |
| | | yl)oxy)methyl)spiro[cyclopropane-1,5′-inden]-7′(6′H)-one |
| 19 | 019 | (6′R)-3′-((2,3-dihydroxypropoxy)methyl)-6′-hydroxy-2′,4′,6′- |
| | | trimethylspiro[cyclopropane-1,5′-inden]-7′(6′H)-one |
| 20 | 020 | (R)-3′-((2-bromoethoxy)methyl)-6′-hydroxy-2′,4′,6′- |
| | | trimethylspiro[cyclopropane-1,5′-inden]-7′(6′H)-one |
| 21 | 021 | (R)-6′-hydroxy-3′-(((2-methoxypropan-2-yl)oxy)methyl)-2′,4′,6′- |
| | | trimethylspiro[cyclopropane-1,5′-inden]-7′(6′H)-one |
| 22 | 022 | (R)-6′-hydroxy-3′-((2-hydroxyethoxy)methyl)-2′,4′,6′- |
| | | trimethylspiro[cyclopropane-1,5′-inden]-7′(6′H)-one |
| 23 | 023 | (R)-6′-hydroxy-3′-(((4-hydroxyphenyl)thio)methyl)-2′,4′,6′- |
| | | trimethylspiro[cyclopropane-1,5′-inden]-7′(6′H)-one |
| 24 | 024 | (R)-3′-((benzylthio)methyl)-6′-hydroxy-2′,4′,6′- |
| | | trimethylspiro[cyclopropane-1,5′-inden]-7′(6′H)-one |
| 25 | 025 | methyl (R)-2-(((6′-hydroxy-2′,4′,6′-trimethyl-7′-oxo-6′,7′- |
| | | dihydrospiro[cyclopropane-1,5′-inden]-3′-yl)methyl)thio)acetate |
| 26 | 026 | (R)-6′-hydroxy-2′,4′,6′-trimethyl-3′-((p-tolylthio)methyl) |
| | | spiro[cyclopropane-1,5′-inden]-7′(6′H)-one |
| 27 | 027 | (R)-(6′-hydroxy-2′,4′,6′-trimethyl-7′-oxo-6′,7′-dihydrospiro |
| | | [cyclopropane-1,5′-inden]-3′-yl)methyl phenyl carbonate |
| 28 | 028 | (R)-(6′-hydroxy-2′,4′,6′-trimethyl-7′-oxo-6′,7′- |
| | | dihydrospiro[cyclopropane-1,5′-inden]-3′-yl)methyl benzoate |
| 29 | 029 | (R)-2-(((6′-hydroxy-2′,4′,6′-trimethyl-7′-oxo-6′,7′- |
| | | dihydrospiro[cyclopropane-1,5′-inden]-3′-yl)methyl)thio)acetic acid |
| 30 | 030 | methyl (R)-2-(((6′-hydroxy-1′4(2-methoxy-2-oxoethyl)thio)- |
| | | 2′,4′,6′-trimethyl-7′-oxo-6′,7′-dihydrospiro[cyclopropane-1,5′- |
| | | inden]-3′-yl)methyl)thio)acetate |
| 31 | 031 | methyl 2-((((6′R)-6′,7a′-dihydroxy-1′4(2-methoxy-2- |
| | | oxoethyl)thio)-2′,4′,6′-trimethyl-7′-oxo-1′,6′,7′,7a′- |
| | | tetrahydrospiro[cyclopropane-1,5′-inden]-3′-yl)methyl)thio)acetate |
| 32 | 032 | (6′R)-3′-(((2,3-dihydroxypropyl)thio)methyl)-6′-hydroxy-2′,4′,6′- |
| | | trimethylspiro[cyclopropane-1,5′-inden]-7′(6′H)-one |
| 33 | 033 | 7′-methyl-4′H-dispiro[cyclopropane-1,6′-indene-5′,2″- |
| | | [1,3]dioxolan1-4′-one |
| 34 | 034 | (R)-(6′-hydroxy-2′,4′,6′-trimethyl-7′-oxo-6′,7′- |
| | | dihydrospiro[cyclopropane-1,5′-inden]-3′-yl)methyl acetate |
| 35 | 035 | 6′-hydroxy-4′-methylspiro[cyclopropane-1,5′-inden]-7′(6′H)-one |
| 36 | 036 | (R)-3′-((1H-imidazol-1-yl)methyl)-6′-hydroxy-2′,4′,6′- |
| | | trimethylspiro[cyclopropane-1,5′-inden]-7′(6′H)-one |
| 37 | 037 | 1-carboxy-2-((((R)-6′-hydroxy-2′,4′,6′-trimethyl-7′-oxo-6′,7′- |
| | | dihydrospiro[cyclopropane-1,5′-inden]-3′-yl)methyl)thio)ethan-1- |
| | | aminium |
| 38 | 038 | (R)-3-(6′-hydroxy-2′,4′,6′-trimethyl-7′-oxo-6′,7′- |
| | | dihydrospiro[cyclopropane-1,5′-inden]-3′-yl)propanoic acid |
| 39 | 039 | (R)-3′-(3,3-dimethoxypropyl)-6′-hydroxy-2′,4′,6′- |
| | | trimethylspiro[cyclopropane-1,5′-inden]-7′(6′H)-one |
| 40 | 040 | (R)-3′-(3,3-diethoxypropyl)-6′-hydroxy-2′,4′,6′- |
| | | trimethylspiro[cyclopropane-1,5′-inden]-7′(6′H)-one |
| 41 | 041 | (R,Z)-3-(6′-hydroxy-2′,4′,6′-trimethyl-7′-oxo-6′,7′- |
| | | dihydrospiro[cyclopropane-1,5′-inden]-3′-yl)acrylaldehyde |
| 42 | 042 | (R)-3′-(hydroxymethyl)-4′,6′-dimethyl-6′-((triethylsilyl)oxy) |
| | | spiro[cyclopropane-1,5′-inden]-7′(6′H)-one |
| 43 | 043 | (R)-6′-hydroxy-2′,4′,6′-trimethyl-3′-(((triethylsilyl)oxy)methyl) |
| | | spiro[cyclopropane-1,5′-inden]-7′(6′H)-one |
| 44 | 044 | (R)-2′,4′,6′-trimethyl-6′-((triethylsilyl)oxy)-3′-(((triethylsilyl) |
| | | oxy)methyl)spiro[cyclopropane-1,5′-inden]-7′(6′H)-one |
| 45 | 045 | methyl 2-((7-hydroxy-5-(2-hydroxyethyl)-3-(hydroxymethyl)- |
| | | 2,4,6-trimethyl-1H-inden-1-yl)thio)acetate |
| 46 | 046 | (R)-3-(6′-hydroxy-2′,4′,6′-trimethyl-7′-oxo-6′,7′- |
| | | dihydrospiro[cyclopropane-1,5′-inden]-3′-yl)propyl acetate |
| 47 | 047 | (6′R)-3′-(2-(1,7-dihydroxy-2,4,6-trimethyl-1H-inden-5-yl)ethyl)-6′- |
| | | hydroxy-2′,4′,6′-trimethylspiro[cyclopropane-1,5′-inden]-7′(6′H)-one |
| 48 | 048 | (R)-6′-hydroxy-2′,4′,6′-trimethyl-1′-(p-tolylthio)-3′-((p-tolylthio) |
| | | methyl)spiro[cyclopropane-1,5′-inden]-7′(6′H)-one |
| 49 | 049 | (R)-6′-hydroxy-2′,4′,6′-trimethyl-3′-(p-tolylthio)spiro[cyclopropane- |
| | | 1,5′-inden]-7′(6′H)-one |
| 50 | 050 | (R)-6′-hydroxy-2′,4′,6′-trimethyl-1′,3′-bis(p-tolylthio)spiro |
| | | [cyclopropane-1,5′-inden]-7′(6′H)-one |
| 51 | 051 | (R)-2-(2-(((6′-hydroxy-2′,4′,6′-trimethyl-7′-oxo-6′,7′- |
| | | dihydrospiro[cyclopropane-1,5′-inden]-3′-yl)methyl)thio)acetoxy) |
| | | ethyl 2-mercaptoacetate |
| 52 | 052 | ethane-1,2-diyl bis(2-((((R)-6′-hydroxy-2′,4′,6′-trimethyl-7′-oxo- |
| | | 6′,7′-dihydrospiro[cyclopropane-1,5′-inden]-3′-yl)methyl)thio)acetate) |
| 53 | 053 | (R)-3′-((2-(2-bromoethoxy)ethoxy)methyl)-6′-hydroxy-2′,4′,6′- |
| | | trimethylspiro[cyclopropane-1,5′-inden]-7′(6′H)-one |
| 54 | 054 | (R)-6′-hydroxy-1′-(((R)-6′-hydroxy-2′,4′,6′-trimethyl-7′-oxo-6′,7′- |
| | | dihydrospiro[cyclopropane-1,5′-inden]-3′-yl)methyl)-3′- |
| | | (hydroxymethyl)-2′,4′,6′-trimethylspiro[cyclopropane-1,5′-inden]- |
| | | 7′(6′H)-one |
| 55 | 055 | 5-(2-hydroxyethyl)-1-((4-hydroxyphenyethio)-3-(((4- |
| | | hydroxyphenyl)thio)methyl)-2,4,6-trimethyl-1H-inden-7-ol |
| 56 | 056 | (R)-6′-hydroxy-3′((4-hydroxyphenyl)thio)-2′,4′,6′- |
| | | trimethylspiro[cyclopropane-1,5′-inden]-7′(6′H)-one |
| 57 | 057 | (R)-6′-hydroxy-1′((4-hydroxyphenyl)thio)-2′,4′,6′- |
| | | trimethylspiro[cyclopropane-1,5′-inden]-7′(6′H)-one |
| 58 | 058 | (R)-6′-hydroxy-1′,3′-bis((4-hydroxyphenyl)thio)-2′,4′,6′- |
| | | trimethylspiro[cyclopropane-1,5′-inden]-7′(6′H)-one |
| 59 | 059 | (6′S,7′R)-4′-methyl-6′-((triethylsilyl)oxy)-6′,7′-dihydrospiro |
| | | [cyclopropane-1,5′-inden]-7′-ol |
| 60 | 060 | (R)-7′-methyl-4′H-dispiro[cyclopropane-1,6′-indene-5′,2″- |
| | | [1,3]dioxolan1-4′-ol |
| 61 | 061 | (S)-4′-methyl-6′-((triethylsilyl)oxy)spiro[cyclopropane-1,5′- |
| | | inden]-7′(6′H)-one |
| 62 | 062 | (R)-6′-hydroxy-2′-(hydroxymethyl)-4′,6′-dimethylspiro |
| | | [cyclopropane-1,5′-inden]-7′(6′H)-one |
| 63 | 063 | (R)-6′-hydroxy-2′,3′-bis(hydroxymethyl)-4′,6′- |
| | | dimethylspiro[cyclopropane-1,5′-inden]-7′(6′H)-one |
| 64 | 064 | N-acetyl-S-(((R)-6′-hydroxy-2′,4′,6′-trimethyl-7′-oxo-6′,7′- |
| | | dihydrospiro[cyclopropane-1,5′-inden]-3′-yl)methyl)-L-cysteine |
| 65 | 065 | (R)-2-acetamido-3-((((R)-6′-hydroxy-4′,6′-dimethyl-7′-oxo-6′,7′- |
| | | dihydrospiro[cyclopropane-1,5′-inden]-3′-yl)methyl)thio)-N-((S)-1- |
| | | phenylethyl)propanamide |
| 66 | 066 | (S)-2-acetamido-3-((((R)-6′-hydroxy-2′,4′,6′-trimethyl-7′-oxo-6′,7′- |
| | | dihydrospiro[cyclopropane-1,5′-inden]-3′-yl)methyl)thio)-N-((S)-1- |
| | | phenylethyl)propanamide |
| 67 | 067 | 4-methyl-2,3-dihydro-5H-indeno[5,6-b]furan-5-one |
| 68 | 068 | 5-hydroxy-6-(2-hydroxyethyl)-7-methyl-1H-inden-1-one |
| 69 | 069 | 5-(2-hydroxyethoxy)-6-(2-hydroxyethyl)-7-methyl-1H-inden-1-one |
| 70 | 070 | (3a′R,4′R)-4′-hydroxy-7′-methyl-3a′,4′-dihydro-1′H- |
| | | dispiro[cyclopropane-1,6′-indene-5′,2″-[1,3]dioxolan]-1′-one |
| 71 | 071 | (3a′R,4′R)-7′-methyl-4′-((triethylsilyl)oxy)-3a′,4′-dihydro-1′H- |
| | | dispiro[cyclopropane-1,6′-indene-5′,2″-[1,3]dioxolan]-1′-one |
| 72 | 072 | (7′R,7a′R)-7′-hydroxy-4′-methyl-7′,7a′-dihydrospiro[cyclopropane- |
| | | 1,5′-indene]-3′,6′-dione |
| 73 | 073 | (7′R,7a′R)-4′-methyl-7′-((triethylsilyl)oxy)-7′,7a′-dihydrospiro |
| | | [cyclopropane-1,5′-indene]-3,6′-dione |
| 74 | 074 | (6′R)-3′-((((2,2-dimethyl-1,3-dioxolan-4-yl)methypthio)methyl)-6′- |
| | | hydroxy-2′,4′,6′-trimethylspiro[cyclopropane-1,5′-inden]-7′(6′H)-one |
| 75 | 075 | (R)-(6′-hydroxy-4′,6′-dimethyl-7′-oxo-6′,7′- |
| | | dihydrospiro[cyclopropane-1,5′-inden]-2′-yl)methyl acetate |
| 76 | 076 | (R)-(6′-hydroxy-4′,6′-dimethyl-7′-oxo-6′,7′-dihydrospiro |
| | | [cyclopropane-1,5′-indenel-2′,3′-diyl)bis(methylene)diacetate |
| 77 | 077 | (R)-(6′-hydroxy-3′-(hydroxymethyl)-4′,6′-dimethyl-7′-oxo-6′,7′- |
| | | dihydrospiro[cyclopropane-1,5′-inden]-2′-yl)methyl acetate |
| 78 | 078 | (R)-(6′-hydroxy-2′-(hydroxymethyl)-4′,6′-dimethyl-7′-oxo-6′,7′- |
| | | dihydrospiro[cyclopropane-1,5′-inden]-3′-yl)methyl acetate |
| 79 | 079 | (R)-6′-hydroxy-2′-(methoxymethyl)-4′,6′-dimethylspiro |
| | | [cyclopropane-1,5′-inden]-7′(6′H)-one |
| 80 | 080 | (R)-6′-hydroxy-3′-(methoxymethyl)-2′,4′,6′-trimethylspiro |
| | | [cyclopropane-1,5′-inden]-7′(6′H)-one |
| 81 | 081 | (R)-6′-hydroxy-2′-(hydroxymethyl)-3′-(methoxymethyl)-4′,6′- |
| | | dimethylspiro[cyclopropane-1,5′-inden]-7′(6′H)-one |
| 82 | 082 | (R)-6′-hydroxy-2′,3′-bis(methoxymethyl)-4′,6′- |
| | | dimethylspiro[cyclopropane-1,5′-inden]-7′(6′H)-one |
| 83 | 083 | (R)-2-acetamido-3-((((R)-6′-hydroxy-2′,4′,6′-trimethyl-7′-oxo-6′,7′- |
| | | dihydrospiro[cyclopropane-1,5′-inden]-3′-yl)methyl)thio)-N-((S)-1- |
| | | ((2-(((S)-4-methyl-1-oxopentan-2-yl)amino)-2-oxoethyl)amino)-1- |
| | | oxo-3-phenylpropan-2-yl)propanamide |
| 84 | 084 | (S)-2-((R)-2-acetamido-3-((((R)-6′-hydroxy-2′,4′,6′-trimethyl-7′- |
| | | oxo-6′,7′-dihydrospiro[cyclopropane-1,5′-inden]-3′-yl)methyl) |
| | | thio)propanamido)-4-methyl-N-(2-oxo-2-(((R)-1-oxo-3- |
| | | phenylpropan-2-yl)amino)ethyl)pentanamide |
| 85 | 085 | (S)-2-(((R)-2-acetamido-3-4((R)-6′-hydroxy-2′,4′,6′-trimethyl-7′- |
| | | oxo-6′,7′-dihydrospiro[cyclopropane-1,5′-inden]-3′- |
| | | yl)methyl)thio)propanamido)-4-methyl-N-((S)-4-methyl-1-oxo-1- |
| | | (((R)-1-oxo-3-phenylpropan-2-yl)amino)pentan-2-yl)pentanamide |
| 86 | 086 | (R)-2-acetamido-3-((((R)-6′-hydroxy-2′,4′,6′-trimethyl-7′-oxo-6′,7′- |
| | | dihydrospiro[cyclopropane-1,5′-inden-3-yl)methyl)thio)-N-(2- |
| | | oxo-2-(((R)-1-oxo-3-phenylpropan-2-yl)amino)ethyl)propanamide |
| 87 | 087 | (S)-2-((R)-2-acetamido-3-((((R)-6′-hydroxy-2′,4′,6′-trimethyl-7′- |
| | | oxo-6′,7′-dihydrospiro[cyclopropane-1,5′-inden-3′-yl)methyl) |
| | | thio)propanamido)-4-methyl-N-((R)-4-methyl-1-(((S)-4- |
| | | methyl-1-oxopentan-2-yl)amino)-1-oxopentan-2-yl)pentanamide |
| 88 | 088 | (R)-(6′-acetoxy-2′,4′,6′-trimethyl-7′-oxo-6′,7′- |
| | | dihydrospiro[cyclopropane-1,5′-inden-3′-yl)methyl acetate |
| 89 | 089 | N5-((R)-1-((carboxymethyl)amino)-3-((((R)-6′-hydroxy-2′,4′,6′- |
| | | trimethyl-7′-oxo-6′,7′-dihydrospiro[cyclopropane-1,5′-inden-3′- |
| | | yl)methyl)thio)-1-oxopropan-2-yl)-D-glutamine |
| 90 | 090 | (R)-2′-(hydroxymethyl)-4′,6′-dimethyl-7′-oxo-6′,7′- |
| | | dihydrospiro[cyclopropane-1,5′-inden]-6′-yl acetate |
| 91 | 091 | (R)-3-(6′-hydroxy-2′,4′,6′-trimethyl-7′-oxo-6′,7′- |
| | | dihydrospiro[cyclopropane-1,5′-inden-3′-yl)propanoic acid |
| 92 | 092 | (R)-6′-hydroxy-3′-(3-methoxypropyl)-2′,4′,6′- |
| | | trimethylspiro[cyclopropane-1,5′-inden]-7′(6′H)-one |
| 93 | 093 | (R)-3′-(3,3-diethoxypropyl)-6′-hydroxy-2′,4′,6′- |
| | | trimethylspiro[cyclopropane-1,5′-inden]-7′(6′H)-one |
| 94 | 094 | (R)-3-(6′-hydroxy-2′,4′,6′-trimethyl-7′-oxo-6′,7′- |
| | | dihydrospiro[cyclopropane-1,5′-inden-3′-yl)propyl acetate |
| 95 | 095 | (R)-6′-hydroxy-3′-(3-methoxypropyl)-2′,4′,6′- |
| | | trimethylspiro[cyclopropane-1,5′-inden]-7′(6′H)-one |
| 96 | 096 | (R)-6′-hydroxy-2′,4′,6′-trimethyl-3′-(((1-methyl-1H-imidazol-2- |
| | | yl)thio)methyl)spiro[cyclopropane-1,5′-inden-7′(6′H)-one |
| 97 | 097 | S-(((R)-6′-hydroxy-2′,4′,6′-trimethyl-7′-oxo-6′,7′- |
| | | dihydrospiro[cyclopropane-1,5′-inden-3′-yl)methyl)homocysteine |
| 98 | 098 | ((S)-3-((((R)-6′-hydroxy-2′,4′,6′-trimethyl-7′-oxo-6′,7′- |
| | | dihydrospiro[cyclopropane-1,5′-inden-3′-yl)methyl)thio)-2- |
| | | methylpropanoyl)proline |
| 99 | 099 | (2′S,6′R)-6′-hydroxy-2′-(hydroxymethyl)-2′,4′,6′- |
| | | trimethylspiro[cyclopropane-1,5′-indene-3′,7′(2′H,6′H)-dione |
| 100 | 100 | S-(((R)-6′-hydroxy-2′,4′,6′-trimethyl-7′-oxo-6′,7′-dihydrospiro |
| | | [cyclopropane-1,5′-inden-3′-yl)methyl)-N-S-(((R)-6′-hydroxy- |
| | | 2′,4′,6′-trimethyl-7′-oxo-6′,7′-dihydrospiro[cyclopropane-1,5′- |
| | | inden-3′-yl)methyl)cysteinyl-L-asparaginylglycyl-L-arginylcysteine |
| 101 | 101 | S-(((R)-6′-hydroxy-2′,4′,6′-trimethyl-7′-oxo-6′,7′-dihydrospiro |
| | | [cyclopropane-1,5′-inden-3′-yl)methyl)-N-S-(((R)-6′-hydroxy- |
| | | 2′,4′,6′-trimethyl-7′-oxo-6′,7′-dihydrospiro[cyclopropane-1,5′-inden- |
| | | 3′-yl)methyl)cysteinyl-L-arginylglycyl-L-asparaginylcysteine |
| 102 | 102 | S-(((R)-6′-hydroxy-2′,4′,6′-trimethyl-7′-oxo-6′,7′- |
| | | dihydrospiro[cyclopropane-1,5′-inden-3′-yl)methyl)cysteinyl-L- |
| | | asparaginylglycyl-L-arginylcysteine |
| 103 | 103 | (R)-(6′-acetoxy-2′-(hydroxymethyl)-4′,6′-dimethyl-7′-oxo-6′,7′- |
| | | dihydrospiro[cyclopropane-1,5′-inden-3′-yl)methyl acetate |
| 104 | 104 | (R)-8′-hydroxy-6′,8′-dimethyl-1,5′-dihydrospiro[cyclopropane-1,7′- |
| | | indeno[1,2-][1,3]dioxepin]-9′(8′H)-one |
| 105 | 105 | (E)-2-((2R,45)-4-hydroxy-2-((1R,25)-2-hydroxy-4,4- |
| | | dimethylcyclopentyl)-2-methylcyclobutylidene)propanal |
| 106 | 106 | 5-(((3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl)oxy)-5- |
| | | oxopentanoic acid |
| 107 | 107 | (3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl ((13S)-13-methyl- |
| | | 17-oxo-7,8,9,11,12,13,14,15,16,17-decahydro-6H- |
| | | cyclopenta[a]phenanthren-3-yl)glutarate |
| 108 | 108 | (13S)-17-hydroxy-13-methyl-7,8,9,11,12,13,14,15,16,17- |
| | | decahydro-6H-cyclopenta[a]phenanthren-3-yl 3-((R)-6′-hydroxy- |
| | | 2′,4′,6′-trimethyl-7′-oxo-6′,7′-dihydrospiro[cyclopropane-1,5′- |
| | | inden]-3′-yl)propanoate |
| 109 | 109 | (13S)-17-hydroxy-13-methyl-7,8,9,11,12,13,14,15,16,17- |
| | | decahydro-6H-cyclopenta[a]phenanthren-3-yl ((3′S,6′R)-6′- |
| | | hydroxy-2′,2′,4′,6′-tetramethyl-7′-oxo-2′,3′,6′,7′- |
| | | tetrahydrospiro[cyclopropane-1,5′-inden]-3′-yl)glutarate |
| 110 | 110 | (13S)-13-methyl-17-oxo-7,8,9,11,12,13,14,15,16,17-decahydro- |
| | | 6H-cyclopenta[a]phenanthren-3-yl 3-((R)-6′-hydroxy-2′,4′,6′- |
| | | trimethyl-7′-oxo-6′,7′-dihydrospiro[cyclopropane-1,5′-inden]-3′- |
| | | yl)propanoate |
| 111 | 111 | (10R,13S)-10,13-dimethyl-3-oxo- |
| | | 2,3,6,7,8,9,10,11,12,13,14,15,16,17-tetradecahydro-1H- |
| | | cyclopenta[a]phenanthren-17-yl 3-(6′-hydroxy-2′,4′-dimethyl-7′- |
| | | oxo-6′,7′-dihydrospiro[cyclopropane-1,5′-inden]-3′-yl)propanoate |
| 112 | 112 | (13S)-10,13-dimethyl-17-oxohexadecahydro-1H- |
| | | cyclopenta[a]phenanthren-3-yl 3-((R)-6′-hydroxy-2′,4′,6′-trimethyl- |
| | | 7′-oxo-6′,7′-dihydrospiro[cyclopropane-1,5′-inden]-3′-yl)propanoate |
| 113 | 113 | (R)-3′-(but-3-en-1-yl)-6′-hydroxy-2′,4′,6′- |
| | | trimethylspiro[cyclopropane-1,5′-inden]-7′(6′H)-one |
| 114 | 114 | (6′R)-6′-hydroxy-2′,4′,6′-trimethyl-3′-(2-(oxiran-2- |
| | | yl)ethyl)spiro[cyclopropane-1,5′-inden]-7′(6′H)-one |
| 115 | 115 | (R)-3-(6′-hydroxy-2′,4′,6′-trimethyl-7′-oxo-6′,7′- |
| | | dihydrospiro[cyclopropane-1,5′-inden]-3′-yl)propanal oxime |
| 116 | 116 | (R)-3′-(tert-butoxymethyl)-6′-hydroxy-2′,4′,6′- |
| | | trimethylspiro[cyclopropane-1,5′-inden]-7′(6′H)-one |
| 117 | 117 | 5-(((2′S,6′R)-3′-((4-carboxybutanoyl)oxy)-6′-hydroxy-2′,4′,6′- |
| | | trimethyl-7′-oxo-2′,3′,6′,7′-tetrahydrospiro[cyclopropane-1,5′- |
| | | inden]-2′-yl)methoxy)-5-oxopentanoic acid |
| 118 | 118 | 5-(((2′S,6′R)-2′-(((3,5-dinitrobenzoyl)oxy)methyl)-6′-hydroxy- |
| | | 2′,4′,6′-trimethyl-7′-oxo-2′,3′,6′,7′-tetrahydrospiro[cyclopropane- |
| | | 1,5′-inden]-3′-yl)oxy)-5-oxopentanoic acid |
| 119 | 119 | (6′R)-3′-(3,4-dihydroxybutyl)-6′-hydroxy-2′,4′,6′- |
| | | trimethylspiro[cyclopropane-1,5′-inden]-7′(6′H)-one |
| 120 | 120 | (R)-6′-hydroxy-3′-(3-((34(S)-6′-hydroxy-2′,4′,6′-trimethyl-7′-oxo- |
| | | 6′,7′-dihydrospiro[cyclopropane-1,5′-inden]-3′-yl)propylidene) |
| | | hydrazineylidene)propyl)-2′,4′,6′-trimethylspiro[cyclopropane- |
| | | 1,5′-inden]-7′(6′H)-one |
| 121 | 121 | (R)-2-(3-(6′-hydroxy-2′,4′,6′-trimethyl-7′-oxo-6′,7′-dihydrospiro |
| | | [cyclopropane-1,5′-inden]-3′-yl)propylidene)hydrazine-1- |
| | | carboxamide |
| 122 | 122 | (R)-6′-hydroxy-2′,4′,6′-trimethyl-3′-(3-(2-phenylhydrazineylidene) |
| | | propyl)spiro[cyclopropane-1,5′-inden]-7′(6′H)-one |
| 123 | 123 | (R)-N′-(3-(6′-hydroxy-2′,4′,6′-trimethyl-7′-oxo-6′,7′- |
| | | dihydrospiro[cyclopropane-1,5′-inden]-3′-yl)propylidene)-4- |
| | | methylbenzenesulfonohydrazide |
| 124 | 124 | (R)-3-(6′-hydroxy-2′,4′,6′-trimethyl-7′-oxo-6′,7′- |
| | | dihydrospiro[cyclopropane-1,5′-inden]-3′-yl)propanal O-acetyl |
| | | oxime |
| 125 | 125 | (R)-3′-(3-(2-(2,4-dinitrophenyl)hydrazineylidene)propyl)-6′- |
| | | hydroxy-2′,4′,6′-trimethylspiro[cyclopropane-1,5′-inden]-7′(6′H)- |
| | | one |
| 126 | 126 | (R)-6′-hydroxy-2′,4′,6′-trimethyl-7′-oxo-6′,7′- |
| | | dihydrospiro[cyclopropane-1,5′-indenel-3′-carbaldehyde oxime |
| 127 | 127 | 2-hydroxy-4(((R)-6′-hydroxy-2′,4′,6′-trimethyl-7′-oxo-6′,7′- |
| | | dihydrospiro[cyclopropane-1,5′-inden]-3′-yl)butanenitrile |
| 128 | 128 | (6′R)-6′-hydroxy-3′-(3-hydroxybutyl)-2′,4′,6′- |
| | | trimethylspiro[cyclopropane-1,5′-inden]-7′(6′H)-one |
| 129 | 129 | (6′R)-2′,4′,6′-trimethyl-6′,7′-dihydrospiro[cyclopropane-1,5′- |
| | | indenel-6′,7′-diol |
| 130 | 130 | (R)-6′-hydroxy-3′-(3-(hydroxyamino)propyl)-2′,4′,6′- |
| | | trimethylspiro[cyclopropane-1,5′-inden]-7′(6′H)-one |
| 131 | 131 | (R)-N-benzyl-3-(6′-hydroxy-2′,4′,6′-trimethyl-7′-oxo-6′,7′- |
| | | dihydrospiro[cyclopropane-1,5′-inden]-3′-yl)propanamide |
| 132 | 133 | (E)-7-(chloromethylene)-5-hydroxy-5,9-dimethylspiro[3.5]non-8- |
| | | en-6-one |
| 133 | 134 | (E)-6-(chloromethylene)-4-hydroxy-4,8-dimethylspiro[2.5]oct-7- |
| | | en-5-one |
| 134 | 135 | ((2′S,6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl 4- |
| | | nitrobenzoate |
| 135 | 136 | ((2′S,6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl 4-(N- |
| | | acetoxyacetamido)benzoate |
| 136 | 137 | ((2′S,6′R)-6′-hydroxy-2′,4′,6′-trimethyl-3′,7′-dioxo-2′,3′,6′,7′- |
| | | tetrahydrospiro[cyclopropane-1,5′-inden]-2′-yl)methyl 4- |
| | | nitrobenzoate |
| 137 | 138 | ((2′S,6′R)-6′-hydroxy-2′,4′,6′-trimethyl-3′,7′-dioxo-2′,3′,6′,7′- |
| | | tetrahydrospiro[cyclopropane-1,5′-inden]-2′-yl)methyl 4-(N- |
| | | acetoxyacetamido)benzoate |
| 138 | 139 | (2′S,6′R)-6′-hydroxy-2′,4′,6′-trimethyl-2′-(((4- |
| | | nitrobenzoyl)oxy)methyl)-7′-oxo-2′,3′,6′,7′- |
| | | tetrahydrospiro[cyclopropane-1,5′-inden]-3′-yl 4-nitrobenzoate |
| 139 | 140 | ((2′S,6′R)-3′-((4-(N-acetoxyacetamido)benzoyl)oxy)-6′-hydroxy- |
| | | 2′,4′,6′-trimethyl-7′-oxo-2′,3′,6′,7′-tetrahydrospiro[cyclopropane- |
| | | 1,5′-inden]-2′-yl)methyl 4-(N-acetoxyacetamido)benzoate |
| 140 | 141 | dimethyl (5′R)-4′,5′-dihydroxy-5′,7′,9′-trimethyl-4′,5′-dihydro-1′H- |
| | | spiro[cyclopropane-1,6′-[1,3a]ethenoindenel-2′,3′-dicarboxylate |
| 141 | 142 | dimethyl (5′R)-5′-hydroxy-5′,7′,9′-trimethyl-4′-oxo-4′,5′-dihydro- |
| | | 1′H-spiro[cyclopropane-1,6′-[1,3a]ethenoindenel-2′,3′-dicarboxylate |
| 142 | 143 | (R)-6′-hydroxy-1′,2′,4′,6′-tetramethylspiro[cyclopropane-1,5′- |
| | | inden]-7′(6′H)-one |
| 143 | 144 | (R)-2-((2′-ethyl-6′-hydroxy-4′,6′-dimethyl-7′-oxo-6′,7′- |
| | | dihydrospiro[cyclopropane-1,5′-inden]-3′-yl)methoxy)ethyl acetate |
| 144 | 145 | (R)-54(6′-hydroxy-2′,4′,6′-trimethyl-7′-oxo-6′,7′- |
| | | dihydrospiro[cyclopropane-1,5′-inden]-3′-yl)methoxy)-5- |
| | | oxopentanoic acid |
| 145 | 146 | (R)-4-(6′-hydroxy-2′,4′,6′-trimethyl-7′-oxo-6′,7′- |
| | | dihydrospiro[cyclopropane-1,5′-inden]-3′-yl)butanenitrile |
| 146 | 147 | (R)-3′-((benzo[d]thiazol-2-ylthio)methyl)-6′-hydroxy-2′,4′,6′- |
| | | trimethylspiro[cyclopropane-1,5′-inden]-7′(6′H)-one |
| 147 | 148 | (R)-3′-((benzo[d]oxazol-2-ylthio)methyl)-6′-hydroxy-2′,4′,6′- |
| | | trimethylspiro[cyclopropane-1,5′-inden]-7′(6′H)-one |
| 148 | 149 | (R)-6′-hydroxy-2′,4′,6′-trimethyl-3′-(((1-methyl-1H-tetrazol-5- |
| | | yl)thio)methyl)spiro[cyclopropane-1,5′-inden]-7′(6′H)-one |
| 149 | 150 | (R)-6′-hydroxy-2′,4′,6′-trimethyl-3′-(((5-methyl-1H-benzo[d] |
| | | imidazol-2-yl)thio)methyl)spiro[cyclopropane-1,5′-inden]-7′(6′H)-one |
| 150 | 151 | (R)-6′-hydroxy-2′,4′,6′-trimethyl-3′-(((1-phenyl-1H-tetrazol-5- |
| | | yl)thio)methyl)spiro[cyclopropane-1,5′-inden]-7′(6′H)-one |
| 151 | 152 | (R)-6′-hydroxy-2′,4′,6′-trimethyl-3′-(((5-nitro-1H-benzo[d]imidazol- |
| | | 2-yl)thio)methyl)spiro[cyclopropane-1,5′-inden]-7′(6′H)-one |
| 152 | 153 | (R)-3′-(((1H-1,2,4-triazol-3-yl)thio)methyl)-6′-hydroxy-2′,4′,6′- |
| | | trimethylspiro[cyclopropane-1,5′-inden]-7′(6′H)-one |
| 153 | 154 | (R)-6′-hydroxy-3′-(((4-hydroxypteridin-2-yl)thio)methyl)-2′,4′,6′- |
| | | trimethylspiro[cyclopropane-1,5′-inden]-7′(6′H)-one |
| 154 | 155 | (R)-6′-hydroxy-3′-(((1-(4-hydroxyphenyl)-1H-tetrazol-5- |
| | | yl)thio)methyl)-2′,4′,6′-trimethylspiro[cyclopropane-1,5′-inden]- |
| | | 7′(6′H)-one |
| 155 | 156 | (R)-4-(5-(((6′-hydroxy-2′,4′,6′-trimethyl-7′-oxo-6′,7′- |
| | | dihydrospiro[cyclopropane-1,5′-inden]-3′-yl)methyl)thio)-1H- |
| | | tetrazol-1-yl)phenyl acetate |
| 156 | 157 | 7′-methyl-4′H-dispiro[cyclobutane-1,6′-indene-5′,2″-[1,3]dioxolan]- |
| | | 4′-one |
| 157 | 158 | 5-hydroxy-2,2,6,8a-tetramethyl-2,3,3a,8,8a,8b-hexahydro-1H- |
| | | cyclobuta[d]cyclopenta[b]oxepin-7(5H)-one |
| 158 | 159 | ((6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl acetate |
| 159 | 160 | 5-(((6′R)-2′-(acetoxymethyl)-6′-hydroxy-2′,4′,6′-trimethyl-7′-oxo- |
| | | 2′,3′,6′,7′-tetrahydrospiro[cyclopropane-1,5′-inden]-3′-yl)oxy)-5- |
| | | oxopentanoic acid |
| 160 | 161 | ((6′R)-6′-hydroxy-2′,4′,6′-trimethyl-3′,7′-dioxo-2′,3′,6′,7′- |
| | | tetrahydrospiro[cyclopropane-1,5′-inden]-2′-yl)methyl acetate |
| 161 | 162 | 5-(((6′R)-6′-hydroxy-2′,4′,6′-trimethyl-3′,7′-dioxo-2′,3′,6′,7′- |
| | | tetrahydrospiro[cyclopropane-1,5′-inden]-2′-yl)methoxy)-5- |
| | | oxopentanoic acid |
| 162 | 163 | (6′R)-2′-(acetoxymethyl)-6′-hydroxy-2′,4′,6′-trimethyl-7′-oxo- |
| | | 2′,3′,6′,7′-tetrahydrospiro[cyclopropane-1,5′-inden]-3′-yl 2- |
| | | chloroacetate |
| 163 | 164 | (R)-(6′-hydroxy-2′,4′,6′-trimethyl-7′-oxo-6′,7′- |
| | | dihydrospiro[cyclopropane-1,5′-inden]-3′-yl)methyl 2-chloroacetate |
| 164 | 165 | (R)-(6′-hydroxy-2′,4′,6′-trimethyl-7′-oxo-6′,7′-dihydrospiro |
| | | [cyclopropane-1,5′-inden]-3′-yl)methyl 2-morpholinoacetate |
| 165 | 166 | (6′R)-2′-(acetoxymethyl)-6′-hydroxy-2′,4′,6′-trimethyl-7′-oxo- |
| | | 2′,3′,6′,7′-tetrahydrospiro[cyclopropane-1,5′-inden]-3′-yl methyl |
| | | glutarate |
| 166 | 167 | (6′R)-6′-hydroxy-2′-(hydroxymethyl)-2′,4′,6′-trimethyl-7′-oxo- |
| | | 2′,3′,6′,7′-tetrahydrospiro[cyclopropane-1,5′-inden]-3′-yl methyl |
| | | glutarate |
| 167 | 168 | ((6′R)-6′-hydroxy-2′,4′,6′-trimethyl-3′,7′-dioxo-2′,3′,6′,7′- |
| | | tetrahydrospiro[cyclopropane-1,5′-inden]-2′-yl)methyl methyl |
| | | glutarate |
| 168 | 169 | ((6′R)-6′-hydroxy-2′,4′,6′-trimethyl-3′,7′-dioxo-2′,3′,6′,7′- |
| | | tetrahydrospiro[cyclopropane-1,5′-inden]-2′-yl)methyl 2- |
| | | chloroacetate |
| 169 | 170 | 5-(2-hydroxyethyl)-3-(hydroxymethyl)-2,4,6-trimethyl-1H-inden- |
| | | 7-ol |
| 170 | 171 | 6-(2-hydroxyethyl)-2,5,7-trimethyl-1-methylene-1H-inden-4-ol |
| 171 | 172 | 6-ethyl-2,5,7-trimethyl-1-methylene-1H-inden-4-ol |
| 172 | 173 | 2-(4-hydroxy-2,5,7-trimethyl-1-methylene-1H-inden-6-yl)ethyl |
| | | acetate |
| 173 | 174 | 5-(2-hydroxyethyl)-3-(hydroxymethyl)-2,4,6-trimethyl-1H-indene- |
| | | 1,7-diol |
| 174 | 175 | (2S,3S,4R,5S,6R)-2-(acetoxymethyl)-6-(((R)-6′-hydroxy-2′,4′,6′- |
| | | trimethyl-7′-oxo-6′,7′-dihydrospiro[cyclopropane-1,5′-inden]-3′- |
| | | yl)methoxy)tetrahydro-2H-pyran-3,4,5-triyltriacetate |
| 175 | 176 | 3-((R)-6′-hydroxy-2′,4′,6′-trimethyl-7′-oxo-6′,7′- |
| | | dihydrospiro[cyclopropane-1,5′-inden]-3′-yl)propyl leucinate |
| 176 | 177 | (R)-5-(3-(6′-hydroxy-2′,4′,6′-trimethyl-7′-oxo-6′,7′- |
| | | dihydrospiro[cyclopropane-1,5′-inden]-3′-yl)propoxy)-5- |
| | | oxopentanoic acid |
| 177 | 178 | (R)-4-(3-(6′-hydroxy-2′,4′,6′-trimethyl-7′-oxo-6′,7′- |
| | | dihydrospiro[cyclopropane-1,5′-inden]-3′-yl)propoxy)-4- |
| | | oxobutanoic acid |
| 178 | 179 | (R)-3-(6′-hydroxy-2′,4′,6′-trimethyl-7′-oxo-6′,7′- |
| | | dihydrospiro[cyclopropane-1,5′-inden]-3′-yl)propyl glycinate |
| 179 | 180 | (1a′R,3′R,7′S,7a′R)-3′,7′-dihydroxy-1a′,3′,6′,6′-tetramethyl-6′,7′- |
| | | dihydro-1a′H-spiro[cyclopropane-1,2′-indeno[3a,4-b]oxiren]- |
| | | 4′(3′H)-one |
| 180 | 181 | ((1a′R,3′R,6′S,7′S,7a′R)-3′,7′-dihydroxy-1a′,3′,6′-trimethyl-4′-oxo- |
| | | 3′,4′,6′,7′-tetrahydro-1a′H-spiro[cyclopropane-1,2′-indeno[3a,4- |
| | | b]oxiren]-6′-yl)methyl acetate |
| 181 | 182 | (2′R,7′S,7a′S)-2′-chloro-7′-hydroxy-2′,4′-dimethyl-1′,2′,7′,7a′- |
| | | tetrahydrospiro[cyclopropane-1,5′-indene]-3′,6′-dione |
| 182 | 183 | (2′S,7′S,7a′S)-7′-hydroxy-2′-isopropoxy-2′,4′-dimethyl-1′,2′,7′,7a′- |
| | | tetrahydrospiro[cyclopropane-1,5′-indene]-3′,6′-dione |
| 183 | 184 | (R)-1-hydroxy-1-((6′-hydroxy-2′,4′,6′-trimethyl-7′-oxo-6′,7′- |
| | | dihydrospiro[cyclopropane-1,5′-inden]-3′-yl)methyl)urea |
| 184 | 185 | (S)-6′-hydroxy-2′,4′,6′-trimethylspiro[cyclopropane-1,5′-inden]- |
| | | 7′(6′H)-one |
| 185 | 186 | (6′S,7′R)-6′,7′-dihydroxy-2′,4′,6′-trimethyl-7′,7a′- |
| | | dihydrospiro[cyclopropane-1,5′-inden]-3′(6′H)-one |
| 186 | 187 | (S)-6′-hydroxy-3′-(hydroxymethyl)-2′,4′,6′- |
| | | trimethylspiro[cyclopropane-1,5′-inden]-7′(6′H)-one |
| 187 | 188 | (6′S,6S)-3′,3-methylenebis(6′-hydroxy-2′,4′,6′- |
| | | trimethylspiro[cyclopropane-1,5′-inden]-7′(6′H)-one) |
| 188 | 189 | (R)-14(6′-hydroxy-2′,4′,6′-trimethyl-7′-oxo-6′,7′- |
| | | dihydrospiro[cyclopropane-1,5′-inden]-3′-yl)methyl)-1H-pyrrole- |
| | | 2,5-dione |
| 189 | 190 | (R)-1-(3-(6′-hydroxy-2′,4′,6′-trimethyl-7′-oxo-6′,7′- |
| | | dihydrospiro[cyclopropane-1,5′-inden]-3′-yl)propyl)-1H-pyrrole- |
| | | 2,5-dione |
| 190 | 191 | 6′-hydroxy-4′,6′-dimethylspiro[cyclobutane-1,5′-inden]-7′(6′H)-one |
| 191 | 192 | (R)-2-((6′-hydroxy-2′,4′,6′-trimethyl-7′-oxo-6′,7′- |
| | | dihydrospiro[cyclopropane-1,5′-inden]-3′-yl)methyl)isoindoline- |
| | | 1,3-dione |
| 192 | 193 | (R)-3′-(azidomethyl)-6′-hydroxy-2′,4′,6′- |
| | | trimethylspiro[cyclopropane-1,5′-inden]-7′(6′H)-one |
| 193 | 194 | (R)-3′-(((R)-3′-(azidomethyl)-6′-hydroxy-2′,4′,6′-trimethyl-7′-oxo- |
| | | 6′,7′-dihydrospiro[cyclopropane-1,5′-inden]-1′-yl)methyl)-6′- |
| | | hydroxy-2′,4′,6′-trimethylspiro[cyclopropane-1,5′-inden]-7′(6′H)- |
| | | one |
| 194 | 195 | (R)-3′-(3-azidopropyl)-6′-hydroxy-4′,6′- |
| | | dimethylspiro[cyclopropane-1,5′-inden]-7′(6′H)-one |
| 195 | 196 | 3-((R)-6′-hydroxy-2′,4′,6′-trimethyl-7′-oxo-6′,7′- |
| | | dihydrospiro[cyclopropane-1,5′-inden]-3′-yl)propyl L-prolinate |
| 196 | 197 | (R)-2-(3-(6′-hydroxy-2′,4′,6′-trimethyl-7′-oxo-6′,7′-dihydrospiro |
| | | [cyclopropane-1,5′-inden]-3′-yl)propyl)isoindoline-1,3-dione |
| 197 | 198 | (R)-6′-hydroxy-2′,4′,6′-trimethyl-3′-((4- |
| | | nitrophenoxy)methyl)spiro[cyclopropane-1,5′-inden]-7′(6′H)-one |
| 198 | 199 | (R)-6′-hydroxy-2′,4′,6′-trimethyl-3′- |
| | | (phenoxymethyl)spiro[cyclopropane-1,5′-inden]-7′(6′H)-one |
| 199 | 200 | (R)-6′-hydroxy-3′-(2-hydroxybenzyl)-2′,4′,6′- |
| | | trimethylspiro[cyclopropane-1,5′-inden]-7′(6′H)-one |
| 200 | 201 | (R)-N-(3-(6′-hydroxy-2′,4′,6′-trimethyl-7′-oxo-6′,7′- |
| | | dihydrospiro[cyclopropane-1,5′-inden]-3′-yl)propyl)acetamide |
| 201 | 202 | (S)-N-(3-((R)-6′-hydroxy-2′,4′,6′-trimethyl-7′-oxo-6′,7′-dihydrospiro |
| | | [cyclopropane-1,5′-inden]-3′-yl)propyl)pyrrolidine-2-carboxamide |
| 202 | 203 | 3-((R)-6′-hydroxy-2′,4′,6′-trimethyl-7′-oxo-6′,7′- |
| | | dihydrospiro[cyclopropane-1,5′-inden]-3′-yl)propyl L-seryl-L- |
| | | prolinate |
| 203 | 204 | 2′-(((tert-butyldimethylsilyl)oxy)methyl)-3′,6′-dihydroxy-2′,4′,6′- |
| | | trimethyl-2′,3′-dihydrospiro[cyclopropane-1,5′-inden]-7′(6′H)-one |
| 204 | 205 | (2′R,3′S,6′R)-3′-amino-2′-(((tert-butyldimethylsilyl)oxy)methyl)-6′- |
| | | hydroxy-2′,4′,6′-trimethyl-2′,3′-dihydrospiro[cyclopropane-1,5′- |
| | | inden]-7′(6′H)-one |
| 205 | 206 | (2′R,3′S,6′R)-3′-amino-6′-hydroxy-2′-(hydroxymethyl)-2′,4′,6′- |
| | | trimethyl-2′,3′-dihydrospiro[cyclopropane-1,5′-inden]-7′(6′H)-one |
| 206 | 207 | (S)-2-amino-N-(3-((R)-6′-hydroxy-2′,4′,6′-trimethyl-7′-oxo-6′,7′- |
| | | dihydrospiro[cyclopropane-1,5′-inden]-3′-yl)propyl)-4- |
| | | methylpentanamide |
| 207 | 208 | 3-((R)-6′-hydroxy-2′,4′,6′-trimethyl-7′-oxo-6′,7′-dihydrospiro |
| | | [cyclopropane-1,5′-inden]-3′-yl)propyl (tert-butoxycarbonyl)- |
| | | L-seryl-L-prolinate |
| 208 | 209 | 3-((R)-6′-hydroxy-2′,4′,6′-trimethyl-7′-oxo-6′,7′- |
| | | dihydrospiro[cyclopropane-1,5′-inden]-3′-yl)propyl L-seryl-L- |
| | | seryl-L-prolinate |
| 209 | 210 | 3-((R)-6′-hydroxy-2′,4′,6′-trimethyl-7′-oxo-6′,7′- |
| | | dihydrospiro[cyclopropane-1,5′-inden]-3′-yl)propyl ((S)-2-((S)-2- |
| | | ((S)-2-((2S,4R)-1-acetyl-4-hydroxypyrrolidine-2-carboxamido)-3- |
| | | hydroxypropanamido)-3-hydroxypropanamido)-2- |
| | | cyclohexylacetyl)-L-glutaminyl-L-seryl-L-seryl-L-prolinate |
| 210 | 211 | (R)-3′-(3-aminopropyl)-6′-hydroxy-2′,4′,6′- |
| | | trimethylspiro[cyclopropane-1,5′-inden]-7′(6′H)-one |
| 211 | 212 | (3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl L-prolinate |
| 212 | 213 | (2′S,3′R,6′R)-2′-(((tert-butyldimethylsilyl)oxy)methyl)-6′-hydroxy- |
| | | 2′,4′,6′-trimethyl-7′-oxo-2′,3′,6′,7′-tetrahydrospiro[cyclopropane- |
| | | 1,5′-inden]-3′-yl L-prolinate |
| 213 | 214 | (2′S,3′R,6′R)-2′-(((tert-butyldimethylsilyl)oxy)methyl)-6′-hydroxy- |
| | | 2′,4′,6′-trimethyl-7′-oxo-2′,3′,6′,7′-tetrahydrospiro[cyclopropane- |
| | | 1,5′-inden]-3′-yl L-seryl-L-seryl-L-prolinate |
| 214 | 215 | (2′S,3′R,6′R)-2′-(((tert-butyldimethylsilyl)oxy)methyl)-6′-hydroxy- |
| | | 2′,4′,6′-trimethyl-7′-oxo-2′,3′,6′,7′-tetrahydrospiro[cyclopropane- |
| | | 1,5′-inden]-3′-yl ((S)-2-((S)-2-((S)-2-((25,4R)-1-acetyl-4- |
| | | hydroxypyrrolidine-2-carboxamido)-3-hydroxypropanamido)-3- |
| | | hydroxypropanamido)-2-cyclohexylacetyl)-L-glutaminyl-L-seryl- |
| | | L-seryl-L-prolinate |
| 215 | 216 | (3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl ((S)-2-((S)-2-((S)-2- |
| | | ((2S,4R)-1-acetyl-4-hydroxypyrrolidine-2-carboxamido)-3- |
| | | hydroxypropanamido)-3-hydroxypropanamido)-2- |
| | | cyclohexylacetyl)-L-glutaminyl-L-seryl-L-seryl-L-prolinate |
| 216 | 217 | (R)-(6′-hydroxy-2′,4′,6′-trimethyl-7′-oxo-6′,7′- |
| | | dihydrospiro[cyclopropane-1,5′-inden]-3′-yl)methyl 6-(2,5-dioxo- |
| | | 2,5-dihydro-1H-pyrrol-1-yl)hexanoate |
| 217 | 218 | (3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl 6-(2,5-dioxo-2,5- |
| | | dihydro-1H-pyrrol-1-yl)hexanoate |
| 218 | 219 | (2′S,3′R,6′R)-2′-(((tert-butyldimethylsilyl)oxy)methyl)-6′-hydroxy- |
| | | 2′,4′,6′-trimethyl-7′-oxo-2′,3′,6′,7′-tetrahydrospiro[cyclopropane- |
| | | 1,5′-inden]-3′-yl 4-(fluorosulfonyl)benzoate |
| 219 | 220 | (R)-1-acetoxy-1-((6′-hydroxy-2′,4′,6′-trimethyl-7′-oxo-6′,7′- |
| | | dihydrospiro[cyclopropane-1,5′-inden]-3′-yl)methyl)urea |
| 220 | 221 | (S)-2-((3S,6S,9S,12S,15S)-3-((1H-imidazol-4-yl)methyl)-12-(4- |
| | | aminobutyl)-6,9-bis(hydroxymethyl)-15-isobutyl-1-morpholino- |
| | | 1,4,7,10,13-pentaoxo-2,5,8,11,14-pentaazahexadecan-16-amido)- |
| | | N1-((S)-1-((3-((R)-6′-hydroxy-2′,4′,6′-trimethyl-7′-oxo-6′,7′- |
| | | dihydrospiro[cyclopropane-1,5′-inden]-3′-yl)propyl)amino)-4- |
| | | methyl-1-oxopentan-2-yl)pentanediamide |
| 221 | 222 | (3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl 4- |
| | | (fluorosulfonyl)benzoate |
| 222 | 223 | 3-((R)-6′-hydroxy-2′,4′,6′-trimethyl-7′-oxo-6′,7′- |
| | | dihydrospiro[cyclopropane-1,5′-inden]-3′-yl)propyl ((4R,7S,13S)- |
| | | 13-(2-amino-2-oxoethyl)-7-(3-guanidinopropyl)-6,9,12,15- |
| | | tetraoxo-1,2-dithia-5,8,14-triazacycloheptadecane-4- |
| | | carbonyl)glycinate |
| 223 | 224 | (R,E)-6′-hydroxy-2′,4′,6′-trimethylspiro[cyclopropane-1,5′-inden]- |
| | | 7′(6′H)-one oxime |
| 224 | 225 | (2′R,3′R,6′R,E)-2′,3′,6′-trihydroxy-2′,4′,6′-trimethyl-2′,3′- |
| | | dihydrospiro[cyclopropane-1,5′-inden]-7′(6′H)-one oxime |
| 225 | 226 | (R)-(6′-hydroxy-2′,4′,6′-trimethyl-7′-oxo-6′,7′-dihydrospiro |
| | | [cyclopropane-1,5′-inden]-3′-yl)methyl (E)-octadec-9-enoate |
| 226 | 227 | (R)-3-(6′-hydroxy-2′,4′,6′-trimethyl-7′-oxo-6′,7′-dihydrospiro |
| | | [cyclopropane-1,5′-inden]-3′-yl)propyl (E)-octadec-9-enoate |
| 227 | 228 | (2′S,6′R)-6′-hydroxy-2′,4′,6′-trimethyl-2′-((((E)-octadec-9- |
| | | enoyl)oxy)methyl)-7′-oxo-2′,3′,6′,7′-tetrahydrospiro[cyclopropane- |
| | | 1,5′-inden]-3′-yl (E)-octadec-9-enoate |
| 228 | 229 | (R,E)-N-(3-(6′-hydroxy-2′,4′,6′-trimethyl-7′-oxo-6′,7′- |
| | | dihydrospiro[cyclopropane-1,5′-inden]-3′-yl)propyl)octadec-9- |
| | | enamide |
| 229 | 230 | N-((3′R,6′R)-6′-hydroxy-2′,2′,4′,6′-tetramethyl-7′-oxo-2′,3′,6′,7′- |
| | | tetrahydrospiro[cyclopropane-1,5′-inden]-3′-yl)methanesulfonamide |
| 230 | 231 | N-((3′R,6′R)-6′-hydroxy-2′,2′,4′,6′-tetramethyl-7′-oxo-2′,3′,6′,7′- |
| | | tetrahydrospiro[cyclopropane-1,5′-inden]-3′-yl)-4- |
| | | methylbenzenesulfonamide |
| 231 | 232 | (R)-3-(6′-hydroxy-2′,4′,6′-trimethyl-7′-oxo-6′,7′- |
| | | dihydrospiro[cyclopropane-1,5′-inden]-3′-yl)propyl |
| | | hydroxycarbamate |
| 232 | 233 | ethyl (R)-hydroxy((6′-hydroxy-2′,4′,6′-trimethyl-7′-oxo-6′,7′- |
| | | dihydrospiro[cyclopropane-1,5′-inden]-3′-yl)methyl)carbamate |
| 233 | 234 | benzyl (R)-hydroxy((6′-hydroxy-2′,4′,6′-trimethyl-7′-oxo-6′,7′- |
| | | dihydrospiro[cyclopropane-1,5′-inden]-3′-yl)methyl)carbamate |
| 234 | 235 | tert-butyl (R)-hydroxy((6′-hydroxy-2′,4′,6′-trimethyl-7′-oxo-6′,7′- |
| | | dihydrospiro[cyclopropane-1,5′-inden]-3′-yl)methyl)carbamate |
| 235 | 236 | (R)-6′-hydroxy-3′-(hydroxymethyl)-1′,2′,4′,6′- |
| | | tetramethylspiro[cyclopropane-1,5′-inden]-7′(6′H)-one |
| 236 | 237 | (R)-3-(6′-hydroxy-2′,4′,6′-trimethyl-7′-oxo-6′,7′- |
| | | dihydrospiro[cyclopropane-1,5′-inden]-3′-yl)propyl (2- |
| | | bromoethyl)carbamate |
| 237 | 238 | (R)-3-(6′-hydroxy-2′,4′,6′-trimethyl-7′-oxo-6′,7′- |
| | | dihydrospiro[cyclopropane-1,5′-inden]-3′-yl)propyl (2- |
| | | chloroethyl)carbamate |
| 238 | 239 | (R)-3-(6′-hydroxy-2′,4′,6′-trimethyl-7′-oxo-6′,7′- |
| | | dihydrospiro[cyclopropane-1,5′-inden]-3′-yl)propyl (2- |
| | | hydroxyethyl)carbamate |
| 239 | 240 | (R)-3-(6′-hydroxy-2′,4′,6′-trimethyl-7′-oxo-6′,7′- |
| | | dihydrospiro[cyclopropane-1,5′-inden]-3′-yl)propyl |
| | | acetoxy(acetyl)carbamate |
| 240 | 241 | (R)-N-(3-(6′-hydroxy-2′,4′,6′-trimethyl-7′-oxo-6′,7′- |
| | | dihydrospiro[cyclopropane-1,5′-inden]-3′-yl)propyl) |
| | | methanesulfonamide |
| 241 | 242 | (R)-N-(3-(6′-hydroxy-2′,4′,6′-trimethyl-7′-oxo-6′,7′- |
| | | dihydrospiro[cyclopropane-1,5′-inden]-3′-yl)propyl)-4- |
| | | methylbenzenesulfonamide |
| 242 | 243 | (R)-3-(6′-hydroxy-2′,4′,6′-trimethyl-7′-oxo-6′,7′- |
| | | dihydrospiro[cyclopropane-1,5′-inden]-3′-yl)propyl (2- |
| | | fluoroethyl)carbamate |
| 243 | 244 | (R)-1-hydroxy-1-(3-(6′-hydroxy-2′,4′,6′-trimethyl-7′-oxo-6′,7′- |
| | | dihydrospiro[cyclopropane-1,5′-inden]-3′-yl)propyl)urea |
| | 245 | (R)-1-hydroxy-1-(3-(6′-hydroxy-2′,4′,6′-trimethyl-7′-oxo-6′,7′- |
| 244 | | dihydrospiro[cyclopropane-1,5′-inden]-3′-yl)propyl)thiourea |
| | 246 | (R)-3-(6′-hydroxy-2′,4′,6′-trimethyl-7′-oxo-6′,7′-dihydrospiro |
| 245 | | [cyclopropane-1,5′-inden]-3′-yl)propyl morpholine-4-carboxylate |
| 246 | 247 | (R)-3-(6′-hydroxy-2′,4′,6′-trimethyl-7′-oxo-6′,7′-dihydrospiro |
| | | [cyclopropane-1,5′-inden]-3′-yl)propyl morpholine-4-carboxylate |
| 247 | 248 | (R)-3-(6′-hydroxy-2′,4′,6′-trimethyl-7′-oxo-6′,7′- |
| | | dihydrospiro[cyclopropane-1,5′-inden]-3′-yl)propyl [1,4′- |
| | | bipiperidine]-1′-carboxylate |
| 248 | 249 | (R)-6′-hydroxy-2′-(hydroxymethyl)-3′,4′,6′- |
| | | trimethylspiro[cyclopropane-1,5′-inden]-7′(6′H)-one |
| 249 | 250 | ((1a′R,2′S,3′R,6′R,7a′S)-3′-acetoxy-6′-hydroxy-2′,4′,6′-trimethyl-7′- |
| | | oxo-1′,1a′,2′,3′,6′,7′-hexahydrospiro[cyclopropane-1,5′- |
| | | cyclopropa[c]inden]-2′-yl)methyl acetate |
| 250 | 251 | ((1a′S,2′S,3′R,6′R,7a′R)-3′-acetoxy-6′-hydroxy-2′,4′,6′-trimethyl-7′- |
| | | oxo-1′,1a′,2′,3′,6′,7′-hexahydrospiro[cyclopropane-1,5′- |
| | | cyclopropa[c]inden]-2′-yl)methyl acetate |
| 251 | 252 | (1a′R,3′S,6′R,7a′S)-3′,6′-dihydroxy-2′,2′,4′,6′-tetramethyl-1′,1a′,2′,3′- |
| | | tetrahydrospiro[cyclopropane-1,5′-cyclopropa[c]inden]-7′(6′H)-one |
| 252 | 253 | (1a′S,3′S,6′R,7a′R)-3′,6′-dihydroxy-2′,2′,4′,6′-tetramethyl-1′,1a′,2′,3′- |
| | | tetrahydrospiro[cyclopropane-1,5′-cyclopropa[c]inden]-7′(6′H)-one |
| 253 | 254 | (R)-3-(6′-hydroxy-2′,4′,6′-trimethyl-7′-oxo-6′,7′- |
| | | dihydrospiro[cyclopropane-1,5′-inden]-3′-yl)propyl 4- |
| | | sulfamoylbenzoate |
| 254 | 255 | (R)-3-(6′-hydroxy-2′,4′,6′-trimethyl-7′-oxo-6′,7′- |
| | | dihydrospiro[cyclopropane-1,5′-inden]-3′-yl)propyl sulfamate |
| 255 | 256 | 3-((R)-6′-hydroxy-2′,4′,6′-trimethyl-7′-oxo-6′,7′- |
| | | dihydrospiro[cyclopropane-1,5′-inden]-3′-yl)propyl 1-(3-((R)-6′- |
| | | hydroxy-2′,4′,6′-trimethyl-7′-oxo-6′,7′-dihydrospiro[cyclopropane- |
| | | 1,5′-inden]-3′-yl)propyl)-1H-1,2,3-triazole-4-carboxylate |
| 256 | 257 | 3-((R)-6′-hydroxy-2′,4′,6′-trimethyl-7′-oxo-6′,7′- |
| | | dihydrospiro[cyclopropane-1,5′-inden]-3′-yl)propyl 1-(((R)-6′- |
| | | hydroxy-2′,4′,6′-trimethyl-7′-oxo-6′,7′-dihydrospiro[cyclopropane- |
| | | 1,5′-inden]-3′-yl)methyl)-1H-1,2,3-triazole-4-carboxylate |
| 257 | 258 | (4-carboxy-4-(4-carboxy-4-((3-((R)-6′-hydroxy-2′,4′,6′-trimethyl-7′- |
| | | oxo-6′,7′-dihydrospiro[cyclopropane-1,5′-inden]-3′- |
| | | yl)propyl)amino)butanamido)butanoyl)glutamic acid |
| 258 | 259 | (R)-3′-((S)-2,2-dioxido-1,2,3-oxathiazinan-4-yl)-6′-hydroxy-2′,4′,6′- |
| | | trimethylspiro[cyclopropane-1,5′-inden]-7′(6′H)-one |
| 259 | 260 | (R)-N-(3-(6′-hydroxy-2′,4′,6′-trimethyl-7′-oxo-6′,7′- |
| | | dihydrospiro[cyclopropane-1,5′-inden]-3′-yl)propyl)-[1,4′- |
| | | bipiperidine]-l′-carboxamide |
| 260 | 261 | (R)-3-(6′-hydroxy-2′,4′,6′-trimethyl-7′-oxo-6′,7′- |
| | | dihydrospiro[cyclopropane-1,5′-inden]-3′-yl)propyl 1H-imidazole- |
| | | 1-carboxylate |
| 261 | 262 | methyl (R)-2-4(6′-hydroxy-2′,4′,6′-trimethyl-7′-oxo-6′,7′- |
| | | dihydrospiro[cyclopropane-1,5′-inden]-3′-yl)methyl)sulfonyl)acetate |
| 262 | 263 | methyl 2-((((R)-6′-hydroxy-2′,4′,6′-trimethyl-7′-oxo-6′,7′- |
| | | dihydrospiro[cyclopropane-1,5′-inden]-3′-yl)methyl)sulfinyl)acetate |
| 263 | 264 | N-[3-(6′-hydroxy-2′,6′-dimethyl-7′-oxo-6′,7′- |
| | | dihydrospiro[cyclopropane-1,5′-inden]-3′-yl)propyl]sulfuric |
| | | diamide |
| 264 | 265 | N-hydroxy-N-l3-(6′-hydroxy-2′,6′-dimethyl-7′-oxo-6′,7′- |
| | | dihydrospiro[cyclopropane-1,5′-inden]-3′-yl)propyl]sulfuric |
| | | diamide |
| 265 | 266 | N-[3-(6′-hydroxy-2′,6′-dimethyl-7′-oxo-6′,7′- |
| | | dihydrospiro[cyclopropane-1,5′-inden]-3′-yl)propyl]-N- |
| | | methoxysulfuric diamide |
| 266 | 267 | (R)-2-amino-N-((6′-hydroxy-2′,4′,6′-trimethyl-7′-oxo-6′,7′- |
| | | dihydrospiro[cyclopropane-1,5′-inden]-3′-yl)methyl)-N- |
| | | methoxyacetamide |
| 267 | 268 | (R)-2,2,2-trifluoro-N-(3-(6′-hydroxy-2′,4′,6′-trimethyl-7′-oxo-6′,7′- |
| | | dihydrospiro[cyclopropane-1,5′-inden]-3′-yl)propyl)acetamide |
| 268 | 269 | (R)-(6′-hydroxy-2′,4′,6′-trimethyl-7′-oxo-6′,7′- |
| | | dihydrospiro[cyclopropane-1,5′-inden]-3′-yl)methyl (4- |
| | | methoxyphenyl)sulfamate |
| 269 | 270 | (R)-3′-(aminomethyl)-6′-hydroxy-2′,4′,6′- |
| | | trimethylspiro[cyclopropane-1,5′-inden]-7′(6′H)-one |
| 270 | 271 | (R)-N-((6′-hydroxy-2′,4′,6′-trimethyl-7′-oxo-6′,7′-dihydrospiro |
| | | [cyclopropane-1,5′-inden]-3′-yl)methyl)methanesulfonamide |
| 271 | 272 | (5S,6S,7S)-3-(((3-((R)-6′-hydroxy-2′,4′,6′-trimethyl-7′-oxo-6′,7′- |
| | | dihydrospiro[cyclopropane-1,5′-inden]-3′-yl)propanoyl)oxy) |
| | | methyl)-8-oxo-7-(2-(thiophen-2-yl)acetamido)-5-thia-1- |
| | | azabicyclo[4.2.0]oct-2-ene-2-carboxylic acid 5-oxide |
| 272 | 273 | (5S,6S,7S)-3-((((3-((R)-6′-hydroxy-2′,4′,6′-trimethyl-7′-oxo-6′,7′- |
| | | dihydrospiro[cyclopropane-1,5′-inden]-3′- |
| | | yl)propyl)carbamoyl)oxy)methyl)-8-oxo-7-(2-(thiophen-2- |
| | | yl)acetamido)-5-thia-1-azabicyclo[4.2.0]oct-2-ene-2-carboxylic |
| | | acid 5-oxide |
| 273 | 274 | (6S,7S)-3-((((3-((R)-6′-hydroxy-2′,4′,6′-trimethyl-7′-oxo-6′,7′- |
| | | dihydrospiro[cyclopropane-1,5′-inden]-3′- |
| | | yl)propyl)carbamoyl)oxy)methyl)-8-oxo-7-(2-(thiophen-2- |
| | | yl)acetamido)-5-thia-1-azabicyclo[4.2.0]oct-2-ene-2-carboxylic |
| | | acid 5,5-dioxide |
| 274 | 275 | N-(((R)-6′-hydroxy-2′,4′,6′-trimethyl-7′-oxo-6′,7′- |
| | | dihydrospiro[cyclopropane-1,5′-inden]-3′-yl)methyl)pyrrolidine-2- |
| | | carboxamide |
| 275 | 276 | 2-amino-N-(((R)-6′-hydroxy-2′,4′,6′-trimethyl-7′-oxo-6′,7′- |
| | | dihydrospiro[cyclopropane-1,5′-inden]-3′-yl)methyl)-4- |
| | | methylpentanamide |
| 276 | 277 | (R)-1-hydroxy-3-((6′-hydroxy-2′,4′,6′-trimethyl-7′-oxo-6′,7′- |
| | | dihydrospiro[cyclopropane-1,5′-inden]-3′-yl)methyl)urea |
| 277 | 278 | (R)-14(6′-hydroxy-2′,4′,6′-trimethyl-7′-oxo-6′,7′- |
| | | dihydrospiro[cyclopropane-1,5′-inden]-3′-yl)methyl)-3- |
| | | methoxyurea |
| 278 | 279 | (R)-14(6′-hydroxy-2′,4′,6′-trimethyl-7′-oxo-6′,7′- |
| | | dihydrospiro[cyclopropane-1,5′-inden]-3-yl)methyl)-3-(2- |
| | | hydroxyethyl)urea |
| 279 | 280 | (R)-1-(3-(6′-hydroxy-2′,4′,6′-trimethyl-7′-oxo-6′,7′- |
| | | dihydrospiro[cyclopropane-1,5′-inden]-3-yl)propyl)-3-(2- |
| | | hydroxyethyl)urea |
| 280 | 281 | (R)-1-(2-chloroethyl)-3-(3-(6′-hydroxy-2′,4′,6′-trimethyl-7′-oxo- |
| | | 6,7′-dihydrospiro[cyclopropane-1,5′-inden]-3-yl)propyl)urea |
| 281 | 282 | (R)-1-(2-chloroethyl)-3-((6′-hydroxy-2′,4′,6′-trimethyl-7′-oxo-6′,7′- |
| | | dihydrospiro[cyclopropane-1,5′-inden]-3′-yl)methyl)urea |
| 282 | 283 | N-1(6′-hydroxy-2′,6′-dimethyl-7′-oxo-6′,7′- |
| | | dihydrospiro[cyclopropane-1,5′-inden]-3′-yl)methyl]sulfuric |
| | | diamide |
| 283 | 284 | (R)-3-((6′-hydroxy-2′,4′,6′-trimethyl-7′-oxo-6′,7′- |
| | | dihydrospiro[cyclopropane-1,5′-inden]-3-yl)methyl)pyrimidine- |
| | | 2,4(1H,3H)-dione |
| 284 | 285 | N-hydroxy-N-1(6′-hydroxy-2′,6′-dimethyl-7′-oxo-6′,7′- |
| | | dihydrospiro[cyclopropane-1,5′-inden]-3′-yl)methyl]sulfuric |
| | | diamide |
| 285 | 286 | (R)-5-fluoro-1-((6′-hydroxy-2′,4′,6′-trimethyl-7′-oxo-6′,7′- |
| | | dihydrospiro[cyclopropane-1,5′-inden]-3′-yl)methyl)pyrimidine- |
| | | 2,4(1H,3H)-dione |
| 286 | 287 | (R)-5-fluoro-3-((6′-hydroxy-2′,4′,6′-trimethyl-7′-oxo-6′,7′- |
| | | dihydrospiro[cyclopropane-1,5′-inden]-3′-yl)methyl)pyrimidine- |
| | | 2,4(1H,3H)-dione |
| 287 | 288 | (R)-1-hydroxy-3-(3-(6′-hydroxy-2′,4′,6′-trimethyl-7′-oxo-6′,7′- |
| | | dihydrospiro[cyclopropane-1,5′-inden]-3-yl)propyl)urea |
| 288 | 289 | (R)-1-((6′-hydroxy-2′,4′,6′-trimethyl-7′-oxo-6′,7′- |
| | | dihydrospiro[cyclopropane-1,5′-inden]-3′-yl)methyl)pyrimidine- |
| | | 2,4(1H,3H)-dione |
| 289 | 290 | ((S)-6′-hydroxy-2′,4′,6′-trimethyl-7′-oxo-6′,7′- |
| | | dihydrospiro[cyclopropane-1,5′-inden]-3′-yl)methyl 5-oxo-5-(((S)- |
| | | 1,2,3,10-tetramethoxy-9-oxo-5,6,7,9-tetrahydrobenzo[a]heptalen-7- |
| | | yl)amino)pentanoate |
| 290 | 291 | 3-((S)-6′-hydroxy-2′,4′,6′-trimethyl-7′-oxo-6′,7′- |
| | | dihydrospiro[cyclopropane-1,5′-inden]-3-yl)propyl 5-oxo-5-(((S)- |
| | | 1,2,3,10-tetramethoxy-9-oxo-5,6,7,9-tetrahydrobenzo[a]heptalen-7- |
| | | yl)amino)pentanoate |
| 291 | 292 | N1-(((S)-6′-hydroxy-2′,4′,6′-trimethyl-7′-oxo-6′,7′- |
| | | dihydrospiro[cyclopropane-1,5′-inden]-3-yl)methyl)-N5-((S)- |
| | | 1,2,3,10-tetramethoxy-9-oxo-5,6,7,9-tetrahydrobenzo[a]heptalen-7- |
| | | yl)glutaramide |
| 292 | 293 | 3-((S)-6′-hydroxy-2′,4′,6′-trimethyl-7′-oxo-6′,7′- |
| | | dihydrospiro[cyclopropane-1,5′-inden]-3-yl)-N-((S)-1,2,3,10- |
| | | tetramethoxy-9-oxo-5,6,7,9-tetrahydrobenzo[a]heptalen-7- |
| | | yl)propanamide |
| 293 | 294 | 2-amino-N-(((R)-6′-hydroxy-2′,4′,6′-trimethyl-7′-oxo-6′,7′- |
| | | dihydrospiro[cyclopropane-1,5′-inden]-3′-yl)methyl)-N- |
| | | methoxypropanamide |
| 294 | 295 | 2-amino-N-(((R)-6′-hydroxy-2′,4′,6′-trimethyl-7′-oxo-6′,7′- |
| | | dihydrospiro[cyclopropane-1,5′-inden]-3′-yl)methyl)-N-methoxy-4- |
| | | methylpentanamide |
| 295 | 296 | 2-amino-N-(((R)-6′-hydroxy-2′,4′,6′-trimethyl-7′-oxo-6′,7′- |
| | | dihydrospiro[cyclopropane-1,5′-inden]-3′-yl)methyl)-N-methoxy-4- |
| | | (methylthio)butanamide |
| 296 | 297 | 2-amino-N-(((R)-6′-hydroxy-2′,4′,6′-trimethyl-7′-oxo-6′,7′- |
| | | dihydrospiro[cyclopropane-1,5′-inden]-3′-yl)methyl)-3-(1H-indol- |
| | | 3-yl)-N-methoxypropanamide |
| 297 | 298 | (R)-3-(6′-hydroxy-2′,4′,6′-trimethyl-7′-oxo-6′,7′- |
| | | dihydrospiro[cyclopropane-1,5′-inden]-3′-yl)propyl (tert- |
| | | butoxycarbonyl)glycinate |
| 298 | 299 | (2′S,3′R,6′R)-2′-(azidomethyl)-3′,6′-dihydroxy-2′,4′,6′-trimethyl- |
| | | 2′,3′-dihydrospiro[cyclopropane-1,5′-inden]-7′(6′H)-one |
| 299 | 300 | (2′R,3′R,6′R)-3′-azido-6′-hydroxy-2′-(hydroxymethyl)-2′,4′,6′- |
| | | trimethyl-2′,3′-dihydrospiro[cyclopropane-1,5′-inden]-7′(6′H)-one |
| 300 | 301 | (R)-6′-hydroxy-2′,4′,6′-trimethyl-7′-oxo-6′,7′- |
| | | dihydrospiro[cyclopropane-1,5′-inden]-3′-yl 6-oxo-6- |
| | | phenylhexanoate |
| 301 | 302 | ((2′S,3′R,6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl 3,5- |
| | | dinitrobenzoate |
| 302 | 303 | (2′S,3′R,6′R)-2′-(((3,5-dinitrocyclohexa-2,4-diene-1- |
| | | carbonyl)oxy)methyl)-6′-hydroxy-2′,4′,6′-trimethyl-7′-oxo- |
| | | 2′,3′,6′,7′-tetrahydrospiro[cyclopropane-1,5′-inden]-3′-yl 3,5- |
| | | dinitrobenzoate |
| 303 | 304 | (R)-3-(6′-hydroxy-2′,4′,6′-trimethyl-7′-oxo-6′,7′- |
| | | dihydrospiro[cyclopropane-1,5′-inden]-3′-yl)propyl propiolate |
| 304 | 305 | (R)-3-(6′-hydroxy-2′,4′,6′-trimethyl-7′-oxo-6′,7′- |
| | | dihydrospiro[cyclopropane-1,5′-inden]-3′-yl)propyl (4-nitrophenyl) |
| | | carbonate |
| 305 | 306 | (R)-3-(6′-hydroxy-2′,4′,6′-trimethyl-7′-oxo-6′,7′- |
| | | dihydrospiro[cyclopropane-1,5′-inden]-3′-yl)propyl 4- |
| | | methylbenzenesulfonate |
| 306 | 307 | (3′R,6′R)-3′-azido-6′-hydroxy-2′,2′,4′,6′-tetramethyl-2′,3′- |
| | | dihydrospiro[cyclopropane-1,5′-inden]-7′(6′H)-one |
| 307 | 308 | (3′R,6′R)-3′-amino-6′-hydroxy-2′,2′,4′,6′-tetramethyl-2′,3′- |
| | | dihydrospiro[cyclopropane-1,5′-inden]-7′(6′H)-one |
| 308 | 309 | (2′R,3′S,6′R)-3′-azido-2′-(((tert-butyldimethylsilyl)oxy)methyl)-6′- |
| | | hydroxy-2′,4′,6′-trimethyl-2′,3′-dihydrospiro[cyclopropane-1,5′- |
| | | inden]-7′(6′H)-one |
| 309 | 310 | (R)-(6′-hydroxy-2′,4′,6′-trimethyl-7′-oxo-6′,7′- |
| | | dihydrospiro[cyclopropane-1,5′-inden]-3′-yl)methyl 4- |
| | | sulfamoylbenzoate |
| 310 | 311 | (3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl 4- |
| | | sulfamoylbenzoate |
| 311 | 312 | (R)-(6′-hydroxy-2′,4′,6′-trimethyl-7′-oxo-6′,7′- |
| | | dihydrospiro[cyclopropane-1,5′-inden]-3′-yl)methyl 4- |
| | | methylbenzenesulfonate |
| 312 | 313 | 2,3,4,5,6-pentafluoro-N-((3′R,6′R)-6′-hydroxy-2′,2′,4′,6′- |
| | | tetramethyl-7′-oxo-2′,3′,6′,7′-tetrahydrospiro[cyclopropane-1,5′- |
| | | inden]-3′-yl)benzenesulfonamide |
| 313 | 314 | (R)-3-(6′-hydroxy-2′,4′,6′-trimethyl-7′-oxo-6′,7′- |
| | | dihydrospiro[cyclopropane-1,5′-inden]-3′-yl)propyl 4- |
| | | methylbenzenesulfonate |
| 314 | 315 | (R)-3′-((2,5-dimethyl-1H-pyrrol-3-yl)methyl)-6′-hydroxy-2′,4′,6′- |
| | | trimethylspiro[cyclopropane-1,5′-inden]-7′(6′H)-one |
| 315 | 316 | ((2′S,3′R,6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl 4- |
| | | (fluorosulfonyl)benzoate |
| 316 | 317 | (2′S,3′R,6′R)-6′-hydroxy-2′-(hydroxymethyl)-2′,4′,6′-trimethyl-7′- |
| | | oxo-2′,3′,6′,7′-tetrahydrospiro[cyclopropane-1,5′-inden]-3′-yl 4- |
| | | (fluorosulfonyl)benzoate |
| 317 | 318 | ((2′S,3′R,6′R)-3′-((4-(fluorosulfonyl)benzoyl)oxy)-6′-hydroxy- |
| | | 2′,4′,6′-trimethyl-7′-oxo-2′,3′,6′,7′-tetrahydrospiro[cyclopropane- |
| | | 1,5′-inden]-2′-yl)methyl 4-(fluorosulfonyebenzoate |
| 318 | 332 | (R)-6′-hydroxy-3′-((methoxyamino)methyl)-2′,4′,6′- |
| | | trimethylspiro[cyclopropane-1,5′-inden]-7′(6′H)-one |
| 319 | 333 | ((2′S,3′R,6′R)-6′-hydroxy-2′,4′,6′-trimethyl-3′-(((4- |
| | | nitrophenoxy)carbonyl)oxy)-7′-oxo-2′,3′,6′,7′- |
| | | tetrahydrospiro[cyclopropane-1,5′-inden]-2′-yl)methyl acetate |
| 320 | 334 | ((2′S,3′R,6′R)-3′-(((2-((tert- |
| | | butoxycarbonyl)(methyl)amino)ethyl)(methyl)carbamoyl)oxy)-6′- |
| | | hydroxy-2′,4′,6′-trimethyl-7′-oxo-2′,3′,6′,7′- |
| | | tetrahydrospiro[cyclopropane-1,5′-inden]-2′-yl)methyl acetate |
| 321 | 335 | tert-butyl ((2′S,3′R,6′R)-6′-hydroxy-2′-(hydroxymethyl)-2′,4′,6′- |
| | | trimethyl-7′-oxo-2′,3′,6′,7′-tetrahydrospiro[cyclopropane-1,5′- |
| | | inden]-3′-yl)ethane-1,2-diylbis(methylcarbamate) |
| 322 | 337 | 4-nitrophenyl (R)-((6′-hydroxy-2′,4′,6′-trimethyl-7′-oxo-6′,7′- |
| | | dihydrospiro[cyclopropane-1,5′-inden]-3′-yl)methyl)carbamate |
| 323 | 338 | 4-nitrophenyl (R)-(3-(6′-hydroxy-2′,4′,6′-trimethyl-7′-oxo-6′,7′- |
| | | dihydrospiro[cyclopropane-1,5′-inden]-3′-yl)propyl)carbamate |
| 324 | 339 | ((2′S,3′R,6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl 4- |
| | | methylbenzenesulfonate |
| 325 | 340 | (R)-N-((6′-hydroxy-2′,4′,6′-trimethyl-7′-oxo-6′,7′- |
| | | dihydrospiro[cyclopropane-1,5′-inden]-3′-yl)methyl) |
| | | methanesulfonamide |
| 326 | 345 | (2′S,3′R,6′R)-2′-(acetoxymethyl)-6′-hydroxy-2′,4′,6′-trimethyl-7′- |
| | | oxo-2′,3′,6′,7′-tetrahydrospiro[cyclopropane-1,5′-inden]-3′-yl 4-(N- |
| | | (2-(2-(2-(2-aminoacetamido)acetamido)acetamido)ethyl)-N- |
| | | methylsulfamoyl)benzoate |
| 327 | 346 | ((2′S,3′R,6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl 4-(N-(2-(2- |
| | | (2-(2-aminoacetamido)acetamido)acetamido)ethyl)-N- |
| | | methylsulfamoyl)benzoate |
| 328 | 347 | ((2′S,3′R,6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl 4- |
| | | sulfamoylbenzoate |
| 330 | 348 | (3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl4- |
| | | sulfamoylbenzoate |
| 331 | 351 | (3′S,6′R)-6′-hydroxy-2′,2′,4′,6′-tetramethyl-7′-oxo-2′,3′,6′,7′- |
| | | tetrahydrospiro[cyclopropane-1,5′-inden]-3′-yl4-(N-(2-(2-(2-(2- |
| | | aminoacetamido)acetamido)acetamido)ethyl)-N- |
| | | methylsulfamoyl)benzoate |
| 332 | 353 | ((6′R)-3′-(((2-((((4-((S)-2-((S)-2-(6-(2,5-dioxo-2,5-dihydro-1H- |
| | | pyrrol-1-yl)hexanamido)-3-methylbutanamido)-5- |
| | | ureidopentanamido)benzyl)oxy)carbonyl)(methyl)amino)ethyl) |
| | | (methyl)carbamoyl)oxy)-6′-hydroxy-2′,4′,6′-trimethyl-7′-oxo- |
| | | 2′,3′,6′,7′-tetrahydrospiro[cyclopropane-1,5′-inden]-2′-yl)methyl |
| | | acetate |
| 333 | 354 | 4-((S)-2-((S)-2-(6-(2,5-dioxo-2,5-dihydro-1H-pyrrol-1- |
| | | yl)hexanamido)-3-methylbutanamido)-5-ureidopentanamido) |
| | | benzyl (2-(34(S)-6′-hydroxy-2′,4′,6′-trimethyl-7′-oxo-6′,7′- |
| | | dihydrospiro[cyclopropane-1,5′-inden]-3′-yl)propylidene) |
| | | hydrazine-1-carbonyl)carbamate |
| 334 | 356 | ((2′S,3′S,6′R)-3′-(((1-(9H-fluoren-9-yl)-13-methyl-3,6,9,12- |
| | | tetraoxo-2-oxa-4,7,10,13-tetraazapentadecan-15- |
| | | yl)(methyl)carbamoyl)oxy)-6′-hydroxy-2′,4′,6′-trimethyl-7′- |
| | | oxo-2′,3′,6′,7′-tetrahydrospiro[cyclopropane-1,5′-inden]-2′- |
| | | yl)methyl acetate |
| 335 | 357 | ((2′S,3′S,6′R)-3′-(((2-(2-(2-(2-aminoacetamido)acetamido)-N- |
| | | methylacetamido)ethyl)(methyl)carbamoyl)oxy)-6′-hydroxy- |
| | | 2′,4′,6′-trimethyl-7′-oxo-2′,3′,6′,7′-tetrahydrospiro |
| | | [cyclopropane-1,5′-inden]-2′-yl)methyl acetate |
| 336 | 359 | ((2′S,3′S,6′R)-6′-hydroxy-2′,4′,6′-trimethyl-3′-((methyl(2,2,14- |
| | | trimethyl-4,7,10,13-tetraoxo-3-oxa-5,8,11,14-tetraazahexadecan- |
| | | 16-yl)carbamoyl)oxy)-7′-oxo-2′,3′,6′,7′- |
| | | tetrahydrospiro[cyclopropane-1,5′-inden]-2′-yl)methyl acetate |
| 337 | 361 | ((2′S,3′R,6′R)-3′-((((2,5-dioxopyrrolidin-1-yl)oxy)carbonyl)oxy)-6′- |
| | | hydroxy-2′,4′,6′-trimethyl-7′-oxo-2′,3′,6′,7′- |
| | | tetrahydrospiro[cyclopropane-1,5′-inden]-2′-yl)methyl acetate |
| 338 | 362 | ((2′S,3′R,6′R)-3′,6′-dihydroxy-2′,4′,6′-trimethyl-7′-oxo-2′,3′,6′,7′- |
| | | tetrahydrospiro[cyclopropane-1,5′-inden]-2′-yl)methyl |
| | | methanesulfonate |
| 339 | 363 | (2′S,3′R,6′R)-2′-(aminomethyl)-3′,6′-dihydroxy-2′,4′,6′-trimethyl- |
| | | 2′,3′-dihydrospiro[cyclopropane-1,5′-inden]-7′(6′H)-one |
| 340 | 364 | (2′R,3′R,6′R)-3′-amino-6′-hydroxy-2′-(hydroxymethyl)-2′,4′,6′- |
| | | trimethyl-2′,3′-dihydrospiro[cyclopropane-1,5′-inden]-7′(6′H)-one |
| 341 | 366 | tert-butyl (R)-((6′-hydroxy-2′,4′,6′-trimethyl-7′-oxo-6′,7′- |
| | | dihydrospiro[cyclopropane-1,5′-inden]-3′-yl)methyl)carbamate |
| 342 | 367 | tert-butyl (R)-(3-(6′-hydroxy-2′,4′,6′-trimethyl-7′-oxo-6′,7′- |
| | | dihydrospiro[cyclopropane-1,5′-inden]-3′-yl)propyl)carbamate |
| 343 | 368 | ((2′S,3′R,6′R)-3′-acetoxy-6′-hydroxy-2′,4′,6′-trimethyl-7′-oxo- |
| | | 2′,3′,6′,7′-tetrahydrospiro[cyclopropane-1,5′-inden]-2′-yl) |
| | | methyl acetate |
| 344 | 369 | (2′S,3′R,6′R)-2′-(((tert-butyldimethylsilyl)oxy)methyl)-3′,6′- |
| | | dihydroxy-2′,4′,6′-trimethyl-2′,3′-dihydrospiro[cyclopropane-1,5′- |
| | | inden]-7′(6′H)-one |
| 345 | 370 | (2′S,3′R,6′R)-2′-(((tert-butyldimethylsilyl)oxy)methyl)-6′-hydroxy- |
| | | 2′,4′,6′-trimethyl-7′-oxo-2′,3′,6′,7′-tetrahydrospiro[cyclopropane- |
| | | 1,5′-inden]-3′-yl acetate |
| 346 | 371 | (2′S,3′R,6′R)-6′-hydroxy-2′-(hydroxymethyl)-2′,4′,6′-trimethyl-7′- |
| | | oxo-2′,3′,6′,7′-tetrahydrospiro[cyclopropane-1,5′-inden]-3′-yl |
| | | acetate |
| 347 | 372 | (2′R,3′R,6′R)-2′-formyl-6′-hydroxy-2′,4′,6′-trimethyl-7′-oxo- |
| | | 2′,3′,6′,7′-tetrahydrospiro[cyclopropane-1,5′-inden]-3′-yl acetate |
| 348 | 373 | (2′5,3′R,6′R)-2′-(((((9H-fluoren-9-yl)methoxy)carbonyl) |
| | | amino)methyl)-6′-hydroxy-2′,4′,6′-trimethyl-7′-oxo-2′,3′,6′,7′- |
| | | tetrahydro spiro lcyclopropane-1,5′-inden]-3′-yl acetate |
| 349 | 374 | (2S,3′R,6′R)-2′-(amino methyl)-6′-hydroxy-2′,4′,6′-trimethyl-7′-oxo- |
| | | 2′,3,6,7′-tetrahydrospiro[cyclopropane-1,5′-inden]-3′-yl acetate |
| 350 | 377 | ((2′S,3′R,6′R)-6′-hydroxy-2′,4′,6′-trimethyl-7′-oxo-3′- |
| | | ((tributylsilyl)oxy)-2′,3′,6′,7′-tetrahydrospiro lcyclopropane-1,5′- |
| | | inden]-2′-yl)methyl acetate |
| 351 | 378 | (2′S′R,6′R)-2′-(((tert-butoxycarbonyl)amino)methyl)-6′-hydroxy- |
| | | 2′,4′,6′-trimethyl-7′-oxo-2′,3,6′,7′-tetrahydrospiro[cyclopropane- |
| | | 1,5′-inden]-3′-yl acetate |
| 352 | 379 | ((2′S,3′R,6′R)-3′-(((2-((((4-((14S,17S)-1-(9H-fluoren-9-yl)-14- |
| | | isopropyl-3,6,9,12,15-pentaoxo-17-(3-ureidopropyl)-2-oxa- |
| | | 4,7,10,13,16-pentaazaoctadecan-18-amido)benzyl)oxy) |
| | | carbonyl)(methyl)amino)ethyl)(methyl)carbamoyl) |
| | | oxy)-6′-hydroxy-2′,4′,6′-trimethyl-7′-oxo-2′,3′,6′,7′- |
| | | tetrahydrospiro[cyclopropane-1,5′-inden]-2′-yl)methyl acetate |
| 353 | 380 | ((2′S′R,6′R)-3′-(((2-((((4-((2S,5S)-14-amino-5-isopropyl- |
| | | 4,7,10,13-tetraoxo-2-(3-ureidopropyl)-3,6,9,12- |
| | | tetraazatetradecanamido)benzyl)oxy)carbonyl)(methyl)amino) |
| | | ethyl)(methyl)carbamoyl)oxy)-6′-hydroxy-2′,4′,6′-trimethyl-7′- |
| | | oxo-2′,3′,6′,7′-tetrahydrospiro[cyclopropane-1,5′-inden]-2′-yl) |
| | | methyl acetate |
| 354 | 381 | (2′S,3′R,6′R)-6′-hydroxy-2′-(hydroxymethyl)-2′,4′,6′-trimethyl-3′- |
| | | ((tributylsilyl)oxy)-2′,3′-dihydrospiro[cyclopropane-1,5′-inden]- |
| | | 7′(6′H)-one |
| 355 | 382 | (2′R,3′R,6′R)-6′-hydroxy-2′,4′,6′-trimethyl-7′-oxo-3′- |
| | | ((tributylsilyl)oxy)-2′,3′,6′,7′-tetrahydrospiro[cyclopropane-1,5′- |
| | | indenel-2′-carbaldehyde |
| 356 | 383 | (2′R,3′R,6′R)-3′,6′-dihydroxy-2′,4′,6′-trimethyl-7′-oxo-2′,3′,6′,7′- |
| | | tetrahydrospiro[cyclopropane-1,5′-indene]-2′-carbaldehyde |
| 357 | 384 | tert-butyl (((2′5,3′R,6′R)-6′-hydroxy-2′,4′,6′-trimethyl-7′-oxo-3′- |
| | | ((tributylsilyl)oxy)-2′,3′,6′,7′-tetrahydrospiro[cyclopropane-1,5′- |
| | | inden]-2′-yl)methyl)carbamate |
| 358 | 389 | tert-butyl (((2′S,3′R,6′R)-3′,6′-dihydroxy-2′,4′,6′-trimethyl-7′-oxo- |
| | | 2′,3′,6′,7′-tetrahydrospiro[cyclopropane-1,5′-inden]-2′- |
| | | yl)methyl)carbamate |
| 359 | 392 | ((2′S,3′S,6′R)-3′-(((2-(2-(2-(2-azidoacetamido)acetamido)-N- |
| | | methylacetamido)ethyl)(methyl)carbamoyl)oxy)-6′-hydroxy- |
| | | 2′,4′,6′-trimethyl-7′-oxo-2′,3′,6′,7′-tetrahydrospiro[cyclopropane- |
| | | 1,5′-inden]-2′-yl)methyl acetate |
| 360 | 393 | ((2′S,3′R,6′R)-3′-(((2-(2,5-dioxo-2,5-dihydro-1H-pyrrol-1- |
| | | yl)ethyl)carbamoyl)oxy)-6′-hydroxy-2′,4′,6′-trimethyl-7′-oxo- |
| | | 2′,3′,6′,7′-tetrahydrospiro[cyclopropane-1,5′-inden]-2′-yl)methyl |
| | | acetate |
| 361 | 394 | (R)-6′-hydroxy-3′-((hydroxyamino)methyl)-2′,4′,6′- |
| | | trimethylspiro[cyclopropane-1,5′-inden]-7′(6′H)-one |

TABLE 15
A summary NCI DTP 60 Cell Line showing Growth Inhibition (GI), Total Growth
Inhibition (TGI, the concentration of test drug where 100 × (T − T0)/(C − T0) = 0, i.e.
TGI signifies a cytostatic effect) and Lethal Concentration Dose (a cytotoxic effect, LD)
Data for selected Other Drugs, naturally occurring illudins, and Illudofulvene Analogs.

| | Mean GI50 | | Mean TGI | | Mean LD50 | |
|---|---|---|---|---|---|---|
| NAME/NSC | inhibition | | cytostatic | | cytotoxic | |
| Pyrrolobenzodiazepines 694501 | 7 | nM | 302 | nM | >23,000 | nM |
| Maytansine 153858 | 19 | nM | 318 | nM | 49,200 | nM |
| Fumagillol 642492 | 6,130 | nM | 9,850 | nM | >50,000 | nM |
| Dolstatin-10376128 | 17 | nM | 2,680 | nM | >50,000 | nM |
| Auristatins 654663 | 1.4 | nM | 902 | nM | >5,000 | nM |
| Enadiyne 157365 | 2,900 | nM | >100,000 | nM | >100,000 | nM |
| Halichondrin B 609395 | 1.2 | nM | 199 | nM | >1,000 | nM |
| Tubulysin A | 12 | nM | 1,318 | nM | >10,000 | nM |
| Illudin S | 10 | nM | 64 | nM | 511 | nM |
| Illudin M | 3 | nM | 20 | nM | 291 | nM |
| Illudofulvene Analog 0016 | 102 | nM | 1,070 | nM | 4,270 | nM |
| Illudofulvene Analog 0142 | 447 | nM | 4,667 | nM | 53,700 | nM |
| Illudofulvene Analog 0159 | 117 | nM | 2,090 | nM | 14,790 | nM |

TABLE 16
A comparison of Illudofulvene Analog 002 growth inhibition values
obtained from cells in the NCI DTP 60 cell line panel to expression
of various DNA repair genes, to an embodiment of the invention.

| GENE | GENE SEQ ID Nos | P value |
|---|---|---|
| ERCC2 | 21, 22, 145 through 155 | P = 0.0246 |
| ERCC8 | 1, 2, 35 through 45 | P = 0.007 |
| GTF2H1 | 5, 6, 57 through 67 | P = 0.0007 |
| PTGR1 | 249, 250, 251 through 261 | P < 0.0001 |
| USP7 | 1, 16, 112 through 122 | P < 0.0001 |
| XPC | none | P > 0.20 |
| XRCC1 | none | P > 0.20 |
| RAD50 | none | P > 0.20 |

TABLE 17
A comparison of Illudofulvene Analog 142 growth inhibition values obtained
from cells in the NCI DTP 60 cell line panel to expression of various
DNA repair genes, according to an embodiment of the invention.

| GENE | GENE SEQ ID Nos | P value |
|---|---|---|
| ERCC2 | 21, 22, 145 through 155 | P = 0.029 |
| ERCC3 | 19, 20, 134 through 144 | P = 0.0167 |
| ERCC6 | 3, 4, 46 through 56 | P = 0.0297 |
| ERCC8 | 1,2, 35 through 45 | P = 0.0159 |
| PTGR1 | 249, 250, 251 through 261 | P < 0.0001 |
| USP7 | 1, 16, 112 through 122 | P < 0.0001 |
| XPC | none | P > 0.20 |
| XRCC1 | none | P > 0.20 |
| RAD50 | none | P > 0.20 |

TABLE 18
A comparison of Illudofulvene Analog 184 growth inhibition values
obtained from cells in the NCI DTP 60 cell line panel to expression of
various DNA repair genes,according to an embodiment of the invention.

| GENE | GENE SEQ ID Nos | P value |
|---|---|---|
| ERCC3 | 19, 20, 134 through 144 | P = 0.0252 |
| ERCC8 | 1,2, 35 through 45 | P = 0.0125 |
| GTF2H2 | 5, 6, 68 through 78 | P = 0.0107 |
| PTGR1 | 249, 250, 251 through 261 | P < 0.0001 |
| USP7 | 15, 16, 112 through 122 | P < 0.0001 |
| MYC | 222, 223, 224 through 235 | P < 0.005 |
| XPA | 27, 28, 178 through 188 | P = 0.0219 |
| XPC | none | P > 0.20 |
| XRCC1 | none | P > 0.20 |
| RAD50 | none | P > 0.20 |

TABLE 19
A comparison of Illudofulvene Analog 176 growth inhibition values
obtained from cells in the NCI DTP 60 cell line panel to expression of
various DNA repair genes, according to an embodiment of the invention.

| GENE | GENE SEQ ID Nos | P value |
|---|---|---|
| ERCC3 | 19,20, 134 through 144 | P = 0.0258 |
| ERCC8 | 1, 2, 35 through 45 | P = 0.042 |
| RAD18 | 29,30, 189 through 199 | P = 0.0147 |
| GTF2H3 | 9, 10, 79 through 89 | P = 0.0037 |
| GTF2H5 | 13,14 101 through 111 | P = 0.0387 |
| PTGR1 | 249, 250, 251 through 261 | P < 0.0001 |
| USP7 | 15, 16, 112 through 122 | P < 0.0001 |
| MYC | 222, 223, 224 through 235 | P < 0.002 |
| XPC | none | P > 0.20 |
| XRCC1 | none | P > 0.20 |
| RAD50 | none | P > 0.20 |

TABLE 20
A comparison of the Illudin S growth inhibition
values obtained from cells in the NCI DTP 60 cell
line panel to expression of various DNA repair genes.

| GENE | GENE SEQ ID Nos. | P value |
|---|---|---|
| ERCC3 | 19, 20, 134 through 144 | P = 0.032 |
| ERCC1 | 236, 237, 238 through 248 | P = 0.0102 |
| GTF2H1 | 5, 6, 57 through 67 | P = 0.0003 |
| PTGR1 | 249, 250, 251 through 261 | P < 0.0244 |
| PTGR2 | 262, 263, 264 through 274 | P = 0.0382 |
| PTGR1 + 2* | 249 through 274 | P = 0.0031 |
| USP7 | 15, 16, 112 through 122 | P < 0.0103 |
| MYC | 222, 223, 224 through 235 | P < 0.0134 |
| XPC | none | P > 0.20 |
| XRCC1 | none | P > 0.20 |
| RAD50 | none | P > 0.20 |

*PTGR1 + 2 refers to the sum of the activity of PTGR1 gene and the PTGR2 gene.

### Analysis of Primary Tumor Cell Sensitivity.

Visible residues of healthy tissue are removed from the tumor sample excised from the patient or obtained from peripheral blood or other fluids. The tumor can be washed with PBS, placed in a petri dish and 2 ml of Primary Cancer Cell Medium D-ACF (PromoCell #C-2081) added and the tumor dissected into small pieces, then homogenized to 1 mm³ particles. The tissue can be pelleted, washed, suspended then plated into NCDD-treated tissue culture vessels (PromoCell #C43080) containing Primary Cancer Cell Medium D-ACF. After 7 days the adherent cells are harvested, split, and plated into NCDD-treated tissue culture vessels (PromoCell #C43080) containing Primary Cancer Cell Medium D-ACF. A portion of the harvested adherent tumor cells are plated into 6 well tissue culture plates containing Primary Cancer Cell Medium D-ACF. Cells are allowed to adhere overnight. An illudofulvene is then added at: 0 ng/mL (control), 30 ng/mL, 100 ng/mL, 300 ng/mL, 1000 ng/mL, 3000 ng/mL. Cell viability at each concentration can then be determined and compared to the control population at 48 hours later by using a viability dye such as MTT, trypan blue or Sulforhodamine B. Tumor cell populations with an IC50 less than 300 ng/mL can be considered sensitive to the illudofulvene, whereas those with an IC50 greater than 3,000 ng/mL can be considered resistant. The illudofulvene can be administered to the patient based on the sensitivity. For example, in an embodiment of the invention, the tumor sample can be exposed to the desired Illudofulvene Analog under study at half-log internal concentrations from 1 ng/mL to 10,000 ng/mL, and cell viability determined using a viability dye (MTT, trypan blue, Sulforhodamine B, etc.) after 48 hours of exposure to the Illudofulvene Analog, and the IC50 determined. Tumor cells with an IC50 less than the median IC50 obtained for that Illudofulvene Analog in the NCI DTP 60 cell line panel can be considered sensitive whereas those with an IC50 greater than the median IC50 obtained for that Illudofulvene in the NCI DTP 60 cell line panel can be considered resistant.

### Further Embodiments

Embodiments contemplated herein include Embodiments P1-P125 following.

Embodiment P1. An illudofulvene for use in the treatment of a cancer expressing an attenuated TCR biomarker, wherein said expression includes: (a) contacting a sample from a patient comprising one or more nucleic acid molecules with a device comprising single-stranded nucleic acid molecules capable of specifically hybridizing with the nucleotides of a TCR gene;

(b) detecting a level of expression of a TCR biomarker by performing microarray analysis or quantitative reverse transcriptase polymerase chain reaction (qRT-PCR) of the sample; and (c) determining that the TCR biomarker is attenuated.

Embodiment P2. The illudofulvene of Embodiment P1, where the TCR gene is selected from the group consisting of an ERCC gene, a XPA gene, a GTF series gene, a UV gene, and a USP gene.

Embodiment P3. The illudofulvene of Embodiment P2, where the ERCC gene is selected from the group consisting of ERCC1 gene, ERCC2 gene, ERCC3 gene, ERCC4 gene, ERCC5 gene, ERCC6 gene, ERCC6L gene, ERCC6L2 gene, and ERCC8 gene.

Embodiment P4. The illudofulvene of Embodiment P1, where the illudofulvene is an acylfulvene.

Embodiment P5. The illudofulvene of Embodiment P1, where the illudofulvene is an illudin.

Embodiment P6. An illudofulvene for use in a method of treating a patient with cancer, wherein the method includes: (a) determining whether a test sample from a patient has an attenuated TCR biomarker; and (b) if the test sample from the patient includes the attenuated TCR biomarker, then administer an effective amount of the illudofulvene.

Embodiment P7. The illudofulvene of Embodiment P6, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment P8. The illudofulvene of Embodiment P6, where the test sample includes one or more nucleic acid molecules.

Embodiment P9. The illudofulvene of Embodiment P6, where the test sample hybridized specifically with the nucleotides of a TCR gene.

Embodiment P10. The illudofulvene of Embodiment P9, where the TCR gene is selected from the group consisting of ERCC gene, XPA gene, GTF series gene, UV gene, and USP gene.

Embodiment P11. The illudofulvene of Embodiment P6, where the TCR biomarker is determined to be attenuated by performing microarray analysis or quantitative reverse transcriptase polymerase chain reaction (qRT-PCR) of the test sample.

Embodiment P12. The illudofulvene of Embodiment P6, where the illudofulvene is an acylfulvene.

Embodiment P13. The illudofulvene of Embodiment P6, where the illudofulvene is an illudin.

Embodiment P14. An illudofulvene for use in the treatment of a cancer expressing an enhanced MYC biomarker, wherein said expression includes: (a) contacting a sample from a patient comprising one or more nucleic acid molecules with a device comprising single-stranded nucleic acid molecules capable of specifically hybridizing with the nucleotides of a MYC gene; (b) detecting a level of expression of a MYC biomarker by performing microarray analysis or quantitative reverse transcriptase polymerase chain reaction (qRT-PCR) of the sample; and (c) determining that the MYC biomarker is enhanced.

Embodiment P15. The illudofulvene of Embodiment P14, further comprising where a PTGR biomarker is enhanced in the sample.

Embodiment P16. The illudofulvene of Embodiment P14, further comprising where a TCR biomarker is attenuated in the sample.

Embodiment P17. The illudofulvene of Embodiment P14, where the illudofulvene is an acylfulvene.

Embodiment P18. The illudofulvene of Embodiment P14, where the illudofulvene is an illudin.

Embodiment P19. An illudofulvene for use in a method of treating a patient with cancer, wherein the method includes: (a) determining whether a test sample from a patient has a MYC biomarker that is enhanced; and (b) if the test sample from the patient includes the enhanced MYC biomarker, then administer an effective amount of the illudofulvene.

Embodiment P20. The illudofulvene of Embodiment P18, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment P21. The illudofulvene of Embodiment P19, where the test sample includes one or more nucleic acid molecules.

Embodiment P22. The illudofulvene of Embodiment P19, where the test sample hybridized specifically with the nucleotides of a MYC gene.

Embodiment P23. The illudofulvene of Embodiment P19, where the MYC biomarker is determined to be enhanced by performing microarray analysis or quantitative reverse transcriptase polymerase chain reaction (qRT-PCR) of the test sample.

Embodiment P24. The illudofulvene of Embodiment P19, where the illudofulvene is an acylfulvene.

Embodiment P25. The illudofulvene of Embodiment P19, where the illudofulvene is an illudin.

Embodiment P26. The illudofulvene of Embodiment P19, further comprising where a PTGR biomarker is enhanced in the test sample.

Embodiment P27. The illudofulvene of Embodiment P26, further comprising where a TCR biomarker is attenuated in the test sample.

Embodiment P28. An illudofulvene for use in the treatment of a cancer expressing an enhanced MYC biomarker and an attenuated TCR biomarker, wherein said expression includes: (a) contacting a sample from a patient comprising one or more nucleic acid molecules with a device comprising single-stranded nucleic acid molecules capable of specifically hybridizing with the nucleotides of a TCR gene and a MYC gene; (b) detecting a level of expression of a TCR biomarker and a MYC biomarker by performing microarray analysis or quantitative reverse transcriptase polymerase chain reaction (qRT-PCR) of the sample; and (c) determining that the MYC biomarker is enhanced and the TCR biomarker is attenuated.

Embodiment P29. The illudofulvene of Embodiment P28, further comprising where a PTGR biomarker is enhanced in the sample.

Embodiment P30. The illudofulvene of Embodiment P28, where the illudofulvene is an acylfulvene.

Embodiment P31. The illudofulvene of Embodiment P28, where the illudofulvene is an illudin.

Embodiment P32. The illudofulvene of Embodiment P28, where the TCR gene is selected from the group consisting of ERCC gene, XPA gene, GTF series gene, UV gene, and USP gene.

Embodiment P33. The illudofulvene of Embodiment P28, where the TCR biomarker is attenuated due to truncating mutations, mutations abolishing enzyme activity, deletions or other chromosomal, transcription, translational and post translation event that interfering with TCR gene expression and/or subsequent TCR gene activity.

Embodiment P34. The illudofulvene of Embodiment P28, where the MYC biomarker is enhanced when cells transform from a normal state to cancer cells, or when the cells are exposed to prior anticancer drug treatment and become upregulated as a response to the prior anticancer drug treatment.

Embodiment P35. An illudofulvene for use in a method of treating a patient with cancer, wherein the method includes: (a) determining whether a test sample from a patient has an enhanced MYC biomarker; (b) determining whether the test sample from a patient has an attenuated TCR biomarker; (c) if the test sample from the patient includes the enhanced MYC biomarker and the attenuated TCR biomarker, then administer an effective amount of the illudofulvene.

Embodiment P36. The illudofulvene of Embodiment P35, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment P37. The illudofulvene of Embodiment P35, where the test sample includes one or more nucleic acid molecules.

Embodiment P38. The illudofulvene of Embodiment P35, where the test sample hybridized specifically with the nucleotides of a MYC gene.

Embodiment P39. The illudofulvene of Embodiment P35, where the test sample hybridized specifically with the nucleotides of a TCR gene.

Embodiment P40. The illudofulvene of Embodiment P35, where the MYC biomarker is determined to be enhanced by performing microarray analysis or quantitative reverse transcriptase polymerase chain reaction (qRT-PCR) of the test sample.

Embodiment P41. The illudofulvene of Embodiment P35, where the TCR biomarker is determined to be attenuated by performing microarray analysis or quantitative reverse transcriptase polymerase chain reaction (qRT-PCR) of the test sample.

Embodiment P42. The illudofulvene of Embodiment P35, where the illudofulvene is an acylfulvene.

Embodiment P43. The illudofulvene of Embodiment P35, where the illudofulvene is an illudin.

Embodiment P44. The illudofulvene of Embodiment P35, further comprising where a PTGR biomarker is enhanced in the test sample.

Embodiment P45. The illudofulvene of Embodiment P35, further comprising where the TCR gene is selected from the group consisting of ERCC gene, XPA gene, GTF series gene, UV gene, and USP gene.

Embodiment P46. The illudofulvene of Embodiment P35, where the MYC biomarker is enhanced when cells transform from a normal state to cancer cells, or when the cells are exposed to prior anticancer drug treatment and become upregulated as a response to the prior anticancer drug treatment.

Embodiment P47. An illudofulvene for use in the treatment of a cancer expressing an enhanced PTGR biomarker, wherein said expression includes: (a) contacting a sample from a patient comprising one or more nucleic acid molecules with a device comprising single-stranded nucleic acid molecules capable of specifically hybridizing with the nucleotides of a PTGR gene; (b) detecting a level of expression of a PTGR biomarker by performing microarray analysis or quantitative reverse transcriptase polymerase chain reaction (qRT-PCR) of the sample; and (c) determining that the PTGR biomarker is attenuated.

Embodiment P48. The illudofulvene of Embodiment P47, where the PTGR gene is selected from the group consisting of PTGR1 and PTGR2.

Embodiment P49. The illudofulvene of Embodiment P47, where the illudofulvene is an acylfulvene.

Embodiment P50. The illudofulvene of Embodiment P47, where the illudofulvene is an illudin.

Embodiment P51. The illudofulvene of Embodiment P47, where the PTGR biomarker is enhanced when cells transform from a normal state to cancer cells, or when the cells are exposed to prior anticancer drug treatment and become upregulated as a response to the prior anticancer drug treatment.

Embodiment P52. An illudofulvene for use in a method of treating a patient with cancer, wherein the method includes: (a) determining whether a test sample from a patient has an enhanced PTGR biomarker; and (b) if the test sample from the patient includes the enhanced PTGR biomarker, then administer an effective amount of the illudofulvene.

Embodiment P53. The illudofulvene of Embodiment P52, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment P54. The illudofulvene of Embodiment P53, where the test sample includes one or more nucleic acid molecules.

Embodiment P55. The illudofulvene of Embodiment P53, where the test sample hybridized specifically with the nucleotides of a PTGR gene.

Embodiment P56. The illudofulvene of Embodiment P53, where the PTGR gene is selected from the group consisting of PTGR1 and PTGR2.

Embodiment P57. The illudofulvene of Embodiment P53, where the PTGR biomarker is determined to be attenuated by performing microarray analysis or quantitative reverse transcriptase polymerase chain reaction (qRT-PCR) of the test sample.

Embodiment P58. The illudofulvene of Embodiment P53, where the illudofulvene is an acylfulvene.

Embodiment P59. The illudofulvene of Embodiment P53, where the illudofulvene is an illudin.

Embodiment P60. The illudofulvene of Embodiment P53, where the PTGR gene includes PTGR1 and PTGR2.

Embodiment P61. The illudofulvene of Embodiment P53, where the PTGR biomarker is enhanced when cells transform from a normal state to cancer cells, or when the cells are exposed to prior anticancer drug treatment and become upregulated as a response to the prior anticancer drug treatment.

Embodiment P62. An illudofulvene for use in the treatment of a cancer expressing an enhanced MYC biomarker, an enhanced PTGR biomarker, and an attenuated TCR biomarker, wherein said expression includes: (a) contacting a sample from a patient comprising one or more nucleic acid molecules with a device comprising single-stranded nucleic acid molecules capable of specifically hybridizing with the nucleotides of a TCR gene, a PTGR gene, and a MYC gene; (b) detecting a level of expression of a TCR biomarker, a PTGR biomarker, and a MYC biomarker by performing microarray analysis or quantitative reverse transcriptase polymerase chain reaction (qRT-PCR) of the sample; and (c) determining that the MYC biomarker is enhanced, the PTGR biomarker is enhanced, and the TCR biomarker is attenuated.

Embodiment P63. The illudofulvene of Embodiment P62, where the PTGR gene is selected from the group consisting of PTGR1 and PTGR2.

Embodiment P64. The illudofulvene of Embodiment P62, where the TCR gene is selected from the group consisting of ERCC gene, XPA gene, GTF series gene, UV gene, and USP gene.

Embodiment P65. The illudofulvene of Embodiment P62, where the TCR biomarker is attenuated due to truncating mutations, mutations abolishing enzyme activity, deletions or other chromosomal, transcription, translational and post translation event that interfering with ERCC gene expression and subsequent gene activity.

Embodiment P66. The illudofulvene of Embodiment P62, where the PTGR biomarker is enhanced when cells transform from a normal state to cancer cells, or when the cells are exposed to prior anticancer drug treatment and become upregulated as a response to the prior anticancer drug treatment.

Embodiment P67. The illudofulvene of Embodiment P62, where the MYC biomarker is enhanced when cells transform from a normal state to cancer cells, or when the cells are exposed to prior anticancer drug treatment and become upregulated as a response to the prior anticancer drug treatment.

Embodiment P68. The illudofulvene of Embodiment P62, where the illudofulvene is an acylfulvene.

Embodiment P69. The illudofulvene of Embodiment P62, where the illudofulvene is an illudin.

Embodiment P70. An illudofulvene for use in a method of treating a patient with cancer, wherein the method includes: (a) determining whether a test sample from a patient has an enhanced MYC biomarker; (b) determining whether the test sample from a patient has an attenuated TCR biomarker; (c) determining whether the test sample from a patient has an enhanced PTGR biomarker; (d) if the test sample from the patient includes the enhanced MYC biomarker, the enhanced PTGR biomarker, and the attenuated TCR biomarker, then administer an effective amount of the illudofulvene.

Embodiment P71. The illudofulvene of Embodiment P70, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment P72. The illudofulvene of Embodiment P70, where the test sample includes one or more nucleic acid molecules.

Embodiment P73. The illudofulvene of Embodiment P70, where the test sample hybridized specifically with the nucleotides of a MYC gene.

Embodiment P74. The illudofulvene of Embodiment P70, where the test sample hybridized specifically with the nucleotides of a TCR gene.

Embodiment P75. The illudofulvene of Embodiment P70, where the test sample hybridized specifically with the nucleotides of a PTGR gene.

Embodiment P76. The illudofulvene of Embodiment P70, where the MYC biomarker is determined to be enhanced by performing microarray analysis or quantitative reverse transcriptase polymerase chain reaction (qRT-PCR) of the test sample.

Embodiment P77. The illudofulvene of Embodiment P70, where the TCR biomarker is determined to be attenuated by performing microarray analysis or quantitative reverse transcriptase polymerase chain reaction (qRT-PCR) of the test sample.

Embodiment P78. The illudofulvene of Embodiment P70, where the illudofulvene is an acylfulvene.

Embodiment P79. The illudofulvene of Embodiment P70, where the illudofulvene is an illudin.

Embodiment P80. The illudofulvene of Embodiment P70, further comprising where the TCR gene is selected from the group consisting of ERCC gene, XPA gene, GTF series gene, UV gene, and USP gene.

Embodiment P81. The illudofulvene of Embodiment P70, where the PTGR gene is selected from the group consisting of PTGR1 and PTGR2.

Embodiment P82. The illudofulvene of Embodiment P70, where the MYC biomarker is enhanced when cells transform from a normal state to cancer cells, or when the cells are exposed to prior anticancer drug treatment and become upregulated as a response to the prior anticancer drug treatment.

Embodiment P83. The illudofulvene of Embodiment P70, where the TCR biomarker is attenuated due to truncating mutations, mutations abolishing enzyme activity, deletions or other chromosomal, transcription, translational and/or post translation event that interfering with TCR gene expression and/or subsequent TCR gene activity.

Embodiment P84. The illudofulvene of Embodiment P70, where the PTGR biomarker is enhanced when cells transform from a normal state to cancer cells, or when the cells are exposed to prior anticancer drug treatment and become upregulated as a response to the prior anticancer drug treatment.

Embodiment P85. An illudofulvene for use in the treatment of a cancer expressing an enhanced PTGR biomarker, and an attenuated TCR biomarker, wherein said expression includes: (a) contacting a sample from a patient comprising one or more nucleic acid molecules with a device comprising single-stranded nucleic acid molecules capable of specifically hybridizing with the nucleotides of a TCR gene, and a PTGR gene; (b) detecting a level of expression of a TCR biomarker and a PTGR biomarker by performing microarray analysis or quantitative reverse transcriptase polymerase chain reaction (qRT-PCR) of the sample; and (c) determining that the PTGR biomarker is enhanced, and the TCR biomarker is attenuated.

Embodiment P86. The illudofulvene of Embodiment P85, where the PTGR gene is selected from the group consisting of PTGR1 and PTGR2.

Embodiment P87. The illudofulvene of Embodiment P85, where the TCR gene is selected from the group consisting of ERCC gene, XPA gene, GTF series gene, UV gene, and USP gene.

Embodiment P88. The illudofulvene of Embodiment P85, where the TCR biomarker is attenuated due to truncating mutations, mutations abolishing enzyme activity, deletions or other chromosomal, transcription, translational and post translation event that interfering with ERCC gene expression and subsequent gene activity.

Embodiment P89. The illudofulvene of Embodiment P85, where the PTGR biomarker is enhanced when cells transform from a normal state to cancer cells, or when the cells are exposed to prior anticancer drug treatment and become upregulated as a response to the prior anticancer drug treatment.

Embodiment P90. The illudofulvene of Embodiment P85, where the illudofulvene is an acylfulvene.

Embodiment P91. The illudofulvene of Embodiment P85, where the illudofulvene is an illudin.

Embodiment P92. An illudofulvene for use in a method of treating a patient with cancer, wherein the method includes: (a) determining whether the test sample from a patient has an attenuated TCR biomarker; (b) determining whether the test sample from a patient has an enhanced PTGR biomarker; (c) if the test sample from the patient includes the enhanced PTGR biomarker, and the attenuated TCR biomarker, then administer an effective amount of the illudofulvene.

Embodiment P93. The illudofulvene of Embodiment P92, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment P94. The illudofulvene of Embodiment P92, where the test sample includes one or more nucleic acid molecules.

Embodiment P95. The illudofulvene of Embodiment P92, where the test sample hybridized specifically with the nucleotides of a TCR gene.

Embodiment P96. The illudofulvene of Embodiment P92, where the test sample hybridized specifically with the nucleotides of a PTGR gene.

Embodiment P97. The illudofulvene of Embodiment P92, where the TCR biomarker is determined to be attenuated by performing microarray analysis or quantitative reverse transcriptase polymerase chain reaction (qRT-PCR) of the test sample.

Embodiment P98. The illudofulvene of Embodiment P92, where the illudofulvene is an acylfulvene.

Embodiment P99. The illudofulvene of Embodiment P92, where the illudofulvene is an illudin.

Embodiment P100. The illudofulvene of Embodiment P92, further comprising where the TCR gene is selected from the group consisting of ERCC gene, XPA gene, GTF series gene, UV gene, and USP gene.

Embodiment P101. The illudofulvene of Embodiment P92, where the PTGR gene is selected from the group consisting of PTGR1 and PTGR2.

Embodiment P102. The illudofulvene of Embodiment P92, where the TCR biomarker is attenuated due to truncating mutations, mutations abolishing enzyme activity, deletions or other chromosomal, transcription, translational and/or post translation event that interfering with TCR gene expression and/or subsequent TCR gene activity.

Embodiment P103. The illudofulvene of Embodiment P92, where the PTGR biomarker is enhanced when cells transform from a normal state to cancer cells, or when the cells are exposed to prior anticancer drug treatment and become upregulated as a response to the prior anticancer drug treatment.

Embodiment P104. An illudofulvene for use in the treatment of a cancer expressing an enhanced MYC biomarker, and an enhanced PTGR biomarker, wherein said expression includes: (a) contacting a sample from a patient comprising one or more nucleic acid molecules with a device comprising single-stranded nucleic acid molecules capable of specifically hybridizing with the nucleotides of a PTGR gene, and a MYC gene; (b) detecting a level of expression of a PTGR biomarker and a MYC biomarker by performing microarray analysis or quantitative reverse transcriptase polymerase chain reaction (qRT-PCR) of the sample; and (c) determining that the MYC biomarker is enhanced, and the PTGR biomarker is enhanced.

Embodiment P105. The illudofulvene of Embodiment P104, where the PTGR gene is selected from the group consisting of PTGR1 and PTGR2.

Embodiment P106. The illudofulvene of Embodiment P104, where the PTGR biomarker is enhanced when cells transform from a normal state to cancer cells, or when the cells are exposed to prior anticancer drug treatment and become upregulated as a response to the prior anticancer drug treatment.

Embodiment P107. The illudofulvene of Embodiment P104, where the MYC biomarker is enhanced when cells transform from a normal state to cancer cells, or when the cells are exposed to prior anticancer drug treatment and become upregulated as a response to the prior anticancer drug treatment.

Embodiment P108. The illudofulvene of Embodiment P104, where the illudofulvene is an acylfulvene.

Embodiment P109. The illudofulvene of Embodiment P104, where the illudofulvene is an illudin.

Embodiment P110. An illudofulvene for use in a method of treating a patient with cancer, wherein the method includes: (a) determining whether a test sample from a patient has an enhanced MYC biomarker; (b) determining whether the test sample from a patient has an enhanced PTGR biomarker; (c) if the test sample from the patient includes the enhanced MYC biomarker, and the enhanced PTGR biomarker, then administer an effective amount of the illudofulvene.

Embodiment P111. The illudofulvene of Embodiment P110, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment P112. The illudofulvene of Embodiment P110, where the test sample includes one or more nucleic acid molecules.

Embodiment P113. The illudofulvene of Embodiment P110, where the test sample hybridized specifically with the nucleotides of a MYC gene.

Embodiment P114. The illudofulvene of Embodiment P110, where the test sample hybridized specifically with the nucleotides of a PTGR gene.

Embodiment P115. The illudofulvene of Embodiment P110, where the MYC biomarker is determined to be enhanced by performing microarray analysis or quantitative reverse transcriptase polymerase chain reaction (qRT-PCR) of the test sample.

Embodiment P116. The illudofulvene of Embodiment P110, where the illudofulvene is an acylfulvene.

Embodiment P117. The illudofulvene of Embodiment P110, where the illudofulvene is an illudin.

Embodiment P118. The illudofulvene of Embodiment P110, where the PTGR gene is selected from the group consisting of PTGR1 and PTGR2.

Embodiment P119. The illudofulvene of Embodiment P110, where the MYC biomarker is enhanced when cells transform from a normal state to cancer cells, or when the cells are exposed to prior anticancer drug treatment and become upregulated as a response to the prior anticancer drug treatment.

Embodiment P120. The illudofulvene of Embodiment P110, where the PTGR biomarker is enhanced when cells transform from a normal state to cancer cells, or when the cells are exposed to prior anticancer drug treatment and become upregulated as a response to the prior anticancer drug treatment.

Embodiment P121. An illudofulvene for use in the treatment of a cancer that is resistant to one or more Other Drugs, comprising: (a) contacting a sample from a patient comprising one or more nucleic acid molecules with a device comprising single-stranded nucleic acid molecules capable of specifically hybridizing with the nucleotides selected from the group consisting of ERCC8 (SEQ ID NOS:1, 2, 35-45), ERCC6 (SEQ ID NOS:3, 4, 46-56), GTF2H1 (SEQ ID NOS:5, 6, 57-67), GTF2H2 (SEQ ID NOS:7, 8, 68-78), GTF2H3 (SEQ ID NOS:9, 10, 79-89), GTF2H4 (SEQ ID NOS:11, 12, 90-100), GTF2H5 (SEQ ID NOS:13, 14, 101-111), USP7 (SEQ ID NOS:15, 16, 112-122), UVSSA (SEQ ID NOS:17, 18, 123-133), ERCC3 (SEQ ID NOS:19, 20, 134-144), ERCC2 (SEQ ID NOS:21, 22, 145-155), ERCC4 (SEQ ID NOS:23, 24, 156-166), ERCC5 (SEQ ID NOS:25, 26, 167-177), XPA (SEQ ID NOS:27, 28, 178-188), RAD18 (SEQ ID NOS:29, 30, 189-199), ERCC6L (SEQ ID NOS:31, 32, 200-210), ERCC6L2 (SEQ ID NOS:33, 34, 211-221), MYC (SEQ ID NOS:222, 223, 224-235), ERCC1 (SEQ ID NOS:236, 237, 238-248), PTGR1 (SEQ ID NOS:249, 250, 251-261), and/or PTGR2 (SEQ ID NOS:262, 263, 264-274); (b) detecting a level of expression of a plurality of biomarkers selected from ERCC8 (SEQ ID NOS:1, 2, 35-45), ERCC6 (SEQ ID NOS:3, 4, 46-56), GTF2H1 (SEQ ID NOS:5, 6, 57-67), GTF2H2 (SEQ ID NOS:7, 8, 68-78), GTF2H3 (SEQ ID NOS:9, 10, 79-89), GTF2H4 (SEQ ID NOS:11, 12, 90-100), GTF2H5 (SEQ ID NOS:13, 14, 101-111), USP7 (SEQ ID NOS:15, 16, 112-122), UVSSA (SEQ ID NOS:17, 18, 123-133), ERCC3 (SEQ ID NOS:19, 20, 134-144), ERCC2 (SEQ ID NOS:21, 22, 145-155), ERCC4 (SEQ ID NOS:23, 24, 156-166), ERCC5 (SEQ ID NOS:25, 26, 167-177), XPA (SEQ ID NOS:27, 28, 178-188), RAD18 (SEQ ID NOS:29, 30, 189-199), ERCC6L (SEQ ID NOS:31, 32, 200-210), ERCC6L2 (SEQ ID NOS:33, 34, 211-221), MYC (SEQ ID NOS:222, 223, 224-235), ERCC1 (SEQ ID NOS:236, 237, 238-248), PTGR1 (SEQ ID NOS:249, 250, 251-261), and/or PTGR2 (SEQ ID NOS:262, 263, 264-274) by performing microarray analysis or quantitative reverse transcriptase polymerase chain reaction (qRT-PCR) of the sample to generate a biomarker profile; and (c) determining to administer the illudofulvene based on the biomarker profile.

Embodiment P122. The illudofulvene of Embodiment P121, further comprising administering one or more additional therapies prior to, concurrently with, or after administration of the illudofulvene.

Embodiment P123. The illudofulvene of Embodiment P122, where one or more additional therapies includes surgery, radiation, or one or more Other Drugs.

Embodiment P124. The illudofulvene of Embodiment P121, where the cancer is selected from the group consisting of prostate cancer, ovarian cancer, hepatocellular carcinoma (HCC), cervical cancer, renal cell carcinoma (RCC), esophageal cancer, melanoma, glioma, pancreatic cancer, gastrointestinal stromal tumors (GIST), sarcoma, estrogen receptor-positive (ERpos) breast cancer, non-small cell lung carcinoma (NSCLC), colon cancer, bladder cancer, squamous cell carcinoma of the head and neck (SCCHN), acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), and Hodgkin's lymphoma.

Embodiment P125. The illudofulvene of Embodiment P121, where the biomarker profile includes (c) calculating a difference score by subtracting the mean expression levels of one or more of the plurality of biomarkers from the mean expression levels of the one or more of the plurality of biomarkers of sensitivity.

Embodiments contemplated herein further include Embodiments Q1-Q54 following.

Embodiment Q1. An illudofulvene for use in the treatment of a cancer expressing an attenuated TCR biomarker, wherein said expression includes: (a) contacting a sample from a human patient with a means for determining the regulation of a PTGR gene, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity; (b) determining the level of regulation of the PTGR gene; (b) determining the level of regulation of the PTGR gene; (c) analyzing the sample from the human patient with a means for determining the regulation of a MYC gene, where the means is selected from the group consisting of (i) quantification of gene mRNA expression by probe analysis, (ii) microarray analysis, (iii) RT-PCR, (iv) TCR Tumor Mutation Panel and (v) primary tumor cell sensitivity; (d) determining the level of regulation of the MYC gene; (e) analyzing the sample from the human patient with a means for determining the regulation of three (3) or more TCR genes, where the means is selected from the group consisting of (i) quantification of gene mRNA expression by probe analysis, (ii) microarray analysis, (iii) RT-PCR, (iv) TCR Tumor Mutation Panel and (v) primary tumor cell sensitivity; where one or more of the following conditions are fulfilled: (I) the PTGR gene is upregulated, the MYC gene is upregulated, and at least one (1) of the three (3) or more TCR genes is downregulated;

(II) the PTGR gene is upregulated, the MYC gene is not upregulated, and at least one (1) of the three (3) or more TCR genes is downregulated; (III) the PTGR gene is not upregulated, the MYC gene is upregulated, and at least two (2) of the three (3) or more TCR genes is downregulated; and (IV) the PTGR gene is not upregulated, the MYC gene is not upregulated, and at least three (3) of the three (3) or more TCR genes is downregulated.

Embodiment Q2. The illudofulvene of Embodiment Q1, where the TCR gene is selected from the group consisting of an ERCC gene, a XPA gene, a GTF series gene, a UV gene, and a USP gene.

Embodiment Q3. The illudofulvene of Embodiment Q1, where the PTGR gene upregulated is PTGR1, where the process of analysis is quantification of gene mRNA expression by probe analysis, where the primers selected to hybridize with the one or more nucleic acid molecules are selected from the group consisting of SEQ ID NO:307-308.

Embodiment Q4. An illudofulvene for use in a method of treating a patient with cancer, wherein the method includes: (a) determining whether a test sample from a patient has an upregulated PTGR gene; (b) analyzing the sample with a means for determining that at least one TCR gene is downregulated, where the test sample is analyzed with one or more methods selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity; and (c) if the test sample from the patient includes the upregulated PTGR gene and at least one downregulated TCR gene, then administer an effective amount of the illudofulvene.

Embodiment Q5. The illudofulvene of Embodiment Q4, where the TCR gene is selected from the group consisting of an ERCC gene, a XPA gene, a GTF series gene, a UV gene, and a USP gene.

Embodiment Q6. The illudofulvene of Embodiment Q4, where the PTGR gene upregulated is PTGR1, where the process of analysis is quantification of gene mRNA expression by probe analysis, where the primers selected to hybridize with the one or more nucleic acid molecules are selected from the group consisting of SEQ ID NO:307-308.

Embodiment Q7. The illudofulvene of Embodiment Q4, where the PTGR gene upregulated is PTGR1, where the process of analysis is microarray analysis, where the primers selected to hybridize with the one or more nucleic acid molecules are selected from the group consisting of SEQ ID NO:251-261.

Embodiment Q8. The illudofulvene of Embodiment Q4, where the PTGR gene upregulated is PTGR1, where the process of analysis is RT-PCR, where one or both the primers and probe are selected to hybridize with the one or more nucleic acid molecules are selected from the group consisting of SEQ ID NO:897-899.

Embodiment Q9. The illudofulvene of Embodiment Q4, where the PTGR gene upregulated is PTGR1, where the process of analysis is TCR Tumor Mutation panel, where the primers selected to hybridize with the one or more nucleic acid molecules are selected from the group consisting of SEQ ID NO:682-696.

Embodiment Q10. The illudofulvene of Embodiment Q4, where the PTGR gene upregulated is PTGR1, where the process of analysis is primary tumor cell sensitivity.

Embodiment Q11. The illudofulvene of Embodiment Q4, where the PTGR gene upregulated is PTGR2, where the process of analysis is quantification of gene mRNA expression by probe analysis, where the primers selected to hybridize with the one or more nucleic acid molecules are selected from the group consisting of SEQ ID NO:309-310.

Embodiment Q12. The illudofulvene of Embodiment Q4, where the PTGR gene upregulated is PTGR2, where the process of analysis is microarray analysis, where the primers selected to hybridize with the one or more nucleic acid molecules are selected from the group consisting of SEQ ID NO:264-274.

Embodiment Q13. The illudofulvene of Embodiment Q4, where the PTGR gene upregulated is PTGR2, where the process of analysis is RT-PCR, where one or both the primers and probe are selected to hybridize with the one or more nucleic acid molecules are selected from the group consisting of SEQ ID NO:900-902.

Embodiment Q14. The illudofulvene of Embodiment Q4, where the PTGR gene upregulated is PTGR2, where the process of analysis is TCR Tumor Mutation panel, where the primers selected to hybridize with the one or more nucleic acid molecules are selected from the group consisting of SEQ ID NO: 715-727.

Embodiment Q15. The illudofulvene of Embodiment Q4, where the PTGR gene upregulated is PTGR2, where the process of analysis is primary tumor cell sensitivity.

Embodiment Q16. The illudofulvene of Embodiment Q4, where the one or more TCR genes determined to be downregulated are analyzed using the TCR Tumor Mutation panel, where the primers selected to hybridize with the one or more nucleic acid molecules are selected from the group consisting of SEQ ID NO: 329-440, and 451-851.

Embodiment Q17. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with a device including single-stranded nucleic acid molecules capable of specifically hybridizing with nucleotides of a PTGR gene; (b) detecting a level of expression of a PTGR mRNA by performing microarray analysis or quantitative reverse transcriptase polymerase chain reaction (qRT-PCR) of the sample; (c) determining that the PTGR mRNA is upregulated in the human patient; (d) contacting a sample from the human patient including one or more nucleic acid molecules with a device including single-stranded nucleic acid molecules capable of specifically hybridizing with nucleotides of a TCR gene; (e) detecting a level of expression of a plurality of TCR mRNAs by performing microarray analysis or quantitative reverse transcriptase polymerase chain reaction (qRT-PCR) of the sample; (f) determining that the level of expression of at least one of the plurality of TCR mRNAs is downregulated in the human patient; and (g) administering the illudofulvene to the human patient, thereby treating cancer in the human patient.

Embodiment Q18. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with a device including a plurality of primers capable of specifically hybridizing with nucleotides of a PTGR gene selected from the group consisting of SEQ ID NO:307-310; (b) detecting a level of expression of a PTGR mRNA by performing microarray analysis or quantitative reverse transcriptase polymerase chain reaction (qRT-PCR) of the sample; (c) determining that the PTGR gene is upregulated in the human patient; (d) contacting a sample from the human patient including one or more nucleic acid molecules with a device including a plurality of primers capable of specifically hybridizing with nucleotides of a TCR gene selected from the group consisting of SEQ ID NO:275-304, and 311-322; (e) detecting a level of expression of a plurality of TCR mRNAs by performing microarray analysis or quantitative reverse transcriptase polymerase chain reaction (qRT-PCR) of the sample; (f) determining that at least one of the plurality of TCR mRNAs is not upregulated in the human patient; and (g) administering the illudofulvene to the human patient, thereby treating cancer in the human patient.

Embodiment Q19. An illudofulvene for use in treating cancer in a human patient in need thereof including administering the illudofulvene to the human patient, where the human patient has been determined to be responsive to the illudofulvene according to a process including: (a) analyzing a sample from the human patient with a means for determining the regulation of a PTGR gene, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity; (b) determining that the PTGR gene is not upregulated; (c) analyzing a sample from the human patient with a means for determining the regulation of a MYC gene, where the means is selected from the group consisting of (i) quantification of gene mRNA expression by probe analysis, (ii) microarray analysis, (iii) RT-PCR, (iv) TCR Tumor Mutation Panel and (v) primary tumor cell sensitivity; (d) determining that the MYC gene is upregulated; (e) analyzing a sample from the human patient with a means for determining the regulation of a plurality of TCR genes, where the means is selected from the group consisting of (i) quantification of gene mRNA expression by probe analysis, (ii) microarray analysis, (iii) RT-PCR, (iv) TCR Tumor Mutation Panel and (v) primary tumor cell sensitivity; (f) determining that at least two of the plurality of TCR genes are downregulated; and (g) administering the illudofulvene to the human patient, thereby treating cancer in the human patient.

Embodiment Q20. The illudofulvene of Embodiment Q19, where the MYC gene determined to be upregulated is analyzed using the TCR Tumor Mutation panel, where the primers selected to hybridize with one or more nucleic acid molecules are selected from the group consisting of SEQ ID NO:669-681.

Embodiment Q21. The illudofulvene of Embodiment Q19, where the process of analysis to determine that the PTGR gene is not upregulated is quantification of gene mRNA expression by probe analysis, where the primers selected to hybridize with one or more nucleic acid molecules are selected from the group consisting of SEQ ID NO:307-310.

Embodiment Q22. The illudofulvene of Embodiment Q19, where the process of analysis to determine that the PTGR gene is not upregulated is microarray analysis, where the primers selected to hybridize with one or more nucleic acid molecules are selected from the group consisting of SEQ ID NO:251-261, 264-274.

Embodiment Q23. The illudofulvene of Embodiment Q19, where the process of analysis to determine that the PTGR gene is not upregulated is RT-PCR, where one or both the primers and probe selected to hybridize with one or more nucleic acid molecules are selected from the group consisting of SEQ ID NO:897-902.

Embodiment Q24. The illudofulvene of Embodiment Q19, where the process of analysis to determine that the PTGR gene is not upregulated is TCR Tumor Mutation panel, where the primers selected to hybridize with one or more nucleic acid molecules are selected from the group consisting of SEQ ID NO:682-696, 715-727.

Embodiment Q25. The illudofulvene of Embodiment Q19, where the process of analysis to determine that the PTGR gene is not upregulated is primary tumor cell sensitivity.

Embodiment Q26. The illudofulvene of Embodiment Q19, where the process of analysis to determine that one or more TCR genes are downregulated is quantification of gene mRNA expression by probe analysis, where the primers selected to hybridize with one or more nucleic acid molecules are selected from the group consisting of SEQ ID NO:275-304 and 311-322.

Embodiment Q27. The illudofulvene of Embodiment Q19, where the process of analysis to determine that one or more TCR genes are downregulated is microarray analysis, where the primers selected to hybridize with one or more nucleic acid molecules are selected from the group consisting of SEQ ID NO:35-248.

Embodiment Q28. The illudofulvene of Embodiment Q19, where the process of analysis to determine that one or more TCR genes are downregulated is RT-PCR, where one or both the primers and probe selected to hybridize with one or more nucleic acid molecules are selected from the group consisting of SEQ ID NO:852-893 and 903-917.

Embodiment Q29. The illudofulvene of Embodiment Q19, where the process of analysis to determine that one or more TCR genes are downregulated is TCR Tumor Mutation panel, where the primers selected to hybridize with one or more nucleic acid molecules are selected from the group consisting of SEQ ID NO:329-440, and 451-851.

Embodiment Q30. The illudofulvene of Embodiment Q19, where the process of analysis to determine that one or more TCR genes are downregulated is primary tumor cell sensitivity.

Embodiment Q31. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with one or more methods selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity to determine the level of expression of PTGR genes; (b) determining that the PTGR gene is not upregulated; (c) contacting a sample from the human patient including one or more nucleic acid molecules with one or more methods selected from the group consisting of (i) quantification of gene mRNA expression by probe analysis, (ii) microarray analysis, (iii) RT-PCR, (iv) TCR Tumor Mutation Panel and (v) primary tumor cell sensitivity to determine the level of expression of a MYC gene; (d) determining that the MYC gene is upregulated; (e) contacting a sample from the human patient including one or more nucleic acid molecules with one or more methods selected from the group consisting of (i) quantification of gene mRNA expression by probe analysis, (ii) microarray analysis, (iii) RT-PCR, (iv) TCR Tumor Mutation Panel and (v) primary tumor cell sensitivity to determine the level of expression of a plurality of TCR genes; (f) determining that the level of expression of at least two of the plurality of TCR genes are downregulated; and (g) administering the illudofulvene to the human patient, thereby treating cancer in the human patient.

Embodiment Q32. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with a device including single-stranded nucleic acid molecules capable of specifically hybridizing with nucleotides of a PTGR gene; (b) detecting a level of expression of a PTGR mRNA by performing microarray analysis or quantitative reverse transcriptase polymerase chain reaction (qRT-PCR) of the sample; (c) determining that the level of expression of the PTGR mRNA is not upregulated in the human patient; (d) contacting a sample from the human patient including one or more nucleic acid molecules with a device including single-stranded nucleic acid molecules capable of specifically hybridizing with nucleotides of a MYC gene; (e) detecting a level of expression of a MYC mRNA by performing microarray analysis or quantitative reverse transcriptase polymerase chain reaction (qRT-PCR) of the sample; and (f) determining that the level of expression of the MYC mRNA is upregulated in the human patient; (g) contacting a sample from the human patient including one or more nucleic acid molecules with a device including single-stranded nucleic acid molecules capable of specifically hybridizing with nucleotides of a plurality of TCR genes; (h) detecting a level of expression of a plurality of TCR mRNAs by performing microarray analysis or quantitative reverse transcriptase polymerase chain reaction (qRT-PCR) of the sample; (i) determining that the level of expression of at least two of the plurality of TCR mRNAs is downregulated in the human patient; and (j) administering the illudofulvene to the human patient, thereby treating cancer in the human patient.

Embodiment Q33. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with a device including single-stranded nucleic acid molecules capable of specifically hybridizing with nucleotides of a PTGR gene selected from the group consisting of SEQ ID NO:307-310; (b) detecting a level of expression of a PTGR mRNA by performing microarray analysis or quantitative reverse transcriptase polymerase chain reaction (qRT-PCR) of the sample; (c) determining that the level of expression of the PTGR mRNA is NOT upregulated in the human patient; (d) contacting a sample from the human patient including one or more nucleic acid molecules with a device including single-stranded nucleic acid molecules capable of specifically hybridizing with nucleotides of a MYC gene selected from the group consisting of SEQ ID NO:305-306; (e) detecting a level of expression of a MYC mRNA by performing microarray analysis or quantitative reverse transcriptase polymerase chain reaction (qRT-PCR) of the sample; and (f) determining that the level of expression of the MYC mRNA is upregulated in the human patient; (g) contacting a sample from the human patient including one or more nucleic acid molecules with a device including single-stranded nucleic acid molecules capable of specifically hybridizing with nucleotides of a plurality of TCR genes selected from the group consisting of SEQ ID NO:275-304, and 311-322; (h) detecting a level of expression of a TCR mRNA by performing microarray analysis or quantitative reverse transcriptase polymerase chain reaction (qRT-PCR) of the sample; (i) determining that the level of expression of at least two of the plurality of TCR mRNA is downregulated in the human patient; and (j) administering the illudofulvene to the human patient, thereby treating cancer in the human patient.

Embodiment Q34. An illudofulvene for use in treating cancer in a human patient in need thereof including administering the illudofulvene to the human patient, where the human patient has been determined to be responsive to the illudofulvene according to a process including: (a) analyzing a sample from the human patient with a means for determining the regulation of a PTGR gene, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity; (b) determining that the PTGR gene is not upregulated; (c) analyzing a sample from the human patient with a means for determining the regulation of a MYC gene, where the means is selected from the group consisting of (i) quantification of gene mRNA expression by probe analysis, (ii) microarray analysis, (iii) RT-PCR, (iv) TCR Tumor Mutation Panel and (v) primary tumor cell sensitivity; (d) determining that the MYC gene is not upregulated; (e) analyzing a sample from the human patient with a means for determining the regulation of three (3) or more TCR genes, where the means is selected from the group consisting of (i) quantification of gene mRNA expression by probe analysis, (ii) microarray analysis, (iii) RT-PCR, (iv) TCR Tumor Mutation Panel and (v) primary tumor cell sensitivity; (f) determining that at least three (3) of the three (3) or more TCR genes are downregulated; and (g) administering the illudofulvene to the human patient, thereby treating cancer in the human patient.

Embodiment Q35. The illudofulvene of Embodiment Q34, where the MYC gene determined to not be upregulated is analyzed using the TCR Tumor Mutation panel, where the primers selected to hybridize with one or more nucleic acid molecules are selected from the group consisting of SEQ ID NO:669-681.

Embodiment Q36. The illudofulvene of Embodiment Q34, where the process of analysis to determine that the PTGR gene is not upregulated is quantification of gene mRNA expression by probe analysis, where the primers selected to hybridize with one or more nucleic acid molecules are selected from the group consisting of SEQ ID NO:307-310.

Embodiment Q37. The illudofulvene of Embodiment Q34, where the process of analysis to determine that the PTGR gene is not upregulated is microarray analysis, where the primers selected to hybridize with one or more nucleic acid molecules are selected from the group consisting of SEQ ID NO:251-261, 264-274.

Embodiment Q38. The illudofulvene of Embodiment Q34, where the process of analysis to determine that the PTGR gene is not upregulated is RT-PCR, where one or both the primers and probe selected to hybridize with one or more nucleic acid molecules are selected from the group consisting of SEQ ID NO:897-902.

Embodiment Q39. The illudofulvene of Embodiment Q34, where the process of analysis to determine that the PTGR gene is not upregulated is TCR Tumor Mutation panel, where the primers selected to hybridize with one or more nucleic acid molecules are selected from the group consisting of SEQ ID NO:682-696, 715-727.

Embodiment Q40. The illudofulvene of Embodiment Q34, where the process of analysis to determine that the PTGR gene is not upregulated is primary tumor cell sensitivity.

Embodiment Q41. The illudofulvene of Embodiment Q34, where the process of analysis to determine that one or more TCR genes are downregulated is quantification of gene mRNA expression by probe analysis, where the primers selected to hybridize with one or more nucleic acid molecules are selected from the group consisting of SEQ ID NO:275-304 and 311-322.

Embodiment Q42. The illudofulvene of Embodiment Q34, where the process of analysis to determine that one or more TCR genes are downregulated is microarray analysis, where the primers selected to hybridize with one or more nucleic acid molecules are selected from the group consisting of SEQ ID NO:35-248.

Embodiment Q43. The illudofulvene of Embodiment Q34, where the process of analysis to determine that one or more TCR genes are downregulated is RT-PCR, where one or both the primers and probe selected to hybridize with one or more nucleic acid molecules are selected from the group consisting of SEQ ID NO:852-893 and 903-917.

Embodiment Q44. The illudofulvene of Embodiment Q34, where the process of analysis to determine that one or more TCR genes are downregulated is TCR Tumor Mutation panel, where the primers selected to hybridize with one or more nucleic acid molecules are selected from the group consisting of SEQ ID NO:329-440, and 451-851.

Embodiment Q45. The illudofulvene of Embodiment Q34, where the process of analysis to determine that one or more TCR genes are downregulated is primary tumor cell sensitivity.

Embodiment Q46. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with one or more methods selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity to determine the level of expression of PTGR genes; (b) determining that the level of expression of the PTGR gene is not upregulated; (c) contacting a sample from the human patient including one or more nucleic acid molecules with one or more methods selected from the group consisting of (i) quantification of gene mRNA expression by probe analysis, (ii) microarray analysis, (iii) RT-PCR, (iv) TCR Tumor Mutation Panel and (v) primary tumor cell sensitivity to determine the level of expression of a MYC gene; (d) determining that the MYC gene is not upregulated; (e) contacting a sample from the human patient including one or more nucleic acid molecules with one or more methods selected from the group consisting of (i) quantification of gene mRNA expression by probe analysis, (ii) microarray analysis, (iii) RT-PCR, (iv) TCR Tumor Mutation Panel and (v) primary tumor cell sensitivity to determine the level of expression of three (3) or more TCR genes; (f) determining that the level of expression of at least three (3) of the three (3) or more TCR genes are downregulated; and (g) administering the illudofulvene to the human patient, thereby treating cancer in the human patient.

Embodiment Q47. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with a device including single-stranded nucleic acid molecules capable of specifically hybridizing with nucleotides of a PTGR gene; (b) detecting a level of expression of a PTGR mRNA by performing microarray analysis or quantitative reverse transcriptase polymerase chain reaction (qRT-PCR) of the sample; (c) determining that the level of expression of the PTGR mRNA is not upregulated in the human patient; (d) contacting a sample from the human patient including one or more nucleic acid molecules with a device including single-stranded nucleic acid molecules capable of specifically hybridizing with nucleotides of a MYC gene; (e) detecting a level of expression of a MYC mRNA by performing microarray analysis or quantitative reverse transcriptase polymerase chain reaction (qRT-PCR) of the sample; and (f) determining that the level of expression of the MYC mRNA is not upregulated in the human patient; (g) contacting a sample from the human patient including one or more nucleic acid molecules with a device including single-stranded nucleic acid molecules capable of specifically hybridizing with nucleotides of a plurality of TCR genes; (h) detecting a level of expression of three (3) or more TCR mRNAs by performing microarray analysis or quantitative reverse transcriptase polymerase chain reaction (qRT-PCR) of the sample; (i) determining that the level of expression of at least three (3) of the three (3) or more TCR mRNAs is downregulated in the human patient; and (j) administering the illudofulvene to the human patient, thereby treating cancer in the human patient.

Embodiment Q48. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with a device including single-stranded nucleic acid molecules capable of specifically hybridizing with nucleotides of a PTGR gene selected from the group consisting of SEQ ID NO:307-310; (b) detecting a level of expression of a PTGR mRNA by performing microarray analysis or quantitative reverse transcriptase polymerase chain reaction (qRT-PCR) of the sample; (c) determining that the level of expression of the PTGR mRNA is not upregulated in the human patient; (d) contacting a sample from the human patient including one or more nucleic acid molecules with a device including single-stranded nucleic acid molecules capable of specifically hybridizing with nucleotides of a MYC gene selected from the group consisting of SEQ ID NO:305-306; (e) detecting a level of expression of a MYC mRNA by performing microarray analysis or quantitative reverse transcriptase polymerase chain reaction (qRT-PCR) of the sample; and (f) determining that the level of expression of the MYC mRNA is not upregulated in the human patient; (g) contacting a sample from the human patient including one or more nucleic acid molecules with a device including single-stranded nucleic acid molecules capable of specifically hybridizing with nucleotides of a plurality of TCR genes selected from the group consisting of SEQ ID NO:275-304, and 311-322; (h) detecting a level of expression of three (3) or more TCR mRNAs by performing microarray analysis or quantitative reverse transcriptase polymerase chain reaction (qRT-PCR) of the sample; (i) determining that the level of expression of at least three (3) of the three (3) or more TCR mRNAs is downregulated in the human patient; and (j) administering the illudofulvene to the human patient, thereby treating cancer in the human patient.

Embodiment Q49. An illudofulvene for use in treating cancer in a human patient in need thereof including administering the illudofulvene to the human patient, where the human patient has been determined to be responsive to the illudofulvene according to a process including: (a) contacting a sample from the human patient including one or more nucleic acid molecules with a device including single-stranded nucleic acid molecules capable of specifically hybridizing with nucleotides of a PTGR gene; (b) detecting a level of expression of a PTGR mRNA by performing microarray analysis or quantitative reverse transcriptase polymerase chain reaction (qRT-PCR) of the sample; (c) determining that the level of expression of the PTGR mRNA is not upregulated in the human patient; (d) contacting a sample from the human patient including one or more nucleic acid molecules with a device including single-stranded nucleic acid molecules capable of specifically hybridizing with nucleotides of a MYC gene; (e) detecting a level of expression of a MYC mRNA by performing microarray analysis or quantitative reverse transcriptase polymerase chain reaction (qRT-PCR) of the sample; (f) determining that the level of expression of the MYC mRNA is not upregulated in the human patient; (g) contacting a sample from the human patient including one or more nucleic acid molecules with a device including single-stranded nucleic acid molecules capable of specifically hybridizing with nucleotides of three (3) or more TCR genes; (h) detecting a level of expression of at least three (3) of the three (3) or more TCR mRNAs by performing microarray analysis or quantitative reverse transcriptase polymerase chain reaction (qRT-PCR) of the sample; and (i) determining that at least three (3) of the three (3) or more TCR mRNAs are upregulated in the human patient.

Embodiment Q50. An illudofulvene for use in treating cancer in a human patient in need thereof including administering the illudofulvene to the human patient, where the human patient has been determined to be responsive to the illudofulvene according to a process including: (a) analyzing a sample from the human patient with a means for determining that a PTGR gene is upregulated; (b) analyzing a sample from the human patient with a means for determining that at least one of the TCR genes is downregulated; and (c) administering the illudofulvene to the human patient, thereby treating cancer in the human patient.

Embodiment Q51. The illudofulvene of Embodiment Q19, where the MYC gene determined to be upregulated is not upregulated is RT-PCR, where one or both the primers and probe selected to hybridize with one or more nucleic acid molecules are selected from the group consisting of SEQ ID NO:894-896.

Embodiment Q52. The illudofulvene of Embodiment Q19, where the MYC gene determined to be upregulated is microarray analysis, where the primers selected to hybridize with one or more nucleic acid molecules are selected from the group consisting of SEQ ID NO:224-235.

Embodiment Q53. The illudofulvene of Embodiment Q19, where the process of analysis to determine that the MYC gene is upregulated is primary tumor cell sensitivity.

Embodiment Q54. The illudofulvene of Embodiment Q19, where the MYC gene determined to be upregulated is quantification of gene mRNA expression by probe analysis, where the primers selected to hybridize with one or more nucleic acid molecules are selected from the group consisting of SEQ ID NO:305-306.

Embodiments contemplated herein further include Embodiments R1-R82 following.

Embodiment R1. An illudofulvene for use in the treatment of a cancer expressing an upregulated PTGR gene and an upregulated MYC gene, wherein said expression includes: (a) contacting a sample from a patient comprising one or more nucleic acid molecules with one or more means for determining the regulation of expression of one or more PTGR genes; (b) contacting the sample from the patient with one or more means for determining the regulation of expression of a MYC gene; (c) contacting the sample from the patient with one or more means for determining the regulation of expression of one or more TCR genes; (d) detecting a level of expression of the one or more PTGR genes that are upregulated; (e) detecting a level of expression of the MYC gene that is upregulated; and (f) detecting a deficiency in a level of expression of one or more TCR genes.

Embodiment R2. The illudofulvene of Embodiment R1, where the one or more TCR genes are selected from the group consisting of an ERCC gene, a XPA gene, a GTF series gene, a UV gene, and a USP gene.

Embodiment R3. The illudofulvene of Embodiments R1 or R2, where the one or more PTGR genes are selected from the group consisting of PTGR1 and PTGR2.

Embodiment R4. The illudofulvene of Embodiments R1-R3, where the one or more PTGR genes include PTGR1.

Embodiment R5. The illudofulvene of Embodiments R1-R4, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R6. The illudofulvene of Embodiments R1-R5, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R7. The illudofulvene of Embodiments R1-R3, where the one or more PTGR genes include PTGR2.

Embodiment R8. The illudofulvene of Embodiments R1-R3 or R7, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R9. The illudofulvene of Embodiments R1-R3 or R7-R8, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R10. An illudofulvene for use in a method of treating a patient with cancer, wherein the method includes: (a) determining whether a test sample from a patient has a level of expression of one or more PTGR genes that are upregulated, a level of expression of a MYC gene that is upregulated, and a deficiency in a level of expression of one or more TCR genes; and (b) if the test sample from the patient includes the one or more upregulated PTGR genes, the upregulated MYC gene, and the deficiency in the level of expression of one or more TCR genes, then administer an effective amount of the illudofulvene.

Embodiment R11. The illudofulvene of Embodiment R10, where the one or more TCR genes are selected from the group consisting of an ERCC gene, a XPA gene, a GTF series gene, a UV gene, and a USP gene.

Embodiment R12. The illudofulvene of Embodiments R10 or R11, where the one or more PTGR genes are selected from the group consisting of PTGR1 and PTGR2.

Embodiment R13. The illudofulvene of Embodiments R10-R12, where the means is where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R14. The illudofulvene of Embodiments R10-R13, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R15. The illudofulvene of Embodiments R10-R14, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment R16. An illudofulvene for use in the treatment of a cancer expressing a PTGR gene and one or more TCR genes, wherein said expression includes: (a) contacting a sample from a patient comprising one or more nucleic acid molecules with one or more means for determining the regulation of expression of one or more PTGR genes; (b) contacting the sample from the patient with one or more means for determining the regulation of expression of one or more TCR genes; (c) detecting a level of expression of the one or more PTGR genes that are upregulated; and (d) detecting a deficiency in a level of expression of one or more TCR genes.

Embodiment R17. The illudofulvene of Embodiment R16, where the one or more TCR genes are selected from the group consisting of an ERCC gene, a XPA gene, a GTF series gene, a UV gene, and a USP gene.

Embodiment R18. The illudofulvene of Embodiments R16 or R17, where the one or more PTGR genes are selected from the group consisting of PTGR1 and PTGR2.

Embodiment R19. The illudofulvene of Embodiments R16-R8, where the one or more PTGR genes include PTGR1.

Embodiment R20. The illudofulvene of Embodiments R16-R19, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R21. The illudofulvene of Embodiments R16-R20, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R22. The illudofulvene of Embodiments R16-R18, where the one or more PTGR genes include PTGR2.

Embodiment R23. The illudofulvene of Embodiments R16-R18 or R22, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R24. The illudofulvene of Embodiments R16-R18 or R22-R23, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R25. An illudofulvene for use in the treatment of a cancer expressing a MYC gene and one or more TCR genes, wherein said expression includes: (a) contacting a sample from a patient comprising one or more nucleic acid molecules with one or more means for determining the regulation of expression of a MYC gene; (b) contacting the sample from the patient with one or more means for determining the regulation of expression of one or more TCR genes; (c) detecting a level of expression of the MYC gene that is upregulated; and (d) detecting a deficiency in a level of expression of one or more TCR genes.

Embodiment R26. The illudofulvene of Embodiment R25, where the one or more TCR genes are selected from the group consisting of an ERCC gene, a XPA gene, a GTF series gene, a UV gene, and a USP gene.

Embodiment R27. The illudofulvene of Embodiments R25 or R26, where the one or more PTGR genes are selected from the group consisting of PTGR1 and PTGR2.

Embodiment R28. The illudofulvene of Embodiments R25-R27, where the one or more PTGR genes include PTGR1.

Embodiment R29. The illudofulvene of Embodiments R25-R28, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R30. The illudofulvene of Embodiments R25-R29, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R31. The illudofulvene of Embodiments R25-R27, where the one or more PTGR genes include PTGR2.

Embodiment R32. The illudofulvene of Embodiments R25-R27 or R31, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R33. The illudofulvene of Embodiments R25-R27 or R31-R32, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R34. An illudofulvene for use in the treatment of a cancer expressing a PTGR gene, a MYC gene and one or more TCR genes, wherein said expression includes: (a) contacting a sample from a patient comprising one or more nucleic acid molecules with one or more means for determining the regulation of expression of one or more PTGR genes; (b) contacting the sample from the patient with one or more means for determining the regulation of expression of a MYC gene; (c) contacting the sample from the patient with one or more means for determining the regulation of expression of one or more TCR genes; (d) detecting either a level of expression of the one or more PTGR genes that are upregulated, or detecting a level of expression of the MYC gene that is upregulated; and (e) detecting a deficiency in a level of expression of one or more TCR genes.

Embodiment R35. The illudofulvene of Embodiment R34, where the one or more TCR genes are selected from the group consisting of one or more ERCC genes, a XPA gene, a GTF series gene, a UV gene, and a USP gene.

Embodiment R36. The illudofulvene of Embodiments R34 or R35, where the one or more PTGR genes are selected from the group consisting of PTGR1 and PTGR2.

Embodiment R37. The illudofulvene of Embodiments R34-R36, where the one or more PTGR genes include PTGR1.

Embodiment R38. The illudofulvene of Embodiments R34-R37, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R39. The illudofulvene of Embodiments R34-R38, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R40. The illudofulvene of Embodiments R34-R37, where the one or more PTGR genes include PTGR2.

Embodiment R41. The illudofulvene of Embodiments R34-R37 or R40, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R42. The illudofulvene of Embodiments R34-R37 or R40-R41, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R43. An illudofulvene for use in the treatment of a cancer expressing three or more TCR genes, wherein said expression includes: (a) contacting a sample from a patient comprising one or more nucleic acid molecules with one or more means for determining the regulation of expression of three or more TCR genes; and (b) detecting a deficiency in a level of expression of three or more TCR genes.

Embodiment R44. The illudofulvene of Embodiment R43, where the three or more TCR genes are selected from the group consisting of one or more ERCC genes, a XPA gene, a GTF series gene, a UV gene, and a USP gene.

Embodiment R45. The illudofulvene of Embodiments R43 or R44, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R46. An illudofulvene for use in the treatment of a cancer expressing a PTGR gene, a MYC gene and one or more TCR genes, wherein said expression includes: (a) contacting a sample from a patient comprising one or more nucleic acid molecules with one or more means for determining the regulation of expression of one or more PTGR genes; (b) contacting the sample from the patient with one or more means for determining the regulation of expression of a MYC gene; (c) contacting the sample from the patient with one or more means for determining the regulation of expression of one or more TCR genes; (d) detecting either a level of expression of the one or more PTGR genes that are upregulated, or detecting a level of expression of the MYC gene that is upregulated; (e) if one or more PTGR genes are upregulated then detecting a deficiency in a level of expression of one or more TCR genes; and (f) if the MYC gene is upregulated then detecting a deficiency in a level of expression of two or more TCR genes.

Embodiment R47. The illudofulvene of Embodiment R45, where the one or more TCR genes are selected from the group consisting of one or more ERCC genes, a XPA gene, a GTF series gene, a UV gene, and a USP gene.

Embodiment R48. The illudofulvene of Embodiment R46 or R47, where the one or more PTGR genes are selected from the group consisting of PTGR1 and PTGR2.

Embodiment R49. The illudofulvene of Embodiment R46 to R48, where the one or more PTGR genes include PTGR1.

Embodiment R50. The illudofulvene of Embodiment R46 to R49, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R51. The illudofulvene of Embodiment R46 to R50, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R52. The illudofulvene of Embodiment R46 to R48, where the one or more PTGR genes include PTGR2.

Embodiment R53. The illudofulvene of Embodiment R46 to R48 or R52, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R54. The illudofulvene of Embodiment R46 to R48 or R52 to 53, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R55. An illudofulvene for use in a method of treating a patient with cancer, wherein the method includes: (a) determining whether a test sample from a patient has a level of expression of one or more PTGR genes that are upregulated, a level of expression of a MYC gene that is upregulated, and a deficiency in a level of expression of one or more TCR genes; (b) if the test sample from the patient includes the one or more upregulated PTGR genes, and the deficiency in the level of expression of one or more TCR genes, then administer an effective amount of the illudofulvene; or (c) if the test sample from the patient includes the upregulated MYC gene, and the deficiency in the level of expression of two or more TCR genes, then administer an effective amount of the illudofulvene.

Embodiment R56. The illudofulvene of Embodiment R55, where the one or more TCR genes are selected from the group consisting of one or more ERCC genes, a XPA gene, a GTF series gene, a UV gene, and a USP gene.

Embodiment R57. The illudofulvene of Embodiment R55 or R56, where the one or more PTGR genes are selected from the group consisting of PTGR1 and PTGR2.

Embodiment R58. The illudofulvene of Embodiment R55 to R57, where the means is where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R59. The illudofulvene of Embodiment R55 to R58, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R60. The illudofulvene of Embodiment R55 to R59, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment R61. An illudofulvene analog for use in the treatment of a cancerous tumor expressing a PTGR gene, a MYC gene or one or more TCR genes, wherein said expression includes: (a) contacting a sample from a patient comprising one or more nucleic acid molecules with one or more means for determining the regulation of expression of one or more PTGR genes, the regulation of expression of a MYC gene, and the regulation of expression of one or more TCR genes; and (b) determining if a cancerous tumor is sensitive to an illudofulvene analog based on a first relative ratio of expression of the one or more PTGR gene compared with a housekeeping gene and a second relative ratio of expression of the TCR genes compared with a housekeeping gene, or a third relative ratio of expression of the MYC gene compared with a housekeeping gene and the second relative ratio of expression.

Embodiment R62. The illudofulvene of Embodiment R61, where the one or more TCR genes are selected from the group consisting of one or more ERCC genes, a XPA gene, a GTF series gene, a UV gene, and a USP gene.

Embodiment R63. The illudofulvene of Embodiment R61 or 62, where the one or more PTGR genes are selected from the group consisting of PTGR1 and PTGR2.

Embodiment R64. The illudofulvene of Embodiment R61 to 63, where the one or more housekeeping genes are selecting from the group consisting of beta actin (ACTB), transferrin receptor (TFRC) and glyceraldehyde-3-phosphate dehydrogenase (GAPD).

Embodiment R65. The illudofulvene of Embodiment R61 to 64, where the one or more PTGR genes include PTGR1.

Embodiment R66. The illudofulvene of Embodiment R61 to 65, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R67. The illudofulvene of Embodiment R61 to 66, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R68. The illudofulvene of Embodiment R61 to 64, where the one or more PTGR genes include PTGR2.

Embodiment R69. The illudofulvene of Embodiment R61 to 44 or 68, where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R70. An illudofulvene for use in a method of treating a patient with cancer, wherein the method includes: (a) determining from a patient test sample a first relative ratio of expression of the one or more PTGR genes compared with at least one of the one or more housekeeping genes or determining a second relative ratio of expression of the MYC gene compared with at least one of the one or more housekeeping genes; (b) determining a third relative ratio of expression of the TCR genes compared with at least one of the one or more housekeeping genes; (c) if the first relative ratio of expression is high and the third relative ratio of expression is deficient or the second relative ratio of expression is high and the third relative ratio of expression is deficient, then administer an effective amount of the illudofulvene.

Embodiment R71. The illudofulvene of Embodiment R70, where the one or more TCR genes are selected from the group consisting of one or more ERCC genes, a XPA gene, a GTF series gene, a UV gene, and a USP gene.

Embodiment R72. The illudofulvene of Embodiment R70 or R71, where the one or more PTGR genes are selected from the group consisting of PTGR1 and PTGR2.

Embodiment R73. The illudofulvene of Embodiment R70 to R72, where the means is where the means is selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment R74. The illudofulvene of Embodiment R70 to R73, where one or more primers selected to hybridize with the one or more nucleic acid molecules used to quantify expression of one or more PTGR genes, the MYC gene and the one or more TCR genes are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, and SEQ ID NO:900-902.

Embodiment R75. The illudofulvene of Embodiment R70 to R74, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment R76. An illudofulvene analog selected from the group consisting of illudin ring derivatives, illudin alkyl derivatives and acyl-fulvenes for use in the treatment of a cancerous tumor expressing a PTGR gene, a MYC gene or one or more TCR genes, wherein said expression includes: (a) contacting a sample from a patient comprising one or more nucleic acid molecules with one or more means for determining: (i) the regulation of expression of one or more PTGR genes; (ii) the regulation of expression of at least one TCR gene; (iii) the regulation of expression of at least one housekeeping gene; (b) determining if a cancerous tumor is sensitive to an illudofulvene analog based on a first relative ratio of expression of the one or more PTGR genes compared with the at least one housekeeping gene, when the first relative ratio of expression is high, and a second relative ratio of expression of the at least one TCR gene compared with the at least one housekeeping gene, when the second relative ratio of expression is deficient; or (c) contacting the sample comprising one or more nucleic acid molecules with one or more means for determining: (iv) the regulation of expression of a MYC gene; (v) the regulation of expression of two or more TCR genes; (vi) the regulation of expression of one or more housekeeping genes; (d) determining if the cancerous tumor is sensitive to the illudofulvene analog based on a third relative ratio of expression of the MYC gene compared with the one or more housekeeping genes, when the third relative ratio of expression is high, and a fourth relative ratio of expression of two or more TCR genes compared with the one or more housekeeping genes, when the fourth relative ratio of expression is deficient.

Embodiment R77. An illudofulvene analog selected from the group consisting of illudin ring derivatives and illudin alkyl derivatives for use in the treatment of a cancerous tumor expressing a PTGR gene, a MYC gene or one or more TCR genes, wherein said expression includes: (a) contacting a sample from a patient comprising one or more nucleic acid molecules with one or more means for determining: (i) the regulation of expression of one or more PTGR genes; (ii) the regulation of expression of at least one TCR gene; (iii) the regulation of expression of at least one housekeeping gene; (b) determining if a cancerous tumor is sensitive to an illudofulvene analog based on a first relative ratio of expression of the one or more PTGR genes compared with the at least one housekeeping gene, when the first relative ratio of expression is high, and a second relative ratio of expression of the at least one TCR gene compared with the at least one housekeeping gene, when the second relative ratio of expression is deficient; or (c) contacting the sample comprising one or more nucleic acid molecules with one or more means for determining: (iv) the regulation of expression of a MYC gene; (v) the regulation of expression of two or more TCR genes; (vi) the regulation of expression of one or more housekeeping genes; (d) determining if the cancerous tumor is sensitive to the illudofulvene analog based on a third relative ratio of expression of the MYC gene compared with the one or more housekeeping genes, when the third relative ratio of expression is high, and a fourth relative ratio of expression of two or more TCR genes compared with the one or more housekeeping genes, when the fourth relative ratio of expression is deficient.

Embodiment R78. An illudofulvene analog selected from the group consisting of illudins and acyl-fulvenes for use in the treatment of a cancerous tumor expressing a PTGR gene, a MYC gene or one or more TCR genes, wherein said expression includes: (a) contacting a sample from a patient comprising one or more nucleic acid molecules with one or more means for determining: (i) the regulation of expression of one or more PTGR genes; (ii) the regulation of expression of at least one TCR gene; (iii) the regulation of expression of at least one housekeeping gene; (b) determining if a cancerous tumor is sensitive to an illudofulvene analog based on a first relative ratio of expression of the one or more PTGR genes compared with the at least one housekeeping gene, when the first relative ratio of expression is high, and a second relative ratio of expression of the at least one TCR gene compared with the at least one housekeeping gene, when the second relative ratio of expression is deficient; or (c) contacting the sample comprising one or more nucleic acid molecules with one or more means for determining: (iv) the regulation of expression of a MYC gene; (v) the regulation of expression of two or more TCR genes; (vi) the regulation of expression of one or more housekeeping genes; (d) determining if the cancerous tumor is sensitive to the illudofulvene analog based on a third relative ratio of expression of the MYC gene compared with the one or more housekeeping genes, when the third relative ratio of expression is high, and a fourth relative ratio of expression of two or more TCR genes compared with the one or more housekeeping genes, when the fourth relative ratio of expression is deficient.

Embodiment R79. An illudofulvene analog selected from the group consisting of illudin ring derivatives, illudin alkyl derivatives and acyl-fulvenes for use in a method of treating a patient with cancer, wherein the method includes: (a) determining from a patient test sample a first relative ratio of expression based on expression of the one or more PTGR genes compared with expression of at least one housekeeping gene; (b) determining from the patient test sample a second relative ratio of expression based on expression of at least one TCR genes compared with expression of at least one housekeeping gene; (c) determining that the patient is sensitive, if the first relative ratio of expression is high and the second relative ratio of expression is deficient, then administer an effective amount of the illudofulvene; or (d) determining from a patient test sample a third relative ratio of expression based on expression of a MYC gene compared with at least one of the housekeeping genes; (e) determining from a patient test sample a fourth relative ratio of expression based on expression of two or more TCR genes compared with at least one of the housekeeping genes; (f) determining that the patient is sensitive, if the third relative ratio of expression is high and the fourth relative ratio of expression is deficient, then administer an effective amount of the illudofulvene.

Embodiment R80. An illudofulvene analog selected from the group consisting of illudin ring derivatives and illudin alkyl derivatives for use in a method of treating a patient with cancer, wherein the method includes: (a) determining from a patient test sample a first relative ratio of expression based on expression of the one or more PTGR genes compared with expression of at least one housekeeping gene; (b) determining from the patient test sample a second relative ratio of expression based on expression of at least one TCR genes compared with expression of at least one housekeeping gene; (c) determining that the patient is sensitive, if the first relative ratio of expression is high and the second relative ratio of expression is deficient, then administer an effective amount of the illudofulvene; or (d) determining from a patient test sample a third relative ratio of expression based on expression of a MYC gene compared with at least one of the housekeeping genes; (e) determining from a patient test sample a fourth relative ratio of expression based on expression of two or more TCR genes compared with at least one of the housekeeping genes; (f) determining that the patient is sensitive, if the third relative ratio of expression is high and the fourth relative ratio of expression is deficient, then administer an effective amount of the illudofulvene.

Embodiment R81. An illudofulvene analog selected from the group consisting of illudins and acyl-fulvenes for use in a method of treating a patient with cancer, wherein the method includes: (a) determining from a patient test sample a first relative ratio of expression based on expression of the one or more PTGR genes compared with expression of at least one housekeeping gene; (b) determining from the patient test sample a second relative ratio of expression based on expression of at least one TCR genes compared with expression of at least one housekeeping gene; (c) determining that the patient is sensitive, if the first relative ratio of expression is high and the second relative ratio of expression is deficient, then administer an effective amount of the illudofulvene; or (d) determining from a patient test sample a third relative ratio of expression based on expression of a MYC gene compared with at least one of the housekeeping genes; (e) determining from a patient test sample a fourth relative ratio of expression based on expression of two or more TCR genes compared with at least one of the housekeeping genes; (f) determining that the patient is sensitive, if the third relative ratio of expression is high and the fourth relative ratio of expression is deficient, then administer an effective amount of the illudofulvene.

Embodiment R82. An illudofulvene analog selected from the group consisting of illudin ring derivatives, illudin alkyl derivatives and acyl-fulvenes for in the treatment of a cancerous tumor expressing a PTGR gene, a MYC gene or one or more TCR genes, wherein said expression includes: (a) contacting a sample from a patient comprising one or more nucleic acid molecules with one or more means for determining: (i) the regulation of expression of one or more PTGR genes; (ii) the regulation of expression of at least one TCR gene; (iii) the regulation of expression of at least one housekeeping gene; (b) determining if a cancerous tumor is sensitive to an illudofulvene analog based on a first relative ratio of expression of the one or more PTGR genes compared with the at least one housekeeping gene, when the first relative ratio of expression is high, and a second relative ratio of expression of the at least one TCR gene compared with the at least one housekeeping gene, when the second relative ratio of expression is deficient; or (c) contacting the sample comprising one or more nucleic acid molecules with one or more means for determining: (iv) the regulation of expression of a MYC gene; (v) the regulation of expression of two or more TCR genes; (vi) the regulation of expression of one or more housekeeping genes; (d) determining if the cancerous tumor is sensitive to the illudofulvene analog based on a third relative ratio of expression of the MYC gene compared with the one or more housekeeping genes, when the third relative ratio of expression is high, and a fourth relative ratio of expression of two or more TCR genes compared with the one or more housekeeping genes, when the fourth relative ratio of expression is deficient; or (e) contacting the sample comprising one or more nucleic acid molecules with one or more means for determining: (vii) the regulation of expression of three or more TCR genes; (viii) the regulation of expression of one or more housekeeping genes; (f) determining if the cancerous tumor is sensitive to the illudofulvene analog based on a fifth relative ratio of expression of the three or more TCR genes compared with the one or more housekeeping genes.

Embodiments contemplated herein further include Embodiments S1-S21 following.

Embodiment S1. An illudofulvene analog for use in the treatment of a cancerous tumor expressing a PTGR gene, a MYC gene or one or more TCR genes in a patient, wherein said expression includes (a) increased expression of one or more PTGR genes compared to a housekeeping gene in the cancerous tumor; (b) increased expression of a MYC gene compared to a housekeeping gene in the cancerous tumor; and/or (c) decreased expression of one or more TCR genes compared with a housekeeping gene.

Embodiment S2. The illudofulvene analog of Embodiment S1, where the illudofulvene is selected from the group consisting of 002, 008, 012, 039, 040, 41, 42, 43, 44, 46, 75, 76, 77, 78, 79, 88, 90, 94, 103, 104, 108, 110, 112, 113, 114, 115, 116, 119, 120, 121, 122, 123, 124, 125, 126, 127, 128, 130, 141, 142, 146, 149, 150, 160, 164, 165, 177, 178, 184. 189, 190, 196, 198, 199, 201, 202, 203, 207, 208, 209, 210, 217, 221, 223, 233, 234, 235, 240, 244, 254, 255, 259, 262, 263, 266, 267, 268, 269, 275, 276, 284, 285, 286, 287, 288, 289, 290, 291, 292, 293, 294, 295, 296, 297, 298, 304, 305, 306, 310, 314, 315, 317, 321, 323, 324, 332, 333, 334, 335, 336, 337, 338, 339, 340, 345, 346, 347, 348, 351, 352, 354, 356, 357, 368, 359, 361, 362, 363, 364, 365, 366, 367, 369, 370, 371, 372, 373, 374, 375, 377, 378, 379, 380, 381, 382, 383, 384, 389, 392, 393, and 394.

Embodiment S3. The illudofulvene analog of Embodiment S1 or S2, where the one or more housekeeping genes are selecting from the group consisting of beta actin (ACTB), transferrin receptor (TFRC) and glyceraldehyde-3-phosphate dehydrogenase (GAPD) and ubiquitin.

Embodiment S4. An illudofulvene analog for use in the treatment of a cancerous tumor expressing a PTGR gene, a MYC gene or one or more TCR genes in a patient, wherein said expression includes (a) messenger RNA and/or gene product (protein) of one or more PTGR genes is increased above a basal level of a housekeeping gene by at least approximately one point nine (1.9) standard deviations in the cancerous tumor; (b) messenger RNA and/or gene product (protein) of a MYC gene compared to a housekeeping gene in increased above a basal level of a housekeeping gene by at least approximately one point nine (1.9) standard deviations in the cancerous tumor; and/or (c) messenger RNA and/or gene product (protein) of one or more TCR genes is decreased below a basal level of a housekeeping gene by at least approximately one point nine (1.9) standard deviations.

Embodiment S5. The illudofulvene analog of Embodiment S4, where the one or more TCR genes are selected from the group consisting of one or more ERCC genes, a XPA gene, a GTF series gene, a UV gene, and a USP gene.

Embodiment S6. The illudofulvene analog of Embodiments S4 or S5, where the one or more PTGR genes are selected from the group consisting of PTGR1 and PTGR2.

Embodiment S7. The illudofulvene analog of Embodiments S4-S6, where the one or more housekeeping genes are selecting from the group consisting of beta actin (ACTB), transferrin receptor (TFRC) and glyceraldehyde-3-phosphate dehydrogenase (GAPD) and ubiquitin.

Embodiment S8. The illudofulvene analog of Embodiments S4-S7, where the gene expression was measured by means selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment S9. The illudofulvene analog of Embodiment S8, where the primers selected to hybridize with the one or more nucleic acid molecules are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, SEQ ID NO:900-902.

Embodiment S10. An illudofulvene analog for use in a method of treating a patient with cancer, wherein the method includes (a) determining from a patient test sample a first relative ratio of expression of messenger RNA and/or gene product (protein) of one or more PTGR genes compared with a basal level of a housekeeping gene or determining a second relative ratio of expression of messenger RNA and/or gene product (protein) of a MYC gene compared with a basal level of a housekeeping gene; and (b) determining a third relative ratio of expression of the TCR genes compared with a basal level of a housekeeping gene; wherein the patient is treated with the illudofulvene analog if the first relative ratio of expression is increased by at least approximately one point nine (1.9) standard deviations and the third relative ratio of expression is decreased by at least approximately one point nine (1.9) standard deviations or the second relative ratio of expression is increased by at least approximately one point nine (1.9) standard deviations and the third relative ratio of expression is decreased by at least approximately one point nine (1.9) standard deviations.

Embodiment S11. The illudofulvene analog for the use of Embodiment S10, where the one or more TCR genes are selected from the group consisting of one or more ERCC genes, a XPA gene, a GTF series gene, a UV gene, and a USP gene.

Embodiment S12. The illudofulvene analog for the use of Embodiment S10 or S11, where the one or more PTGR genes are selected from the group consisting of PTGR1 and PTGR2.

Embodiment S13. The illudofulvene analog for the use of Embodiment S10 to S12, where the ratio of gene expression is measured by means selected from the group consisting of (i) quantification of gene mRNA expression by probe analysis, (ii) microarray analysis, (iii) Real Time-Polymerase Chain Reaction (RT-PCR), (iv) Transcription Coupled Repair (TCR) Tumor Mutation Panel and (v) primary tumor cell sensitivity.

Embodiment S14. The illudofulvene analog for the use of Embodiment S10 to S13, where the primers selected to hybridize with the one or more nucleic acid molecules are selected from the group consisting of SEQ ID NO:251-261, SEQ ID NO:264-274, SEQ ID NO:307-308, SEQ ID NO:309-310, SEQ ID NO: 329-440, SEQ ID NO: 451-475, SEQ ID NO:682-696, SEQ ID NO: 715-727, SEQ ID NO:897-899, SEQ ID NO:900-902.

Embodiment S15. The illudofulvene analog for the use of Embodiment S10 to S14, where the effective amount is administered in a dosage between: a lower limit of approximately 0.1 mg of the illudofulvene per kilogram of body weight of the patient; and an upper limit of approximately 5 mg of the illudofulvene per kilogram of body weight of the patient.

Embodiment S16. The illudofulvene analog for the use of Embodiment S10 to S15, where the illudofulvene is selected from the group consisting of 002, 008, 012, 039, 040, 41, 42, 43, 44, 46, 75, 76, 77, 78, 79, 88, 90, 94, 103, 104, 108, 110, 112, 113, 114, 115, 116, 119, 120, 121, 122, 123, 124, 125, 126, 127, 128, 130, 141, 142, 146, 149, 150, 160, 164, 165, 177, 178, 184. 189, 190, 196, 198, 199, 201, 202, 203, 207, 208, 209, 210, 217, 221, 223, 233, 234, 235, 240, 244, 254, 255, 259, 262, 263, 266, 267, 268, 269, 275, 276, 284, 285, 286, 287, 288, 289, 290, 291, 292, 293, 294, 295, 296, 297, 298, 304, 305, 306, 310, 314, 315, 317, 321, 323, 324, 332, 333, 334, 335, 336, 337, 338, 339, 340, 345, 346, 347, 348, 351, 352, 354, 356, 357, 368, 359, 361, 362, 363, 364, 365, 366, 367, 369, 370, 371, 372, 373, 374, 375, 377, 378, 379, 380, 381, 382, 383, 384, 389, 392, 393, and 394.

Embodiment S17. An illudofulvene analog for use in the treatment of a cancerous tumor expressing a PTGR gene, a MYC gene or one or more TCR genes, wherein said expression includes (a) contacting a sample from a patient comprising one or more nucleic acid molecules with one or more means for determining (i) the regulation of expression of one or more PTGR genes; (ii) the regulation of expression of at least one TCR gene; (iii) the regulation of expression of at least one housekeeping gene; (b) determining if a cancerous tumor is sensitive to an illudofulvene analog based on a first relative ratio of expression of the one or more PTGR genes compared with the at least one housekeeping gene, when the first relative ratio of expression is high, and a second relative ratio of expression of the at least one TCR gene compared with the at least one housekeeping gene, when the second relative ratio of expression is deficient; or

(c) contacting the sample comprising one or more nucleic acid molecules with one or more means for determining (iv) the regulation of expression of a MYC gene; (v) the regulation of expression of two or more TCR genes; (vi) the regulation of expression of one or more housekeeping genes; and (d) determining if the cancerous tumor is sensitive to the illudofulvene analog based on a third relative ratio of expression of the MYC gene compared with the one or more housekeeping genes, when the third relative ratio of expression is high, and a fourth relative ratio of expression of two or more TCR genes compared with the one or more housekeeping genes, when the fourth relative ratio of expression is deficient.

Embodiment S18. An illudofulvene analog for use in a method of treating a patient with cancer, wherein the method includes (a) determining from a patient test sample a first relative ratio of expression based on expression of the one or more PTGR genes compared with expression of at least one housekeeping gene; (b) determining from the patient test sample a second relative ratio of expression based on expression of at least one TCR genes compared with expression of at least one housekeeping gene; and (c) determining that the patient is sensitive, if the first relative ratio of expression is high and the second relative ratio of expression is deficient, then administer an effective amount of the illudofulvene; or (d) determining from a patient test sample a third relative ratio of expression based on expression of a MYC gene compared with at least one of the housekeeping genes; (e) determining from a patient test sample a fourth relative ratio of expression based on expression of two or more TCR genes compared with at least one of the housekeeping genes; and (f) determining that the patient is sensitive, if the third relative ratio of expression is high and the fourth relative ratio of expression is deficient, then administer an effective amount of the illudofulvene.

Embodiment S20. A method of treating a cancerous tumor in a patient including (a) determining gene expression of at least two genes compared with one or more housekeeping genes selected from the group consisting of PTGR1 gene, PTGR2 gene, MYC gene and one or more TCR genes and (b) treating with an effective amount of an illudofulvene analog if: (I) the expression of one of the one or more PTGR genes compared to a housekeeping gene in the cancerous tumor is upregulated and the expression of one of the one or more TCR genes compared with a housekeeping gene is downregulated, or (II) the expression of the MYC gene compared to a housekeeping gene in the cancerous tumor is upregulated and the expression of two of the one or more TCR genes compared with at least one housekeeping gene is downregulated.

Embodiment S21. The method of Embodiment 20, where the patient is treated with an effective amount of an illudofulvene analog when the gene expression of PTGR1 gene compared with at least one housekeeping gene is upregulated, the gene expression of PTGR2 gene compared with at least one housekeeping gene is upregulated and the gene expression of one TCR gene of the one or more TCR genes compared with at least one housekeeping gene is downregulated.

Embodiment S22. A method of treating a cancerous tumor in a patient including: (a) determining from a patient test sample a first relative ratio of expression of a MYC gene compared with a housekeeping gene, (b) determining a second relative ratio of expression of a first TCR gene of one or more TCR genes compared with at least one housekeeping gene; (c) determining a third relative ratio of expression of a second TCR gene of the one or more TCR genes compared with at least one housekeeping gene, and (d) treating with an effective amount of an illudofulvene analog if the first relative ratio of expression is upregulated, the second relative ratio of expression is downregulated, and the third relative ratio of expression is downregulated.

The foregoing description of embodiments of the methods, systems, and components of the present invention has been provided for the purposes of illustration and description. It is not intended to be exhaustive or to limit the invention to the precise forms disclosed. Many modifications and variations will be apparent to one of ordinary skill in the relevant arts. For example, steps performed in the embodiments of the invention disclosed can be performed in alternate orders, certain steps can be omitted, and additional steps can be added. The embodiments were chosen and described in order to best explain the principles of the invention and its practical application, thereby enabling others skilled in the art to understand the invention for various embodiments and with various modifications that are suited to the particular used contemplated. Other embodiments are possible and are covered by the invention. Such embodiments will be apparent to persons skilled in the relevant art(s) based on the teachings contained herein. The breadth and scope of the present invention should not be limited by any of the above-described exemplary embodiments, but should be defined only in accordance with the following claims and their equivalents.

What is claimed is:

1. A composition comprising:
(a) a first plurality of first single stranded nucleic acid molecules generated using a pair of primers comprising SEQ ID NO:305 and SEQ ID NO:306, where the first single stranded nucleic acid molecules are labelled using qRT-PCR (quantitative reverse transcriptase polymerase chain reaction), where the first plurality of first single stranded nucleic acid molecules are used for determining a first level of a MYC gene present in a sample;
(b) a second plurality of second single stranded nucleic acid molecules generated using one or more pairs of primers selected from the group consisting of SEQ ID NO:275 paired with SEQ ID NO:276, SEQ ID NO:277 paired with SEQ ID NO:278, SEQ ID NO:279 paired with SEQ ID NO:280, SEQ ID NO:281 paired with SEQ ID NO:282, SEQ ID NO:283 paired with SEQ ID NO:284, SEQ ID NO:285 paired with SEQ ID NO:286, SEQ ID NO:287 paired with SEQ ID NO:288, SEQ ID NO:289 paired with SEQ ID NO:290, SEQ ID NO:291 paired with SEQ ID NO:292, SEQ ID NO:293 paired with SEQ ID NO:294, SEQ ID NO:295 paired with SEQ ID NO:296, SEQ ID NO:297 paired with SEQ ID NO:298, SEQ ID NO:299 paired with SEQ ID NO:300, SEQ ID NO:301 paired with SEQ ID NO:302, SEQ ID NO:303 paired with SEQ ID NO:304, SEQ ID NO:311 paired with SEQ ID NO:312, SEQ ID NO:313 paired with SEQ ID NO:314, SEQ ID NO:315 paired with SEQ ID NO:316, SEQ ID NO:317 paired with SEQ ID NO:318, SEQ ID NO:319 paired with SEQ ID NO:320, and SEQ ID NO:321 paired with SEQ ID NO:322, where the second single stranded nucleic acid molecule are labelled using qRT-PCR, where the second plurality of second single stranded nucleic acid molecules are used for determining a second level of a TCR gene present in the sample; and
(c) a third plurality of third single stranded nucleic acid molecules generated using one or more pair of primers selected from the group consisting of SEQ ID NO:249 paired with SEQ ID NO:250, and SEQ ID NO:262 paired with SEQ ID NO:263, where the third single stranded nucleic acid molecules are labelled using PCR, where the third plurality of third single stranded nucleic acid molecules are used for determining a third level of a PTGR gene present in the sample.

2. The composition of claim 1, further comprising where the sample is a cancerous tissue from a patient.

3. The composition of claim 1, where one or more of the first level, the second level, and the third level are compared with one or more housekeeping genes selected from the group consisting of beta actin (ACTB), transferrin receptor (TFRC), glyceraldehyde-3-phosphate dehydrogenase (GAPD) and ubiquitin.

4. The composition of claim 1, where the composition is used to determine whether to administer a compound selected from the group consisting of 6′-hydroxy-3′-(hydroxymethyl)-2′,4′,6′-trimethylspiro[cyclopropane-1,5′-inden]-7′(6′H)-one, 3-(6′-hydroxy-2′,4′,6′-trimethyl-7′-oxo-6′,7′-dihydrospiro[cyclopropane-1,5′-inden]-3′-yl)propanal oxime, 2-(3-(6′-hydroxy-2′,4′,6′-trimethyl-7′-oxo-6′,7′-dihydrospiro[cyclopropane-1,5′-inden]-3′-yl)propylidene)hydrazine-1-carboxamide, 6′-hydroxy-2′,4′,6′-trimethyl-3′-(3-(2-phenylhydrazineylidene)propyl)spiro[cyclopropane-1,5′-inden]-7′(6′H)-one, 2-hydroxy-4-(6′-hydroxy-2′,4′,6′-trimethyl-7′-oxo-6′,7′-dihydrospiro[cyclopropane-1,5′-inden]-3′-yl)butanenitrile, 4-(6′-hydroxy-2′,4′,6′-trimethyl-7′-oxo-6′,7′-dihydrospiro[cyclopropane-1,5′-inden]-3′-yl)butanenitrile, 1-hydroxy-1-((6′-hydroxy-2′,4′,6′-trimethyl-7′-oxo-6′,7′-dihydrospiro[cyclopropane-1,5′-inden]-3′-yl)methyl)urea, 3-(6′-hydroxy-2′,4′,6′-trimethyl-7′-oxo-6′,7′-dihydrospiro[cyclopropane-1,5′-inden]-3′-yl)propyl sulfamate and 1-((6′-hydroxy-2′,4′,6′-trimethyl-7′-oxo-6′,7′-dihydrospiro[cyclopropane-1,5′-inden]-3′-yl)methyl)pyrimidine-2,4(1H,3H)-dione to a patient suffering from cancer.

5. The composition of claim 4, where the composition is administered if the first level is enhanced in the sample.

6. The composition of claim 4, where the composition is administered if the second level is attenuated in the sample.

7. The composition of claim 4, where the composition is administered if the third level is enhanced in the sample.

8. The composition of claim 1, where the composition is administered if the first level and the third level are enhanced in the sample and the second level is attenuated in the sample.

9. The composition of claim 1, where the composition is used to determine whether to administer an acylfulvene analog, where the acylfulvene analog has a structural formula I: 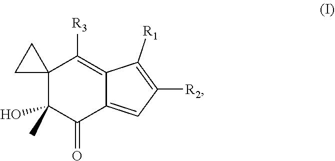
where R₁ represents —H, —CR₉R₈OH, —CHR₉CHR₈OH, —CHR₁₀CHR₉CHR₈OH, —CH₂CHR₁₀CHR₉CHR₈OH, —CR₉R₈OH, —CHR₉CHR₈OH, —CHR₁₀CHR₉CHR₈OH, —CH₂CHR₁₀CHR₉CHR₈OH, —C(═O)H, —CH₂(C═O)H, —CH₂CH₂(C═O)H, —CH₂CH₂CH₂(C═O)H, —CH₂CH₂CH₂CH₂(C═O)H, —CR₉R₈(C═O) H, —CHR₉CHR₈(C═O)H, —CH₂CHR₉CHR₈(C═O)H, —CR₉R₈(C═O)R₁₀, —CHR₉CHR₈(C═O)R₁₀, —CH₂CHR₉CHR₈(C═O)R₁₀, —CH₂CH₂CHR₉CHR₈(C═O)R₁₀, —CO₂H, —CHR₉CO₂H, —CHR₈CHR₉CO₂H, —CHR₁₀CHR₈CHR₉CO₂H, —CO₂R₁₀, —CHR₉CO₂R₁₀, —CHR₈CHR₉CO₂R₁₀, —CH₂CHR₈CHR₉CO₂R₁₀, —CHR₉CH₂CH₂CHR₈CO₂H, —CHR₉CH₂CH₂CHR₈CO₂R₁₀, —CR₈═CH₂, —CHR₈CH═CH₂, —CH₂CHR₈CH═CH₂, —CH₂CH₂CHR₈CH═CH₂, —CR₈═CHR₉, —CHR₈CR₉═CH₂, —CH₂CHR₈CR₉═CH₂, —CH₂CH₂CHR₈CR₉=CH₂, —CR₈═CR₉R₁₀, —CHR₈CH═CR₉R₁₀, —CH₂CHR₈CH═CHR₉R₁₀, —CH₂CH₂CHR₈CH═CHR₉R₁₀, —Cl, —Br, —I, —F, —NO₂, —NR₈R₉, —CH₂Cl, —CH₂Br, —CH₂I, —CH₂F, —CR₈Cl₂, —CR₈Br₂, —CR₈I₂, —CR₈F₂, —CCl₃, —CBr₃, —CI₃, —CF₃, —CHR₈Cl, —CR₈R₉Cl, —CHR₈CHR₉C1, —CHR₁₀CHR₈CHR₉Cl, —CH₂CHR₁₀CHR₈CHR₉C1, —CHR₈Br, —CR₈R₉Br, —CHR₈CHR₉Br, —CHR₁₀CHR₈CHR₉Br, —CH₂CHR₁₀CHR₈CHR₉Br, —CHR₈I, —CR₈R₉I, —CHR₈CHR₉Br, —CHR₁₀CHR₈CHR₉I, —CH₂CHR₁₀CHR₈CHR₉I, —CR₈R₉NH₂, —CHR₈CHR₉NH₂, —CHR₁₀CHR₈CHR₉NH₂, —CH₂CHR₁₀CHR₈CHR₉NH₂, —CR₉R₁₀NHR₈, —CHR₉CHR₁₀NHR₈, —CH₂CHR₉CHR₁₀NHR₈, —CH₂CH₂CHR₉CHR₁₀NHR₈, —CHR₉NHR₈R₁₀, —CHR₉CH₂NHR₈R₁₀, —CH₂CH₂CHR₉NR₁₀R₈, —CH₂CH₂CH₂CH₂NHR₈, —CR₉R₈OR₁₀, —CH₂CR₈R₉OR₁₀, —CH₂CH₂CR₈R₉OR₁₀, —CH₂CH₂CH₂R₈R₉OR₁₀, —CH₂CH₂CH₂CH₂R₈R₉OR₁₀, —CHR₈OC(═O)CHR₉R₁₀, —CHR₈OC(═O)CHR₉R₁₀, —CH₂CH₂OC(═O)CHR₉R₁₀, —CH₂CH₂CHR₈OC(═O)CHR₉R₁₀, —CH₂CH₂CH₂CH₂OC(═O)CHR₉R₁₀, —CHR₈(═O)OCHR₉R₁₀, —CHR₈(═O)OCHR₉R₁₀, —CH₂CHR₈(═O)OCHR₉R₁₀, —CH₂CH₂CHR₈(═O)OCHR₉R₁₀, —CH₂CH₂CH₂CHR₈(═O)OCHR₉R₁₀, —R₁₂C═CR₉CH₂OH, —R₁₂C═CR₉C(═O)H, —R₁₂C═CR₉CH₂OR₁₀, —R₁₂C═CR₉C(═O)R₁₀, —CH₃, —CH₂CH₂, —CHR₈CH₂, —CHR₈CH₂CH₂, —CHR₈CHR₉CH₃, —OCH₃, —OCR₈R₉R₁₀, —OCH₂CR₈R₉R₁₀, —OCR₉R₈CHR₁₀, —OCHR₈CH₂CH₃, —OCHR₈CHR₉CH₃, —NR₈CH₃, —NR₈CH₂CH₃, —NR₉CHR₈CH₃, —NR₉CHR₈CH₂CH₃, —NR₁₀CHR₈CHR₉CH₃, —OCH₂OR₈, —OCHR₈OR₉, —OCHR₈CH₂OR₉, —OCHR₈CHR₉OR₁₀, —OC(═O)OR₈, —OCH₂C(═O)OR₈, —OCHR₉C(═O)OR₈, —CR₈(═N)H, —CH₂CR₈(═N)H, —CH₂CR₈(═N)H, —CH₂CH₂CR₈(═N)H, —CH₂CH₂CH₂CR₈(═N)H, —CH₂CH₂CH₂CH₂CR₈(═N)H, —CR₈(═N)OH, —CH₂CR₈(═N)OH, —CH₂CR₈(═N)OH, —CH₂CH₂CR₈(═N)OH, —CH₂CH₂CH₂CR₈(═N)OH, —CH₂CH₂CH₂CH₂CR₈(═N)OH, —CR₈(═N)R₉, —CH₂CR₈(═N)R₉, —CH₂CR₈(═N)R₉, —CH₂CH₂CR₈(═N)R₉, —CH₂CH₂CH₂CR₈(═N)R₉, —CH₂CH₂CH₂CH₂CR₈(═N)R₉, —CR₈(═N)OR₉, —CH₂CR₈(═N)OR₉, —CH₂CR₈(═N)O R₉, —CH₂CH₂CR₈(═N)OR₉, —CH₂CH₂CH₂CR₈(═N)OR₉, —CH₂CH₂CH₂CH₂CR₈ (═N)OR₉, —CR₈(═N)NR₉, —CH₂CR₈(═N)NR₉, —CH₂CR₈(═N)NR₉, —CH₂CH₂CR₈ (═N)NR₉, —CH₂CH₂CH₂—CR₈(═N)NR₉, —CH₂CH₂CH₂CH₂CR₈ (═N)NR₉, —CR₈(═N)NR₉S(═O)₂R₁₀, —CH₂C(R₈)(═N)NR₉S(═O)₂R₁₀, —CH₂CH₂C(R₈)(═N)NR₉S(═O)₂R₁₀, —CH₂CH₂CH₂C(R₈)(═N)NR₉S(═O)₂R₁₀, CH₂CH₂CH₂CH₂C(R₈)(═N)NR₉S(═O)₂R₁₀, —R₁₂N(R₈)C(═O)NR₉R₁₀, —R₁₂N(R₈)C(═S)NR₉R₁₀, —R₁₂N(OR₈)C(═O)NR₉R₁₀, —R₁₂N(OR₈)C(═S) NR₉R₁₀, —R₁₂OS(O₂)NH₂, —R₁₂NHS(O₂)NH₂, —R₁₂OS(O₂)NR₈R₉, —R₁₂NHS(O₂)NR₈R₉, —CH₂N(R₈)S(O₂)NR₉R₁₀, —CH₂CH₂N(R₈)S(O₂)NR₉R₁₀, —CH₂CH₂CH₂N(R₈)S(O₂)NR₉R₁₀, —CH₂N(R₈)S(O₂) CR₉R₁₀R₁₁, —CH₂CH₂N(R₈)S(O₂)CR₉R₁₀R₁₁, —CH₂CH₂CH₂N(R₈)S(O₂)CR₉R₁₀R₁₁, —N(R₈)C(═O) R₉, —CH₂N(R₈)C(═O)R₉, —CH₂CH₂N(R₈)C(═O)R₉, —CH₂CH₂CH₂N(R₈)C(═O)R₉, —CH₂N(R₈)(C═O)NR₉R₁₀, —CH₂CH₂N(R₈)(C═O)NR₉R₁₀, —CH₂CH₂N(R₈)(C═O)NR₉R₁₀, —CH₂N(R₈)(C═O)CR₉R₁₀R₁₁, —CH₂CH₂N(R₈)(C═O)CR₉R₁₀R₁₁, —CH₂CH₂N(R₈)(C═O)CR₉R₁₀R₁₁, —R₁₂N(OH)C(═O)NHOH, —R₁₂N(OH)C(═S)NHOH, —R₁₂N(OR₈)C(═O)NHOR₉, —R₁₂N(OR₈)C(═S)NHOR₉, —R₁₂OS(O₂)N HOH, —R₁₂NHS(O₂)NHOH, —R₁₂OS(O₂)NHOR₉, —R₁₂OS(O₂)N(R₈)OR₉, —R₁₂NR₈S(O₂)NHOR₉, —CR₉(═N)OR₈, —NH(OR₈), —C(C═O)NHR₈, —C(C═O)NR₉R₈, —NR₁₀(OR₈)C(═O)R₉, —N(OR₈)C(═O)NR₉, —NR₈(R₉)S—, —N(R₈)S(═O)R₉, —NR(R₈)S(═O)₂R₉, —OC(═O)NR₈, —N(OR₈)C(═O)OR₉, —N(R₈)C(═S)R₉, —O(S(═O)₂R₈, —R₁₂O(S(═O)₂R₈, —O(S(═O)₂NR₈, —R₁₂O(S(═O)₂NR₈, —S(═O)R₈, —R₁₂S(═O)R₈, —S(═O)₂R₈, —R₁₂S(═O)₂R₈, —NR₁₀(R₉)S(═O)₂NHR₈, —NR₉(C═O)R₈, —NR₉(C═O)OR₈, —NR₉O(C═O)OR₈, —NR₉(C═O)NR₈R₁₀, —R₁₂N(R₉)S(═O)₂NHR₈, —R₁₂N(R₉)(C═O)R₈, —R₁₂N(R₉)(C═O)OR₈, —R₁₂N(R₉)(C═O)NR₈, —N(═NR₁₀)R₈, —R₁₂—N(═NR₁₀)R₈, —C(R₁₀)(═N—N═)CR₈R₉; —N₃, —CH₂N₃, —CH₂CH₂N₃, —CH₂CH₂CH₂N₃, —CH₂CH₂CH₂CH₂N₃, —CHR₈N₃, —CR₉R₈N₃, —CHR₉CHR₈N₃, —CH₂CHR₉CHR₈N₃, —CH₂CH₂CH₂CHR₈N₃, —C(R₈)═N—R₉, —CH₂C(R₈)═N—R₉, —CH₂CH₂C(R₈)═N—R₉, —CH₂CH₂CH₂C(R₈)═N—R₉, —N₃, —CH₂CH₂N₃, —CH₂CH₂CH₂N₃, —CH₂CH₂CH₂CH₂N₃; where R₂ and R₃ each independently represent —H, —OH, —CH₃, —OCH₃, —C(═O)CH₃, —OC(═O)CH₃, —C(═O)OCH₃, —C(═O)CH₂CH₃, —OC(═O)CH₂CH₃, —C(═O)OCH₂CH₃, —CH₂CH₃, —CH(CH₃)₂, —C(CH₃)₃, —CH₂CH₂CH₃, —CH₂CH(CH₃)₂, —CHC(CH₃)₃, —CH₂OH, —NH₂, —CH₂NH₂, —N₃, and (C1-C4)alkyl; where R₈, R₉, R₁₀, R₁₁ each independently represent —H, —OH, —CH₃, —CH₂CH₃, —CH₂═CH₂, —CH₂CH₂═CH₂, —CH₂CH₂CH₂═CH₂, —C(H)═O, —CH₂C(H)═O, —CH₂CH₂C(H)═O, —CH(CH₃)CH₃, —CH₂CH₂CH₃, —CH₂CH(CH₃)CH₃, —CHC(CH₃)₂CH₃, —C(═O)CH₃, —C(═O)CH₂CH₃, —C(═O)NH₂, —CH₂OH, —CH₂CH₂OH, —C(H)(OH)C(H₂)(OH), —OCH₃, —OC(CH₃)₃, —OCH₂CH₃, —OCH(CH₃)₂, —CO₂H, —C(═O)OCH₃, —C(═O)OCH₂CH₃, —OC(═O)CH₃, —OC(═O)CH₂CH₃, —OC(═O)OCH₃, —OC(═O)OCH₂CH₃, —C(CH₃)₃, —CH₂CH₂(CH₃)₂, —CHC(CH₃)₃, —CH₂CH₂OH, CH₂CH₂CH₂OH, —Cl, —Br, —I, —F, —CH₂Cl, —CH₂Br, —CH₂I, —CH₂F, —CHCl₂, —CHBr₂, —CHI₂, —CHF₂, —CCl₃, —CBr₃, —CI₃, —CF₃, —NH₂, —CH₂NH₂, —NH(OH), —CH₂N(OH), —NH(OCH₃), —N(OCH₂CH₃), —CH₂NH(OCH₃), —CH₂N(OCH₂CH₃), —N₃, —CH₂N₃, —CH₂CH₂N₃, —CH═NH, —CH═NOH, —CH═NOCH₃, —SH, —SCH₃, —SCH₂CH₃, —NO₂, —CN, cyclopropane ring, saturated or unsaturated cyclobutane ring, saturated or unsaturated cyclopentane ring, saturated or unsaturated cyclohexane ring, benzene ring, phenolic ring, xylene ring, an amino acid(s), (C₁-C₄)alkyl, and R₁₂ represents —CH₂—, —CH₂CH₂—, —CH(CH₃)CH₂—, —C(CH₃)CH₂—, —CH═CH—, —O—, —S—, —O(C═O)—, —(C═O)O—, —NH—, —N(R₈)—, —N(OH)—, —CH₂—O—, —O—CH₂—, —CH₂CH₂—O—, —O—CH₂CH₂—, —S(═O)—, —(S═O)₂—, —NH(S═O)₂—, —N(OH)S(═O)₂—, —S(═O)₂NH—, —S(═O)₂N(OH)—, —NHS(═O)₂—, —N(OH)S(═O)₂—, —CH₂NH—, —CH₂N(R₈)—, —CH₂N(OH)—, —NHCH₂—, —N(R₈)CH₂—, —N(OH)CH₂—, —OC(C═O)O—, —OC(═O)NR₈—, —NR₈C(═O)O—, to a patient suffering from cancer.

10. The composition of claim 9, where the composition is administered if the first level and the third level are enhanced in the sample.

11. The composition of claim 9, where the composition is administered if the second level is attenuated in the sample.

12. A composition comprising:
(a) a first plurality of first single stranded nucleic acid molecules generated using a pair of primers comprising SEQ ID NO:305 and SEQ ID NO:306, where the first single stranded nucleic acid molecules are labelled using qRT-PCR (quantitative reverse transcriptase polymerase chain reaction), where the first plurality of first single stranded nucleic acid molecules are used for determining a first level of a MYC gene present in a sample; and
(b) a second plurality of second single stranded nucleic acid molecules generated using one or more pairs of primers selected from the group consisting of SEQ ID NO:275 paired with SEQ ID NO:276, SEQ ID NO:277 paired with SEQ ID NO:278, SEQ ID NO:279 paired with SEQ ID NO:280, SEQ ID NO:281 paired with SEQ ID NO:282, SEQ ID NO:283 paired with SEQ ID NO:284, SEQ ID NO:285 paired with SEQ ID NO:286, SEQ ID NO:287 paired with SEQ ID NO:288, SEQ ID NO:289 paired with SEQ ID NO:290, SEQ ID NO:291 paired with SEQ ID NO:292, SEQ ID NO:293 paired with SEQ ID NO:294, SEQ ID NO:295 paired with SEQ ID NO:296, SEQ ID NO:297 paired with SEQ ID NO:298, SEQ ID NO:299 paired with SEQ ID NO:300, SEQ ID NO:301 paired with SEQ ID NO:302, SEQ ID NO:303 paired with SEQ ID NO:304, SEQ ID NO:311 paired with SEQ ID NO:312, SEQ ID NO:313 paired with SEQ ID NO:314, SEQ ID NO:315 paired with SEQ ID NO:316, SEQ ID NO:317 paired with SEQ ID NO:318, SEQ ID NO:319 paired with SEQ ID NO:320, and SEQ ID NO:321 paired with SEQ ID NO:322, where the second single stranded nucleic acid molecule are labelled using qRT-PCR, where the second plurality of second single stranded nucleic acid molecules are used for determining a second level of a TCR gene present in the sample.

13. The composition of claim 12, further comprising where the sample is a cancerous tissue from a patient.

14. The composition of claim 12, where one or more of the first level, and the second level are compared with one or more housekeeping genes selected from the group consisting of beta actin (ACTB), transferrin receptor (TFRC), glyceraldehyde-3-phosphate dehydrogenase (GAPD) and ubiquitin.

15. The composition of claim 12, where the composition is used to determine whether to administer a compound selected from the group consisting of 6′-hydroxy-3′-(hydroxymethyl)-2′,4′,6′-trimethylspiro[cyclopropane-1,5′-inden]-7′(6′H)-one, 3-(6′-hydroxy-2′,4′,6′-trimethyl-7′-oxo-6′,7′-dihydrospiro[cyclopropane-1,5′-inden]-3′-yl)propanal oxime, 2-(3-(6′-hydroxy-2′,4′,6′-trimethyl-7′-oxo-6′,7′-dihydrospiro[cyclopropane-1,5′-inden]-3′-yl)propylidene)hydrazine-1-carboxamide, 6′-hydroxy-2′,4′,6′-trimethyl-3′-(3-(2-phenylhydrazineylidene)propyl)spiro[cyclopropane-1,5′-inden]-7′(6′H)-one, 2-hydroxy-4-(6′-hydroxy-2′,4′,6′-trimethyl-7′-oxo-6′,7′-dihydrospiro[cyclopropane-1,5′-inden]-3′-yl)butanenitrile, 4-(6′-hydroxy-2′,4′,6′-trimethyl-7′-oxo-6′,7′-dihydrospiro[cyclopropane-1,5′-inden]-3′-yl)butanenitrile, 1-hydroxy-1-((6′-hydroxy-2′,4′,6′-trimethyl-7′-oxo-6′,7′-dihydrospiro[cyclopropane-1,5′-inden]-3′-yl)methyl)urea, 3-(6′-hydroxy-2′,4′,6′-trimethyl-7′-oxo-6′,7′-dihydrospiro[cyclopropane-1,5′-inden]-3′-yl)propyl sulfamate and 1-((6′-hydroxy-2′,4′,6′-trimethyl-7′-oxo-6′,7′-dihydrospiro[cyclopropane-1,5′-inden]-3′-yl)methyl)pyrimidine-2,4(1H,3H)-dione to a patient suffering from cancer.

16. The composition of claim 15, where the composition is administered if the first level is enhanced in the sample.

17. The composition of claim 15, where the composition is administered if the second level is attenuated in the sample.

18. The composition of claim 15, where the composition is administered if the first level is enhanced in the sample and the second level is attenuated in the sample.

19. The composition of claim 12, where the composition is used to determine whether to administer an acylfulvene analog, where the acylfulvene analog has a structural formula I: 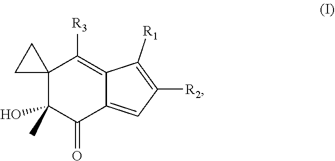
where R₁ represents —H, —CR₉R₈OH, —CHR₉CHR₈OH, —CHR₁₀CHR₉CHR₈OH, —CH₂CHR₁₀CHR₉CHR₈OH, —CR₉R₈OH, —CHR₉CHR₈OH, —CHR₁₀CHR₉CHR₈OH, —CH₂CHR₁₀CHR₉CHR₈OH, —C(═O)H, —CH₂(C═O)H, —CH₂CH₂(C═O)H, —CH₂CH₂CH₂(C═O)H, —CH₂CH₂CH₂CH₂(C═O)H, —CR₉R₈(C═O) H, —CHR₉CHR₈(C═O)H, —CH₂CHR₉CHR₈(C═O)H, —CR₉R₈(C═O)R₁₀, —CHR₉CHR₈(C═O)R₁₀, —CH₂CHR₉CHR₈(C═O)R₁₀, —CH₂CH₂CHR₉CHR₈(C═O)R₁₀, —CO₂H, —CHR₉CO₂H, —CHR₈CHR₉CO₂H, —CHR₁₀CHR₈CHR₉CO₂H, —CO₂R₁₀, —CHR₉CO₂R₁₀, —CHR₈CHR₉CO₂R₁₀, —CH₂CHR₈CHR₉CO₂R₁₀, —CHR₉CH₂CH₂CHR₈CO₂H, —CHR₉CH₂CH₂CHR₈CO₂R₁₀, —CR₈═CH₂, —CHR₈CH═CH₂, —CH₂CHR₈CH═CH₂, —CH₂CH₂CHR₈CH═CH₂, —CR₈═CHR₉, —CHR₈CR₉═CH₂, —CH₂CHR₈CR₉═CH₂, —CH₂CH₂CHR₈CR₉=CH₂, —CR₈═CR₉R₁₀, —CHR₈CH═CR₉R₁₀, —CH₂CHR₈CH═CHR₉R₁₀, —CH₂CH₂CHR₈CH═CHR₉R₁₀, —Cl, —Br, —I, —F, —NO₂, —NR₈R₉, —CH₂Cl, —CH₂Br, —CH₂I, —CH₂F, —CR₈Cl₂, —CR₈Br₂, —CR₈I₂, —CR₈F₂, —CCl₃, —CBr₃, —CI₃, —CF₃, —CHR₈Cl, —CR₈R₉Cl, —CHR₈CHR₉Cl, —CHR₁₀CHR₈CHR₉Cl, —CH₂CHR₁₀CHR₈CHR₉Cl, —CHR₈Br, —CR₈R₉Br, —CHR₈CHR₉Br, —CHR₁₀CHR₈CHR₉Br, —CH₂CHR₁₀CHR₈CHR₉Br, —CHR₈I, —CR₈R₉I, —CHR₈CHR₉Br, —CHR₁₀CHR₈CHR₉I, —CH₂CHR₁₀CHR₈CHR₉I, —CR₈R9NH₂, —CHR₈CHR₉NH₂, —CHR₁₀CHR₈CHR₉NH₂, —CH₂CHR₁₀CHR₈CHR₉NH₂, —CR₉R₁₀NHR₈, —CHR₉CHR₁₀NHR₈, —CH₂CHR₉CHR₁₀NHR₈, —CH₂CH₂CHR₉CHR₁₀NHR₈, —CHR₉NHR₈R₁₀, —CHR₉CH₂NHR₈R₁₀, —CH₂CH₂CHR₉NR₁₀R₈, —CH₂CH₂CH₂CH₂NHR₈, —CR₉R₈OR₁₀, —CH₂CR₈R₉OR₁₀, —CH₂CH₂CR₈R₉OR₁₀, —CH₂CH₂CH₂R₈R₉OR₁₀, —CH₂CH₂CH₂CH₂R₈R₉OR₁₀, —CHR₈OC(═O)CHR₉R₁₀, —CHR₈OC(═O)CHR₉R₁₀, —CH₂CH₂OC(═O)CHR₉R₁₀, —CH₂CH₂CHR₈OC(═O)CHR₉R₁₀, —CH₂CH₂CH₂CH₂OC(═O)CHR₉R₁₀, —CHR₈(═O)OCHR₉R₁₀, —CHR₈(═O)OCHR₉R₁₀, —CH₂CHR₈(═O)OCHR₉R₁₀, —CH₂CH₂CHR₈(═O)OCHR₉R₁₀, —CH₂CH₂CH₂CHR₈(═O)OCHR₉R₁₀, —R₁₂C═CR₉CH₂OH, —R₁₂C═CR₉C(═O)H, —R₁₂C═CR₉CH₂OR₁₀, —R₁₂C═CR₉C(═O)R₁₀, —CH₃, —CH₂CH₂, —CHR₈CH₂, —CHR₈CH₂CH₂, —CHR₈CHR₉CH₃, —OCH₃, —OCR₈R₉R₁₀, —OCH₂CR₈R₉R₁₀, —OCR₉R₈CHR₁₀, —OCHR₈CH₂CH₃, —OCHR₈CHR₉CH₃, —NR₈CH₃, —NR₈CH₂CH₃, —NR₉CHR₈CH₃, —NR₉CHR₈CH₂CH₃, —NR₁₀CHR₈CHR₉CH₃, —OCH₂OR₈, —OCHR₈OR₉, —OCHR₈CH₂OR₉, —OCHR₈CHR₉OR₁₀, —OC(═O)OR₈, —OCH₂C(═O)OR₈, —OCHR₉C(═O)OR₈, —CR₈ (═N)H, —CH₂CR₈(═N)H, —CH₂CR₈(═N)H, —CH₂CH₂CR₈(═N)H, —CH₂CH₂CH₂CR₈(═N)H, —CH₂CH₂CH₂CH₂CR₈(═N)H, —CR₈(═N)OH, —CH₂CR₈(═N)OH, —CH₂CR₈(═N)OH, —CH₂CH₂CR₈(═N)OH, —CH₂CH₂CH₂CR₈(═N)OH, —CH₂CH₂CH₂CH₂CR₈ (═N)OH, —CR₈(═N)R₉, —CH₂CR₈(═N)R₉, —CH₂CR₈(═N)R₉, —CH₂CH₂CR₈(═N)R₉, —CH₂CH₂CH₂CR₈(═N)R₉, —CH₂CH₂CH₂CH₂CR₈(═N)R₉, —CR₈(═N)OR₉, —CH₂CR₈(═N)OR₉, —CH₂CR₈(═N)O R₉, —CH₂CH₂CR₈(═N)OR₉, —CH₂CH₂CH₂CR₈(═N)OR₉, —CH₂CH₂CH₂CH₂CR₈ (═N)OR₉, —CR₈(═N)NR₉, —CH₂CR₈(═N)NR₉, —CH₂CR₈(═N)NR₉, —CH₂CH₂CR₈ (═N)NR₉, —CH₂CH₂CH₂—CR₈(═N)NR₉, —CH₂CH₂CH₂CH₂CR₈ (═N)NR₉, —CR₈(═N)NR₉S(═O)₂R₁₀, —CH₂C(R₈)(═N)NR₉S(═O)₂R₁₀, —CH₂CH₂C(R₈)(═N)NR₉S(═O)₂R₁₀, —CH₂CH₂CH₂C(R₈)(═N)NR₉S(═O)₂R₁₀, CH₂CH₂CH₂CH₂C(R₈)(═N)NR₉S(═O)₂R₁₀, —R₁₂N(R₈)C(═O)NR₉R₁₀, —R₁₂N(R₈)C(═S)NR₉R₁₀, —R₁₂N(OR₈)C(═O)NR₉R₁₀, —R₁₂N(OR₈)C(═S) NR₉R₁₀, —R₁₂OS(O₂)NH₂, —R₁₂NHS(O₂)NH₂, —R₁₂OS(O₂)NR₈R₉, —R₁₂NHS(O₂)NR₈R₉, —CH₂N(R₈)S(O₂)NR₉R₁₀, —CH₂CH₂N(R₈)S(O₂)NR₉R₁₀, —CH₂CH₂CH₂N(R₈)S(O₂)NR₉R₁₀, —CH₂N(R₈)S(O₂) CR₉R₁₀R₁₁, —CH₂CH₂N(R₈)S(O₂)CR₉R₁₀R₁₁, —CH₂CH₂CH₂N(R₈)S(O₂)CR₉R₁₀R₁₁, —N(R₈)C(═O) R₉, —CH₂N(R₈)C(═O)R₉, —CH₂CH₂N(R₈)C(═O)R₉, —CH₂CH₂CH₂N(R₈)C(═O)R₉, —CH₂N(R₈)(C═O)NR₉R₁₀, —CH₂CH₂N(R₈)(C═O)NR₉R₁₀, —CH₂CH₂N(R₈)(C═O)NR₉R₁₀, —CH₂N(R₈)(C═O)CR₉R₁₀R₁₁, —CH₂CH₂N(R₈)(C═O)CR₉R₁₀R₁₁, —CH₂CH₂N(R₈)(C═O)CR₉R₁₀R₁₁, —R₁₂N(OH)C(═O)NHOH, —R₁₂N(OH)C(═S)NHOH, —R₁₂N(OR₈)C(═O)NHOR₉, —R₁₂N(OR₈)C(═S)NHOR₉, —R₁₂OS(O₂)N HOH, —R₁₂NHS(O₂)NHOH, —R₁₂OS(O₂)NHOR₉, —R₁₂OS(O₂)N(R₈)OR₉, —R₁₂NR₈S(O₂)NHOR₉, —CR₉(═N)OR₈, —NH(OR₈), —C(C═O)NHR₈, —C(C═O)NR₉R₈, —NR₁₀(OR₈)C(═O)R₉, —N(OR₈)C(═O)NR₉, —NR₈(R₉)S—, —N(R₈)S(═O)R₉, —NR(R₈)S(═O)₂R₉, —OC(═O)NR₈, —N(OR₈)C(═O)OR₉, —N(R₈)C(═S)R₉, —O(S(═O)₂R₈, —R₁₂O(S(═O)₂R₈, —O(S(═O)₂NR₈, —R₁₂O(S(═O)₂NR₈, —S(═O)R₈, —R₁₂S(═O)R₈, —S(═O)₂R₈, —R12S(═O)₂R₈, —NR₁₀(R₉)S(═O)₂NHR₈, —NR₉(C═O)R₈, —NR₉(C═O)OR₈, —NR₉O(C═O)OR₈, —NR₉(C═O)NR₈R₁₀, —R₁₂N(R₉)S(═O)₂NHR₈, —R₁₂N(R₉)(C═O)R₈, —R₁₂N(R₉)(C═O)OR₈, —R₁₂N(R₉)(C═O)NR₈, —N(═NR₁₀)R₈, —R₁₂—N(═NR₁₀)R₈, —C(R₁₀)(═N—N═)CR₈R₉; —N₃, —CH₂N₃, —CH₂CH₂N₃, —CH₂CH₂CH₂N₃, —CH₂CH₂CH₂CH₂N₃, —CHR₈N₃, —CR₉R₈N₃, —CHR₉CHR₈N₃, —CH₂CHR₉CHR₈N₃, —CH₂CH₂CH₂CHR₈N₃, —C(R₈)═N—R₉, —CH₂C(R₈)═N—R₉, —CH₂CH₂C(R₈)═N—R₉, —CH₂CH₂CH₂C(R₈)═N—R₉, —N₃, —CH₂CH₂N₃, —CH₂CH₂CH₂N₃, —CH₂CH₂CH₂CH₂N₃; where R₂ and R₃ each independently represent —H, —OH, —CH₃, —OCH₃, —C(═O)CH₃, —OC(═O)CH₃, —C(═O)OCH₃, —C(═O)CH₂CH₃, —OC(═O)CH₂CH₃, —C(═O)OCH₂CH₃, —CH₂CH₃, —CH(CH₃)₂, —C(CH₃)₃, —CH₂CH₂CH₃, —CH₂CH(CH₃)₂, —CHC(CH₃)₃, —CH₂OH, —NH₂, —CH₂NH₂, —N₃, and (C1-C4)alkyl; where R₈, R₉, R₁₀, R₁₁ each independently represent —H, —OH, —CH₃, —CH₂CH₃, —CH₂═CH₂, —CH₂CH₂═CH₂, —CH₂CH₂CH₂═CH₂, —C(H)═O, —CH₂C(H)═O, —CH₂CH₂C(H)═O, —CH(CH₃)CH₃, —CH₂CH₂CH₃, —CH₂CH(CH₃)CH₃, —CHC(CH₃)₂CH₃, —C(═O)CH₃, —C(═O)CH₂CH₃, —C(═O)NH₂, —CH₂OH, —CH₂CH₂OH, —C(H)(OH)C(H₂)(OH), —OCH₃, —OC(CH₃)₃, —OCH₂CH₃, —OCH(CH₃)₂, —CO₂H, —C(═O)OCH₃, —C(═O)OCH₂CH₃, —OC(═O)CH₃, —OC(═O)CH₂CH₃, —OC(═O)OCH₃, —OC(═O)OCH₂CH₃, —C(CH₃)₃, —CH₂CH₂(CH₃) 2, —CHC(CH₃)₃, —CH₂CH₂OH, CH₂CH₂CH₂OH, —Cl, —Br, —I, —F, —CH₂Cl, —CH₂Br, —CH₂I, —CH₂F, —CHCl₂, —CHBr₂, —CHI₂, —CHF₂, —CCl₃, —CBr₃, —CI₃, —CF₃, —NH₂, —CH₂NH₂, —NH(OH), —CH₂N(OH), —NH(OCH₃), —N(OCH₂CH₃), —CH₂NH(OCH₃), —CH₂N(OCH₂CH₃), —N₃, —CH₂N₃, —CH₂CH₂N₃, —CH═NH, —CH═NOH, —CH═NOCH₃, —SH, —SCH₃, —SCH₂CH₃, —NO₂, —CN, cyclopropane ring, saturated or unsaturated cyclobutane ring, saturated or unsaturated cyclopentane ring, saturated or unsaturated cyclohexane ring, benzene ring, phenolic ring, xylene ring, an amino acid(s), (C₁-C₄)alkyl, and R₁₂ represents —CH₂—, —CH₂CH₂—, —CH(CH₃)CH₂—, —C(CH₃)CH₂—, —CH═CH—, —O—, —S—, —O(C═O)—, —(C═O)O—, —NH—, —N(R₈)—, —N(OH)—, —CH₂—O—, —O—CH₂—, —CH₂CH₂—O—, —O—CH₂CH₂—, —S(═O)—, —(S═O)₂—, —NH(S═O)₂—, —N(OH)S(═O)₂—, —S(═O)₂NH—, —S(═O)₂N(OH)—, —NHS(═O)₂—, —N(OH)S(═O)₂—, —CH₂NH—, —CH₂N(R₈)—, —CH₂N(OH)—, —NHCH₂—, —N(R₈)CH₂—, —N(OH)CH₂—, —OC(C═O)O—, —OC(═O)NR₈—, —NR₈C(═O)O—, to a patient suffering from cancer.

20. The composition of claim 19, where the composition is administered if the first level is enhanced in the sample and the second level is attenuated in the sample.


SEQUENCE LISTING (926 sequences)

SEQ ID NO: 1 - DNA, 1962 bp, Homo sapiens
  source 1..1962: mol_type = other DNA
gctgttccagtcccgcccctgagctctgtcatggcgacgtccagtgctccagccggtgtgaggacacgatatgctggggtttttgtccgcacgccaaacgggtttggaggaccctcttcgccttcggagagcagagtcaacacggagagttttgggactggaattaaataaagacagagatgttgaaagaatccacggcggtggaattaacacccttgacattgaacctgttgaagggagatacatgttatcaggtggttcagatggtgtgattgtactttatgaccttgagaactccagcagacaatcttattacacatgtaaagcagtgtgttccattggcagagatcatcctgatgttcacagatacagtgtggagactgtacagtggtatcctcatgacactggcatgttcacatcaagctcatttgataaaactctgaaagtatgggatacaaatacattacaattggtactagaggacccaaagtacaactttgtgacttgaagtctggatcctgttctcacattctacagggtcacagacaagaaatattagcagtttcctggtctccacgttatgactatatcttggcaacagcaagtgctgacagtagagtaaaattatgggatgtgagaagagcatcaggatgtttgattactcttgatcaacataatgggaaaaagtcacaagctgttgaatcagcaaacactgctcataatgggaaagttaatggcttatgttttacaagtgatggacttcacctcctcactgttggtacagataatcgaatgaggctctggaatagttccaatggagaaaacacacttgtgaactatggaaaagtttgtaataacagtaaaaaaggattgaaattcactgtctcctgtggctgcagttcagaatttgtttttgtaccatatggtagcaccattgctgtttatacagtttactcaggagaacagataactatgcttaagggacattataaaactgttgactgctgtgtatttcagtcaaatttccaggaactttatagtggtagcagagactgcaacattctggcttgggttccatccttatatgaaccagttcctgatgatgatgagactacaacaaaatcacaattaaatccggcctttgaagatgcctggagcagcagtgatgaagaaggatgaatatcatctttagtacctttttgtctctgctgaaactttttaaatgagactgtgtttttttcaactgtatggtctattcctgacagctaaattagccctaaatgtgggtaatatttttcctcatgttttaaaatgaggttaatatttgcataaaatcctaaaacagacttctgtatagtttatttagtcaaaatgtgttccttgatcccagatgttgtggcctgggaaagccctcattgctacagtacaagtaacacaagtcgttgtacctcagttgtgaccttcagcagattttatgaactataagatgcagtctcagaggatcagcaagtggaggccatcagtattgactttctcttacttgctgtactatcagcctgctcgtttccacctttaagaatgattttgccaagaatgattatatcaaaaatagtagttgaaatggtaacatcaaaattattttattctttcttcttcatgtattcacatttttcagtggtttcatttaattaaccatgctttatgttaaacattttggggctcaatgtctcctactatccaaaatgtgcatcacaggaggcttttaactttgtgaaaatcccatgtttgctttattttattttaatgtcagaaggcagtttgcgctaatgcttgaactctttttctgtgaaactcattaaggtatgaccaaatcctgcctcattaattcaagcagaaaatatcctggcagggaatctggcttaaacatgaaatgctgtaataaaatttctatgttattgtctc

SEQ ID NO: 2 - protein, 396 aa, Homo sapiens
MLGFLSARQTGLEDPLRLRRAESTRRVLGLELNKDRDVERIHGGGINTLDIEPVEGRYMLSGGSDGVIVLYDLENSSRQSYYTCKAVCSIGRDHPDVHRYSVETVQWYPHDTGMFTSSSFDKTLKVWDTNTLQTADVFNFEETVYSHHMSPVSTKHCLVAVGTRGPKVQLCDLKSGSCSHILQGHRQEILAVSWSPRYDYILATASADSRVKLWDVRRASGCLITLDQHNGKKSQAVESANTAHNGKVNGLCFTSDGLHLLTVGTDNRMRLWNSSNGENTLVNYGKVCNNSKKGLKFTVSCGCSSEFVFVPYGSTIAVYTVYSGEQITMLKGHYKTVDCCVFQSNFQELYSGSRDCNILAWVPSLYEPVPDDDETTTKSQLNPAFEDAWSSSDEEG

SEQ ID NO: 3 - DNA, 3590 bp, Homo sapiens
  source 1..3590: mol_type = other DNA
gcgccacgggcctccccgcaccagcagaagtcggagtcgctgttgggggcggtgtctatggttgagctgagggcgcaggcgccacggcccgtcgagctgggttccaaggcggctggcggcggtagcgtctctgtttccttgtgggcgctcgcgcggccctgggtagtctgtagagaatgccaaatgagggaatcccccactcaagtcaaactcaggagcaagactgtttacagagtcaacctgtcagtaataatgaagaaatggcaatcaagcaagaaagtggtggtgatggggaggtggaggagtacctctcctttcgttctgtgggtgacgggctgtccacctctgctgtggggtgcgcatcagcagctccgaggagagggccagccctgctgcacatcgaccgacatcagatccaggcagtagagcctagcgcccaggcccttgagctgcagggtttgggtgtggacgtctatgaccaggacgtgctggaacagggagtgcttcagcaggtggacaatgccatccatgaggccagccgtgcctcccagctcgttgacgtggagaaggagtatcggtcggtcctggatgacctcacgtcatgtacgacatccctaaggcaaatcaataaaattattgaacagcttagccctcaagctgccaccagcagagacatcaacaggaaactagattctgtaaaacgacagaagtataataaggaacaacagctaaaaaagatcactgcaaaacaaaagcatctccaggccatccttggaggagcagaggtgaaaattgaactagatcacgccagtctggaggaggatgcagagccggggccatccagtcttggcagcatgctcatgcctgtccaggagactgcctgggaagagctcatccgcactggccagatgacaccttttggtacccagatccctcagaaacaggagaaaaagcccagaaaaatcatgcttaatgaagcatcaggcttcgaaaagtatttggcagatcaagcaaaactgtcttttgaaaggaagaagcaaggttgtaataaaagagcagctagaaaagctccagccccagtcacgcctccagccccagtgcaaaataaaaacaaaccaaacaagaaagccagagttctgtccaaaaaagaggagcgtttgaaaaagcacatcaagaaactccagaagagggctttgcagttccaggggaaagtgggattgccaaaggcaaggagaccttgggagtcagacatgaggccagaggcagagggagactctgagggtgaagagtctgagtatttccccacagaggaggaggaagaggaggaagatgacgaggtggagggggcagaggcggacctgtctggagatggtactgactatgagctgaagcctctgcccaagggcgggaaacggcagaagaaagtgccagtgcaggagattgatgatgactttttcccaagttctggggaagaagctgaagctgcttctgtaggagaaggaggaggaggaggtcggaaagtgggaagataccgagatgatggagatgaagattattataagcagcggttaagtcccaagatgcctcgaacactaagtttacatgaaataactgaccttttagagacagatgacagcatagaagcaagtgctatagtgatacaaccacctgaaaatgctacagcacctgtttctgatgaggaatcaggagatgaagaaggtggaacaataaataatctgccaggttctttgttgcacacagctgcgtatcttattcaagatggctctgatgctgagtctgactcagatgatccctcatacgcacctaaagatgactctcctgatgaagttccatctacgtttactgtgcagcaacctccaccatcaaggaggaggaaaatgacaaaaattctttgcaaatggaaaaaagccgacctaactgtacaacccgtagcaggtagagttacagcaccaccaaacgatttcttcaccgtaatgagaactcccacagaaattcttgaactttttcttgatgacgaggtcattgaactcattgtcaagtactccaacttatatgcttgcagtaaaggtgtacatcttggcttgactagctctgaattcaaatgttttctgggaattatttttctgagtggttatgtctcagttcctagaaggcgtatgttttgggaacaaagaacagatgtgcataatgtactggttagtgctgccatgagacgtgaccggtttgaaactatattttctaatttgcatgttgctgacaatgcaaatttggatccagtggacaaattttccaaattgcgacctctcataagcaaacttaatgagagatgcatgaaatttgttccaaatgaaacatatttcagctttgatgaattcatggttccttattttggtcgtcacgggtgcaaacaatttattcggggaaagcccattcggtttggctataagttttggtgtggtgccacctgtctgggctacatttgctggtttcagccgtatcagggtaaaaacccaaatactaaacatgaggaatatggtgtcggtgcgtcacttgtccttcagtttagtgaggcacttacagaggcacaccctggacaataccattttgtattcaataactttttcaccagtattgcacttcttgataagctcagttcaatgggacatcaggcaacaggtacagtgagaaaggatcacattgacagagttccactggaatcagatgtagctttaaagaaaaaagaaagaggcacatttgattatcgaattgatggcaaaggcaatattgtctgcagatggaatgataacagtgttgtcactgttgcctcatctggtgctggtatccatcccctgtgtcttgtcagtcgttactcccagaaactgaaaaagaagatacaagttcagcagccaaacatgatcaaagtgtataaccagttcatgggaggcgtagacagagctgatgaaaacattgataagtatcgggcatcaatccgtggaaagaaatggtattcaagccctcttttgttctgtttcgaactggtcttacaaaatgcttggcaattgcataaaacatatgatgagaaaccagtggattttctggagtttcgtcgacgtgtggtatgccattatctggagacccatggtcatcctccagaacctggccaaaaaggaagacctcagaagcgtaacattgactcacgttatgatggcataaatcatgtgatagtcaaacagggaaagcaaacgcgatgcgctgaatgtcataagaacacaacttttcgatgtgaaaaatgtgatgttgccttacatgtgaagtgttccgttgaatatcacactgaatagcaggtgtcaccacctcctgagataagaaacatagttttatacattatgtacagtgtagcagtggttttgcctagtgttccaattttggaacgtcacataacaatggaacataataaatttttttttctcttcaaatttttgttccttgaatttttctaggtaacatatatgaatttcatgcaaaaattcaaaaaattgtaacctcagacataaatgggttaaagatgt

SEQ ID NO: 4 - protein, 1493 aa, Homo sapiens
MPNEGIPHSSQTQEQDCLQSQPVSNNEEMAIKQESGGDGEVEEYLSFRSVGDGLSTSAVGCASAAPRRGPALLHIDRHQIQAVEPSAQALELQGLGVDVYDQDVLEQGVLQQVDNAIHEASRASQLVDVEKEYRSVLDDLTSCTTSLRQINKIIEQLSPQAATSRDINRKLDSVKRQKYNKEQQLKKITAKQKHLQAILGGAEVKIELDHASLEEDAEPGPSSLGSMLMPVQETAWEELIRTGQMTPFGTQIPQKQEKKPRKIMLNEASGFEKYLADQAKLSFERKKQGCNKRAARKAPAPVTPPAPVQNKNKPNKKARVLSKKEERLKKHIKKLQKRALQFQGKVGLPKARRPWESDMRPEAEGDSEGEESEYFPTEEEEEEEDDEVEGAEADLSGDGTDYELKPLPKGGKRQKKVPVQEIDDDFFPSSGEEAEAASVGEGGGGGRKVGRYRDDGDEDYYKQRLRRWNKLRLQDKEKRLKLEDDSEESDAEFDEGFKVPGFLFKKLFKYQQTGVRWLWELHCQQAGGILGDEMGLGKTIQIIAFLAGLSYSKIRTRGSNYRFEGLGPTVIVCPTTVMHQWVKEFHTWWPPFRVAILHETGSYTHKKEKLIRDVAHCHGILITSYSYIRLMQDDISRYDWHYVILDEGHKIRNPNAAVTLACKQFRTPHRIILSGSPMQNNLRELWSLFDFIFPGKLGTLPVFMEQFSVPITMGGYSNASPVQVKTAYKCACVLRDTINPYLLRRMKSDVKMSLSLPDKNEQVLFCRLTDEQHKVYQNFVDSKEVYRILNGEMQIFSGLIALRKICNHPDLFSGGPKNLKGLPDDELEEDQFGYWKRSGKMIVVESLLKIWHKQGQRVLLFSQSRQMLDILEVFLRAQKYTYLKMDGTTTIASRQPLITRYNEDTSIFVFLLTTRVGGLGVNLTGANRVVIYDPDWNPSTDTQARERAWRIGQKKQVTVYRLLTAGTIEEKIYHRQIFKQFLTNRVLKDPKQRRFFKSNDLYELFTLTSPDASQSTETSAIFAGTGSDVQTPKCHLKRRIQPAFGADHDVPKRKKFPASNISVNDATSSEEKSEAKGAEVNAVTSNRSDPLKDDPHMSSNVTSNDRLGEETNAVSGPEELSVISGNGECSNSSGTGKTSMPSGDESIDEKLGLSYKRERPSQAQTEAFWENKQMENNFYKHKSKTKHHSVAEEETLEKHLRPKQKPKNSKHCRDAKFEGTRIPHLVKKRRYQKQDSENKSEAKEQSNDDYVLEKLFKKSVGVHSVMKHDAIMDGASPDYVLVEAEANRVAQDALKALRLSRQRCLGAVSGVPTWTGHRGISGAPAGKKSRFGKKRNSNFSVQHPSSTSPTEKCQDGIMKKEGKDNVPEHFSGRAEDADSSSGPLASSSLLAKMRARNHLILPERLESESGHLQEASALLPTTEHDDLLVEMRNFIAFQAHTDGQASTREILQEFESKLSASQSCVFRELLRNLCTFHRTSGGEGIWKLKPEYC

SEQ ID NO: 5 - DNA, 3515 bp, Homo sapiens
  source 1..3515: mol_type = other DNA
ctccttccgcgtggtccctccccctcaggccgcggtcgcgattacgctctctacggcctgcgaccgcagggccgttgcgggctggagacacggcgccgactggaaccggaggagctctaggccaaatggttgggccagccaggatcccaggacccttcgccgctcgagaccggagagaggaaacgaaacaggcgggaacccgtgggggagggagggaactagcggaaggtgtcatggcggccgcgctcttgagtcacgtgcccagggcccgccttgctacttccggtcacgtgccctcagactcctcgcagccagcgatggaggcgagaccccctagtaacagaggcggtggctactgctgcggccactgggtttcggcctcttcccagcagcggctctaagaagcgcagcggaactcgaccggatccaacccagttagttacttcctgtctagagttgtagcttccacctgagttctcctgggaagaagtgtttgccactcaaggaatacactcaagaaaaacctggaaatcacagaaggcattgtaagatagctacagtgattctgttaaacctcctgaaattggtccttgaacaattaagaggtgctacagatgaccaggaaaatttaccaggcaaattagctgtgagacaattgaccatttgtgcccaatgttcgcaccttctagccaccatggcaacctcatctgaagaagttttgctgattgtaaagaaagtgcgtcaaaagaagcaggatggagctctgtacctcatggcagaaagaattgcttgggcacctgaaggcaaagatagatttacaatcagccatatgtatgcagatattaaatgccagaaaattagtccagaaggaaaagctaaaattcagcttcagctggtcctacatgcaggggacacaactaacttccatttttccaatgaaagcacagcagtgaaagagcgagatgcagtaaaagaccttcttcagcagctgctgcccaaattcaagaggaaagcaaataaagaactggaagagaagaacagaatgctgcaagaagatcctgttttgtttcagctttataaagaccttgttgtgagtcaagtgatcagtgctgaggaattctgggccaatcgtttaaatgtgaatgcaacagatagttcttccacatccaatcataagcaggatgttggcatttctgctgcatttctggctgatgtccggccccaaactgatggctgtaacggtctaagatataatttaacttctgatatcattgagtccatatttaggacctatccagcagtaaaaatgaaatatgcagaaaatgttccccacaacatgacagagaaggaattctggacacgttttttccagtcccattattttcacagggatcggctgaatacagggtcaaaggatctctttgcagaatgtgccaaaatagatgaaaaaggcctaaaaacaatggtttcattaggagtgaaaaacccactactagatttaacagctttggaagataaaccattagatgagggctatggcatttcctctgtgccatctgcttccaattctaaatccataaaagagaatagtaatgctgccatcatcaagagatttaaccatcacagtgccatggtcctggcagctggactcagaaaacaagaagcacaaaatgaacaaactagtgagcccagcaacatggatggaaattccggagatgcagactgctttcagccagcagtcaaaagggcgaaattacaagagtccattgaatatgaagacttggggaaaaataattctgtaaaaacgattgcactaaacctcaagaagtcagataggtattatcatggtccaactccaatccagtcactacagtatgcaacaagtcaggacattattaattcttttcaaagtattagacaagaaatggaagcttatacacccaagttaactcaggttctctcaagtagtgctgccagtagtaccatcacagcactgtcacctggaggggcacttatgcagggaggaacacagcaagccataaaccagatggtgccaaatgatattcaatctgaattgaaacacttatatgtagctgttggagaacttctacgacatttctggtcctgctttcctgttaatacgccattcctagaagaaaaggtagtgaaaatgaaaagtaatttggaacgattccaagttacgaagctctgtccattccaagaaaagattcggagacagtatttaagcacaaatttggtaagtcacatagaagagatgctccagacagcctacaacaagctccacacatggcagtcacggcgtctgatgaagaaaacgtgaggtggccatgatgcttacaggttttgtgagattgagagaactatgacctgcagcaactctggaaacctggcctgacagacaagcagatgacctcacaggagtgataagaaacatctgctccacgccaactcccagagctgatgctattgtacttgcacattggagactgaaaggaaagaagggactaaatgctggggaggtaaattaagacagaaccaaatgagctaagttgcaaatatatatatatacacacacacacatatatgtacatgtgtatgtacatatatattttaaaagactgtttactgcagttgctcaggaactgcttttgattcacattaagctgctttcagaaattaaaaaaacactttttaaagggtgcattgataaaatctgaggttttttggttgtcgtttttttctgtgtacatttttttcctaagtttatggcacagggtagaccttaagtattcctcctccatccttcattcttcaccctccattggatcctcaagttttaatgaattccaattataccttacatcagcaagttaaaaaaagtactttaaaataaagcaaagggagactgttgctcaaccatcaggaaacagttgtcagaagacatcattggttctgtgtttcctacggaaataagaaacgataaatattgcactgaatgtttgtggtttggagtccctgaataataaagagggaatatatttgcagaaagtcgcatagggttttttaatgcagaattttgtcagaagacaatggcgctgcatgtttttctttgagtgcaaatgtacattgctaagatttttttaagatggcatgtgctttgaaaagaagatattgcatttttaagagtttaaaaatcttatgagtgagaaatattaaaaaaatcttattttcacctctttagaagaaataaaagatgtttctcctatctccttttctctagtatttgactgttactgtccttggcgaatcgataatcattgcatagtgactgaaaagcctaagtgcaaaaaaaaaaaaaaaagatgttcttgtttctgaacttcgtgccatattttgttcctgatgggatcaacttaatgtttaagactttagatgtcttgtattaaaaattacacaaaaaaagtaaaactttttatacttacccttttaactct

SEQ ID NO: 6 - protein, 548 aa, Homo sapiens
MATSSEEVLLIVKKVRQKKQDGALYLMAERIAWAPEGKDRFTISHMYADIKCQKISPEGKAKIQLQLVLHAGDTTNFHFSNESTAVKERDAVKDLLQQLLPKFKRKANKELEEKNRMLQEDPVLFQLYKDLVVSQVISAEEFWANRLNVNATDSSSTSNHKQDVGISAAFLADVRPQTDGCNGLRYNLTSDIIESIFRTYPAVKMKYAENVPHNMTEKEFWTRFFQSHYFHRDRLNTGSKDLFAECAKIDEKGLKTMVSLGVKNPLLDLTALEDKPLDEGYGISSVPSASNSKSIKENSNAAIIKRFNHHSAMVLAAGLRKQEAQNEQTSEPSNMDGNSGDADCFQPAVKRAKLQESIEYEDLGKNNSVKTIALNLKKSDRYYHGPTPIQSLQYATSQDIINSFQSIRQEMEAYTPKLTQVLSSSAASSTITALSPGGALMQGGTQQAINQMVPNDIQSELKHLYVAVGELLRHFWSCFPVNTPFLEEKVVKMKSNLERFQVTKLCPFQEKIRRQYLSTNLVSHIEEMLQTAYNKLHTWQSRRLMKKT

SEQ ID NO: 7 - DNA, 1951 bp, Homo sapiens
  source 1..1951: mol_type = other DNA
ccttctagcggcgccggtgagtccgcgtgtggaagtctgtgaggcgcagaggtggggcaggccgtctggctagctaggcggctgggagcgttttcgtggcggggaacggaggttgaattgccctgcctgggctcatagggaaggaggatgtgaaggagcttgtgaaggcagaggaagattattgaataataaaatacagttttgaaaaaaatggatgaagaacctgaaagaactaagcgatgggaaggaggctatgaaagaacatgggagattcttaaagaagatgaatctggatcacttaaagctacaatagaagacattctattcaaggcaaagagaaaaagagtatttgagcaccatggacaagttcgacttggaatgatgcgccacctttatgtggtagtagatggatcaagaacaatggaagaccaagatttaaagcctaatagactgacgtgtactttaaagttgttggaatactttgtagaggaatattttgatcaaaatcctattagtcagattggaataattgtaactaagagtaaaagagctgaaaaattgactgaactttcaggaaacccaagaaaacatataacgtctttgaagaaagctgtggatatgacctgccatggagagccatctctttataattccctaagcatagctatgcagactctaaaacacatgcctggacatacaagtcgagaagtactaatcatctttagcagccttacaacttgcgatccatctaatatttatgatctaatcaagaccctaaaggcagctaaaattagagtatctgttattggattgtctgcagaagttcgcgtttgcactgtacttgctcgtgaaactggtggcacgtaccatgttattttagatgaaagccattacaaagagttgctcacacatcatgttagtcctcctcctgctagctcaagttctgaatgctcacttattcgtatgggatttcctcagcacaccattgcttctttatctgaccaggatgcaaaaccctctttcagcatggcgcatttggatggcaatactgagccagggcttacattaggaggctatttctgcccacagtgtcgggcaaagtactgtgagctacctgttgagtgtaaaatctgtggtcttactttggtgtctgctccccacttggcacggtcttaccatcatttgtttcctttggatgcttttcaagaaattcccctagaagaatataatggagaaagattttgttatggatgtcagggggaattgaaagaccaacatgtttatgtttgtgctgtgtgccaaaatgttttctgtgtggactgtgatgtttttgttcatgattctctacactgttgccctggctgtattcataagattccagctccttcaggtgtttgattccagcatgtagtatacattgtatgtgttaaaaagaaatttgcaactgtgaataaaaggacttctttagaagaagcttcatttaaaacatgaaaggataatctgacttaagaaactttttgctaagaaaaggtaatattttattaaattttaaatttgtgttgtcacagaaatacctgaaattcagtagtacttcattcaattaattttgttttctattattttgagttatactgttttcaaagtcattatgcagtatgtataaacttataagaattaaattgatgtgataattttatgtttttataattaaatatagaatctttatgatttatgttaattcattaatttagtgtaagaagaaagttaagtctgaatgtaaattcagtgtaagatgaaaatttatcaatacttatgaaattaggctgggcgctgtggctcacacctgtaatcccaacactttgggaggctgaggtgggcagatcacctgaggtcaggagttcgagaccagcctggccaacatggtgaaaccccgtcactactaaaaatacaa

SEQ ID NO: 8 - protein, 395 aa, Homo sapiens
MDEEPERTKRWEGGYERTWEILKEDESGSLKATIEDILFKAKRKRVFEHHGQVRLGMMRHLYVVVDGSRTMEDQDLKPNRLTCTLKLLEYFVEEYFDQNPISQIGIIVTKSKRAEKLTELSGNPRKHITSLKKAVDMTCHGEPSLYNSLSIAMQTLKHMPGHTSREVLIIFSSLTTCDPSNIYDLIKTLKAAKIRVSVIGLSAEVRVCTVLARETGGTYHVILDESHYKELLTHHVSPPPASSSSECSLIRMGFPQHTIASLSDQDAKPSFSMAHLDGNTEPGLTLGGYFCPQCRAKYCELPVECKICGLTLVSAPHLARSYHHLFPLDAFQEIPLEEYNGERFCYGCQGELKDQHVYVCAVCQNVFCVDCDVFVHDSLHCCPGCIHKIPAPSGV

SEQ ID NO: 9 - DNA, 3434 bp, Homo sapiens
  source 1..3434: mol_type = other DNA
gggggctggcacgagtcacagcacctcgtgcgtcacttggcggcgccggacgttgggctgcgcaggatttgagacgctcccctgaccaccacttgctctgcgctgaggtgctgggacagccatggtttcagacgaagatgaattgaatcttctggttattgtagttgatgccaacccaatttggtggggaaagcaagcattaaaggaatctcagttcactttatccaaatgcatagatgccgtgatggtgctgggaaattcgcatttattcatgaatcgttccaacaaacttgctgtgatagcaagtcacattcaagaaagccgattcttatatcctggaaagaatggcagacttggagacttcttcggagaccctggcaaccctcctgaatttaatccctctgggagtaaagatggaaaatacgaacttttaacctcagcaaatgaagttattgttgaagagattaaagatctaatgaccaaaagtgacataaagggtcaacatacagaaactttgctggcaggatccctggccaaagccctttgctacattcatagaatgaacaaggaagttaaagacaatcaggaaatgaaatcaaggatattggtgattaaggctgcagaagacagtgcgttgcagtatatgaacttcatgaatgtcatctttgcagcacagaaacagaatattttgattgatgcctgtgttttagactccgactcagggctcctccaacaggcttgtgacatcacgggaggactgtacctgaaggtgcctcagatgccttctcttctgcagtatttgctgtgggtgtttcttcccgatcaagatcagagatctcagttaatcctcccacccccagttcatgttgactacagggctgcttgcttctgtcatcgaaatctcattgaaattggttatgtctgttctgtgtgtttgtcaatattctgcaatttcagccccatttgtactacgtgcgagacagcctttaaaatttctctgcctccagtgctgaaagccaagaaaaagaaactgaaagtgtctgcctgaggataaaatattttccccatcttttagagctgttaatagaaattatatagcagattctttgttgggaagactgaaaaaaataaagataggtataggataatttttaatatggtgaccttacagaaaatatttcccaaacatccttttcatcctgtgcttctggaggactgatttgtttgagggaatcattctatgcattatatcctaaaatattctatgactggtttctgtccatgtttgtggctttcatttttttaatgggatgactattagtcaaagtcagcttgtcatgactcatcataggctttctaacctactccctgaatccgggtcctcattgtgaaatgcatgccatacgaaatttgaacgtagctttggaaaaagggactatttgtggagtaatggcattaatcaacatagaacatcttatttgaatcaacagttaacttcagtagtcatgtgaataaaattcttattgtctaaattgagacagcctcagatatttgcagatatttactttttgtctgatatcagtacatatttggacaaagtcatctaaataatagtttgtcaccaaataactacaaaatctcattttaaatgagtaaggagaacgtgtacagaagcaaattttcttcaaaatagttgtgggaagagcttatatgtgaaagcttatgactggttttgagggagaacttactggagaaaatggactctatgttaagtatggttttcagatagaattctttccttttttaatgaggaaaaaaaatccacattaatattgaaactgcacctgtaatcccagcactttgggaggctgaggacagaggattgcttgagcccaggagttcgagagcagcctgggcagcaaagtgagaccccatctctactaaaaatttaaatgtatttattaaaactgttctctagaagctttggactgaatcccaaaagtgtttataagttcaaaagcaaaagtatttgtaatttcaacaacaaaaaatgtatttctttatgtaatcttgaaattattaaaagtccttttagcttctagcacatatttgtacaaagagtttaaggaatggtggctggtttggtttgttttttaaaaatgtttactgacgaggccgggcgtggtggctcacccctgcaatcccagcactttgggaggccgaggcaggcagatcacaaggtcaggagttcaagatcagcctggccagtatggtgaaaccctgtctctactaaaaatagaaaaattagccatgcgaagtagcaggtgcctgtagttccagctactcgggaggctgaggcaagagaattgcttgaatccaggaggcagaggttgcagtgagccaagatagcgcctctgtactccagcctgggtgacagagcgagactctgtatcaaaaaaaaaaaagatcgggcacgttggctcacgcctgtaatcccagcactttgggaggccaaggtgggtggatcacgaggtcaggagtgatcaggctgggtttctgttttgttttgttttgtgagacagagtctcgctctgttgcccaggctggggtgcagtggtgcaagctcggctctctgcaagctctgtctcctgggttcacgccattctcctgcctcagcctcccaagtagctgggattactactctactgaacccatgaggcagaaatttgagaccatcctgcccaacgtggtgaaaacatggtgaaaccctgtctctactaaaaatacaaaaattagctgggcgtggtggcaggtgcctgtaatcccagctactcaggagactgaggcaggagaattgcttgaacccaggaggtggaggttgcagtgagccaagatcaagattgcgccattgcactccagcctgggcgacaagaacaaaactctgtctcaaaaaaaaaaaaaaaaaaaaagtctgtactgataaaacccattgtgtacaaaactttgtatgtaaggaagattttaattttctctttatacaagctgagtcatatttaaataatttgatgttggcttagataatttcagatagattttatattctggatttgtgtttttgttaacaaatatacaaagactttggtgatcactttgcaaatatttgttaatccttgagtttgagaacctgtcttttaaaaataatattttgtatactaattaagtgtaatggaaatcacaattttaagtctaggaaataaagtattatatatactttcaaacaagctagcaaggcttttattattacttttttatttgaaatatcttatatatttggttagttctgtttaacttgtttttaactgttgcccttatgagttattttatataaatttttacaataaaataatttgattttcaaaaaaaaaaaaaaaaaa

SEQ ID NO: 10 - protein, 308 aa, Homo sapiens
MVSDEDELNLLVIVVDANPIWWGKQALKESQFTLSKCIDAVMVLGNSHLFMNRSNKLAVIASHIQESRFLYPGKNGRLGDFFGDPGNPPEFNPSGSKDGKYELLTSANEVIVEEIKDLMTKSDIKGQHTETLLAGSLAKALCYIHRMNKEVKDNQEMKSRILVIKAAEDSALQYMNFMNVIFAAQKQNILIDACVLDSDSGLLQQACDITGGLYLKVPQMPSLLQYLLWVFLPDQDQRSQLILPPPVHVDYRAACFCHRNLIEIGYVCSVCLSIFCNFSPICTTCETAFKISLPPVLKAKKKKLKVSA

SEQ ID NO: 11 - DNA, 1736 bp, Homo sapiens
  source 1..1736: mol_type = other DNA
cttttccactcctccccttacctcccttctcttctgaattctccattctgggctcttgcctgtgaaatctttctttgctttccccatcttttcctcgcattttttcaccatctttccctcaatctccaggagccaatgcgagactttggctccgattaagcgacggcccgagactcggggtgcgcgaggaggatcgacagagtggtgatggagagcaccccttcaaggggactgaaccgagtacacctacaatgcaggaatctgcaggaattcttagggggcctgagccctggggtattggaccgattgtatgggcaccctgccacatgtctggctgtcttcagggagctcccatccttggctaagaactgggtgatgcggatgctctttctggagcagcctttgccacaggctgctgtagctctgtgggtaaagaaggaattcagcaaggctcaggaggaaagtacagggctgctgagcggcctccggatctggcacacacagctgctcccaggcgggctccagggcctcatcctcaaccccattttccgccagaacctccgcattgcccttctgggtggggggaaggcctggtctgatgacacaagtcagctgggaccagacaagcatgcccgggacgttccctcccttgacaagtacgccgaggagcgatgggaggtggtcttgcacttcatggtgggctcccccagtgcagctgtcagccaggacttggctcagctcctcagccaggctgggctcatgaagagtactgaacctggagagccgccctgcattacttccgctggcttccagttcctgttgctggacaccccggctcagctctggtactttatgttgcagtatttgcagacagcccagagccggggcatggacctggtagagattctctccttcctcttccagctcagcttctctactctgggcaaggattactctgtggaaggtatgagtgattctctgttgaacttcctgcaacatctgcgtgagtttgggcttgttttccagaggaagaggaaatctcggcgttactaccccacacgcctggccatcaatctctcatcaggtgtctctggagctgggggcactgtgcatcagccaggtttcattgtcgtggaaaccaattaccgactgtatgcctacacggagtcggagctgcagattgccctcattgccctcttctctgagatgctctatcggttccccaacatggtggtggcgcaggtgacccgggagagtgtgcagcaggcaatcgccagtggcatcacagcccagcagataatccatttcctaaggacaagagcccacccagtgatgctcaaacagacacctgtgctgccccccaccatcaccgaccagatccggctctgggagctggaaagggacagactccggttcactgagggtgtcctgtataaccagttcctgtcgcaagtggactttgagctgctgctggcccacgcgcgggagctgggcgtgctcgtgttcgagaactcggccaagcggctcatggtggtgaccccggccgggcacagcgacgtcaagcgcttttggaagcggcagaaacatagctcctgagagcgcgggacttggacacggacctcggcgggcgggactgggcggggcggggcatcagaactcaggtgttttttatttacgcgtcagggcttttcttgtttaataaagttatgatagctaaaaaaaaaaaaaaaaaaaaa

SEQ ID NO: 12 - protein, 462 aa, Homo sapiens
MESTPSRGLNRVHLQCRNLQEFLGGLSPGVLDRLYGHPATCLAVFRELPSLAKNWVMRMLFLEQPLPQAAVALWVKKEFSKAQEESTGLLSGLRIWHTQLLPGGLQGLILNPIFRQNLRIALLGGGKAWSDDTSQLGPDKHARDVPSLDKYAEERWEVVLHFMVGSPSAAVSQDLAQLLSQAGLMKSTEPGEPPCITSAGFQFLLLDTPAQLWYFMLQYLQTAQSRGMDLVEILSFLFQLSFSTLGKDYSVEGMSDSLLNFLQHLREFGLVFQRKRKSRRYYPTRLAINLSSGVSGAGGTVHQPGFIVVETNYRLYAYTESELQIALIALFSEMLYRFPNMVVAQVTRESVQQAIASGITAQQIIHFLRTRAHPVMLKQTPVLPPTITDQIRLWELERDRLRFTEGVLYNQFLSQVDFELLLAHARELGVLVFENSAKRLMVVTPAGHSDVKRFWKRQKHSS

SEQ ID NO: 13 - DNA, 7503 bp, Homo sapiens
  source 1..7503: mol_type = other DNA
ggcgcactctgccggcaacgccgaggcgcttctgcatctgtgggccgagcattcttcaggtcatctgaaccttctgagaaaacatggtcaacgtcttgaaaggagtgcttatagaatgtgatcctgccatgaagcagtttctgctgtacttggatgagtccaatgccctggggaagaagttcatcattcaagacattgatgacactcacgtctttgtaatagcagaattggttaatgtcctccaggagcgagtgggtgaattaatggaccaaaatgctttttcccttacccagaaatgaaaatactcaatatggaccatttaggaattataagcagcaactgtgaaagacttgccactcaatatcttaggtgactgattagacatagagggttgttttaggagcatgccacgggaaagactgagggatcatgatcatttgttcagaaaaaaagcccctgaactgattttgttaccatagaatttaaaaaaaaaaaaagctttaacagttggctgtaatttggcttttattatcctttattaaaatacaaatgtcaatgcttttcctgcctttttaataccatgtcagtgtaacataggtatttattttgctcatccctgtgatctgtgcatttttgctgcgtggatgtgattgtttggtttcagttagaaacgtcatagatttgctgtttgaatatgccaaggtggggacttagacattatgtacgtctcacaaatcctacctgcataccagtcagctctattgaggaagacaatgtaaaactaatgtaaactacagtttgcatttccctgaaaacagaatattgtttttaagagggttagaaacaaccagtgggaaaggcacatgctgctttgtttagtttttccttgttcaaactttgttggtcacattttcccatctgtattcttttttattaagaaaacagcttagggaggctgaggtgggaggattgcttgagcccaggagtccgaggttgctacaaaaacaccattgcactctggcctggacaacagagagaccctgcctcaaaaaaaaaaaaaaaaaaaaaaaaaattctacttgtaccttattccctatgagaatacttatcaaacttatctaaaaaagaaaaataggaacagccatatgcaaagtcagcccaacaggaaaggacatattagattaaaatacattgcggcctgggcttagtggctcacacctgtaatcccagcactttgggaggctgaggcaggcagatcgcttgagtccaggagtcgaaaccctgtctctacaaaaaaaaaaatacaaaaatctgcctgttgtcccagccactggggaggctgaggtaggaggtcaaggctgcagtgaaccttgatcattgctactgccactccagcctaggtgacagagtgagaccctgtctccaaaaaaaaaagtgtatatgtatgtgtgtatatatagcaagagagagttctgtgatcaattgaaggcaaaaacagtaacactgagaggggctttgcttcccttcctgaagatcatgtcattggggtgggttcctgtaaagggaattttccaagagaaaagagaattttcatgacctgtagactcttacaaatccattttacctgcttccttatggtatgtttattcaaagcacctgtgtaccatatttattcacttacacagcattactgaatctggaaattttcagttaggtatattttacataattcccacccatataactcagtccatacagttcacagttttcattccctcatagcaatgcaaaaaattttgatgagttactactactaaaactagttaagtaaaagatgattcttaagaatttccaggctgggcacggtggctcacgcctgtaatcccagcactttgggaggctgaggcaggcagctcacgaggtcaggagatcgagaccatcctgctcggtgaaaccccatctctattaaaaatacaaaaaattagccgggcctggtggcgggtgcctgtagtcccatctactcaggaggctgaggcaggagaatggcgtgaacccgggaggcggagcttgcagtaagctgagattgtgccactgcactccagcctgggcgacagagggagactccatctcaaaaaaaaaaaaaaaaaaaaatgaatttccagctataccaaaatgttactaatgtatcttatatttcaggcatgtctaaatcaacttttagtatgcctcaggcatttgtcaccaaccttttatattaacatgaaacttagaaaatttttgatagaatcccagagttcaaaaggtattaggatttttgccactgaagtaaaaaagagaaaaaaagatatttatgagaaaactattgaacagtgactgtgttgtacccgagcaagttagaggaacgccacactttgagacgaatttaaaagtcctttatttagctggcagccaagaggtggctcacacttggaattctctcagctccgaggaaggggcttgattttcctttatactttggtttaggaaggggacgggagctcagttgcaacaattctacagaagtaaaaacatgcaaaaaattaaaaagacaaatggttacagagaaacaaacagttccaggtgcaggggctctaaatctatcataagatgtcaggtgtgggggctctgccagacacaaactcaaggctttatggtgtatctcttgagcgaaatcctgggaacttcgtacattgcttgcttcagtaccttatgagttaattggactctttgatatgttgagagtcagcttacacaagttaactccttgaggaaagggggtgggtaaggagtccttgatgtcctgtaaatgaaggaaccaaatggagttcctccggctttctcagctaagggagagcttattcacatggaaacaaggctaggtgattaagggagaaagggacagtctgaaaacaaggttagtaaaaacaaggttaggtattagaactgcaaagagtattaagtgaaataagtgaaggaaaatatgagctcttatttttaaaaagttgtgttggggcaaatgttaaatatctttctttctctggacattattaagaaagatagagtgatgtgaatgataggaagcccacctgtaaccacttagctgacatgtccaactatgggtgaacaggaaataagcgacattcacctaggctgtttattggaatctgactagcattatttgatttggcatactttacatgtccagaacaaggctgttggtgcttggcaccaagaaatttatgactttttttttttttttaaataaagtcctaaaggtagcattaaaatgacttaactggaggcaggaggatcactgaagcccaggagtttgaagctgcagtaagctaagaaggtgccactgcactgcagcctggatgacagagtgaaactttgtctcttaatagaactatggactgaagttgtgatattgagtgttatatttctcaataaaggttttttcttttaatactgtattaggtcaaattcaaggtcttaattccttgaattccaatttccttgtgaaatttcatgtgattaatttaaaattccagggatcctatctaacatcacagttgatggatataacaatgtattttgagaaatcagttttggtgaacagtaacagcattactgataataggcaacaaacactactgcattttatttctgataggcattataggtggtaatccaaatgattaatgattttattgctctttacattttctagaacaggtgatttgacataattgtacttttttctgagaagttggtagtgtacttactactctattttgcagttatacaataaatcttacttagctgacaactaggagtaaaagctctgacaaaaagcctctgctgtaactaaaatactgagatcattctatcttaacaaacttaacagatggggaaggggtatattttaaagatggagaaatgatgcagtttgcaaatatagtcaataaatattctcaagtgtacgtagctcaatttcatttgcagcaaagataacactgaaatgtctatggatgagattattggataaacttttttaaaaacactgttctcaagataggcaagtgattaagagcactgactgggccgggcgcggtggctcacgtctataatcctagcactttgggaggccgaggcgggcaaatcacaaggtcaggagatcaaaacaatcctggctaacaaggtgaaaccccgtctctactaaaaatacaaaaaaattagctgggcatggtggtggacgcctgtagtcccagctactcgggaggctgaggcaggagaatggtgtaatcccgggaggtggaggttgcagtgagccgagatcgtgccactgcactccagccggggtgacagagcgagactcccacccatctcaaaaaaaataaaaatgaaaagcactgattgaatctagattccttgagtccaagtgtcagatctctgcttactagctctgggatctcagatgtttcgttgattggatttctttgtgctcatttgtgtaaacaggaatgcccgtctttaaagactattaaaatgaatgtgagttaacacacaagaggtcttcaaaaagttgatggaaaatgcatattatgaaaaaaattatgcatagatttcaaaaatctttgcagcaaaataaactcatcattataacatgtctgaacaggatctagtttgaggcactaagaaggatatcagtttaaatagagcccctgtcagaacaatatgaattctgctaaaactgaagcaagaacaaacatcaaatttatggcaaagcttaggtggaagaatggtgcaatcattgatgctttgcaaaaagtttatgggaacaacgccccaaggaaatcagtttacaaatggataattcattttaagaaaggacaagacaatgttgaagatgaaacctacagcagcagaccatgccacttcgtgaggaaaaaattatctcattcatgccctacttgaagaggaccaatgattaacagcagaaacgatagccaacacaatatgctcctcagttggttcagcttttataaatctgactgaaaaattaaagttgagcacactttccactcaatggataccaaaaccatccagcccaggtcagctgcagacaaaagcagagctttcgatggaaatgtcaaacaagtgagatcaagatcctgaagcatttcttcaaagaattgtagcaaaaacagacttttaaaaccttgaattcagagacaggtataaagactgcttggtgattttgaaaggactgagatcattttgggagagatcatcttcagaaaatgaccagatacaacaagaatttaacatggcagccaaaccaacccactaacacacatttaaataagatacccaaatgcagaaagaaaagcacagtcgtcatgcaaaatagtcactactcattcaaaaactctatagttgtctgagtcgttatgctcaaaatatccaataatatttcttttgctgagctgtaaaataattattttgcaaacataaaagaaaaaaaaaagaattgtaacagaaacatggctttaccagtatgaccctcaagacaaaatacaaaacaatggctaccaagaggtggaagtggcccagtcacagcaagagcagattgatcaagagcaaaggtcatggtaacagttttttggatcctcaagtcattttgcttgttgactttttggagggccaaataaagatatcatctgcttattatgagagtgtttcgagagaaatccaaagcttagcagagaaacgcctgggaaagcttcaccagagggtcttccaccgtgacagttcccctgcgcattcctctcataaagcaagggcagtttagctagaatttctatcaaaaatcattaggtatccatctcccagtcctgatttggctccttctgacttctttttgtttcccaatcttaaaaaaatccctgaagggcacctgtttttcttcagctaataaaaaagacatggttaatttcccaggaccctcagttctttagggatagactgaatggctggtatcattgcttgaacttaatggagcttatgttgagaaataaaagaaattatattttatttttatcttttaattctatttttccatgaactttttgaagtcccctcagatataaagcagaacgttatgaaactgagttaccttgaatctagaagagtacaatgataaaggggaagatagaaacacacacatatgcgttgttttccccactttcacaatgccgtgcagaaactaacagatgaaggaggagaatccctaaaaacagagcattgggtagctagtccaccagcggtctgatgctttcattgatctcaaaactgtctctgagtaactctaccaggaaagttgtctggagggttaacatttctgaaaccatttttatctttgccttcattcttgaatgataattagtctcgaatgtttggatcacaatccattgaaagactaggctattttttcactgtcctcagacatttactgagtccagtttggactttattttattttattttttttgaaacaagatctcgctctgttgctcaggctgaagtgcaatagcgcaaactcggctcactgcaacctcccaggttcaagcaattctcgtgcctcagccatccctataggcatgcgccaccacactttgctaatcttgtatttttggtagagacggggtttgcaccatgttggccaggctggtctcgaactcccaacctcaagtaatctggcctcccaaagtggtgggattacaggcgtgagccagcgcgcccggcctggacttaacttctgaagaactctttctgtttagattgcaggagcgttctctcggctcatctcttgttttctcacccagctgttcttcttctagttggccatcattcttcatccctccctccctttcctgcagttaattatttctttgaaattttaaaacttcttaatgttgcctggaatacatgagtcttcctgatctcttcaaacggggtttgttttgttttgctttggggagaattcttttgacgtttttgaataagctgttaacaactttaatacctgtcttgtttgttttcttcagaaatttgttaattctccatgattagcacctttatgtggttatctcattattttcattcctttttaattctctgttttaggaatgcttgtcaaattcgtcactaaaattgatttactttcttgaaaacctgtttctgctgctcccaacatgatttaatcttggctactgagcttttggttacgttgcactccctttaaaattactccgtttaagtacagtgttcactctttgggtaatgtgtacaatagaagcccaatccccacctatacccaatatatccatgggacaagcatgtacatgcaccccctgagtctaaaataaaaattttaaaaaaactaaaaaaaaaaaaaaaaaa

SEQ ID NO: 14 - protein, 71 aa, Homo sapiens
MVNVLKGVLIECDPAMKQFLLYLDESNALGKKFIIQDIDDTHVFVIAELVNVLQERVGELMDQNAFSLTQK

SEQ ID NO: 15 - DNA, 5831 bp, Homo sapiens
  source 1..5831: mol_type = other DNA
gacatttcacgccgccgccattttgagagcgagccgagccgagctgccgggcgccgcgtccgctgccggagccccgacgacgacgccgaggaggcggaggccgcggctctcggaacgcggccggcccgtcgcccgcccgctccgccgctcccgccggccccagggcccgcaggcccgccgcggccggccaggcctcccgtccgccgcgcccggcccgaggcggggctgactccgcggcccccgaccggccgccctccgcccccggccggcccgcggccccgcagccccggccccggccccggcggcgggaggcgggccccgcggcggcggcggcggcggccgcagcgagcgacgaggccgcccggcccggcccgccggccgcccgcccgcctcggcgccgagattgggcgaatcgcgagcaagtacgtgcgcgtctccctgccgccgccgccgcccgccgcgggccgccccggggccgccgtcgccgacgacgcgcgggaggaggaggaggaggccgccccgccgccgccgccgccgccgccgccccggctcgccgccgcccgcccgccgggctcgcagccccggcccccggccgcaggcgaggcccaggccgcggccgacatgaaccaccagcagcagcagcagcagcagaaagcgggcgagcagcagttgagcgagcccgaggacatggagatggaagcgggagatacagatgacccaccaagaattactcagaaccctgtgatcaatgggaatgtggccctgagtgatggacacaacaccgcggaggaggacatggaggatgacaccagttggcgctccgaggcaacctttcagttcactgtggagcgcttcagcagactgagtgagtcggtccttagccctccgtgttttgtgcgaaatctgccatggaagattatggtgatgccacgcttttatccagacagaccacaccaaaaaagcgtaggattctttctccagtgcaatgctgaatctgattccacgtcatggtcttgccatgcacaagcagtgctgaagataataaattacagagatgatgaaaagtcgttcagtcgtcgtattagtcatttgttcttccataaagaaaatgattggggattttccaattttatggcctggagtgaagtgaccgatcctgagaaaggatttatagatgatgacaaagttacctttgaagtctttgtacaggcggatgctccccatggagttgcgtgggattcaaagaagcacacaggctacgtcggcttaaagaatcagggagcgacttgttacatgaacagcctgctacagacgttatttttcacgaatcagctacgaaaggctgtgtacatgatgccaaccgagggggatgattcgtctaaaagcgtccctttagcattacaaagagtgttctatgaattacagcatagtgataaacctgtaggaacaaaaaagttaacaaagtcatttgggtgggaaactttagatagcttcatgcaacatgatgttcaggagctttgtcgagtgttgctcgataatgtggaaaataagatgaaaggcacctgtgtagagggcaccatacccaaattattccgcggcaaaatggtgtcctatatccagtgtaaagaagtagactatcggtctgatagaagagaagattattatgatatccagctaagtatcaaaggaaagaaaaatatatttgaatcatttgtggattatgtggcagtagaacagctcgatggggacaataaatacgacgctggggaacatggcttacaggaagcagagaaaggtgtgaaattcctaacattgccaccagtgttacatctacaactgatgagatttatgtatgaccctcagacggaccaaaatatcaagatcaatgataggtttgaattcccagagcagttaccacttgatgaatttttgcaaaaaacagatcctaaggaccctgcaaattatattcttcatgcagtcctggttcatagtggagataatcatggtggacattatgtggtttatctaaaccccaaaggggatggcaaatggtgtaaatttgatgacgacgtggtgtcaaggtgtactaaagaggaagcaattgagcacaattatgggggtcacgatgacgacctgtctgttcgacactgcactaatgcttacatgttagtctacatcagggaatcaaaactgagtgaagttttacaggcggtcaccgaccatgatattcctcagcagttggtggagcgattacaagaagagaaaaggatcgaggctcagaagcggaaggagcggcaggaagcccatctctatatgcaagtgcagatagtcgcagaggaccagttttgtggccaccaagggaatgacatgtacgatgaagaaaaagtgaaatacactgtgttcaaagtattgaagaactcctcgcttgctgagtttgttcagagcctctctcagaccatgggatttccacaagatcaaattcgattgtggcccatgcaagcaaggagtaatggaacaaaacgaccagcaatgttagataatgaagccgacggcaataaaacaatgattgagctcagtgataatgaaaacccttggacaatattcctggaaacagttgatcccgagctggctgctagtggagcgaccttacccaagtttgataaagatcatgatgtaatgttatttttgaagatgtatgatcccaaaacgcggagcttgaattactgtgggcatatctacacaccaatatcctgtaaaatacgtgacttgctcccagttatgtgtgacagagcaggatttattcaagatactagccttatcctctatgaggaagttaaaccgaatttaacagagagaattcaggactatgacgtgtctcttgataaagcccttgatgaactaatggatggtgacatcatagtatttcagaaggatgaccctgaaaatgataacagtgaattacccaccgcaaaggagtatttccgagatctctaccaccgcgttgatgtcattttctgtgataaaacaatccctaatgatcctggatttgtggttacgttatcaaatagaatgaattattttcaggttgcaaagacagttgcacagaggctcaacacagatccaatgttgctgcagtttttcaagtctcaaggttatagggatggcccaggtaatcctcttagacataattatgaaggtactttaagagatcttctacagttcttcaagcctagacaacctaagaaactttactatcagcagcttaagatgaaaatcacagactttgagaacaggcgaagttttaaatgtatatggttaaacagccaatttagggaagaggaaataacactatatccagacaagcatgggtgtgtccgggacctgttagaagaatgtaaaaaggccgtggagcttggggagaaagcatcagggaaacttaggctgctagaaattgtaagctacaaaatcattggtgttcatcaagaagatgaactattagaatgtttatctcctgcaacgagccggacgtttcgaatagaggaaatccctttggaccaggtggacatagacaaagagaatgagatgcttgtcacagtggcgcatttccacaaagaggtcttcggaacgttcggaatcccgtttttgctgaggatacaccagggcgagcattttcgagaagtgatgaagcgaatccagagcctgctggacatccaggagaaggagtttgagaagtttaaatttgcaattgtaatgatgggccgacaccagtacataaatgaagacgagtatgaagtaaatttgaaagactttgagccacagcccggtaatatgtctcatcctcggccttggctagggctcgaccacttcaacaaagccccaaagaggagtcgctacacttaccttgaaaaggccattaaaatccataactgatttccaagctggtgtgttcaaggcgaggacggtgtgtgggtggccccttaacagcctagaactttggtgcacgtgccctctagccgaagtcttcagcaagaggattcgctgctggtgttaattttattttattgaggctgttcagtttggcttctctgtatctattgactgccctttttgagcaaaatgaagatgtttttataaagcttggatgccaatgagagttattttatggtaaccacagtgcaaggcaactgtcagcgcaatgggggagaagaggttagtggatcgggggtccctggctcaaggtctctgggctgtccctagtgggcacgagtggctcggctgccttcctggggtcccgtgcaccagccctgcagctagcaagtcttgtgtttaggctcgtctgacctatttccttcagttatactttcaatgaccttttgtgcatctgttaaggcaaaacagagaaactcacaacctaataaatagcgctcttcccttcattgtgtgcattgtcggcccttcctcgggttctcctcctccagctgcctgggggctttttaataaacttgtctcacctcgtcagccactactgtctgcagcccctttgcaaagtggatgcactgaatacagtccggacagacattgtgggggtctttttattaaatcaagaacattgttaaattcaattaaggtttactctgctgccttggcagacttacgatctcaacagttcatacgagcaggtgaaaggattataaatagaatttcgttaaagtggaacagacgacaagaaagccttttagcaagagggcatgctcactagtggttagtaagctgtcgactttgtaaaaaagttaaaaatgaaaaaaaaaggaaaaatgaattgtatatttaatgaatgaacatgtacaatttgccactgggaggaggttcctttttgttgggtgagtctgcaagtgaatttcactgatgttgatattcattgtgtgtagttttatttcggtcccagccccgtttccttttattttggagctaatgccagctgcgtgtctagttttgagtgcagtaaaatagaatcagcaaatcactcttatttttcatccttttccggtattttttgggttgtttctgtgggagcagtgtacaccaactcttcctgtatattgcctttttgctggaaaatgttgtatgttgaataaaattttctataaaaattataattcagtgagttacgtggaagtggaggaagatttctactctccctggaaacaggcctgggaaaccttggcatttgtaacaaggtttcactgagatgtacttttccttctaattccgttttgcgggggcagggtctcttgtttctttttttttttttttttttttttagcctctaactagtcacatttactcttaagaaatgaaaggttttccaggagagaactgtgtacaaataaggtgactggagatgtgacctgatgtgtcacgaggcccttcggggcggcaggcgctatcgtgggcgtggtccttgcaccgtcccatcggccttgccttccagctccgtggcacggtttcctggtctttgggccagtgtgtaccttggagtgacttcctttctcaacttccactgcagtgtgtgtgccttctgctctgagagctgccttgtgacccgtgtgatagaaagcagggagtgagggtccccgcggacctggcccttccctccttcctcccccagaaagaggagttagagcaggggtgcgagagccgttcgctgtgggtttgtctttgaacaaacattaaggtgtcttgtttttgttctgggctgggggttggctgtagtcttaggtaactgaaagttcctactctcccttaaggtattaaatgactctttttccaaa

SEQ ID NO: 16 - protein, 1102 aa, Homo sapiens
MNHQQQQQQQKAGEQQLSEPEDMEMEAGDTDDPPRITQNPVINGNVALSDGHNTAEEDMEDDTSWRSEATFQFTVERFSRLSESVLSPPCFVRNLPWKIMVMPRFYPDRPHQKSVGFFLQCNAESDSTSWSCHAQAVLKIINYRDDEKSFSRRISHLFFHKENDWGFSNFMAWSEVTDPEKGFIDDDKVTFEVFVQADAPHGVAWDSKKHTGYVGLKNQGATCYMNSLLQTLFFTNQLRKAVYMMPTEGDDSSKSVPLALQRVFYELQHSDKPVGTKKLTKSFGWETLDSFMQHDVQELCRVLLDNVENKMKGTCVEGTIPKLFRGKMVSYIQCKEVDYRSDRREDYYDIQLSIKGKKNIFESFVDYVAVEQLDGDNKYDAGEHGLQEAEKGVKFLTLPPVLHLQLMRFMYDPQTDQNIKINDRFEFPEQLPLDEFLQKTDPKDPANYILHAVLVHSGDNHGGHYVVYLNPKGDGKWCKFDDDVVSRCTKEEAIEHNYGGHDDDLSVRHCTNAYMLVYIRESKLSEVLQAVTDHDIPQQLVERLQEEKRIEAQKRKERQEAHLYMQVQIVAEDQFCGHQGNDMYDEEKVKYTVFKVLKNSSLAEFVQSLSQTMGFPQDQIRLWPMQARSNGTKRPAMLDNEADGNKTMIELSDNENPWTIFLETVDPELAASGATLPKFDKDHDVMLFLKMYDPKTRSLNYCGHIYTPISCKIRDLLPVMCDRAGFIQDTSLILYEEVKPNLTERIQDYDVSLDKALDELMDGDIIVFQKDDPENDNSELPTAKEYFRDLYHRVDVIFCDKTIPNDPGFVVTLSNRMNYFQVAKTVAQRLNTDPMLLQFFKSQGYRDGPGNPLRHNYEGTLRDLLQFFKPRQPKKLYYQQLKMKITDFENRRSFKCIWLNSQFREEEITLYPDKHGCVRDLLEECKKAVELGEKASGKLRLLEIVSYKIIGVHQEDELLECLSPATSRTFRIEEIPLDQVDIDKENEMLVTVAHFHKEVFGTFGIPFLLRIHQGEHFREVMKRIQSLLDIQEKEFEKFKFAIVMMGRHQYINEDEYEVNLKDFEPQPGNMSHPRPWLGLDHFNKAPKRSRYTYLEKAIKIHN

SEQ ID NO: 17 - DNA, 4674 bp, Homo sapiens
  source 1..4674: mol_type = other DNA
gccagggcgtgtcccgtgtgcccgagagcgccggcggcggggccggagggcgggccctgggccgagtgacaggccgcagccccccgagcaccgtcgaagccggcgcgcccgcccggtgcagccgctgtgcgagagcgaccccgcggtccaagcctcgcgtggccctgccgggccccttctttccccgagtagggcgcagcttcccagcctccgccccggcctcgcctggcgcttccttccgggtccttcggcctttccctggcggtgcggcaggccctcgcctcatcccaccaggcacgccgcggtgctcggccctgggtatccgggcaggctgcctccgttagggccgcccctgctctccggacgcgacttttcattggtctcagaatttcttggctcctcttggcctctgcagccttgctggaggctgccctgcggaatctgaatatggatcagaaactttcgaagttggtagaagagctcacaacttcaggagaaccccgactaaatcctgagaaaatgaaggaactgaagaaaatttgcaagtcttcagaggagcagctgagccgcgcctaccgcctgctgatagcacagctgacccaggagcacgccgagatccgtctctcagccttccagattgtggaggaactcttcgtcaggtctcaccagttccggatgctggttgtttccaacttccaggagttcctggagctcacgctgggcacagaccccgcacagcctctgccgccccccagggaggcggcacagaggctgaggcaggcgaccacccgggccgtggaagggtggaatgagaagtttggggaggcctacaagaagcttgccttgggctaccacttcttaagacacaacaaaaaggtggattttcaagacacgaatgctcggagtctggcagaaaggaagagagaagaggagaagcagaagcacttggataaaatttatcaagaaagagccagccaggcggagagggagatgcaagaaatgtctggagaaattgaatcctgcttgacggaggtagagagctgctttaggctgctggtgccttttgactttgacccgaacccggagacggaatcccttggcatggcttctggcatgtccgatgcccttcgctcctcctgcgcgggccaggtgggcccctgccggtctggcacccctgacccccgggacggggagcagccctgctgcagtagagacctgcctgcctctgcaggccaccccagagcgggcggcggggcacagccatcccagacagccacaggtgacccctcagatgaggacgaggacagcgacctcgaggagtttgtgcggagccacgggctgggctcgcacaagtacacgctggatgtggagctctgctcagagggcctgaaggtgcaggagaacgaggacaaccttgctctcatccacgccgcccgcgacacactcaagctcatccggaacaagttcctgccggctgtgtgctcgtggatccagcgcttcacccgcgtcgggacccacggtggatgtttaaagcgtgccattgacctgaaggctgaattggagctcgtactgagaaaatacaaggagctggacatcgagcctgagggaggggaaaggcgcaggacagaagccctgggggatgcggaggaagatgaggacgatgaggactttgtggaggtccctgagaaggaggggtatgagccacacatccccgaccacttgcggcctgagtatgggctggaggcagcaccagagaaagacacagttgtgcggtgcttgcggacgaggacgaggatggacgaggaggtgtcggaccccacctctgcggctgctcagctgcggcagctccgggaccacttgcctccaccctcatctgccagcccctccagagcgttgccagagccacaggaggcccagaagctggcagcagagcgggcccgggcgcctgtggtgccctacggcgtggacctgcactactggggccaggagctccccacagccgggaagattgtcaagtctgactcccagcaccgcttctggaagcccagcgaggtggaggaggaagtggtcaatgccgacatctccgagatgctccggagccgccacatcacttttgccgggaagtttgagcctgtgcagcactggtgccgtgccccgaggccagacggccggctctgtgagcgccaagaccggctgaagtgccctttccatgggaagattgttccacgggacgacgaaggacggccgctcgacccggaagacagggctcgtgagcagcggcggcagctgcagaagcaggagcgcccggaatggcaggaccctgagttgatgagagacgtggaagcagccacagggcaggatctcggctcatccaggtacagcgggaaaggcagggggaagaagaggaggtaccccagcctcaccaacctgaaggctcaggctgataccgcccgcgctcgcattgggagaaaagtcttcgccaaggcagctgtgcggagggtagtggcagccatgaaccggatggaccagaagaagcacgagaagttttcaaaccagtttaactacgcactgaactagagagcggggcccagtgcactggccatcagcactttctccctctgccagtgtctcaggacagcagagtgggcgtgggtctgggcagtaaccatgctttgtctattactgtgtttgatgtaaagaaatggtgtgttgcaatgccctgaaggtacggccgctctgctgctacagggttcggcatcgtctggtgatgggtctggcctcgcagaagaggccctcgggcctggagatgtgaacacaggcagcgaccctgttccagagggcttctgcgagtcctcgtgagaccagtgcttgtcgtggtgtggggttcagcacggacggaatgtgtgtgcagctcagccttcagagcgtgcattccccagccaggaggcgaccactcagaggaactctgggaaacccacttttgtgcaaatgctgtttttaacacaaacacaaaggctagtgaaccgttcagtcatctgctttctgtttctggatgtgccttttcatacatgtgtcctcttgtccctggcttcttacacttggcgttgtttttaaggtttatccatgcagttcccctccaatgtataaaacaaaggaggtgaaaacctgtccatgcggaagcttgtgcacccatgttcacagcagcattggagaaggataccgaacatcttctcgtgggcttactggccatttgtgtatctcctttggagaggagtctattcaaacctcttgcctatttttaattgaatgatttatcttgagttgtaacagttgttttttttgtttttccttttagagatgggcttttgctcttacccaggctgagtgcagtggcagaatcatagctcagtgcagcctcaaactcctgggctcaagccatccttccacgtcagcctcccaggtagctgggactacaggcacacaccacaacacctggctaattttttaaattttttgtagagatgaggtctcactgtgttgcccacggtgatctcgaactcctagcctcaagcgatcctcctgcttcggtctcccaaagtgttgggattacaggcatgagccaccactcctggccaggagtttatatgtatactgtggatactagatcctcatgtgatttgcaaaaactttatttatttatttatttatttttgaaatggaatcttgctctgtcgccaggctggagtgcaatggcgtgatctcggctcactgcaactaccacctcctgggttcaagcgattcgcctgcgtcagcgtcctgagtagctgggactacaggcgcgcaccaccacgcccagctaatttttgtatttttagtagagacgggatttcactgtgttggctgggatggtcttgatctcttgacctcgtgatccacccacctcggcctcccatagtgctgggattacaggtgtgagccaccgcactcagctgatttgcaaaaactttcttccatactgttgtttcactttctggatagtgtcctctgaagcacaaaagttaattttgatgaagtacagcaatttatctgtttttttcttttgttgcttatgcttttgctgtcacatttcagaagccatcacctaatccaagatcacagagatttacacctagtttctaataagcattttataatttcagctcttatgtgtagatctttgatccattgtgaattaagttttgtgtgtggtgtaagagcccacattcttttgcatgtggatatccagttgtcccagcaccatttgttgaaaacattatttttccccattgaattatcttcgcaccattattgataatcagttgaccaaaaatgtacaggcttatttctgggcacttgactctattccattgacctgtttaaccttatgccagtgccacagtgccttgatcagtgctgctttgttgtgagaagttttactttgttctgcttcaaggctgttttgactcttctgcatctcttgcatttccaaatgaattttaggatccactcatcagtttcttcaaaaaaaaaaaaaaagcagctgagatttttgataggaattacattaaatctagagattgctttggaatgtattaccgtcttaatattaaatcttctgagtcatgaaaaaaaaaa

SEQ ID NO: 18 - protein, 709 aa, Homo sapiens
MDQKLSKLVEELTTSGEPRLNPEKMKELKKICKSSEEQLSRAYRLLIAQLTQEHAEIRLSAFQIVEELFVRSHQFRMLVVSNFQEFLELTLGTDPAQPLPPPREAAQRLRQATTRAVEGWNEKFGEAYKKLALGYHFLRHNKKVDFQDTNARSLAERKREEEKQKHLDKIYQERASQAEREMQEMSGEIESCLTEVESCFRLLVPFDFDPNPETESLGMASGMSDALRSSCAGQVGPCRSGTPDPRDGEQPCCSRDLPASAGHPRAGGGAQPSQTATGDPSDEDEDSDLEEFVRSHGLGSHKYTLDVELCSEGLKVQENEDNLALIHAARDTLKLIRNKFLPAVCSWIQRFTRVGTHGGCLKRAIDLKAELELVLRKYKELDIEPEGGERRRTEALGDAEEDEDDEDFVEVPEKEGYEPHIPDHLRPEYGLEAAPEKDTVVRCLRTRTRMDEEVSDPTSAAAQLRQLRDHLPPPSSASPSRALPEPQEAQKLAAERARAPVVPYGVDLHYWGQELPTAGKIVKSDSQHRFWKPSEVEEEVVNADISEMLRSRHITFAGKFEPVQHWCRAPRPDGRLCERQDRLKCPFHGKIVPRDDEGRPLDPEDRAREQRRQLQKQERPEWQDPELMRDVEAATGQDLGSSRYSGKGRGKKRRYPSLTNLKAQADTARARIGRKVFAKAAVRRVVAAMNRMDQKKHEKFSNQFNYALN

SEQ ID NO: 19 - DNA, 2947 bp, Homo sapiens
  source 1..2947: mol_type = other DNA
tgctgtagctgccatgggcaaaagagaccgagcggaccgcggtgagacgttgcgcgggcacgctcagccacgactgcccttgccggccctgccccccgctctgcctcggagctgctccgggcctctgcgccggccgaccctgctggccctcccgcgcgcaccccgttgggacaggcctttgggcgggagagatgctggacctgggcgcagcccagcgaactcggcctaggggagacgggtgaggggcgcaacgcctgcgggatgcaggtggctcttagctaggagtttgcggcggcgcagacaagaagaaatccaggaagcggcactatgaggatgaagaggatgatgaagaggacgccccggggaacgaccctcaggaagcggttccctcggcggcggggaagcaggtggatgagtcaggcaccaaagtggatgaatatggagccaaggactacaggctgcaaatgccgctgaaggacgaccacacctccaggcccctctgggtggctcccgatggccatatcttcttggaagccttctctccagtttacaaatatgcccaagacttcttggtggctattgcagagccagtgtgccgaccaacccatgtgcatgagtacaaactaactgcctactccttgtatgcagctgtcagcgttgggctgcaaaccagtgacatcaccgagtacctcaggaagctcagcaagactggagtccctgatggaattatgcagtttattaagttgtgtactgtcagctatggaaaagtcaagctggtcttgaagcacaacagatacttcgttgaaagttgccaccctgatgtaatccagcatcttctccaggaccccgtgatccgagaatgccgcttaagaaactctgaaggggaggccactgagctcatcacagagactttcacaagcaaatctgccatttctaagactgctgaaagcagtggtgggccctccacttcccgagtgacagatccacagggtaaatctgacatccccatggacctgtttgacttctatgagcaaatggacaaggatgaagaagaagaagaagagacacagacagtgtcttttgaagtcaagcaggaaatgattgaggaactccagaaacgttgcatccacctggagtaccctctgttggcagaatatgacttccggaatgattctgtcaaccctgatatcaacattgacctaaagcccacagctgtcctcagaccctatcaggagaagagcttgcgaaagatgtttggaaacgggcgtgcacgttcgggggtcattgttcttccctgcggtgctggaaagtccctggttggtgtgactgctgcatgcactgtcagaaaacgctgtctggtgctgggcaactcagctgtttctgtggagcagtggaaagcccagttcaagatgtggtccaccattgacgacagccagatctgccggttcacctccgatgccaaggacaagcccatcggctgctccgttgccattagcacctactccatgctgggccacaccaccaaaaggtcctgggaggccgagcgagtcatggagtggctcaagacccaggagtggggcctcatgatcctggatgaagtgcacaccataccagccaagatgttccgaagggtgctcaccatcgtgcaggcccactgtaagctgggtttgactgcgaccctcgtccgcgaagatgacaaaattgtggatttaaattttctgattgggcctaagctctacgaagccaactggatggagctgcagaataatggctacatcgccaaagtccagtgtgctgaggtctggtgccctatgtctcctgaattttaccgggaatatgtggcaatcaaaaccaagaaacgaatcttgctgtacaccatgaaccccaacaaatttagagcttgccagtttctgatcaagtttcatgaaaggaggaatgacaagattattgtctttgctgacaatgtgtttgccctaaaggaatatgccattcgactgaacaaaccctatatctacggacctacgtctcagggggaaaggatgcaaattctccagaatttcaagcacaaccccaaaattaacaccatcttcatatccaaggtaggtgacacttcgtttgatctgccggaagcaaatgtcctcattcagatctcatcccatggtggctccaggcgtcaggaagcccaaaggctagggcgggtgcttcgagctaaaaaagggatggttgcagaagagtacaatgcctttttctactcactggtatcccaggacacacaggaaatggcttactcaaccaagcggcagagattcttggtagatcaaggttatagcttcaaggtgatcacgaaactcgctggcatggaggaggaagacttggcgttttcgacaaaagaagagcaacagcagctcttacagaaagtcctggcagccactgacctggatgccgaggaggaggtggtggctggggaatttggctccagatccagccaggcatctcggcgctttggcaccatgagttctatgtctggggccgacgacactgtgtacatggagtaccactcatcgcggagcaaggcgcccagcaaacatgtacacccgctcttcaagcgctttaggaaatgatgcttaggcagggtacttcgttcaagaccggcgcttggcacccttgttggaaagggattttcagcataacattttccttccacctctttgaccttccctccagcgttggccaaattgtgctgaggaagatgcatcaagggcttggctgtgccttcataggtcatctagggttttataaaggaggaggagacaatattttttcaaactttttggggagtggggtcatttctgtatataaaaaatgttaatatttaaggtgtatttatgttaccgttctgaataaacagaatggaccattgaaccagtaaaaaaaaaaaaaaaaaaaa

SEQ ID NO: 20 - protein, 718 aa, Homo sapiens
MPLKDDHTSRPLWVAPDGHIFLEAFSPVYKYAQDFLVAIAEPVCRPTHVHEYKLTAYSLYAAVSVGLQTSDITEYLRKLSKTGVPDGIMQFIKLCTVSYGKVKLVLKHNRYFVESCHPDVIQHLLQDPVIRECRLRNSEGEATELITETFTSKSAISKTAESSGGPSTSRVTDPQGKSDIPMDLFDFYEQMDKDEEEEEETQTVSFEVKQEMIEELQKRCIHLEYPLLAEYDFRNDSVNPDINIDLKPTAVLRPYQEKSLRKMFGNGRARSGVIVLPCGAGKSLVGVTAACTVRKRCLVLGNSAVSVEQWKAQFKMWSTIDDSQICRFTSDAKDKPIGCSVAISTYSMLGHTTKRSWEAERVMEWLKTQEWGLMILDEVHTIPAKMFRRVLTIVQAHCKLGLTATLVREDDKIVDLNFLIGPKLYEANWMELQNNGYIAKVQCAEVWCPMSPEFYREYVAIKTKKRILLYTMNPNKFRACQFLIKFHERRNDKIIVFADNVFALKEYAIRLNKPYIYGPTSQGERMQILQNFKHNPKINTIFISKVGDTSFDLPEANVLIQISSHGGSRRQEAQRLGRVLRAKKGMVAEEYNAFFYSLVSQDTQEMAYSTKRQRFLVDQGYSFKVITKLAGMEEEDLAFSTKEEQQQLLQKVLAATDLDAEEEVVAGEFGSRSSQASRRFGTMSSMSGADDTVYMEYHSSRSKAPSKHVHPLFKRFRK

SEQ ID NO: 21 - DNA, 2568 bp, Homo sapiens
  source 1..2568: mol_type = other DNA
ttcatgagggaggcgggtcgaccccgctgcacagtccggccggcgccatgaagctcaacgtggacgggctcctggtctacttcccgtacgactacatctaccccgagcagttctcctacatgcgggagctcaaacgcacgctggacgccaagggtcatggagtcctggagatgccctcaggcaccgggaagacagtatccctgttggccctgatcatggcataccagagagcatatccgctggaggtgaccaaactcatctactgctcaagaactgtgccagagattgagaaggtgattgaagagcttcgaaagttgctcaacttctatgagaagcaggagggcgagaagctgccgtttctgggactggctctgagctcccgcaaaaacttgtgtattcaccctgaggtgacacccctgcgctttgggaaggacgtcgatgggaaatgccacagcctcacagcctcctatgtgcgggcgcagtaccagcatgacaccagcctgccccactgccgattctatgaggaatttgatgcccatgggcgtgaggtgcccctccccgctggcatctacaacctggatgacctgaaggccctggggcggcgccagggctggtgcccatacttccttgctcgatactcaatcctgcatgccaatgtggtggtttatagctaccactacctcctggaccccaagattgcagacctggtgtccaaggaactggcccgcaaggccgtcgtggtcttcgacgaggcccacaacattgacaacgtctgcatcgactccatgagcgtcaacctcacccgccggacccttgaccggtgccagggcaacctggagaccctgcagaagacggtgctcaggatcaaagagacagacgagcagcgcctgcgggacgagtaccggcgtctggtggaggggctgcgggaggccagcgccgcccgggagacggacgcccacctggccaaccccgtgctgcccgacgaagtgctgcaggaggcagtgcctggctccatccgcacggccgagcatttcctgggcttcctgaggcggctgctggagtacgtgaagtggcggctgcgtgtgcagcatgtggtgcaggagagcccgcccgccttcctgagcggcctggcccagcgcgtgtgcatccagcgcaagcccctcagattctgtgctgaacgcctccggtccctgctgcatactctggagatcaccgaccttgctgacttctccccgctcaccctccttgctaactttgccacccttgtcagcacctacgccaaaggcttcaccatcatcatcgagccctttgacgacagaaccccgaccattgccaaccccatcctgcacttcagctgcatggacgcctcgctggccatcaaacccgtatttgagcgtttccagtctgtcatcatcacatctgggacactgtccccgctggacatctaccccaagatcctggacttccaccccgtcaccatggcaaccttcaccatgacgctggcacgggtctgcctctgccctatgatcatcggccgtggcaatgaccaggtggccatcagctccaaatttgagacccgggaggatattgctgtgatccggaactatgggaacctcctgctggagatgtccgctgtggtccctgatggcatcgtggccttcttcaccagctaccagtacatggagagcaccgtggcctcctggtatgagcaggggatccttgagaacatccagaggaacaagctgctctttattgagacccaggatggtgccgaaaccagtgtcgccctggagaagtaccaggaggcctgcgagaatggccgcggggccatcctgctgtcagtggcccggggcaaagtgtccgagggaatcgactttgtgcaccactacgggcgggccgtcatcatgtttggcgtcccctacgtctacacacagagccgcattctcaaggcgcggctggaatacctgcgggaccagttccagattcgtgagaatgactttcttaccttcgatgccatgcgccacgcggcccagtgtgtgggtcgggccatcaggggcaagacggactacggcctcatggtctttgccgacaagcggtttgcccgtggggacaagcgggggaagctgccccgctggatccaggagcacctcacagatgccaacctcaacctgaccgtggacgagggtgtccaggtggccaagtacttcctgcggcagatggcacagcccttccaccgggaggatcagctgggcctgtccctgctcagcctggagcagctagaatcagaggagacgctgaagaggatagagcagattgctcagcagctctgagtggggcgggtggggccataaacggttcctggtgactcctgagtcttgcctggccctggttcccagcggcggtggtgctagaaggtcttatgaagtcaggtgacatttctcactgtcacgtccacagcctttaatcgcaggagaaggcagctatccaccaggtacccagaggcaagggggggccaggagatgatagaccccctctcaccccaccagcccatccctcctgcactgttcc

SEQ ID NO: 22 - protein, 760 aa, Homo sapiens
MKLNVDGLLVYFPYDYIYPEQFSYMRELKRTLDAKGHGVLEMPSGTGKTVSLLALIMAYQRAYPLEVTKLIYCSRTVPEIEKVIEELRKLLNFYEKQEGEKLPFLGLALSSRKNLCIHPEVTPLRFGKDVDGKCHSLTASYVRAQYQHDTSLPHCRFYEEFDAHGREVPLPAGIYNLDDLKALGRRQGWCPYFLARYSILHANVVVYSYHYLLDPKIADLVSKELARKAVVVFDEAHNIDNVCIDSMSVNLTRRTLDRCQGNLETLQKTVLRIKETDEQRLRDEYRRLVEGLREASAARETDAHLANPVLPDEVLQEAVPGSIRTAEHFLGFLRRLLEYVKWRLRVQHVVQESPPAFLSGLAQRVCIQRKPLRFCAERLRSLLHTLEITDLADFSPLTLLANFATLVSTYAKGFTIIIEPFDDRTPTIANPILHFSCMDASLAIKPVFERFQSVIITSGTLSPLDIYPKILDFHPVTMATFTMTLARVCLCPMIIGRGNDQVAISSKFETREDIAVIRNYGNLLLEMSAVVPDGIVAFFTSYQYMESTVASWYEQGILENIQRNKLLFIETQDGAETSVALEKYQEACENGRGAILLSVARGKVSEGIDFVHHYGRAVIMFGVPYVYTQSRILKARLEYLRDQFQIRENDFLTFDAMRHAAQCVGRAIRGKTDYGLMVFADKRFARGDKRGKLPRWIQEHLTDANLNLTVDEGVQVAKYFLRQMAQPFHREDQLGLSLLSLEQLESEETLKRIEQIAQQL

SEQ ID NO: 23 - DNA, 6765 bp, Homo sapiens
  source 1..6765: mol_type = other DNA
agagcttccatggagtcagggcagccggctcgacggattgccatggcgccgctgctggagtacgagcgacagctggtgctggaactgctcgacactgacgggctagtagtgtgcgcccgcgggctcggcgcggaccggctcctctaccactttctccagctgcactgccacccagcctgcctggtgctggtgctcaacacgcagccggccgaggaggagtattttatcaatcagctgaagatagaaggagttgaacacctccctcgccgtgtaacaaatgaaatcacaagcaacagtcgctatgaagtttacacacaaggtggtgttatatttgcgacaagtaggatacttgtggttgacttcttgactgatagaataccttcagatttaattactggcatcttggtgtatagagcccacagaataatcgagtcttgtcaagaagcattcatcttgcgcctctttcgccagaaaaacaaacgtggttttattaaagctttcacagacaatgctgttgcctttgatactggtttttgtcatgtggaaagagtgatgagaaatctttttgtgaggaaactgtatctgtggccaaggttccatgtagcagtaaactcatttttagaacagcacaaacctgaagttgtagaaatccatgtttctatgacacctaccatgcttgctatacagactgctatactggacattttaaatgcatgtctaaaggaactaaaatgccataacccatcgcttgaagtggaagatttatctttagaaaatgctattggaaaaccttttgacaagacaatccgccattatctggatcctttgtggcaccagcttggagccaagactaaatccttagttcaggatttgaagatattacgaactttgctgcagtatctctctcagtatgattgtgtcacatttcttaatcttctggaatctctgagagcaacggaaaaagcttttggtcagaattcaggttggctgtttcttgactccagcacctcgatgtttataaatgctcgagcaagggtttatcatcttccagatgccaaaatgagtaaaaaagaaaaaatatctgaaaaaatggaaattaaagaaggggaagaaacaaaaaaggaactggtcctagaaagcaacccaaagtgggaggcactgactgaagtattaaaagaaattgaggcagaaaataaggagagtgaagctcttggtggtccaggtcaagtactgatttgtgcaagtgatgaccgaacatgttcccagctgagagactatatcactcttggagcggaggccttcttattgaggctctacaggaaaacctttgagaaggatagcaaagctgaagaagtctggatgaaatttaggaaggaagacagttcaaagagaattaggaaatctcacaaaagacctaaagacccccaaaacaaagaacgggcttctaccaaagaaagaaccctcaaaaagaaaaaacggaagttgaccttaactcaaatggtaggaaaacctgaagaactggaagaggaaggagatgtcgaggaaggatatcgtcgagaaataagcagtagcccagaaagctgcccggaagaaattaagcatgaagaatttgatgtaaatttgtcatcggatgctgctttcggaatcctgaaagaacccctcactatcatccatccgcttctgggttgcagcgacccctatgctctgacaagggtactacatgaagtggagccaagatacgtggttctttatgacgcagagctaacctttgttcggcagcttgaaatttacagggcgagtaggcctgggaaacctctgagggtttactttcttatatacggaggttcaactgaggaacaacgctatctcactgctttgcggaaagaaaaggaagcttttgaaaaactcataagggaaaaagcaagcatggttgtccctgaagaaagagaaggcagagatgaaacaaacttagacctagtaagaggcacagcatctgcagatgtttccactgacactcggaaagccggtggccaggaacagaatggtacacagcaaagcatagttgtggatatgcgtgaatttcgaagtgagcttccatctctgatccatcgtcggggcattgacattgaacccgtgactttagaggttggagattacatcctcactccagaaatgtgcgtggagcgcaagagtatcagtgatttaatcggctctttaaataacggccgcctctacagccagtgcatctccatgtcccgctactacaagcgtcccgtgcttctgattgagtttgaccctagcaagcctttctctctcacttcccgaggtgccttgtttcaggagatctccagcaatgacattagttccaaactcactcttcttacacttcacttccccagactacggattctctggtgcccctctcctcatgcaacggcggagttgtttgaggagctgaaacaaagcaagccacagcctgatgcggcgacagcactggccattacagcagattctgaaacccttcccgagtcagagaagtataatcctggtccccaagacttcttgttaaaaatgccaggggtgaatgccaaaaactgccgctccttgatgcaccacgttaagaacatcgcagaattagcagccctgtcacaagacgagctcacgagtattctggggaatgctgcaaatgccaaacagctttatgatttcattcacacctcttttgcagaagtcgtatcaaaaggaaaagggaaaaagtgaacagtgatggctgttttcttatcccatgcctgtacttttcagcggctccttgccagacatcataggtcattattaattattggtttgctatttcattcttttccaatgctcttaatgattgtacggtggaccagaagccaggattcctctctgaactctgcagttaggcatcacttgaacttgcctgtgcctgctctttttcctccctgcaccgtctatgccgggcttagcatgtttctttttaaatgaggtttgtcaggatcaggtaaagttcctacaagtgattacagaaggtagaaactttacctgatcctaacagatctcatttagaaaggaatatgctaagcctggcatggacggtgcagggagggaaaagagcaggcacaagaaagctaccatttttaacagtccttgttatctagtgcaacataaataacagtcttaattgcacttatacccatgtcctgtggctctccaaatctggtctttgctgttgtgtctgctggacgcttgaactgatgtttgtgtaggaaatcatgttctgaccctttgtctacaaaggagccttctggaacactgagaagaaacatctctttgccattcctgaccagttctctctaccacattttcttcagctccatacttctgcctgtctgctctaaggaaatttcatggagccttcctactactaattcaagacagtctcctcaaaaactggttgactagtcttctaatgaccctaacatatgtagcatatactataatttcattgttccaaattagtatttttaaagcaaaatgaattacctgtttgcaaaagttaatgatgaaggagctcttagaattctcaatttttgcacatattcagtctcctaatatcagagatccctaagtccagctggctagttacagagttttttcagacttcctcgtttctcagctcttatatcctaagacaccagcatcatatcctctagaaatacaacctaattggcagtgagccgagatcgcaccactgcacccctgcctgggcgacagagtgagactttgtctctattacaaaaagaaaagaaaagaaatacaacctaagctcacctgcctgtgattcctcatttctcaccatcctgtgccagggtggctacttctctctgtgaggactcaaatacaagccaatgagtggcccactaagcttttaagatttgattttcctgccttgagataaaaaggagtgtaggtaaatgaaagatcaatgtatggaatatataaaaatacgaaagaaatatatacgtttaaaaatccataaagaaaaaaatctcattctaaacctgattaagttggctttttacgtaagtgtacaaataggatattcacagcatctttgtgcagtttttaaacttttatatttaaacattattaagttggcttttgttcacatgttgagtaatgggtagtaaattttctacctcaggagctgatatagacatcagttctgctagccatatcacatattttaatgtttcatcaacatcagctgtttttttgtttgctacactatttgaactaatagacagtggatcatgtaacacaaaattgtcttcaactttaacaaaattgtcattgttatttttttttgagacagagtctccctctgttgcccaggctggagtgcagtggcgcaatctcggctcactgcaacctcgcctgctgggttcaagcagttctcccacctcagcctcccaagtagctgggattataggtgtgcaccaccagacccggctaatttttgtatttttagtagagacggggtttcaccatgttggccaggctggtctcaaactactgacctcaggtgatccacccaccttggcctcccaaagtgctgagataacaggcgtgagccaccgcacccacccaacaaaattatttttaactgtcgtttctgaactgaacctctcacattggcatcttgatttattggtacttaacagtatatatggattttgaactctacacaagggtataaccagaatgaattgaggggtatcaagaaccagattggttacagtagaactctgtaagatgatgtggagagtaaggaaaaggagaagaaataaaaattagtttaagatggaataaagatttgggctgctaattttttcccaggattcaaaatatacccgctagaatggaaaacaaaaatttgaatgattacaacattatcgttaacattcaattttgtatttattgttttattgaatatagttcagagtatattaaaatagacctaccacttcaaatgataatgattattataatgtctcttcaccctattctagcacttctgcttgcagtatgtggtttctatttttttcctcttgtataattccacttgcttttaattgttgtttcattatataaaaggaacatcttcccatagcatattctatgaaaggggtttcattccaagttgagttttcaaaaaaaaggtcttcctaaagctaccattttcaaccgtccttgttatctagtacaacataaataacagtcttaaaaattgcactaataccagtgcccccctggctctccaaatctgttctttgctcttgtatctgctggacgcttgaagacaggtgcactgtctcgtatgtatttgaattatgaacagtaatttctaatgaattctaaaatggtcattgtaagtgaaagcctctcgctaccacttcctcttccaactacataaatatatttcaatgtatttccagttttggaaagttttcaatacatacatcaagtgtttacttagatttttataaaaattttttttacaatctaataatctttggtaaaggaactagagatgcatgcagttgcaaaattaatgtatttattttccagcataattttattaacttcactttttttctctctagtaaatatccagtgtacttatgaactcatgtttggctcttttaaaaccttttctaaaagctagatcagcatttttctattttacaagttttttgtataaaaaggtgaacatatgagatattgtgagaaatcattttaagtttcatttaaatcatggtgcctcttttgatactttcttaaaattgtgcaagaagaaatcatttttagtagtggtcataaatattatccttttggcagtaagctattactaattcagcctgaagctcggtagacaatatgtctacatgtgtttgagtacatcctggatacagtttcccagctcatgagggactgaaaatagtcttattcatcaacccactaggatgtgaagggttaagtctagatttggtcgcattgaaaccccacaatatagaattaataaatggccttcagtaggaaaacctacactaaagcaaatccgaagaagtggggcagggggaaagaggcattactggtctttccttttgttttgcaagcataatttgattttcctttggctcagaaaactcatttggggaaattctcttttgtgttcagtttaacctagaaaggtcctcttgaaaaaccaacattttaggaaagttcttttttcaggatggagtatattaaaattaagccaggctttgacgtgaattatcacttttctttattattttgttttctattttggtttatagctatttctggttcagttctgaacttcagcacttaatcatccttatcaaccaggcttttggtagcctaaaccgctatgctgttgtttttttaatttaaagatgtataagccaaaatttggatgggagtgagacataactgatttatatgaattttaacagagttgtatttgtgtgtgtttaataaaatatatatttattcagtactttcctcagtattttatgggcaaagtaaaaataacaatgcatagtgaaagggcatatattaccagcagtaataattcaaaatcctgaaaatgtttcattttttttgtttttgttatgcagaataaacaaggcagaaatgctctttgaaccactaaaa

SEQ ID NO: 24 - protein, 916 aa, Homo sapiens
MESGQPARRIAMAPLLEYERQLVLELLDTDGLVVCARGLGADRLLYHFLQLHCHPACLVLVLNTQPAEEEYFINQLKIEGVEHLPRRVTNEITSNSRYEVYTQGGVIFATSRILVVDFLTDRIPSDLITGILVYRAHRIIESCQEAFILRLFRQKNKRGFIKAFTDNAVAFDTGFCHVERVMRNLFVRKLYLWPRFHVAVNSFLEQHKPEVVEIHVSMTPTMLAIQTAILDILNACLKELKCHNPSLEVEDLSLENAIGKPFDKTIRHYLDPLWHQLGAKTKSLVQDLKILRTLLQYLSQYDCVTFLNLLESLRATEKAFGQNSGWLFLDSSTSMFINARARVYHLPDAKMSKKEKISEKMEIKEGEETKKELVLESNPKWEALTEVLKEIEAENKESEALGGPGQVLICASDDRTCSQLRDYITLGAEAFLLRLYRKTFEKDSKAEEVWMKFRKEDSSKRIRKSHKRPKDPQNKERASTKERTLKKKKRKLTLTQMVGKPEELEEEGDVEEGYRREISSSPESCPEEIKHEEFDVNLSSDAAFGILKEPLTIIHPLLGCSDPYALTRVLHEVEPRYVVLYDAELTFVRQLEIYRASRPGKPLRVYFLIYGGSTEEQRYLTALRKEKEAFEKLIREKASMVVPEEREGRDETNLDLVRGTASADVSTDTRKAGGQEQNGTQQSIVVDMREFRSELPSLIHRRGIDIEPVTLEVGDYILTPEMCVERKSISDLIGSLNNGRLYSQCISMSRYYKRPVLLIEFDPSKPFSLTSRGALFQEISSNDISSKLTLLTLHFPRLRILWCPSPHATAELFEELKQSKPQPDAATALAITADSETLPESEKYNPGPQDFLLKMPGVNAKNCRSLMHHVKNIAELAALSQDELTSILGNAANAKQLYDFIHTSFAEVVSKGKGKK

SEQ ID NO: 25 - DNA, 4091 bp, Homo sapiens
  source 1..4091: mol_type = other DNA
atttttcatgggtttgcggacccaccagcgaaggcgggaggtgtcgcagggacatcttctggctgtttccgtcgcctgcgtggcccttgcaccccggtcttccattagcggcgcagacgtttgggcctaagcgctgggcgaggcgaggccctgcccctccccgccaacggccattctctggacctgtctttcttccgggaggcggtgacagctgctgagacgtgttgcagccagagtctctccgctttaatgcgctcccattagtgccgtcccccactggaaaaccgtggcttctgtattatttgccatctttgttgtgtaggagcagggagggcttcctcccggggtcctaggcggcggtgcagtccgtcgtagaagaattagagtagaagttgtcggggtccgctcttaggacgcagccgcctcatgggggtccaggggctctggaagctgctggagtgctccgggcggcaggtcagccccgaagcgctggaagggaagatcctggctgttgatattagcatttggttaaaccaagcacttaaaggagtccgggatcgccatgggaactcaatagaaaatcctcatcttctcactttgtttcatcggctctgcaaactcttattttttcgaattcgtcctatttttgtgtttgatggggatgctccactattgaagaaacagactttggtgaagagaaggcagagaaaggacttagcgtccagtgactccaggaaaacgacagagaagcttctgaaaacatttttgaaaagacaagccatcaaaactgccttcagaagcaaaagagatgaagcactacccagtcttacccaagttcgaagagaaaacgacctctatgttttgcctcctttacaagaggaagaaaaacacagttcagaagaggaagatgaaaaagaatggcaagaaagaatgaatcaaaaacaagcattacaggaagagttctttcataatcctcaagcgatagatattgagtctgaggacttcagcagcctgccccctgaagtaaagcatgaaatcttgactgatatgaaagagttcaccaagcgcagaagaacattatttgaagcaatgccagaggagtctgatgacttttcacagtaccaactcaaaggcttgcttaaaaagaactatctgaaccagcatatagaacatgtccaaaaggaaatgaatcagcaacattcaggacacatccgaaggcagtatgaagatgaagggggctttctgaaggaggtagagtcaaggagagtggtctctgaagacacttcacattacatcttgataaaaggtattcaagctaagacagttgcagaagtggattcagagtctcttccttcttccagcaaaatgcacggcatgtcttttgacgtgaagtcatctccatgtgaaaaactgaagacagagaaagagcctgatgctacccctccttctccaagaactttactagctatgcaagctgccctgctgggaagtagctcagaagaggagctggagagtgaaaatcgaaggcaggcccgtgggaggaacgcacctgctgctgtagacgaaggctccatatcaccccggactctttcagccattaagagagctcttgacgatgacgaagatgtaaaagtgtgtgctggggatgatgtgcagacgggagggccaggagcagaagaaatgcgtataaacagctccaccgagaacagtgatgaaggacttaaagtgagagatggaaaaggaataccgtttactgcaacacttgcgtcatctagtgtgaactctgcagaggagcacgtagccagcactaatgaggggagagagcccacagactcagttccaaaagaacaaatgtcacttgttcacgtggggactgaagcctttccgataagtgatgagtctatgattaaggacagaaaagatcggctgcctctggagagtgcagtggttagacatagtgacgcacctgggctcccgaatggaagggaactgacaccggcatctccaacttgtacaaattctgtgtcaaagaatgaaacacatgctgaagtgcttgagcagcagaacgaactttgcccatatgagagtaaattcgattcttctcttctttcaagtgatgatgaaacaaaatgtaaaccgaattctgcttctgaagtcattggccctgtcagtttgcaagaaacaagtagcatagtaagtgtcccttcagaggcagtagataatgtggaaaatgtggtgtcatttaatgctaaagagcatgagaattttctggaaaccatccaagaacagcagaccactgaatctgcaggccaggatttaatttccattccaaaggccgtggaaccaatggaaattgactcggaagaaagtgaatctgatggaagtttcattgaagtgcaaagtgtgattagtgatgaggaacttcaagcagaattccctgaaacttccaaacctccctcagaacaaggcgaagaggaactggtaggaactagggagggagaagcccctgctgagtccgagagcctcctgagggacaactctgagagggacgacgtggatggtgagccacaggaagctgagaaagatgcggaagattcgctccatgaatggcaagatattaatttggaggagttggaaactctggagagcaacctcttagcacagcagaattcactgaaagctcaaaaacagcagcaagaacggatcgctgctactgtcaccggacagatgttcctggaaagccaggaactcctgcgcctgttcggcattccctacatccaggctcccatggaagcagaggcgcagtgcgccatcctggacctgactgatcagacttccggaaccatcactgatgacagtgatatctggctgtttggagcgcggcatgtctatagaaacttttttaataaaaacaagtttgtagaatattatcaatatgtggactttcacaatcaattgggattggaccggaataagttaataaatttggcttatttgcttggaagtgattataccgaaggaataccaactgtgggttgtgtaaccgccatggaaattctcaatgaattccctgggcatggcctggaacctctcctaaaattctcagaatggtggcatgaagctcaaaaaaatccaaagataagacctaatcctcatgacaccaaagtgaaaaaaaaattacggacattgcaactcacccctggctttcctaacccagctgttgccgaggcctacctcaaacccgtggtggatgactcgaagggatcctttctgtgggggaaacctgatctcgacaaaattagagaattttgtcagcggtatttcggctggaacagaacgaagacagatgaatctctgtttcctgtattaaagcaactcgatgcccagcagacacagctccgaattgattccttctttagattagcacaacaggagaaagaagatgctaaacgtattaagagccagagactaaacagagctgtgacatgtatgctaaggaaagagaaagaagcagcagccagcgaaatagaagcagtttctgttgccatggagaaagaatttgagctacttgataaggcaaaacgaaaaacccagaagagaggcataacaaataccttagaagagtcatcaagcctgaaaagaaagaggctttcagattctaaacgaaagaatacatgcggtggatttttgggggagacctgcctctcagaatcatctgatggatcttcaagtgaagatgctgaaagttcatctttaatgaatgtacaaaggagaacagctgcgaaagagccaaaaaccagtgcttcagattcgcagaactcagtgaaggaagctcccgtgaagaatggaggtgcgaccaccagcagctctagtgatagtgatgacgatggagggaaagagaagatggtcctcgtgaccgccagatctgtgtttgggaagaaaagaaggaaactaagacgtgcgaggggaagaaaaaggaaaacctaattaaaaaatatgtatcctctataattagttatgacagccatttgtaatgaatttgtcgcaaagacgtaataaaattaactggtggcacggtctttgtaaaaaaa

SEQ ID NO: 26 - protein, 1186 aa, Homo sapiens
MGVQGLWKLLECSGRQVSPEALEGKILAVDISIWLNQALKGVRDRHGNSIENPHLLTLFHRLCKLLFFRIRPIFVFDGDAPLLKKQTLVKRRQRKDLASSDSRKTTEKLLKTFLKRQAIKTAFRSKRDEALPSLTQVRRENDLYVLPPLQEEEKHSSEEEDEKEWQERMNQKQALQEEFFHNPQAIDIESEDFSSLPPEVKHEILTDMKEFTKRRRTLFEAMPEESDDFSQYQLKGLLKKNYLNQHIEHVQKEMNQQHSGHIRRQYEDEGGFLKEVESRRVVSEDTSHYILIKGIQAKTVAEVDSESLPSSSKMHGMSFDVKSSPCEKLKTEKEPDATPPSPRTLLAMQAALLGSSSEEELESENRRQARGRNAPAAVDEGSISPRTLSAIKRALDDDEDVKVCAGDDVQTGGPGAEEMRINSSTENSDEGLKVRDGKGIPFTATLASSSVNSAEEHVASTNEGREPTDSVPKEQMSLVHVGTEAFPISDESMIKDRKDRLPLESAVVRHSDAPGLPNGRELTPASPTCTNSVSKNETHAEVLEQQNELCPYESKFDSSLLSSDDETKCKPNSASEVIGPVSLQETSSIVSVPSEAVDNVENVVSFNAKEHENFLETIQEQQTTESAGQDLISIPKAVEPMEIDSEESESDGSFIEVQSVISDEELQAEFPETSKPPSEQGEEELVGTREGEAPAESESLLRDNSERDDVDGEPQEAEKDAEDSLHEWQDINLEELETLESNLLAQQNSLKAQKQQQERIAATVTGQMFLESQELLRLFGIPYIQAPMEAEAQCAILDLTDQTSGTITDDSDIWLFGARHVYRNFFNKNKFVEYYQYVDFHNQLGLDRNKLINLAYLLGSDYTEGIPTVGCVTAMEILNEFPGHGLEPLLKFSEWWHEAQKNPKIRPNPHDTKVKKKLRTLQLTPGFPNPAVAEAYLKPVVDDSKGSFLWGKPDLDKIREFCQRYFGWNRTKTDESLFPVLKQLDAQQTQLRIDSFFRLAQQEKEDAKRIKSQRLNRAVTCMLRKEKEAAASEIEAVSVAMEKEFELLDKAKRKTQKRGITNTLEESSSLKRKRLSDSKRKNTCGGFLGETCLSESSDGSSSEDAESSSLMNVQRRTAAKEPKTSASDSQNSVKEAPVKNGGATTSSSSDSDDDGGKEKMVLVTARSVFGKKRRKLRRARGRKRKT

SEQ ID NO: 27 - DNA, 2492 bp, Homo sapiens
  source 1..2492: mol_type = other DNA
cactcagaaaggccgctgggtgcggggagcgcagaggcggtgcagggcggctggctcgcctcggcgtgcagtgcgcgtgcgtggagctgggagctaggtcctcggagtgggccagagatggcggcggccgacggggctttgccggaggcggcggctttagagcaacccgcggagctgcctgcctcggtgcgggcgagtatcgagcggaagcggcagcgggcactgatgctgcgccaggcccggctggctgcccggccctactcggcgacggcggctgcggctactggaggtttgggccgcgtccgcgctttccccttccctctccccgcctccccggtccccagactggctcgtgcaagccgagtcccggggcccgggggtcgcgtcaactccgctggcgtatgtgtgcagattctccccgagtcggagagggaatccgcccagccagccgccttgtcaaagcgtcctgtccacgaccacagagcgttcctctgtcgcacgcgggcctcctgacccccagccccgggccttcttcgctgcacctcggctgctggcagcttcgatttttcgtttagggatgcagccgccccggccgggaggtgtcagccactgccaggtgtcagggcctcagctgtcccggaaagaggagttagactagttcttgattctggcctctatacctagaactagttgatggaagaggcagatcttggtgtttgttagggagagctttcaacaattagagttgccaaaagtgcaatggattgcttctcatgagcttcccgttactagaagcgctcaaataggaactggaagaacgcttacttatgtggaggagaggagattcagcaatcagaatctcctactcagcaatcagaagaagggactggatagtaaacctttaagattctaatccatagattccatgagtctatttcttggacatgtgacctctgcgatttcttggcaaaaactagttgctcaaaacgtgtttattgattgaagtaaattaaggactgatctctcattgcagattattctcttatagaatttgccactggggtcaactaataatctttaacaggatacttaaaatatagtttggttgacaggcagttcttcagggctgcctctataacatgaagtacatgtgttcctaaagctctatactgccctttgtcgagcacagtttagagctgtattactcttttttatcagccagatgtagtgagagtgagaagagtctgaggctggcagcatgtcttgatgttcatactaacaccgaaactgcaaattccagctacttttgatggcatggctaatgtaaaagcagccccaaagataattgacacaggaggaggcttcattttagaagaggaagaagaagaagaacagaaaattggaaaagttgttcatcaaccaggacctgttatggaatttgattatgtaatatgcgaagaatgtgggaaagaatttatggattcttatcttatgaaccactttgatttgccaacttgtgataactgcagagatgctgatgataaacacaagcttataaccaaaacagaggcaaaacaagaatatcttctgaaagactgtgatttagaaaaaagagagccacctcttaaatttattgtgaagaagaatccacatcattcacaatggggtgatatgaaactctacttaaagttacagattgtgaagaggtctcttgaagtttggggtagtcaagaagcattagaagaagcaaaggaagtccgacaggaaaaccgagaaaaaatgaaacagaagaaatttgataaaaaagtaaaagaattgcggcgagcagtaagaagcagcgtgtggaaaagggagacgattgttcatcaacatgagtatggaccagaagaaaacctagaagatgacatgtaccgtaagacttgtactatgtgtggccatgaactgacatatgaaaaaatgtgattttttagttcagtgacctgttttatagaattttatatttaaataaaggaaatttagattggtccttttcaaaattcaaaaaaaaaagcaacatcttcatagatgaatgaaacccttgtataagtaatacttcagtaataattatgtatgttatggcttaaaagcaagtttcagtgaaggtcacctggcctggttgtgtgcacaatgtcatgtctgtgattgccttcttacaacagagatgggagctgagtgctagagtaggtgcagaagtggtaggtcagctacaaatttgaggacaagataccaaggcaaaccctagattggggtagagggaaaagggttcaacaaaggctgaactggattcttaaccaagaaacaaataatagcaatggtggtgcaccactgtaccccaggttctagtcatgtgttttttaggacgatttctgtctccacgatggtggaaacagtggggaactactgctggaaaaagccctaatagcagaaataaacattgagttgtacgagtctga

SEQ ID NO: 28 - protein, 273 aa, Homo sapiens
MAAADGALPEAAALEQPAELPASVRASIERKRQRALMLRQARLAARPYSATAAAATGGMANVKAAPKIIDTGGGFILEEEEEEEQKIGKVVHQPGPVMEFDYVICEECGKEFMDSYLMNHFDLPTCDNCRDADDKHKLITKTEAKQEYLLKDCDLEKREPPLKFIVKKNPHHSQWGDMKLYLKLQIVKRSLEVWGSQEALEEAKEVRQENREKMKQKKFDKKVKELRRAVRSSVWKRETIVHQHEYGPEENLEDDMYRKTCTMCGHELTYEKM

SEQ ID NO: 29 - DNA, 5739 bp, Homo sapiens
  source 1..5739: mol_type = other DNA
gacgtaatgcggtagcgcggggaatttcgagtggtgttggagcgccggaggctagtgggtggctgacccccagcatcctcgggagcgaccatggactccctggccgagtctcggtggcctccgggcctggcagtcatgaagacaatagatgatttgctgcggtgtggaatttgcttcgagtatttcaacattgcaatgataatacctcagtgttcacataactactgctctctctgtataagaaaatttctgtcctataaaactcagtgtccaacttgctgtgtgactgtcacagagccggatctgaaaaataaccgcatattagatgaactggtaaaaagcttgaattttgcacggaatcatctgctgcagtttgctttagagtcaccagccaaatctcctgcttcttcctcttcaaagaatcttgctgtcaaagtatatactcctgtagcctccagacagtctttaaagcaggggagcaggttaatggataatttcttgatcagagaaatgagtggttctacatcagagttgttgataaaagaaaataaaagcaaattcagccctcaaaaagaggcgagccctgctgcaaagaccaaagagacacgttctgtagaagagatcgctccagatccctcagaggctaagcgtcctgagccaccctcgacatccactttgaaacaagttactaaagtggattgtcctgtttgcggggttaacattccagaaagtcacattaataagcatttagacagctgtttatcacgcgaagagaagaaggaaagcctcagaagttctgttcacaaaaggaagccgctgcccaaaactgtatataatttgctctctgatcgtgatttaaagaaaaagctaaaagagcatggattatctattcaaggaaataaacaacagctcattaaaaggcaccaagaatttgtacacatgtacaatgcccaatgcgatgctttgcatcctaaatcagctgctgaaatagttcgagaaatcgaaaatatagagaagactaggatgcgtcttgaagctagtaaactcaatgaaagtgtaatggtttttacaaaggaccaaacagaaaaggaaatagatgaaatccacagtaaatatcgtaaaaaacataagagtgaatttcagcttctggtggatcaggctagaaaaggatacaagaaaattgctggaatgtcacaaaaaacagtaacaataacaaaagaagatgaatctacagaaaagctatcttctgtatgcatgggacaggaagataatatgacctcagtaacaaaccacttttctcaatcaaagctggactccccagaggaattggaacctgacagagaagaggattcttctagctgtattgatattcaagaagttctttcttcatcagaatcagattcatgcaatagttccagttcagacatcataagagatcttttagaagaagaggaagcctgggaagcatcacataaaaacgatcttcaagacacagaaataagtccaagacagaatcgccgcacaagagccgctgaaagtgctgagattgaaccaagaaacaagcgtaataggaattaatgtgggcttttgctgacttttcaaatgcattgattagaataccgtacttttggttgccacagatagattttctatttataaatgcccaaggaaagatgctaaattctaaatattacggttagctgatattcattcttttctgcttttccagaggggaaaaatgttacaaaatatccttacttggtcagttgcctcctgcctctaaaacatctctctctaaaaatactgacatttcacacaggtaccagctttgcagaggaggtagactctttggcactttggcacagggatttggtttggtttggtttggtttgataattaaatttcagaatgtccttaggccattctccttctcttccatggagaatccagcctcacaaaaggattctttcaacttctttattgcaagaaacagttgagttaaatgtttgtttttggaaaggcgggatgtcagttcacatccttggtgtgtgtaagtaccgatgcacgccaccaccatgcctgtgttacagcagctccatgatggttgttgcccgaggttaatgtagttgtttgttagacctgtgtcttacacatttctcagagtaggatattagtattataatttaaagctacgacagtcacaaagtcacaataacttagaaacattgcgcatattcttctgaaatagcacttaaaaatgattagtgtcagtattttttcacttgggtcaatcaaatctgtaacactgaatccaagctattaaacaaaaagtatgcaatgaatgaattttgtaaatgaatagagagtatcagtttacaataatgttcttaaaacagtatcctctggatagttgagtatgggttagaaatcattgaaatggattggtcagaaattgctatctgtgtaaaatgtctaccagtagccagatgcttccagagttcttaatgcctctctggcatttcagagccagcatcccccaactcccacccctctgccatcacccaacccaaacacatcagctttcaaatgagatgatagtaaatgcggcaatgttaagacaagaaatttatgatttgccagattcaacatttatgacctccccttccaaagactgtctccgttgaccttgtctttttggtatgccttggggtttctgataatgtgtggagtctcattatggctgagagtttagtgttttcacagtgaagtgcagacatttgatttctttatgagttccctgtgttagaaatggctatagaaaaatttgtcataataatttcatttgcatgaaatcctgaggggtgcattaaggaaactaaaagcaccacttaccaaatctatcggcagaactgatgtgaggtaagtgagcatgtcaaacaaaataggagctcacatggatatatttatgtcactgagttgtcagaaattatgtcaaaatgaaaactgtttgtttcatgacaaattatatagtctataaattaaactggaagtaattattactttaattgcagcaaaaggagtttgtgagggagcggtgagacccaagattgggaaagtaggcacatgagttcattcagcaaatatttggttatctatgtctgtcactgtgctgacactgggaatacaaaggtggccaaagatcatctagaacaatggttcccagtggggtgggcagaaagattttgccccccaggagacagctggcaatgtgtggagacacttttggaggtggagggtggtgaggggtactaccagtatcaatgggtggaggccagggatgaggctaaacacccaaccctcatggattagtttgccagggctgccgtcacaagatactgcagacggggaagcttacacaacagaaatgtgtattctcagaattctggaggctggaagtccaagatcagtagggtttcttcttcggcctctctccttggcttccactcatggtgtccttgcatggtcttttctctgtgtgcacatgactgtgcaggactggtgtcccgatttcttgtagggacagcagtcattggatcagggcccatgcgtatggcctcattttacttcagttacctcttttttagatagagggccctatctctaaatactgtcacattctgagataccggcagttaagacttccaacatatttagtggtaggggttaaggggcacgctttagactacaacactccacagtaaagaattatccagtctaacatgtcagttgtgccaagactgagaaacctgtaatgtaaaggaactcccaagtctacctgaaaagtaaggtctaaagagcatttattgagcactatctgttagcacttagcatatgtttaatattcatacattgaaagaaatgtatgttcatatccatctgacaaagattaaggaacttagagtaagtaatagatccaaaattgaaactgataaacttctggttctaaaattcaccttttagtgaaaatacagtttctcaccaaacttacagtaattcagagttattaactctttctacctcctcactcccctcaaagttataacatctcccatcaacatcagctcagaatggcagtgatgcttatggtttgtgggggtgcaggtgagctgctggatgcactttagatggcctagtatgccaggccgtcctttcatcctctactctcgtctttctttttcgacctcatgttgaggattactggtctagttgaaaaaagaattcatacacataagaccatgtatataatagagggaggtatgcctttaaaaacaaaattacatggtaaagactgatgtgtcaacagggatagacaacatagagaaggctagaactgtttctggaaaaaaacccaaaaacaatggatttcaaacttcttagcatccgataaaagaaagtagcaattattcaaatgagaaacacttctgtctgttatgtacatattatgaaagtatgaacataagagggaaaaccacaaccattacattttttaccatttcagtaatttttttttggtttttagtctgtttttagacatccttaagaacaactatctgaggctgtaaacaagtatttacattaacacccatgatactgactttatactccctacctgcattgagaccttaaagggggcatttctccattccttctgtgtatttctgggtatccccaggttcagatattcagaatacagatacaagtccatgcttgcatatgttgctgctccccccgacagacttctaccagcatctgccctcctcaccgctgctctgtcacgtgcactgcggctggccctgctggaaagtcctaccttgcctaggacgccatccccgatatagccccaagatgcaaacctgccaagtgctagggcagccctgccttcctgtccatttcagtctcttggccttgctgtgttccaagtggaaatgggagtgtgctttcctggtttgctagtgtactatcacccggaccacagcgtcctgtgcaaaaatttgcttcctctctcttcccctttcactctttctcttttctcttccccacccccttttttttctctttccccttacctttctttcctttccactcttcctgtccttccttccttcaactattaggactatatgtcaagtaatgtgcaaaacaaagacagttcctgccctgatggagcttgtagatcagaagggaagatgagcattaagtaattatatagattaatgtacaactgtgatgagtgcaacaaaagagaggttcatggcactatcaaacatgcaatggacggattttgtttttgcctataaactcttgccatagaaagcttgaaaatcagtccagggggacagcattaatcaccatgttctttccttatccctgtcttccccaaaattcatgtgttgaaacttaacaagaggtgggaactttaggatgtgattaagtcgtgagggcacagctcttatagatggggtcacggtccttctaaaagggcttgagggagtggttttgtttccttccatcccttccgccacatgaggacacagtcttcgttccctctggaggactcagcaacaacacaccatcttggaagcagagagcagtcctcaccagacacggaatctgccagtgccttgatcttggacttaccagcctccatacctgtgagaaataaatttctattgtctataa

SEQ ID NO: 30 - protein, 495 aa, Homo sapiens
MDSLAESRWPPGLAVMKTIDDLLRCGICFEYFNIAMIIPQCSHNYCSLCIRKFLSYKTQCPTCCVTVTEPDLKNNRILDELVKSLNFARNHLLQFALESPAKSPASSSSKNLAVKVYTPVASRQSLKQGSRLMDNFLIREMSGSTSELLIKENKSKFSPQKEASPAAKTKETRSVEEIAPDPSEAKRPEPPSTSTLKQVTKVDCPVCGVNIPESHINKHLDSCLSREEKKESLRSSVHKRKPLPKTVYNLLSDRDLKKKLKEHGLSIQGNKQQLIKRHQEFVHMYNAQCDALHPKSAAEIVREIENIEKTRMRLEASKLNESVMVFTKDQTEKEIDEIHSKYRKKHKSEFQLLVDQARKGYKKIAGMSQKTVTITKEDESTEKLSSVCMGQEDNMTSVTNHFSQSKLDSPEELEPDREEDSSSCIDIQEVLSSSESDSCNSSSSDIIRDLLEEEEAWEASHKNDLQDTEISPRQNRRTRAAESAEIEPRNKRNRN

SEQ ID NO: 31 - DNA, 4263 bp, Homo sapiens
  source 1..4263: mol_type = other DNA
ctctccacggcgcacagagcatgcgcggggcgggagtgagcgaaattcaagctccaaactctaagctccaagctccaagctccaagctccaagctccaaactcccgccggggtaactggaacccaatccgagggtcatggaggcatcccgaaggtttccggaagccgaggccttgagcccagagcaggctgctcattacctaagatatgtgaaagaggccaaagaagcaactaagaatggagacctggaagaagcatttaaacttttcaatttggcaaaggacatttttcccaatgaaaaagtgctgagcagaatccaaaaaatacaggaagccttggaggagttggcagaacagggagatgatgaatttacagatgtgtgcaactctggcttgctactttatcgagaactgcacaaccaactctttgagcaccagaaggaaggcatagctttcctctatagcctgtatagggatggaagaaaaggtggtatattggctgatgatatgggattagggaagactgttcaaatcattgctttcctttccggtatgtttgatgcatcacttgtgaatcatgtgctgctgatcatgccaaccaatcttattaacacatgggtaaaagaattcatcaagtggactccaggaatgagagtcaaaacctttcatggtcctagcaaggatgaacggaccagaaacctcaatcggattcagcaaaggaatggtgttattatcactacataccaaatgttaatcaataactggcagcaactttcaagctttaggggccaagagtttgtgtgggactatgtcatcctcgatgaagcacataaaataaaaacctcatctactaagtcagcaatatgtgctcgtgctattcctgcaagtaatcgcctcctcctcacaggaaccccaatccagaataatttacaagaactatggtccctatttgattttgcttgtcaagggtccctgctgggaacattaaaaacttttaagatggagtatgaaaatcctattactagagcaagagagaaggatgctaccccaggagaaaaagccttgggatttaaaatatctgaaaacttaatggcaatcataaaaccctattttctcaggaggactaaagaagacgtacagaagaaaaagtcaagcaacccagaggccagacttaatgaaaagaatccagatgttgatgccatttgtgaaatgccttccctttccaggaaaaatgatttaattatttggatacgacttgtgcctttacaagaagaaatatacaggaaatttgtgtctttagatcatatcaaggagttgctaatggagacgcgctcacctttggctgagctaggtgtcttaaagaagctgtgtgatcatcctaggctgctgtctgcacgggcttgttgtttgctaaatcttgggacattctctgctcaagatggaaatgagggggaagattccccagatgtggaccatattgatcaagtaactgatgacacattgatggaagaatctggaaaaatgatattcctaatggacctacttaagaggctgcgagatgagggacatcaaactctggtgttttctcaatcgaggcaaattctaaacatcattgaacgcctcttaaagaataggcactttaagacattgcgaatcgatgggacagttactcatcttttggaacgagaaaaaagaattaacttattccagcaaaataaagattactctgtttttctgcttaccactcaagtaggtggtgtcggtttaacattaactgcagcaactagagtggtcatttttgaccctagctggaatcctgcaactgatgctcaagctgtggatagagtttaccgaattggacaaaaagagaatgttgtggtttataggctaatcacttgtgggactgtagaggaaaaaatatacagaagacaggttttcaaggactcattaataagacaaactactggtgaaaaaaagaaccctttccgatattttagtaaacaagaattaagagagctctttacaatcgaggatcttcagaactctgtaacccagctgcagcttcagtctttgcatgctgctcagaggaaatctgatataaaactagatgaacatattgcctacctgcagtctttggggatagctggaatctcagaccatgatttgatgtacacatgtgatctgtctgttaaagaagagcttgatgtggtagaagaatctcactatattcaacaaagggttcagaaagctcaattcctcgttgaattcgagtctcaaaataaagagttcctgatggaacaacaaagaactagaaatgagggggcctggctaagagaacctgtatttccttcttcaacaaagaagaaatgccctaaattgaataaaccacagcctcagccttcacctcttctaagtactcatcatactcaggaagaagatatcagttccaaaatggcaagtgtagtcattgatgatctgcccaaagagggtgagaaacaagatctctccagtataaaggtgaatgttaccaccttgcaagatggtaaaggtacaggtagtgctgactctatagctactttaccaaaggggtttggaagtgtagaagaactttgtactaactcttcattgggaatggaaaaaagctttgcaactaaaaatgaagctgtacaaaaagagacattacaagaggggcctaagcaagaggcactgcaagaggatcctctggaaagttttaattatgtacttagcaaatcaaccaaagctgatattgggccaaatttagatcaactaaaggatgatgagattttacgtcattgcaatccttggcccattatttccataacaaatgaaagtcaaaatgcagaatcaaatgtatccattattgaaatagctgatgacctttcagcatcccatagtgcactgcaggatgctcaagcaagtgaggccaagttggaagaggaaccttcagcatcttcaccacagtatgcatgtgatttcaatcttttcttggaagactcagcagacaacagacaaaatttttccagtcagtctttagagcatgttgagaaagaaaatagcttgtgtggctctgcacctaattccagagcagggtttgtgcatagcaaaacatgtctcagttgggagttttctgagaaagacgatgaaccagaagaagtagtagttaaagcaaaaatcagaagtaaagctagaaggattgtttcagatggcgaagatgaagatgattcttttaaagatacctcaagcataaatccattcaacacatctctctttcaattctcatctgtgaaacaatttgatgcttcaactcccaaaaatgacatcagtccaccaggaaggttcttttcatctcaaatacccagtagtgtaaataagtctatgaactctagaagatctctggcttctaggaggtctcttattaatatggttttagaccacgtggaggacatggaggaaagacttgacgacagcagtgaagcaaagggtcctgaagattatccagaagaaggggtggaggaaagcagtggcgaagcctccaagtatacagaagaggatccttccggagaaacactgtcttcagaaaacaagtccagctggttaatgacgtctaagcctagtgctctagctcaagagacctctcttggtgcccctgagcctttgtctggtgaacagttggttggttctccccaggataaggcggcagaggctacaaatgactatgagactcttgtaaagcgtggaaaagaactaaaagagtgtggaaaaatccaggaggccctaaactgcttagttaaagcgcttgacataaaaagtgcagatcctgaagttatgctcttgactttaagtttgtataagcaacttaataacaattgagaatgtaacctgtttattgtattttaaagtgaaactgaatatgagggaatttttgttcccataattggattctttgggaacatgaagcattcaggcttaaggcaagaaagatctcaaaaagcaacttctgccctgcaacgccccccactccatagtctggtattctgagcactagcttaatatttcttcacttgaatattcttatattttaggcatattctataaatttaactgtgttgtttcttggaaagttttgtaaaattattctggtcattcttaattttactctgaaagtgatcatctttgtatataacagttcagataagaaaattaaagttacttttctcaagtgttttccaaaaaaaaaaaaaaaa

SEQ ID NO: 32 - protein, 1250 aa, Homo sapiens
MEASRRFPEAEALSPEQAAHYLRYVKEAKEATKNGDLEEAFKLFNLAKDIFPNEKVLSRIQKIQEALEELAEQGDDEFTDVCNSGLLLYRELHNQLFEHQKEGIAFLYSLYRDGRKGGILADDMGLGKTVQIIAFLSGMFDASLVNHVLLIMPTNLINTWVKEFIKWTPGMRVKTFHGPSKDERTRNLNRIQQRNGVIITTYQMLINNWQQLSSFRGQEFVWDYVILDEAHKIKTSSTKSAICARAIPASNRLLLTGTPIQNNLQELWSLFDFACQGSLLGTLKTFKMEYENPITRAREKDATPGEKALGFKISENLMAIIKPYFLRRTKEDVQKKKSSNPEARLNEKNPDVDAICEMPSLSRKNDLIIWIRLVPLQEEIYRKFVSLDHIKELLMETRSPLAELGVLKKLCDHPRLLSARACCLLNLGTFSAQDGNEGEDSPDVDHIDQVTDDTLMEESGKMIFLMDLLKRLRDEGHQTLVFSQSRQILNIIERLLKNRHFKTLRIDGTVTHLLEREKRINLFQQNKDYSVFLLTTQVGGVGLTLTAATRVVIFDPSWNPATDAQAVDRVYRIGQKENVVVYRLITCGTVEEKIYRRQVFKDSLIRQTTGEKKNPFRYFSKQELRELFTIEDLQNSVTQLQLQSLHAAQRKSDIKLDEHIAYLQSLGIAGISDHDLMYTCDLSVKEELDVVEESHYIQQRVQKAQFLVEFESQNKEFLMEQQRTRNEGAWLREPVFPSSTKKKCPKLNKPQPQPSPLLSTHHTQEEDISSKMASVVIDDLPKEGEKQDLSSIKVNVTTLQDGKGTGSADSIATLPKGFGSVEELCTNSSLGMEKSFATKNEAVQKETLQEGPKQEALQEDPLESFNYVLSKSTKADIGPNLDQLKDDEILRHCNPWPIISITNESQNAESNVSIIEIADDLSASHSALQDAQASEAKLEEEPSASSPQYACDFNLFLEDSADNRQNFSSQSLEHVEKENSLCGSAPNSRAGFVHSKTCLSWEFSEKDDEPEEVVVKAKIRSKARRIVSDGEDEDDSFKDTSSINPFNTSLFQFSSVKQFDASTPKNDISPPGRFFSSQIPSSVNKSMNSRRSLASRRSLINMVLDHVEDMEERLDDSSEAKGPEDYPEEGVEESSGEASKYTEEDPSGETLSSENKSSWLMTSKPSALAQETSLGAPEPLSGEQLVGSPQDKAAEATNDYETLVKRGKELKECGKIQEALNCLVKALDIKSADPEVMLLTLSLYKQLNNN

SEQ ID NO: 33 - DNA, 10324 bp, Homo sapiens
  source 1..10324: mol_type = other DNA
caattcgggagcccgttctcgcgggattttccaagaccgggttggaatttgcgggggttaggagacggaagtcagagcctaggaagatttgggggtcgccttgccggcctcctgtcctcctccggcggcggcggagcccgagagaactaggtgaacaccgctttgccagcctcacacagcgtcccctggctctgccgccgctccggacgtcgccctcccgttctgcttgggtccccttagtcgctacctttgctgggatccccctcctccatcctgtggcttcgggttgccgaagagcgatgctcggagggcggccggaagtggcgttggccgccattggcctgccggccagccaccttgctgtcctccgccgccttccgggtgttacatgcagccgggctcggcccctccccctggccggatggatccgtcggcgccacagccccgcgcggaaacctcaggcaaagacatatggcatccaggagaaagatgtcttgccccttctccagataatggaaaactttgtgaagcaagcataaaatctatcacagtggatgaaaatggcaagtcatttgcagtcgtcttatatgcagattttcaagaaaggaaaatacctcttaaacagcttcaagaagtgaaatttgttaaagattgccctaggaatcttatatttgatgatgaagatttagaaaaaccttatttcccaaaccgaaaatttccatcatcttctgttgcttttaaattatctgacaatggagactctattccttataccatcaataggtatttgagagactaccaaagagaaggaacccggtttctttatggacactacatccatggaggagggtgcattctgggtgatgacatgggacttggaaaaacagtacaggttatttcatttctggctgcagttttgcataaaaagggaactcgtgaggatattgaaaataacatgccagagtttttactaagaagtatgaaaaaggaacccctttcttctacagcaaaaaagatgttcttaatagttgctcctctttctgtcctctacaactggaaggatgaattggacacctggggatatttcagagtcactgttttacatggaaacagaaaagataatgaattaattcgtgtaaagcagaggaaatgtgaaattgctctaacaacttatgaaacactacgcttatgcctggatgaacttaacagtttggaatggtcagctgtcattgtggatgaagctcatagaatcaagaatccaaaagctagagtaacagaagttatgaaagctttgaaatgtaatgtccgcattggcctcactggaaccatccttcagaacaacatgaaggaactgtggtgtgttatggactgggctgtgccaggccttttagggagtgggacctacttcaagaagcagttttctgacccagtagaacatggtcagagacacacggcaacaaagagagaactagccactggccgaaaggccatgcaaagacttgccaaaaagatgtctggctggtttctcaggcgcaccaagactcttatcaaggatcagttgcctaagaaggaagaccggatggtgtattgttctttgacagatttccagaaagctgtctatcaaacagtgttagaaacagaggacgtgactttgatacttcaatcttctgagccttgtacctgtaggagtggccaaaaaaggagaaattgttgttataagaccaattctcatggtgaaacagtgaaaaccttgtatctcagttaccttacagtccttcagaaggtagctaaccatgtcgcgctactgcaagctgctagtacttccaaacaacaggaaacacttatcaaaaggatatgtgatcaggtattttccagattcccagattttgtgcagaaaagcaaagatgcagcctttgaaacactttctgaccctaaatacagtggaaaaatgaaggtccttcagcagcttttaaatcattgcaggaaaaacagagataaagttcttctcttttctttttccaccaagttgcttgacgtgctacagcagtactgtatggcgtctgggcttgattaccgacgacttgatggaagtacaaaatcagaggaaagactcaagattgtaaaagagttcaacagtacacaagatgttaacatttgccttgtctctacaatggctggtggactaggcctcaattttgtcggtgccaatgttgttgtattatttgatcctacttggaatccagccaatgatcttcaagccattgacagagcatataggattggacaatgtagagatgtcaaagtgcttaggctgatatccttgggaactgtggaggaaatcatgtatttacgacagatatacaagcagcaacttcactgtgtggtggttggaagtgaaaatgccaaacgatattttgaagcagttcaaggatctaaagagcatcaaggagagctttttgggatccataacctcttcaaatttaggtcccaagggtcttgtcttacgaaggacatcctggagagagaaggccaagtagaagcagggatcatgacagccacaacatggttgaaagagggacctccagcacacaaactggaaatgcctagacagcctgactgtcaggaatgcagaggtacagaacaagctgcagagccactggcaaaggaagcatgtgatctctgcagtgacttcagtgatgaagagccagtgggagccacaggaataaagactgccaaaaacaaagcacccgattcaagtaaagcttccagctctccaggacagcttaccttactccagtgtggtttctcgaaattgcttgaaacaaaatgtaaagcagttgaggatagtgatggaaatactgcctctgatgatgaaagttctgatgagcagcccacatgcctttcaacagaagccaaagatgctggttgtgagaaaaatcaggactctcttggtacttcaaaacatcagaaattagataacatcctaaatccaaaagaaaagcatattttttataaaagtgagaagattttagaacagaatatttcttccaagtctgacgagaaaaaaattaaaaatacagataaacattgcattttacagaatgtcacagaatcagaagatagtgatgtcatctgtcctacacaatacacaactgagagattccccgacaatagtataaggtttaagccacccttggaaggatctgaggattctgaaacagaacacactgtaaaaacaagaaataatgataatagtcgaaacactgatgacaaaagaaatggaataatttcaaaaaagttaagtcctgagaacacaaccctgaaatctattttgaaaagaaaaggcaccagtgatatcagtgatgaatctgatgacattgaaatttcttccaagtcaagagtaagaaagagagctagttcattgaggtttaagagaataaaagaaaccaaaaaagaacttcacaattctcccaaaacaatgaacaaaacaaaccaagtgtatgcagcaaatgaggatcataactctcagtttattgatgattattcatcctcagatgagagtttatccgtcagccacttcagtttctctaaacagagccacagaccaagaactataagagacagaactagtttttcttcaaaattgcctagccataataagaaaaatagcacttttattccaagaaaaccaatgaaatgttcaaatgagaaagttgttaatcaagagcagtcgtatgaatcaatggataaatttttagatggcgttcaggaagtggcttatattcactcaaaccagaatgtaattggatcgagcaaagctgaaaatcacatgagccgatgggcagcacatgacgtatttgagttgaagcagttttctcagctgcctgctaacatagctgtttgcagttctaagacatataaagaaaaagtggatgcagatacattgccacacacaaagaaaggccagcaaccgagtgaaggcagcatttcacttcctctttacatttcaaatcctgtaaaccagaagaagaaaaaagtctaccatacaaaccagaccaccttcataattggagaaacaccaaaaggaatccgcagaaaacaatttgaagaaatggcctcttattttaactcgtcttctgtaaacgaatttgctaaacatataaccaatgccacatcagaagaacgacagaaaatgctaagagacttttatgcttctcaatatccagaggtaaaagaattttttgtggattctgtgtcacaattcaacaattcttcctttgagaaaggagagcagcgcacccggaagaaatctgataaaagagaatctcttataaaaccaaggctgtcagattctgaaaccttgtcatttaaagattctaccaacaaaatttctcaagtttgcagcctaaaaacatataaaagaaaatcagttaagtttcagaatcatatttcctatagagaagaggtgttttttaatgatgcagaaactaagaaatcacctgttagttctactcaagagattgacagtgggaaaaacagccaggcatccgaagatactgtgacatcccgttctctgaacagtgagtctgaaacacgtgagagaaggttagaaaataccatgaaagaccaacaggacctcacaagaacgggcatttcaagaaaagaaccccttctcaaattggaaaacaaaaagatagaaaatccagtgctggaaaatacttctgtgataagcttacttggtgatacctctattcttgatgacctttttaaaagtcatgggaacagtcccacacaactgccaaagaaagttctttcagggcccatggaaaaagcaaaacagagaccaaaagatttctgggacatcttgaatgagcagaatgatgagagtcttagtaaactcacagacttggcagtaatagagactctgtgtgaaaaagcacctctagcagcaccctttaaaaggagagaagagccagcaacttctctttggaaatcaaatgagaaatttttatggaagaaatttagcccaagtgatacagatgaaaacgcaaccaatacacagagtaccacataagcatataaatgaattactgcaccagtaaactgctgccatcactgtttacggcactggattccacactgattctattatcttgaacacagttgttgacatatatttttattaaattattgctttaggattttttgaagtctaaagtattgtcatggatctgtttttcttgatatttgatttgatctttcaagaatatgattgtatttatagtataaacctctgttatgaattagaaaagattctaggtttgttaataggagacctgggacatctttcttactatattacataatgatgtgacacttgccccggtgagcattgtttcccagtatgaaagatgaagagtctgtaccgaatcagcatgagtgtccttccagtttaaaaaagctttgcttcgctctcctaatggctcataggctgaatcatgtctgcccctcaaatcaggtgtataccaatgtgttttttactagcacttgggaaagttattaagtattttctttttccctgggcatcatgttctattattattttagaaaaaagtcataattggtactgaatatatggtatatataatattaaaatggtaattttgcaacagctcaaaattaaaaggttaatgttatacactttactatatgagctgtgattactaccattagccacagataccagtgcctcaactttttatgtacctattgtgatttaatgtaaataaaggtttgtatagtacttttgtagttcttaagtatgaagaaatgggtaaactttttattttgttagaaactgttatattttgagtgtaatatttatggtttatagcaaaatgaatgtgcttattgttgaatgcatgtatttagaagcctttactcagcccctgtgttctgtgctaggagcttgagctctacaggtaaggcagagctaccggtgaatgaaaggaaatcatgtcagtgaaaaatcatggtggaaagcccctggcatcacatgtgcatgctgtaggcaggacctgagctgcctccgctgcaggttcagatgcaccgctgcagctgtccttcagttagttcacagggctgcaagaggaggacacatccctccagaaaacagcctgagccgggaactggctgtgctaaagagcactgctatcaagttgaggagagagggcttccgtgtactcaggatgtagagtcattgctcagaagtgaacaaaaaatcaaaaacaaaagtcttctcaagggactgatcggccaagtatgcttttctttagagcaatgttttgccctagagaattgtaaaatttatgtcatgactcagtacatatgtgttcgtacatatatgattggaataaaatgtttatgaaatatttactcataagccatgtaacatactttgacatttttctcttctaggattgtgtttggtagggcagtgggtttgtgtgtgtgttaatcctctcacaacccactgaattgaatttcatggcccatggttaagatccacagtgtgaaaacgctgttttaaattatatgagttcgtcatttgtttgctctgtgcagctgagttgtgtccagacttaccagatggtatgttttgccattgaggggccttctacacaatgagtgcatgatatggtccttgatagacttgacttgtagatgttttcagcctacaatgtgatcagctatctgaggaactccagtaagtagataccacttcatttcagtttatatacaagacaatgtagttcaaacattttaataccttgtaaattatgatattcatataaatattagctctatagtcttcatatatgtacagtttttttttttttttttttttttttgagattgagtctcactctgtcacccaggctggagtacagtggcatgatcttggctcactgcaacctccacctccctcccaaacaactcttatgcctcagcctcctgagtagctaggattacaggtgtgtaccaccacactcagctaaacatttttttttttttttgagacggagtctcactctgtcgccaggctggagtgcagtggctcgatcttggctcactgcagcctccgccccccgagtcaagcaattctcctgcctcagcctcccgagtagctgggactacaggtgcgtgccaccacgcccagctaatttttttgtgtttttagtagggatggggtttcaccatgttggccaggctggtctcaatctcctggctgcaagcgatccacctgccttgtcctcccaaagtgctgggattacaggtgtgagccaccatgcccagccaataaatgctattaaagaaacttttaggccgggcacagtggctcacgcctgtaatcccagcactttgggaggccgaggcaggcggatcacaaggtcatgagatcgagactatcctggctaacgcggtgaaaccccgtctctactaaaaaaaaaaaaaatacaaaaattagccaggcgtggtggtgggcgcctgtagtcccagctactcgggaggctgaggcaggagaatggcgtgaacctgggaggcagagcttgcagtgagccaggattgtgcccctgcaccccagcctgggtgacagagcaagactccatctcaaaaaaggaaacttttatcaatagtagtcaaaatgggagcaaaaatgcagaactggagtttaaaaattagtttccaacagttgtcagtgaaatatttatcactattatgtaggaaagtgtgtcaggtggaatgcagagtccagtatgaaaaggagcctgtttcagaacggaaactgtcaggtggagtggactccctgcaatggagtgatggaaagatttggtacaaccattgatgacctgagaacagacactaggaaggtttgacaagatccttcctacccccactccactggtgatgtgtcaccaagttctgctaactaaatgggtggcctcaagcaaatcccttacctccttttagccttagtttcccctttaaattggtgatggattagatcagtggtggtcagccattttcttaaagcaactgaactgtatcttaatcagaaacttaactagtaaaacaattaaaatgcaagtctgttgcttcaagcaacaccactgtggctccatggagcactaatttgaaaaccacagagatgagatcatttctaagaaaacctttcaactttgtttccatgatgctacttgccacaatcttttttacttaagcaatgataccgtttctggctgaacactcaccacacagctattaatattggaaactgaggcaagataccaagagatttttgcacttgaattcctcagggttctcttacagccctgaaaacacctcttcaaggaaataaataacatctgcaaatggtgcagctgcccctaagtattcaaggcaaagaagccttagtttgcagagatgatgataaacttttcatccttccacagaaaatgagaaagccacaggaaatacacggttattttctaattaaagttcaatgtgtacgcttttaaattctgaagttatccttcttttatgatatcaaaatagccctcttttttgaaatgatggtagatcataccttttggtctttttcattgtccttacataaaagttggtaatcatatgtcagtcccagaagttttatttgacacttactggtctataaaatccaaaggaatggggatcactgcctgccttaaaggtttttttgtgtgtttgtttgtttgtttgtttgcatgtgaaggatttttatgtcacaaatccagctaacagtggatctaggatctaggcctcaccgaggatgtcactgaacctgtcagccccaatgacaggcacacaccttcaaagagcaagagtgcccatcactttctaggctccttgcctgctgctcattccatcatcacggatacagacttgtttctggtttcatcttccaccagagattgcaaagcatcctctgcccagtgcttctaggaggccttactgggatggttcataagccactttcctcttggtgacagcccctgtacactgtggcatgtacggctggctccacttggtatttgttgcaggagggaagaacatctcacttttgcttggctaaagggccaccccaggagagctattttgcctcagggtcttgcagggtttctctaggaaaacagtgcatctgatagttactctctgcccctaggaggaaagacagattcgcccttccttcccaagtcccaggctgtgtggttcccagccccaagggtagcaagtcagaacctccggagaggcttttaagcatgcacatgtccagggtcctcatcaagagactgaatcagggggtctagggctcagcacaggcaccctttttgtttttgcagtttcacaggaattctgatgtgccagtgattgtcaggtttgtccctgggcaggacccacctgcctgaccttctgtccccctgggacacagtatttccagagaggtgcttcagttgccccaccacgacttccagcgccccccatctctgtatgcagctgagctcatgatggagcccactgtgtgctttttgttgttgttacattcctataaaaatgtccacgcatatttgaacatctgtgttcatagtggcattattcacaatggctacaacacgaagaaagcattccaagtacctatcgatgaataagcaaaatattacacacacaggaatattattcagccttgaaaggaaattcggacacgtgctacaacatagatgaatcttgaggacggtatgcaaagtgaaataaatcagacacagaaggacaaatactgtatgattccacttatataaagtacctagattagtcagattcatagagacaaagtagaatggtggttgctagacgctggagggaggggagaaaggggagttattaatgggtgtagagtttctgttacacaggatgcaaagtggcctgaagatggatggtcgtgatggttgcacagcaatgtgaatttaatgacactgaagtgtacacttaaaagtcattaaaatgctaaattttgtatgtgttttacaattttaaaaatggaaaaaaagtaagaccaaaagcaacacctctccagaatgtgtgttatataccaaattttgatgtatgtgagattgctgactatattttacttatcaatttgaaattatattgtttttatgttgacactccatataagccattattttgaaaaaaacattttccttattaaaagattggaaaaaaattcaca

SEQ ID NO: 34 - protein, 1561 aa, Homo sapiens
MQPGSAPPPGRMDPSAPQPRAETSGKDIWHPGERCLAPSPDNGKLCEASIKSITVDENGKSFAVVLYADFQERKIPLKQLQEVKFVKDCPRNLIFDDEDLEKPYFPNRKFPSSSVAFKLSDNGDSIPYTINRYLRDYQREGTRFLYGHYIHGGGCILGDDMGLGKTVQVISFLAAVLHKKGTREDIENNMPEFLLRSMKKEPLSSTAKKMFLIVAPLSVLYNWKDELDTWGYFRVTVLHGNRKDNELIRVKQRKCEIALTTYETLRLCLDELNSLEWSAVIVDEAHRIKNPKARVTEVMKALKCNVRIGLTGTILQNNMKELWCVMDWAVPGLLGSGTYFKKQFSDPVEHGQRHTATKRELATGRKAMQRLAKKMSGWFLRRTKTLIKDQLPKKEDRMVYCSLTDFQKAVYQTVLETEDVTLILQSSEPCTCRSGQKRRNCCYKTNSHGETVKTLYLSYLTVLQKVANHVALLQAASTSKQQETLIKRICDQVFSRFPDFVQKSKDAAFETLSDPKYSGKMKVLQQLLNHCRKNRDKVLLFSFSTKLLDVLQQYCMASGLDYRRLDGSTKSEERLKIVKEFNSTQDVNICLVSTMAGGLGLNFVGANVVVLFDPTWNPANDLQAIDRAYRIGQCRDVKVLRLISLGTVEEIMYLRQIYKQQLHCVVVGSENAKRYFEAVQGSKEHQGELFGIHNLFKFRSQGSCLTKDILEREGQVEAGIMTATTWLKEGPPAHKLEMPRQPDCQECRGTEQAAEPLAKEACDLCSDFSDEEPVGATGIKTAKNKAPDSSKASSSPGQLTLLQCGFSKLLETKCKAVEDSDGNTASDDESSDEQPTCLSTEAKDAGCEKNQDSLGTSKHQKLDNILNPKEKHIFYKSEKILEQNISSKSDEKKIKNTDKHCILQNVTESEDSDVICPTQYTTERFPDNSIRFKPPLEGSEDSETEHTVKTRNNDNSRNTDDKRNGIISKKLSPENTTLKSILKRKGTSDISDESDDIEISSKSRVRKRASSLRFKRIKETKKELHNSPKTMNKTNQVYAANEDHNSQFIDDYSSSDESLSVSHFSFSKQSHRPRTIRDRTSFSSKLPSHNKKNSTFIPRKPMKCSNEKVVNQEQSYESMDKFLDGVQEVAYIHSNQNVIGSSKAENHMSRWAAHDVFELKQFSQLPANIAVCSSKTYKEKVDADTLPHTKKGQQPSEGSISLPLYISNPVNQKKKKVYHTNQTTFIIGETPKGIRRKQFEEMASYFNSSSVNEFAKHITNATSEERQKMLRDFYASQYPEVKEFFVDSVSQFNNSSFEKGEQRTRKKSDKRESLIKPRLSDSETLSFKDSTNKISQVCSLKTYKRKSVKFQNHISYREEVFFNDAETKKSPVSSTQEIDSGKNSQASEDTVTSRSLNSESETRERRLENTMKDQQDLTRTGISRKEPLLKLENKKIENPVLENTSVISLLGDTSILDDLFKSHGNSPTQLPKKVLSGPMEKAKQRPKDFWDILNEQNDESLSKLTDLAVIETLCEKAPLAAPFKRREEPATSLWKSNEKFLWKKFSPSDTDENATNTQSTT

SEQ ID NO: 35 - DNA, 25 bp, synthetic construct
  misc_feature 1..25: note = Synthetic polynucleotide
  source 1..25: mol_type = other DNA
ccttgatcccagatgttgtggcctg

SEQ ID NO: 36 - DNA, 25 bp, synthetic construct
  misc_feature 1..25: note = Synthetic polynucleotide
  source 1..25: mol_type = other DNA
ctgggaaagccctcattgctacagt

SEQ ID NO: 37 - DNA, 25 bp, synthetic construct
  misc_feature 1..25: note = Synthetic polynucleotide
  source 1..25: mol_type = other DNA
tacctcagttgtgaccttcagcaga

SEQ ID NO: 38 - DNA, 25 bp, synthetic construct
  misc_feature 1..25: note = Synthetic polynucleotide
  source 1..25: mol_type = other DNA
gtggaggccatcagtattgactttc

SEQ ID NO: 39 - DNA, 25 bp, synthetic construct
  misc_feature 1..25: note = Synthetic polynucleotide
  source 1..25: mol_type = other DNA
tattgactttctcttacttgctgta

SEQ ID NO: 40 - DNA, 25 bp, synthetic construct
  misc_feature 1..25: note = Synthetic polynucleotide
  source 1..25: mol_type = other DNA
cttgctgtactatcagcctgctcgt

SEQ ID NO: 41 - DNA, 24 bp, synthetic construct
  misc_feature 1..24: note = Synthetic polynucleotide
  source 1..24: mol_type = other DNA
tgctcgtttccacctttaagaatg

SEQ ID NO: 42 - DNA, 25 bp, synthetic construct
  misc_feature 1..25: note = Synthetic polynucleotide
  source 1..25: mol_type = other DNA
ctcaatgtctcctactatccaaaat

SEQ ID NO: 43 - DNA, 25 bp, synthetic construct
  misc_feature 1..25: note = Synthetic polynucleotide
  source 1..25: mol_type = other DNA
atgtgcatcacaggaggctcttaac

SEQ ID NO: 44 - DNA, 25 bp, synthetic construct
  misc_feature 1..25: note = Synthetic polynucleotide
  source 1..25: mol_type = other DNA
ttgcgctaatgcttgaactcttttt

SEQ ID NO: 45 - DNA, 25 bp, synthetic construct
  misc_feature 1..25: note = Synthetic polynucleotide
  source 1..25: mol_type = other DNA
gtatgaccaaatcctgcctcattaa

SEQ ID NO: 46 - DNA, 25 bp, synthetic construct
  misc_feature 1..25: note = Synthetic polynucleotide
  source 1..25: mol_type = other DNA
gctagaaaccacctgattctgccag

SEQ ID NO: 47 - DNA, 25 bp, synthetic construct
  misc_feature 1..25: note = Synthetic polynucleotide
  source 1..25: mol_type = other DNA
tgaaagcgggcacctgcaggaagct

SEQ ID NO: 48 - DNA, 25 bp, synthetic construct
  misc_feature 1..25: note = Synthetic polynucleotide
  source 1..25: mol_type = other DNA
gaacacgatgaccttctggtggaga

SEQ ID NO: 49 - DNA, 25 bp, synthetic construct
  misc_feature 1..25: note = Synthetic polynucleotide
  source 1..25: mol_type = other DNA
tgagaaacttcatcgctttccaggc

SEQ ID NO: 50 - DNA, 25 bp, synthetic construct
  misc_feature 1..25: note = Synthetic polynucleotide
  source 1..25: mol_type = other DNA
tccaggcccacactgatggccaggc

SEQ ID NO: 51 - DNA, 25 bp, synthetic construct
  misc_feature 1..25: note = Synthetic polynucleotide
  source 1..25: mol_type = other DNA
caggccagcaccagggagatactgc

SEQ ID NO: 52 - DNA, 25 bp, synthetic construct
  misc_feature 1..25: note = Synthetic polynucleotide
  source 1..25: mol_type = other DNA
aagttatctgcatcacagtcttgtg

SEQ ID NO: 53 - DNA, 25 bp, synthetic construct
  misc_feature 1..25: note = Synthetic polynucleotide
  source 1..25: mol_type = other DNA
cagtcttgtgtcttccgagaactat

SEQ ID NO: 54 - DNA, 25 bp, synthetic construct
  misc_feature 1..25: note = Synthetic polynucleotide
  source 1..25: mol_type = other DNA
atctgtgcactttccatagaacttc

SEQ ID NO: 55 - DNA, 25 bp, synthetic construct
  misc_feature 1..25: note = Synthetic polynucleotide
  source 1..25: mol_type = other DNA
aacaacattgcttcctaaactttca

SEQ ID NO: 56 - DNA, 25 bp, synthetic construct
  misc_feature 1..25: note = Synthetic polynucleotide
  source 1..25: mol_type = other DNA
ggaagttggctgcacttgatgtttg

SEQ ID NO: 57 - DNA, 25 bp, synthetic construct
  misc_feature 1..25: note = Synthetic polynucleotide
  source 1..25: mol_type = other DNA
gagactgttgctcaaccatcaggaa

SEQ ID NO: 58 - DNA, 25 bp, synthetic construct
  misc_feature 1..25: note = Synthetic polynucleotide
  source 1..25: mol_type = other DNA
gagactgttgctcaaccatcaggaa

SEQ ID NO: 59 - DNA, 25 bp, synthetic construct
  misc_feature 1..25: note = Synthetic polynucleotide
  source 1..25: mol_type = other DNA
gcagaaagtcgcatagggtttttta

SEQ ID NO: 60 - DNA, 25 bp, synthetic construct
  misc_feature 1..25: note = Synthetic polynucleotide
  source 1..25: mol_type = other DNA
gacaatggcgctgcatgtttttctt

SEQ ID NO: 61 - DNA, 25 bp, synthetic construct
  misc_feature 1..25: note = Synthetic polynucleotide
  source 1..25: mol_type = other DNA
tatctccttttctctagtatttgac

SEQ ID NO: 62 - DNA, 25 bp, synthetic construct
  misc_feature 1..25: note = Synthetic polynucleotide
  source 1..25: mol_type = other DNA
tttgactgttactgtccttggcgaa

SEQ ID NO: 63 - DNA, 25 bp, synthetic construct
  misc_feature 1..25: note = Synthetic polynucleotide
  source 1..25: mol_type = other DNA
gttactgtccttggcgaatcgataa

SEQ ID NO: 64 - DNA, 25 bp, synthetic construct
  misc_feature 1..25: note = Synthetic polynucleotide
  source 1..25: mol_type = other DNA
ggcgaatcgataatcattgcatagt

SEQ ID NO: 65 - DNA, 25 bp, synthetic construct
  misc_feature 1..25: note = Synthetic polynucleotide
  source 1..25: mol_type = other DNA
tagtgactgaaaagcctaagtgcaa

SEQ ID NO: 66 - DNA, 25 bp, synthetic construct
  misc_feature 1..25: note = Synthetic polynucleotide
  source 1..25: mol_type = other DNA
ttcttgtttctgaacttcgtgccat

SEQ ID NO: 67 - DNA, 25 bp, synthetic construct
  misc_feature 1..25: note = Synthetic polynucleotide
  source 1..25: mol_type = other DNA
acttcgtgccatattttgttcctga

SEQ ID NO: 68 - DNA, 25 bp, synthetic construct
  misc_feature 1..25: note = Synthetic polynucleotide
  source 1..25: mol_type = other DNA
tctattagagacttaccctcctgac

SEQ ID NO: 69 - DNA, 25 bp, synthetic construct
  misc_feature 1..25: note = Synthetic polynucleotide
  source 1..25: mol_type = other DNA
cttaccctcctgacctgataaaaag

SEQ ID NO: 70 - DNA, 25 bp, synthetic construct
  misc_feature 1..25: note = Synthetic polynucleotide
  source 1..25: mol_type = other DNA
aaagggatacccatgtctctattaa

SEQ ID NO: 71 - DNA, 25 bp, synthetic construct
  misc_feature 1..25: note = Synthetic polynucleotide
  source 1..25: mol_type = other DNA
tacccatgtctctattaacagcttt

SEQ ID NO: 72 - DNA, 25 bp, synthetic construct
  misc_feature 1..25: note = Synthetic polynucleotide
  source 1..25: mol_type = other DNA
ttagttatgcttaaggaggagttct

SEQ ID NO: 73 - DNA, 25 bp, synthetic construct
  misc_feature 1..25: note = Synthetic polynucleotide
  source 1..25: mol_type = other DNA
taatgtaagacagttttggccttta

SEQ ID NO: 74 - DNA, 25 bp, synthetic construct
  misc_feature 1..25: note = Synthetic polynucleotide
  source 1..25: mol_type = other DNA
attatcatgagagatccttctgaat

SEQ ID NO: 75 - DNA, 25 bp, synthetic construct
  misc_feature 1..25: note = Synthetic polynucleotide
  source 1..25: mol_type = other DNA
ccttctgaataggatgtctttctga

SEQ ID NO: 76 - DNA, 25 bp, synthetic construct
  misc_feature 1..25: note = Synthetic polynucleotide
  source 1..25: mol_type = other DNA
tctttctgagttccactattcagtt

SEQ ID NO: 77 - DNA, 25 bp, synthetic construct
  misc_feature 1..25: note = Synthetic polynucleotide
  source 1..25: mol_type = other DNA
cagttacaaaactccttaatgctta

SEQ ID NO: 78 - DNA, 25 bp, synthetic construct
  misc_feature 1..25: note = Synthetic polynucleotide
  source 1..25: mol_type = other DNA
tactgtgtaccttttatatttaata

SEQ ID NO: 79 - DNA, 25 bp, synthetic construct
  misc_feature 1..25: note = Synthetic polynucleotide
  source 1..25: mol_type = other DNA
aagtctgtactgataaaacccattg

SEQ ID NO: 80 - DNA, 25 bp, synthetic construct
  misc_feature 1..25: note = Synthetic polynucleotide
  source 1..25: mol_type = other DNA
actgataaaacccattgtgtacaaa

SEQ ID NO: 81 - DNA, 25 bp, synthetic construct
  misc_feature 1..25: note = Synthetic polynucleotide
  source 1..25: mol_type = other DNA
ttctctttatacaagctgagtcata

SEQ ID NO: 82 - DNA, 25 bp, synthetic construct
  misc_feature 1..25: note = Synthetic polynucleotide
  source 1..25: mol_type = other DNA
gattttatattctggatttgtgttt

SEQ ID NO: 83 - DNA, 25 bp, synthetic construct
  misc_feature 1..25: note = Synthetic polynucleotide
  source 1..25: mol_type = other DNA
agactttggtgatcactttgcaaat

SEQ ID NO: 84 - DNA, 25 bp, synthetic construct
  misc_feature 1..25: note = Synthetic polynucleotide
  source 1..25: mol_type = other DNA
tggtgatcactttgcaaatatttgt

SEQ ID NO: 85 - DNA, 25 bp, synthetic construct
  misc_feature 1..25: note = Synthetic polynucleotide
  source 1..25: mol_type = other DNA
atatttgttaatccttgagtttgag

SEQ ID NO: 86 - DNA, 25 bp, synthetic construct
  misc_feature 1..25: note = Synthetic polynucleotide
  source 1..25: mol_type = other DNA
tgagtttgagaacctgtcttttaaa

SEQ ID NO: 87 - DNA, 25 bp, synthetic construct
  misc_feature 1..25: note = Synthetic polynucleotide
  source 1..25: mol_type = other DNA
atactttcaaacaagctagcaaggc

SEQ ID NO: 88 - DNA, 25 bp, synthetic construct
  misc_feature 1..25: note = Synthetic polynucleotide
  source 1..25: mol_type = other DNA
ttatatatttggttagttctgttta

SEQ ID NO: 89 - DNA, 25 bp, synthetic construct
  misc_feature 1..25: note = Synthetic polynucleotide
  source 1..25: mol_type = other DNA
ctgttgcccttatgagttattttat

SEQ ID NO: 90 - DNA, 25 bp, synthetic construct
  misc_feature 1..25: note = Synthetic polynucleotide
  source 1..25: mol_type = other DNA
attaccgactgtatgcctacacgga

SEQ ID NO: 91 - DNA, 25 bp, synthetic construct
  misc_feature 1..25: note = Synthetic polynucleotide
  source 1..25: mol_type = other DNA
tacacggagtcggagctgcagattg

SEQ ID NO: 92 - DNA, 25 bp, synthetic construct
  misc_feature 1..25: note = Synthetic polynucleotide
  source 1..25: mol_type = other DNA
ttccccaacatggtggtggcgcagg

SEQ ID NO: 93 - DNA, 25 bp, synthetic construct
  misc_feature 1..25: note = Synthetic polynucleotide
  source 1..25: mol_type = other DNA
gcagcaggcaatcgccagtggcatc

SEQ ID NO: 94 - DNA, 25 bp, synthetic construct
  misc_feature 1..25: note = Synthetic polynucleotide
  source 1..25: mol_type = other DNA
cagcccagcagataatccatttcct

SEQ ID NO: 95 - DNA, 25 bp, synthetic construct
  misc_feature 1..25: note = Synthetic polynucleotide
  source 1..25: mol_type = other DNA
cagactccggttcactgagggtgtc

SEQ ID NO: 96 - DNA, 25 bp, synthetic construct
  misc_feature 1..25: note = Synthetic polynucleotide
  source 1..25: mol_type = other DNA
gagggtgtcctgtataaccagttcc

SEQ ID NO: 97 - DNA, 25 bp, synthetic construct
  misc_feature 1..25: note = Synthetic polynucleotide
  source 1..25: mol_type = other DNA
aaccagttcctgtcgcaagtggact

SEQ ID NO: 98 - DNA, 25 bp, synthetic construct
  misc_feature 1..25: note = Synthetic polynucleotide
  source 1..25: mol_type = other DNA
ttcgagaactcggccaagcggctca

SEQ ID NO: 99 - DNA, 25 bp, synthetic construct
  misc_feature 1..25: note = Synthetic polynucleotide
  source 1..25: mol_type = other DNA
ggcacagcgacgtcaagcgcttttg

SEQ ID NO: 100 - DNA, 25 bp, synthetic construct
  misc_feature 1..25: note = Synthetic polynucleotide
  source 1..25: mol_type = other DNA
gctcctgagagcgcgggacttggac

SEQ ID NO: 101 - DNA, 25 bp, synthetic construct
  misc_feature 1..25: note = Synthetic polynucleotide
  source 1..25: mol_type = other DNA
gagcattcttcaggtcatctgaacc

SEQ ID NO: 102 - DNA, 25 bp, synthetic construct
  misc_feature 1..25: note = Synthetic polynucleotide
  source 1..25: mol_type = other DNA
gagaaaacatggtcaacgtcttgaa

SEQ ID NO: 103 - DNA, 25 bp, synthetic construct
  misc_feature 1..25: note = Synthetic polynucleotide
  source 1..25: mol_type = other DNA
gtgcttatagaatgtgatcctgcca

SEQ ID NO: 104 - DNA, 25 bp, synthetic construct
  misc_feature 1..25: note = Synthetic polynucleotide
  source 1..25: mol_type = other DNA
cagtttctgctgtacttggatgagt

SEQ ID NO: 105 - DNA, 25 bp, synthetic construct
  misc_feature 1..25: note = Synthetic polynucleotide
  source 1..25: mol_type = other DNA
ttggatgagtccaatgccctgggga

SEQ ID NO: 106 - DNA, 25 bp, synthetic construct
  misc_feature 1..25: note = Synthetic polynucleotide
  source 1..25: mol_type = other DNA
gatgacactcacgtctttgtaatag

SEQ ID NO: 107 - DNA, 25 bp, synthetic construct
  misc_feature 1..25: note = Synthetic polynucleotide
  source 1..25: mol_type = other DNA
gcagaattggttaatgtcctccagg

SEQ ID NO: 108 - DNA, 25 bp, synthetic construct
  misc_feature 1..25: note = Synthetic polynucleotide
  source 1..25: mol_type = other DNA
tgtcctccaggagcgagtgggtgaa

SEQ ID NO: 109 - DNA, 25 bp, synthetic construct
  misc_feature 1..25: note = Synthetic polynucleotide
  source 1..25: mol_type = other DNA
aactgtgaaagacttgccactcaat

SEQ ID NO: 110 - DNA, 25 bp, synthetic construct
  misc_feature 1..25: note = Synthetic polynucleotide
  source 1..25: mol_type = other DNA
gccactcaatatcttaggtgactga

SEQ ID NO: 111 - DNA, 25 bp, synthetic construct
  misc_feature 1..25: note = Synthetic polynucleotide
  source 1..25: mol_type = other DNA
gggttgttttaggagcatgccacgg

SEQ ID NO: 112 - DNA, 25 bp, synthetic construct
  misc_feature 1..25: note = Synthetic polynucleotide
  source 1..25: mol_type = other DNA
agtccggacagacattgtgggggtc

SEQ ID NO: 113 - DNA, 25 bp, synthetic construct
  misc_feature 1..25: note = Synthetic polynucleotide
  source 1..25: mol_type = other DNA
attaaggtttactctgctgccttgg

SEQ ID NO: 114 - DNA, 25 bp, synthetic construct
  misc_feature 1..25: note = Synthetic polynucleotide
  source 1..25: mol_type = other DNA
gccttggcagacttacgatctcaac

SEQ ID NO: 115 - DNA, 25 bp, synthetic construct
  misc_feature 1..25: note = Synthetic polynucleotide
  source 1..25: mol_type = other DNA
cgatctcaacagttcatacgagcag

SEQ ID NO: 116 - DNA, 25 bp, synthetic construct
  misc_feature 1..25: note = Synthetic polynucleotide
  source 1..25: mol_type = other DNA
gagggcatgctcactagtggttagt

SEQ ID NO: 117 - DNA, 25 bp, synthetic construct
  misc_feature 1..25: note = Synthetic polynucleotide
  source 1..25: mol_type = other DNA
gtggttagtaagctgtcgactttgt

SEQ ID NO: 118 - DNA, 25 bp, synthetic construct
  misc_feature 1..25: note = Synthetic polynucleotide
  source 1..25: mol_type = other DNA
gaacatgtacaatttgccactggga

SEQ ID NO: 119 - DNA, 25 bp, synthetic construct
  misc_feature 1..25: note = Synthetic polynucleotide
  source 1..25: mol_type = other DNA
aatgccagctgcgtgtctagttttg

SEQ ID NO: 120 - DNA, 25 bp, synthetic construct
  misc_feature 1..25: note = Synthetic polynucleotide
  source 1..25: mol_type = other DNA
agcaaatcactcttatttttcatcc

SEQ ID NO: 121 - DNA, 25 bp, synthetic construct
  misc_feature 1..25: note = Synthetic polynucleotide
  source 1..25: mol_type = other DNA
gtgggagcagtgtacaccaactctt

SEQ ID NO: 122 - DNA, 25 bp, synthetic construct
  misc_feature 1..25: note = Synthetic polynucleotide
  source 1..25: mol_type = other DNA
aactcttcctgtatattgccttttt

SEQ ID NO: 123 - DNA, 25 bp, synthetic construct
  misc_feature 1..25: note = Synthetic polynucleotide
  source 1..25: mol_type = other DNA
taacagtaactgactggcccactga

SEQ ID NO: 124 - DNA, 25 bp, synthetic construct
  misc_feature 1..25: note = Synthetic polynucleotide
  source 1..25: mol_type = other DNA
ccacctgggagagaaccacccgagg

SEQ ID NO: 125 - DNA, 25 bp, synthetic construct
  misc_feature 1..25: note = Synthetic polynucleotide
  source 1..25: mol_type = other DNA
gtacccggctctgacgggtggtcat

SEQ ID NO: 126 - DNA, 25 bp, synthetic construct
  misc_feature 1..25: note = Synthetic polynucleotide
  source 1..25: mol_type = other DNA
ttgctgctggacttttcgaagggaa

SEQ ID NO: 127 - DNA, 25 bp, synthetic construct
  misc_feature 1..25: note = Synthetic polynucleotide
  source 1..25: mol_type = other DNA
gagcttgctgatgggcgagttggtc

SEQ ID NO: 128 - DNA, 25 bp, synthetic construct
  misc_feature 1..25: note = Synthetic polynucleotide
  source 1..25: mol_type = other DNA
ggtccatcgcctttagggtatgtcc

SEQ ID NO: 129 - DNA, 25 bp, synthetic construct
  misc_feature 1..25: note = Synthetic polynucleotide
  source 1..25: mol_type = other DNA
ctttagggtatgtccacctctgttc

SEQ ID NO: 130 - DNA, 25 bp, synthetic construct
  misc_feature 1..25: note = Synthetic polynucleotide
  source 1..25: mol_type = other DNA
ctctgttcacttcctccaaggaaaa

SEQ ID NO: 131 - DNA, 25 bp, synthetic construct
  misc_feature 1..25: note = Synthetic polynucleotide
  source 1..25: mol_type = other DNA
caccctcaggcagccagagattcat

SEQ ID NO: 132 - DNA, 25 bp, synthetic construct
  misc_feature 1..25: note = Synthetic polynucleotide
  source 1..25: mol_type = other DNA
aacttcccctcggagggtggagcat

SEQ ID NO: 133 - DNA, 25 bp, synthetic construct
  misc_feature 1..25: note = Synthetic polynucleotide
  source 1..25: mol_type = other DNA
gattattctgtatcctgtgtttgta

SEQ ID NO: 134 - DNA, 25 bp, synthetic construct
  misc_feature 1..25: note = Synthetic polynucleotide
  source 1..25: mol_type = other DNA
ggtgatcacgaaactcgctggcatg

SEQ ID NO: 135 - DNA, 25 bp, synthetic construct
  misc_feature 1..25: note = Synthetic polynucleotide
  source 1..25: mol_type = other DNA
ggaggaagacttggcgttttcgaca

SEQ ID NO: 136 - DNA, 25 bp, synthetic construct
  misc_feature 1..25: note = Synthetic polynucleotide
  source 1..25: mol_type = other DNA
ggctggggaatttggctccagatcc

SEQ ID NO: 137 - DNA, 25 bp, synthetic construct
  misc_feature 1..25: note = Synthetic polynucleotide
  source 1..25: mol_type = other DNA
gcgctttggcaccatgagttctatg

SEQ ID NO: 138 - DNA, 25 bp, synthetic construct
  misc_feature 1..25: note = Synthetic polynucleotide
  source 1..25: mol_type = other DNA
ttctatgtctggggccgacgacact

SEQ ID NO: 139 - DNA, 25 bp, synthetic construct
  misc_feature 1..25: note = Synthetic polynucleotide
  source 1..25: mol_type = other DNA
taccactcatcgcggagcaaggcgc

SEQ ID NO: 140 - DNA, 25 bp, synthetic construct
  misc_feature 1..25: note = Synthetic polynucleotide
  source 1..25: mol_type = other DNA
acccgctcttcaagcgctttaggaa

SEQ ID NO: 141 - DNA, 25 bp, synthetic construct
  misc_feature 1..25: note = Synthetic polynucleotide
  source 1..25: mol_type = other DNA
gcagggtacttcgttcaagaccggc

SEQ ID NO: 142 - DNA, 25 bp, synthetic construct
  misc_feature 1..25: note = Synthetic polynucleotide
  source 1..25: mol_type = other DNA
cctccagcgttggccaaattgtgct

SEQ ID NO: 143 - DNA, 25 bp, synthetic construct
  misc_feature 1..25: note = Synthetic polynucleotide
  source 1..25: mol_type = other DNA
ggctgtgccttcataggtcatctag

SEQ ID NO: 144 - DNA, 25 bp, synthetic construct
  misc_feature 1..25: note = Synthetic polynucleotide
  source 1..25: mol_type = other DNA
ggggagtggggtcatttctgtatat

SEQ ID NO: 145 - DNA, 25 bp, synthetic construct
  misc_feature 1..25: note = Synthetic polynucleotide
  source 1..25: mol_type = other DNA
gaaaccagtgtcgccctggagaagt

SEQ ID NO: 146 - DNA, 25 bp, synthetic construct
  misc_feature 1..25: note = Synthetic polynucleotide
  source 1..25: mol_type = other DNA
aggaggcctgcgagaatggccgcgg

SEQ ID NO: 147 - DNA, 25 bp, synthetic construct
  misc_feature 1..25: note = Synthetic polynucleotide
  source 1..25: mol_type = other DNA
tgctgtcagtggcccggggcaaagt

SEQ ID NO: 148 - DNA, 25 bp, synthetic construct
  misc_feature 1..25: note = Synthetic polynucleotide
  source 1..25: mol_type = other DNA
gagggaatcgactttgtgcaccact

SEQ ID NO: 149 - DNA, 25 bp, synthetic construct
  misc_feature 1..25: note = Synthetic polynucleotide
  source 1..25: mol_type = other DNA
tctacacacagagccgcattctcaa

SEQ ID NO: 150 - DNA, 25 bp, synthetic construct
  misc_feature 1..25: note = Synthetic polynucleotide
  source 1..25: mol_type = other DNA
gcattctcaaggcgcggctggaata

SEQ ID NO: 151 - DNA, 25 bp, synthetic construct
  misc_feature 1..25: note = Synthetic polynucleotide
  source 1..25: mol_type = other DNA
gggaccagttccagattcgtgagaa

SEQ ID NO: 152 - DNA, 25 bp, synthetic construct
  misc_feature 1..25: note = Synthetic polynucleotide
  source 1..25: mol_type = other DNA
agaatgactttcttaccttcgatgc

SEQ ID NO: 153 - DNA, 25 bp, synthetic construct
  misc_feature 1..25: note = Synthetic polynucleotide
  source 1..25: mol_type = other DNA
gtcgggccatcaggggcaagacgga

SEQ ID NO: 154 - DNA, 25 bp, synthetic construct
  misc_feature 1..25: note = Synthetic polynucleotide
  source 1..25: mol_type = other DNA
ggtctttgccgacaagcggtttgcc

SEQ ID NO: 155 - DNA, 25 bp, synthetic construct
  misc_feature 1..25: note = Synthetic polynucleotide
  source 1..25: mol_type = other DNA
tcagcagctctgagtggggcgggtg

SEQ ID NO: 156 - DNA, 25 bp, synthetic construct
  misc_feature 1..25: note = Synthetic polynucleotide
  source 1..25: mol_type = other DNA
ttagttccaaactcactcttcttac

SEQ ID NO: 157 - DNA, 25 bp, synthetic construct
  misc_feature 1..25: note = Synthetic polynucleotide
  source 1..25: mol_type = other DNA
cgacagcactggccattacagcaga

SEQ ID NO: 158 - DNA, 25 bp, synthetic construct
  misc_feature 1..25: note = Synthetic polynucleotide
  source 1..25: mol_type = other DNA
ttacagcagattccgaaacccttcc

SEQ ID NO: 159 - DNA, 25 bp, synthetic construct
  misc_feature 1..25: note = Synthetic polynucleotide
  source 1..25: mol_type = other DNA
gagaagtataatcctggtccccaag

SEQ ID NO: 160 - DNA, 25 bp, synthetic construct
  misc_feature 1..25: note = Synthetic polynucleotide
  source 1..25: mol_type = other DNA
ggtgaatgccaaaaactgccgctcc

SEQ ID NO: 161 - DNA, 25 bp, synthetic construct
  misc_feature 1..25: note = Synthetic polynucleotide
  source 1..25: mol_type = other DNA
gctccttgatgcaccacgttaagaa

SEQ ID NO: 162 - DNA, 25 bp, synthetic construct
  misc_feature 1..25: note = Synthetic polynucleotide
  source 1..25: mol_type = other DNA
tagcagccctgtcacaagacgagct

SEQ ID NO: 163 - DNA, 25 bp, synthetic construct
  misc_feature 1..25: note = Synthetic polynucleotide
  source 1..25: mol_type = other DNA
tatgatttcattcacacctcttttg

SEQ ID NO: 164 - DNA, 25 bp, synthetic construct
  misc_feature 1..25: note = Synthetic polynucleotide
  source 1..25: mol_type = other DNA
tgatggctgttttcttatcccatgc

SEQ ID NO: 165 - DNA, 25 bp, synthetic construct
  misc_feature 1..25: note = Synthetic polynucleotide
  source 1..25: mol_type = other DNA
tcccatgcctgtacttttcagcggc

SEQ ID NO: 166 - DNA, 25 bp, synthetic construct
  misc_feature 1..25: note = Synthetic polynucleotide
  source 1..25: mol_type = other DNA
gctccttgccagacatcataggtca

SEQ ID NO: 167 - DNA, 25 bp, synthetic construct
  misc_feature 1..25: note = Synthetic polynucleotide
  source 1..25: mol_type = other DNA
gaagcagcagccagcgaaatagaag

SEQ ID NO: 168 - DNA, 25 bp, synthetic construct
  misc_feature 1..25: note = Synthetic polynucleotide
  source 1..25: mol_type = other DNA
aagaggctttcagattctaaacgaa

SEQ ID NO: 169 - DNA, 25 bp, synthetic construct
  misc_feature 1..25: note = Synthetic polynucleotide
  source 1..25: mol_type = other DNA
atacatgcggtggatttttggggga

SEQ ID NO: 170 - DNA, 25 bp, synthetic construct
  misc_feature 1..25: note = Synthetic polynucleotide
  source 1..25: mol_type = other DNA
tcatctgatggatcttcaagtgaac

SEQ ID NO: 171 - DNA, 25 bp, synthetic construct
  misc_feature 1..25: note = Synthetic polynucleotide
  source 1..25: mol_type = other DNA
gccaaaaaccagtgcttcagattcg

SEQ ID NO: 172 - DNA, 25 bp, synthetic construct
  misc_feature 1..25: note = Synthetic polynucleotide
  source 1..25: mol_type = other DNA
gcttcagattcgcagaactcagtga

SEQ ID NO: 173 - DNA, 25 bp, synthetic construct
  misc_feature 1..25: note = Synthetic polynucleotide
  source 1..25: mol_type = other DNA
gaatggaggtgcgaccaccagcagc

SEQ ID NO: 174 - DNA, 25 bp, synthetic construct
  misc_feature 1..25: note = Synthetic polynucleotide
  source 1..25: mol_type = other DNA
accagcagctctagtgatagtgatg

SEQ ID NO: 175 - DNA, 25 bp, synthetic construct
  misc_feature 1..25: note = Synthetic polynucleotide
  source 1..25: mol_type = other DNA
agagaagatggtcctcgtgaccgcc

SEQ ID NO: 176 - DNA, 25 bp, synthetic construct
  misc_feature 1..25: note = Synthetic polynucleotide
  source 1..25: mol_type = other DNA
gtgaccgccagatctgtgtttggga

SEQ ID NO: 177 - DNA, 25 bp, synthetic construct
  misc_feature 1..25: note = Synthetic polynucleotide
  source 1..25: mol_type = other DNA
gaaggaaactaagacgtgcgagggg

SEQ ID NO: 178 - DNA, 25 bp, synthetic construct
  misc_feature 1..25: note = Synthetic polynucleotide
  source 1..25: mol_type = other DNA
taagacttgtactatgtgtggccat

SEQ ID NO: 179 - DNA, 25 bp, synthetic construct
  misc_feature 1..25: note = Synthetic polynucleotide
  source 1..25: mol_type = other DNA
gtgtggccatgaactgacatatgaa

SEQ ID NO: 180 - DNA, 25 bp, synthetic construct
  misc_feature 1..25: note = Synthetic polynucleotide
  source 1..25: mol_type = other DNA
tcagtgaaggtcacctggcctggtt

SEQ ID NO: 181 - DNA, 25 bp, synthetic construct
  misc_feature 1..25: note = Synthetic polynucleotide
  source 1..25: mol_type = other DNA
gttgtgtgcacaatgtcatgtctgt

SEQ ID NO: 182 - DNA, 25 bp, synthetic construct
  misc_feature 1..25: note = Synthetic polynucleotide
  source 1..25: mol_type = other DNA
atgtctgtgattgccttcttacaac

SEQ ID NO: 183 - DNA, 25 bp, synthetic construct
  misc_feature 1..25: note = Synthetic polynucleotide
  source 1..25: mol_type = other DNA
aggtgcagaagtggtaggtcagcta

SEQ ID NO: 184 - DNA, 25 bp, synthetic construct
  misc_feature 1..25: note = Synthetic polynucleotide
  source 1..25: mol_type = other DNA
ggacaagataccaaggcaaacccta

SEQ ID NO: 185 - DNA, 25 bp, synthetic construct
  misc_feature 1..25: note = Synthetic polynucleotide
  source 1..25: mol_type = other DNA
ggctgaactggattcttaaccaaga

SEQ ID NO: 186 - DNA, 25 bp, synthetic construct
  misc_feature 1..25: note = Synthetic polynucleotide
  source 1..25: mol_type = other DNA
gcaatggtggtgcaccactgtaccc

SEQ ID NO: 187 - DNA, 25 bp, synthetic construct
  misc_feature 1..25: note = Synthetic polynucleotide
  source 1..25: mol_type = other DNA
actgtaccccaggttctagtcatgt

SEQ ID NO: 188 - DNA, 25 bp, synthetic construct
  misc_feature 1..25: note = Synthetic polynucleotide
  source 1..25: mol_type = other DNA
ttaggacgatttctgtctccacgat

SEQ ID NO: 189 - DNA, 25 bp, synthetic construct
  misc_feature 1..25: note = Synthetic polynucleotide
  source 1..25: mol_type = other DNA
cccttccaaagactgtctccgttga

SEQ ID NO: 190 - DNA, 25 bp, synthetic construct
  misc_feature 1..25: note = Synthetic polynucleotide
  source 1..25: mol_type = other DNA
tccaaagactgtctccgttgacctt

SEQ ID NO: 191 - DNA, 25 bp, synthetic construct
  misc_feature 1..25: note = Synthetic polynucleotide
  source 1..25: mol_type = other DNA
tgaccttgtctttttggtatgcctt

SEQ ID NO: 192 - DNA, 25 bp, synthetic construct
  misc_feature 1..25: note = Synthetic polynucleotide
  source 1..25: mol_type = other DNA
ccttgtctttttggtatgccttggg

SEQ ID NO: 193 - DNA, 25 bp, synthetic construct
  misc_feature 1..25: note = Synthetic polynucleotide
  source 1..25: mol_type = other DNA
ttttggtatgccttggggtttctga

SEQ ID NO: 194 - DNA, 25 bp, synthetic construct
  misc_feature 1..25: note = Synthetic polynucleotide
  source 1..25: mol_type = other DNA
ggtatgccttggggtttctgataat

SEQ ID NO: 195 - DNA, 25 bp, synthetic construct
  misc_feature 1..25: note = Synthetic polynucleotide
  source 1..25: mol_type = other DNA
gccttggggtttctgataatgtgtg

SEQ ID NO: 196 - DNA, 25 bp, synthetic construct
  misc_feature 1..25: note = Synthetic polynucleotide
  source 1..25: mol_type = other DNA
aggtaagtgagcatgtcaaacaaaa

SEQ ID NO: 197 - DNA, 25 bp, synthetic construct
  misc_feature 1..25: note = Synthetic polynucleotide
  source 1..25: mol_type = other DNA
gtcaaacaaaataggagctcacatg

SEQ ID NO: 198 - DNA, 25 bp, synthetic construct
  misc_feature 1..25: note = Synthetic polynucleotide
  source 1..25: mol_type = other DNA
aggagctcacatggatatatttatg

SEQ ID NO: 199 - DNA, 25 bp, synthetic construct
  misc_feature 1..25: note = Synthetic polynucleotide
  source 1..25: mol_type = other DNA
atggatatatttatgtcactgagtt

SEQ ID NO: 200 - DNA, 25 bp, synthetic construct
  misc_feature 1..25: note = Synthetic polynucleotide
  source 1..25: mol_type = other DNA
gagcctttgtctggtgaacagttgg

SEQ ID NO: 201 - DNA, 25 bp, synthetic construct
  misc_feature 1..25: note = Synthetic polynucleotide
  source 1..25: mol_type = other DNA
tctccccaggataaggcggcagagg

SEQ ID NO: 202 - DNA, 25 bp, synthetic construct
  misc_feature 1..25: note = Synthetic polynucleotide
  source 1..25: mol_type = other DNA
ggcggcagaggctacaaatgactat

SEQ ID NO: 203 - DNA, 25 bp, synthetic construct
  misc_feature 1..25: note = Synthetic polynucleotide
  source 1..25: mol_type = other DNA
aatccaggaggccctaaactgctta

SEQ ID NO: 204 - DNA, 25 bp, synthetic construct
  misc_feature 1..25: note = Synthetic polynucleotide
  source 1..25: mol_type = other DNA
gcagatcctgaagttatgctcttga

SEQ ID NO: 205 - DNA, 25 bp, synthetic construct
  misc_feature 1..25: note = Synthetic polynucleotide
  source 1..25: mol_type = other DNA
gttatgctcttgactttaagtttgt

SEQ ID NO: 206 - DNA, 25 bp, synthetic construct
  misc_feature 1..25: note = Synthetic polynucleotide
  source 1..25: mol_type = other DNA
ggaatttttgttcccataattggat

SEQ ID NO: 207 - DNA, 25 bp, synthetic construct
  misc_feature 1..25: note = Synthetic polynucleotide
  source 1..25: mol_type = other DNA
tcaaaaagcaacttctgccctgcaa

SEQ ID NO: 208 - DNA, 25 bp, synthetic construct
  misc_feature 1..25: note = Synthetic polynucleotide
  source 1..25: mol_type = other DNA
ccccactccatagtctggtattctg

SEQ ID NO: 209 - DNA, 25 bp, synthetic construct
  misc_feature 1..25: note = Synthetic polynucleotide
  source 1..25: mol_type = other DNA
gtctggtattctgagcactagctta

SEQ ID NO: 210 - DNA, 25 bp, synthetic construct
  misc_feature 1..25: note = Synthetic polynucleotide
  source 1..25: mol_type = other DNA
gagcactagcttaatatttcttcac

SEQ ID NO: 211 - DNA, 25 bp, synthetic construct
  misc_feature 1..25: note = Synthetic polynucleotide
  source 1..25: mol_type = other DNA
tatttagaagcctttactcagcccc

SEQ ID NO: 212 - DNA, 25 bp, synthetic construct
  misc_feature 1..25: note = Synthetic polynucleotide
  source 1..25: mol_type = other DNA
ggagcttgagctctacaggtaaggc

SEQ ID NO: 213 - DNA, 25 bp, synthetic construct
  misc_feature 1..25: note = Synthetic polynucleotide
  source 1..25: mol_type = other DNA
taaggcagagctaccggtgaatgaa

SEQ ID NO: 214 - DNA, 25 bp, synthetic construct
  misc_feature 1..25: note = Synthetic polynucleotide
  source 1..25: mol_type = other DNA
cccctggcatcacatgtgcatgctg

SEQ ID NO: 215 - DNA, 25 bp, synthetic construct
  misc_feature 1..25: note = Synthetic polynucleotide
  source 1..25: mol_type = other DNA
gcatgctgtaggcaggacctgagct

SEQ ID NO: 216 - DNA, 25 bp, synthetic construct
  misc_feature 1..25: note = Synthetic polynucleotide
  source 1..25: mol_type = other DNA
tccgctgcaggttcagatgcaccgc

SEQ ID NO: 217 - DNA, 25 bp, synthetic construct
  misc_feature 1..25: note = Synthetic polynucleotide
  source 1..25: mol_type = other DNA
tgcagctgtccttcagttagttcac

SEQ ID NO: 218 - DNA, 25 bp, synthetic construct
  misc_feature 1..25: note = Synthetic polynucleotide
  source 1..25: mol_type = other DNA
agcctgagccgggaactggctgtgc

SEQ ID NO: 219 - DNA, 25 bp, synthetic construct
  misc_feature 1..25: note = Synthetic polynucleotide
  source 1..25: mol_type = other DNA
agggcttccgtgtactcaggatgta

SEQ ID NO: 220 - DNA, 25 bp, synthetic construct
  misc_feature 1..25: note = Synthetic polynucleotide
  source 1..25: mol_type = other DNA
gtcttctcaagggactgatcggcca

SEQ ID NO: 221 - DNA, 25 bp, synthetic construct
  misc_feature 1..25: note = Synthetic polynucleotide
  source 1..25: mol_type = other DNA
gatcggccaagtatgcttttcttta

SEQ ID NO: 222 - DNA, 4518 bp, Homo sapiens
  source 1..4518: mol_type = other DNA
ggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 223 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 224 - DNA, 25 bp, synthetic construct
  misc_feature 1..25: note = Synthetic polynucleotide
  source 1..25: mol_type = other DNA
gcaacaaccgaaaatgcaccagccc

SEQ ID NO: 225 - DNA, 25 bp, synthetic construct
  misc_feature 1..25: note = Synthetic polynucleotide
  source 1..25: mol_type = other DNA
ccccaggtcctcggacaccgaggag

SEQ ID NO: 226 - DNA, 25 bp, synthetic construct
  misc_feature 1..25: note = Synthetic polynucleotide
  source 1..25: mol_type = other DNA
cgaacacacaacgtcttggagcgcc

SEQ ID NO: 227 - DNA, 25 bp, synthetic construct
  misc_feature 1..25: note = Synthetic polynucleotide
  source 1..25: mol_type = other DNA
ggagcgccagaggaggaacgagcta

SEQ ID NO: 228 - DNA, 25 bp, synthetic construct
  misc_feature 1..25: note = Synthetic polynucleotide
  source 1..25: mol_type = other DNA
gctaaaacggagcttttttgccctg

SEQ ID NO: 229 - DNA, 25 bp, synthetic construct
  misc_feature 1..25: note = Synthetic polynucleotide
  source 1..25: mol_type = other DNA
aaaagccacagcatacatcctgtcc

SEQ ID NO: 230 - DNA, 25 bp, synthetic construct
  misc_feature 1..25: note = Synthetic polynucleotide
  source 1..25: mol_type = other DNA
catcctgtccgtccaagcagaggag

SEQ ID NO: 231 - DNA, 25 bp, synthetic construct
  misc_feature 1..25: note = Synthetic polynucleotide
  source 1..25: mol_type = other DNA
catcctgtccgtccaagcagaggag

SEQ ID NO: 232 - DNA, 25 bp, synthetic construct
  misc_feature 1..25: note = Synthetic polynucleotide
  source 1..25: mol_type = other DNA
gaggacttgttgcggaaacgacgag

SEQ ID NO: 233 - DNA, 25 bp, synthetic construct
  misc_feature 1..25: note = Synthetic polynucleotide
  source 1..25: mol_type = other DNA
gaacagctacggaactcttgtgcgt

SEQ ID NO: 234 - DNA, 25 bp, synthetic construct
  misc_feature 1..25: note = Synthetic polynucleotide
  source 1..25: mol_type = other DNA
gaaatgtcctgagcaatcacctatg

SEQ ID NO: 235 - DNA, 25 bp, synthetic construct
  misc_feature 1..25: note = Synthetic polynucleotide
  source 1..25: mol_type = other DNA
gcaacctcacaaccttggctgagtc

SEQ ID NO: 236 - DNA, 1291 bp, Homo sapiens
  source 1..1291: mol_type = other DNA
gagccgaaggtggaggtcaaaggggcgtggcgttacagagcctctagcgctgggtgttggggacctgacgctatggagctctcggagttttgtgggggacggctgtgagtggggggttcctgctgcgggatgagaacgtagacgccagtggctcactcgctcctggcaccttccctttcaggctccagatggaccctgggaaggacaaagagggggtgccccagccctcagggccgccagcaaggaagaaatttgtgatacccctcgacgaggatgaggtccctcctggagtggccaagcccttattccgatctacacagagccttcccactgtggacacctcggcccaggcggcccctcagacctacgccgaatatgccatctcacagcctctggaaggggctggggccacgtgccccacagggtcagagcccctggcaggagagacgcccaaccaggccctgaaacccggggcaaaatccaacagcatcattgtgagccctcggcagaggggcaatcccgtactgaagttcgtgcgcaatgtgccctgggaatttggcgacgtaattcccgactatgtgctgggccagagcacctgtgccctgttcctcagcctccgctaccacaacctgcacccagactacatccatgggcggctgcagagcctggggaagaacttcgccttgcgggtcctgcttgtccaggtggatgtgaaagatccccagcaggccctcaaggagctggctaagatgtgtatcctggccgactgcacattgatcctcgcctggagccccgaggaagctgggcggtacctggagacctacaaggcctatgagcagaaaccagcggacctcctgatggagaagctagagcaggacttcgtctcccgggtgactgaatgtctgaccaccgtgaagtcagtcaacaaaacggacagtcagaccctcctgaccacatttggatctctggaacagctcatcgccgcatcaagagaagatctggccttatgcccaggcctgggccctcagaaagtaagagctctgggaaagaacccaaggagttgggggaaggagagagccccaaataaacacaacctgagaccccaaagttttaaggtgaaaaaagaaccaaagaccagacacagtggcttccgcctgtaatcccaacattttgggaggccaaggcgggaggactgcttgaggccagaagttggagaccagcctgggcaagtggacacctcatttttactaaaaataaaaaaaactagctgggcaaaaaaaaaaaaaaaaaa

SEQ ID NO: 237 - protein, 323 aa, Homo sapiens
MDPGKDKEGVPQPSGPPARKKFVIPLDEDEVPPGVAKPLFRSTQSLPTVDTSAQAAPQTYAEYAISQPLEGAGATCPTGSEPLAGETPNQALKPGAKSNSIIVSPRQRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQKVRALGKNPRSWGKERAPNKHNLRPQSFKVKKEPKTRHSGFRL

SEQ ID NO: 238 - DNA, 25 bp, synthetic construct
  misc_feature 1..25: note = Synthetic polynucleotide
  source 1..25: mol_type = other DNA
aagatctggccttatgcccaggcct

SEQ ID NO: 239 - DNA, 25 bp, synthetic construct
  misc_feature 1..25: note = Synthetic polynucleotide
  source 1..25: mol_type = other DNA
ccctcagaaagcccggaggctgttt

SEQ ID NO: 240 - DNA, 25 bp, synthetic construct
  misc_feature 1..25: note = Synthetic polynucleotide
  source 1..25: mol_type = other DNA
ctcagaaagcccggaggctgtttga

SEQ ID NO: 241 - DNA, 25 bp, synthetic construct
  misc_feature 1..25: note = Synthetic polynucleotide
  source 1..25: mol_type = other DNA
gaaagcccggaggctgtttgatgtc

SEQ ID NO: 242 - DNA, 25 bp, synthetic construct
  misc_feature 1..25: note = Synthetic polynucleotide
  source 1..25: mol_type = other DNA
aagcccggaggctgtttgatgtcct

SEQ ID NO: 243 - DNA, 25 bp, synthetic construct
  misc_feature 1..25: note = Synthetic polynucleotide
  source 1..25: mol_type = other DNA
ggctgtttgatgtcctgcacgagcc

SEQ ID NO: 244 - DNA, 25 bp, synthetic construct
  misc_feature 1..25: note = Synthetic polynucleotide
  source 1..25: mol_type = other DNA
tgcacgagcccttcttgaaagtacc

SEQ ID NO: 245 - DNA, 25 bp, synthetic construct
  misc_feature 1..25: note = Synthetic polynucleotide
  source 1..25: mol_type = other DNA
gcccttcttgaaagtaccctgatga

SEQ ID NO: 246 - DNA, 25 bp, synthetic construct
  misc_feature 1..25: note = Synthetic polynucleotide
  source 1..25: mol_type = other DNA
ccttcttgaaagtaccctgatgacc

SEQ ID NO: 247 - DNA, 25 bp, synthetic construct
  misc_feature 1..25: note = Synthetic polynucleotide
  source 1..25: mol_type = other DNA
ttcttgaaagtaccctgatgacccc

SEQ ID NO: 248 - DNA, 25 bp, synthetic construct
  misc_feature 1..25: note = Synthetic polynucleotide
  source 1..25: mol_type = other DNA
tcttgaaagtaccctgatgacccca

SEQ ID NO: 249 - DNA, 1220 bp, Homo sapiens
  source 1..1220: mol_type = other DNA
atgcacacatacgcgcgcgcacatgcacataggcacacagagatacgcgcacacacacacacaaacgcactcagatttcccggaccctggttttcctcctgtgaccctttcggggccgggctctcaccctaaagaagcagccccgccctggggtggccccaccctctcttgggacctgtcataagtcggaccccgggcgcccggctgcgcagtcccagccgccttccccaggcagtggaaccttcgggctcctgagcttcaggatggttcgtactaagacatggaccctgaagaagcactttgttggctatcctactaatagtgactttgagttgaagacagctgagctcccacccttaaaaaatggagaggtcctgcttgaagctttgttcctcaccgtggatccctacatgagagtggcagccaaaagattgaaggaaggtgatacaatgatggggcagcaagtggccaaagttgtggaaagtaaaaatgtagccctaccaaaaggaactattgtactggcttctccaggctggacaacgcactccatttctgatgggaaagatctggaaaagctgctgacagagtggccagacacaataccactgtctttggctctggggacagttggcatgccaggcctgactgcctactttggcctacttgaaatctgtggtgtgaagggtggagaaacagtgatggttaatgcagcagctggagctgtgggctcagtcgtggggcagattgcaaagctcaagggctgcaaagttgttggagcagtagggtctgatgaaaaggttgcctaccttcaaaagcttggatttgatgtcgtctttaactacaagacggtagagtctttggaagaaaccttgaagaaagcgtctcctgatggttatgattgttattttgataatgtaggtggagagttttcaaacactgttatcggccagatgaagaaatttggaaggattgccatatgtggagccatctctacatataacagaaccggcccacttcccccaggcccacccccagagattgttatctatcaggagcttcgcatggaagcttttgtcgtctaccgctggcaaggagatgcccgccaaaaagctctgaaggacttgctgaaatgggtcttagagatcaaaagagaaaatgaagaagattgaagcttcaaagcagaaaaatgaagggaatatgtatcattcaccattacctta

SEQ ID NO: 250 - protein, 301 aa, Homo sapiens
MVRTKTWTLKKHFVGYPTNSDFELKTAELPPLKNGEVLLEALFLTVDPYMRVAAKRLKEGDTMMGQQVAKVVESKNVALPKGTIVLASPGWTTHSISDGKDLEKLLTEWPDTIPLSLALGTVGMPGLTAYFGLLEICGVKGGETVMVNAAAGAVGSVVGQIAKLKGCKVVGAVGSDEKVAYLQKLGFDVVFNYKTVESLEETLKKASPDGYDCYFDNVGGEFSNTVIGQMKKFGRIAICGAISTYNRTGPLPPGPPPEIVIYQELRMEAFVVYRWQGDARQKALKDLLKWVLEIKRENEED

SEQ ID NO: 251 - DNA, 25 bp, synthetic construct
  misc_feature 1..25: note = Synthetic polynucleotide
  source 1..25: mol_type = other DNA
gcttggatttgatgtcgtctttaac

SEQ ID NO: 252 - DNA, 25 bp, synthetic construct
  misc_feature 1..25: note = Synthetic polynucleotide
  source 1..25: mol_type = other DNA
gaaagcgtctcctgatggttatgat

SEQ ID NO: 253 - DNA, 25 bp, synthetic construct
  misc_feature 1..25: note = Synthetic polynucleotide
  source 1..25: mol_type = other DNA
ttcaaacactgttatcggccagatg

SEQ ID NO: 254 - DNA, 25 bp, synthetic construct
  misc_feature 1..25: note = Synthetic polynucleotide
  source 1..25: mol_type = other DNA
atgtggagccatctctacatataac

SEQ ID NO: 255 - DNA, 25 bp, synthetic construct
  misc_feature 1..25: note = Synthetic polynucleotide
  source 1..25: mol_type = other DNA
tacatataacagaaccggcccactt

SEQ ID NO: 256 - DNA, 25 bp, synthetic construct
  misc_feature 1..25: note = Synthetic polynucleotide
  source 1..25: mol_type = other DNA
cccacccccagagattgttatctat

SEQ ID NO: 257 - DNA, 25 bp, synthetic construct
  misc_feature 1..25: note = Synthetic polynucleotide
  source 1..25: mol_type = other DNA
gttatctatcaggagcttcgcatgg

SEQ ID NO: 258 - DNA, 25 bp, synthetic construct
  misc_feature 1..25: note = Synthetic polynucleotide
  source 1..25: mol_type = other DNA
catggaagcttttgtcgtctaccgc

SEQ ID NO: 259 - DNA, 25 bp, synthetic construct
  misc_feature 1..25: note = Synthetic polynucleotide
  source 1..25: mol_type = other DNA
taccgctggcaaggagatgcccgcc

SEQ ID NO: 260 - DNA, 25 bp, synthetic construct
  misc_feature 1..25: note = Synthetic polynucleotide
  source 1..25: mol_type = other DNA
aggatttgaaaacatgccagctgca

SEQ ID NO: 261 - DNA, 25 bp, synthetic construct
  misc_feature 1..25: note = Synthetic polynucleotide
  source 1..25: mol_type = other DNA
gaggacacatggaatctggaggcca

SEQ ID NO: 262 - DNA, 2520 bp, Homo sapiens
  source 1..2520: mol_type = other DNA
gcgcgcctgcgtactgcggtcagagggccgaggcctgggggcgagctggggtcgtgcagtacagcctctttccggcaaatcacgcgagatttcgttcacccgggctccacagggagtattttatacagaaatcttgtgaaaccactgcccaaccagagcaatgattgttcaaagagtggtattgaattctcgacctggaaaaaatggtaatccagtggcagagaatttccgaatggaagaagtctatttaccagataatattaatgaaggacaagtacaagttagaactctttatctttctgtggatccttacatgcgttgtagaatgaatgaagacactggcactgattatataacaccttggcagctatctcaagtcgttgatggaggaggtattggaattatagaagaaagcaaacacacaaatttgactaaaggcgattttgtgacttctttctattggccctggcaaaccaaggttattctggatggaaatagccttgaaaaggtagacccacaacttgtggatggacacctttcatattttcttggagctataggtatgcctggtttgacttccttgattgggatacaggaaaaaggtcatataactgctggatctaataagacaatggttgtcagtggggccgcaggtgcctgtggatctgtggctgggcagattggccatttcttaggttgttccagagtggtgggaatttgtggaacacatgagaaatgcatcctcttgacctcagaactgggctttgatgctgcaattaattataaaaaagacaatgtggcagaacagctccgtgaatcatgcccagctggagtggatgtttattttgacaatgttggtggtaacatcagtgatacagtgataagtcagatgaatgagaacagccacatcatcctgtgtggtcaaatttctcagtacaacaaagatgtgccttatcctcccccgctatcccctgctatagaggcaatccagaaagaaagaaacatcacaagggaaagatttctggtattaaattataaagacaaatttgagcctggcattctacagctgagtcagtggtttaaagaaggaaagctaaagattaaagagacggtaataaatgggttggaaaacatgggagctgcattccagtccatgatgacaggaggtaacattggaaagcagatagtttgcatttcagaagaaatctctttgtaattgctgtaaatgtcatcaaggcaatcatagatttcttttccattttgcatattttcaaagatatgttaaaaaatccttagactatacatagctcttgatttaaatgtgatcataggtgttatttttagttgcatagggtatttgatacaatcattaatggatcatacacaataggtttttaaaaattaataacttttagtaattacttttattaatttaaaatagaacgcttgagaggcactttgtaaagatttgttaaactggaaacgttttacatgatctgatacaaccattaatgaatcatacacaataggttttttaaaattaatattaataacttttattaatttaaaatagaatgcttaaaataaaatagaatgcttgagaggcactgagtaaagatttgttgaactggaaatgttttacatgattcttaaactgaaacttggtgtaaaaatagaattgagatggcctttttttcacattgtagactgaaaagagacttaatggtatgatgtgtacatagggactgggggcaggattgggggtttcggagcttgtgtaacagtttttgggataggagaccagcggttttgggttgggattagtatgggaagataaataacttaggttggttaagttgacaagatttactccagaggatcatctctttgtatttgccaaataatttactgtatagcctaaaaactccatatatattgagaaaagcatatgtttattttaggttagcaggcacatactgtcaagttgtaaagattgagagggcaaacagatgtaaacatcacttgtaggtgattaaaaagattgacagccgggcctgatgtctcaagcctgtaatcctagcactttgggaggcagaggcgggcagatcacttgaggtcaggagttcaagaccagcctggccaacacggtgaaaccccatctctctaaaaatacaaaaattagctgggcgtggtggcacacgcctgtaatcccagctactcaggaggctgaggctggagaatcatttgaacctgggagggggaggttgccgtgagctgagcttgcaccattgcactccagcctgggcgacaagagtgaaattccatctcaaaaaacaaaaacagattgacaacaaagacagtttcagaaaatgacaggactgggcaaattaacaaatgtttgtaaacatgaatgttcaggaactactgatgtacctcaaaagtttgttttattaattgtactcaaccctcgcagaacagtaaaactgaagattattgtttctgaaaaaaaaaaaaaaaaaa

SEQ ID NO: 263 - protein, 351 aa, Homo sapiens
MIVQRVVLNSRPGKNGNPVAENFRMEEVYLPDNINEGQVQVRTLYLSVDPYMRCRMNEDTGTDYITPWQLSQVVDGGGIGIIEESKHTNLTKGDFVTSFYWPWQTKVILDGNSLEKVDPQLVDGHLSYFLGAIGMPGLTSLIGIQEKGHITAGSNKTMVVSGAAGACGSVAGQIGHFLGCSRVVGICGTHEKCILLTSELGFDAAINYKKDNVAEQLRESCPAGVDVYFDNVGGNISDTVISQMNENSHIILCGQISQYNKDVPYPPPLSPAIEAIQKERNITRERFLVLNYKDKFEPGILQLSQWFKEGKLKIKETVINGLENMGAAFQSMMTGGNIGKQIVCISEEISL

SEQ ID NO: 264 - DNA, 25 bp, synthetic construct
  misc_feature 1..25: note = Synthetic polynucleotide
  source 1..25: mol_type = other DNA
ggaaagcagatagtttgcatttcag

SEQ ID NO: 265 - DNA, 25 bp, synthetic construct
  misc_feature 1..25: note = Synthetic polynucleotide
  source 1..25: mol_type = other DNA
aaaatccttagactatacatagctc

SEQ ID NO: 266 - DNA, 25 bp, synthetic construct
  misc_feature 1..25: note = Synthetic polynucleotide
  source 1..25: mol_type = other DNA
agactatacatagctcttgatttaa

SEQ ID NO: 267 - DNA, 25 bp, synthetic construct
  misc_feature 1..25: note = Synthetic polynucleotide
  source 1..25: mol_type = other DNA
gatcataggtgttatttttagttgc

SEQ ID NO: 268 - DNA, 25 bp, synthetic construct
  misc_feature 1..25: note = Synthetic polynucleotide
  source 1..25: mol_type = other DNA
ttagttgcatagggtatttgataca

SEQ ID NO: 269 - DNA, 25 bp, synthetic construct
  misc_feature 1..25: note = Synthetic polynucleotide
  source 1..25: mol_type = other DNA
gatacaatcattaatggatcataca

SEQ ID NO: 270 - DNA, 25 bp, synthetic construct
  misc_feature 1..25: note = Synthetic polynucleotide
  source 1..25: mol_type = other DNA
ggatcatacacaataggtttttaaa

SEQ ID NO: 271 - DNA, 25 bp, synthetic construct
  misc_feature 1..25: note = Synthetic polynucleotide
  source 1..25: mol_type = other DNA
agaacgcttgagaggcactttgtaa

SEQ ID NO: 272 - DNA, 25 bp, synthetic construct
  misc_feature 1..25: note = Synthetic polynucleotide
  source 1..25: mol_type = other DNA
ggaaacgttttacatgatctgatac

SEQ ID NO: 273 - DNA, 25 bp, synthetic construct
  misc_feature 1..25: note = Synthetic polynucleotide
  source 1..25: mol_type = other DNA
tgatctgatacaaccattaatgaat

SEQ ID NO: 274 - DNA, 25 bp, synthetic construct
  misc_feature 1..25: note = Synthetic polynucleotide
  source 1..25: mol_type = other DNA
gaatcatacacaataggttttttaa

SEQ ID NO: 275 - DNA, 20 bp, synthetic construct
  misc_feature 1..20: note = Synthetic polynucleotide
  source 1..20: mol_type = other DNA
gcagtctttcccttgaggct

SEQ ID NO: 276 - DNA, 20 bp, synthetic construct
  misc_feature 1..20: note = Synthetic polynucleotide
  source 1..20: mol_type = other DNA
tcgggaattacgtcgccaaa

SEQ ID NO: 277 - DNA, 23 bp, synthetic construct
  misc_feature 1..23: note = Synthetic polynucleotide
  source 1..23: mol_type = other DNA
taccccgagcagttctcctacat

SEQ ID NO: 278 - DNA, 25 bp, synthetic construct
  misc_feature 1..25: note = Synthetic polynucleotide
  source 1..25: mol_type = other DNA
gccagttccttggacaccaggtctg

SEQ ID NO: 279 - DNA, 20 bp, synthetic construct
  misc_feature 1..20: note = Synthetic polynucleotide
  source 1..20: mol_type = other DNA
gcatccacctggagtaccct

SEQ ID NO: 280 - DNA, 20 bp, synthetic construct
  misc_feature 1..20: note = Synthetic polynucleotide
  source 1..20: mol_type = other DNA
tcccaggaccttttggtggt

SEQ ID NO: 281 - DNA, 25 bp, synthetic construct
  misc_feature 1..25: note = Synthetic polynucleotide
  source 1..25: mol_type = other DNA
tgaggcagaaaataaggagagtgaa

SEQ ID NO: 282 - DNA, 25 bp, synthetic construct
  misc_feature 1..25: note = Synthetic polynucleotide
  source 1..25: mol_type = other DNA
tggggaagtgaagtgtaagaagagt

SEQ ID NO: 283 - DNA, 21 bp, synthetic construct
  misc_feature 1..21: note = Synthetic polynucleotide
  source 1..21: mol_type = other DNA
aggcagaaaataaggagagtg

SEQ ID NO: 284 - DNA, 21 bp, synthetic construct
  misc_feature 1..21: note = Synthetic polynucleotide
  source 1..21: mol_type = other DNA
aagcggatggatgatagtgag

SEQ ID NO: 285 - DNA, 27 bp, synthetic construct
  misc_feature 1..27: note = Synthetic polynucleotide
  source 1..27: mol_type = other DNA
gcgaggtagagtcaaggagagtggtct

SEQ ID NO: 286 - DNA, 26 bp, synthetic construct
  misc_feature 1..26: note = Synthetic polynucleotide
  source 1..26: mol_type = other DNA
gcgcaaagttcgttctgctgctcaag

SEQ ID NO: 287 - DNA, 25 bp, synthetic construct
  misc_feature 1..25: note = Synthetic polynucleotide
  source 1..25: mol_type = other DNA
gccgagatgatggagatgaagatta

SEQ ID NO: 288 - DNA, 25 bp, synthetic construct
  misc_feature 1..25: note = Synthetic polynucleotide
  source 1..25: mol_type = other DNA
gcgcagtaggtatggatttatgtga

SEQ ID NO: 289 - DNA, 27 bp, synthetic construct
  misc_feature 1..27: note = Synthetic polynucleotide
  source 1..27: mol_type = other DNA
gtagtcattgatgatctgcccaaagag

SEQ ID NO: 290 - DNA, 27 bp, synthetic construct
  misc_feature 1..27: note = Synthetic polynucleotide
  source 1..27: mol_type = other DNA
gagccacacaagctattttctttctca

SEQ ID NO: 291 - DNA, 27 bp, synthetic construct
  misc_feature 1..27: note = Synthetic polynucleotide
  source 1..27: mol_type = other DNA
gcagttcaaggatctaaagagcatcaa

SEQ ID NO: 292 - DNA, 27 bp, synthetic construct
  misc_feature 1..27: note = Synthetic polynucleotide
  source 1..27: mol_type = other DNA
aacatctgtatgctgaggatgaacaca

SEQ ID NO: 293 - DNA, 23 bp, synthetic construct
  misc_feature 1..23: note = Synthetic polynucleotide
  source 1..23: mol_type = other DNA
tgatgatgagactaaaacaaaat

SEQ ID NO: 294 - DNA, 23 bp, synthetic construct
  misc_feature 1..23: note = Synthetic polynucleotide
  source 1..23: mol_type = other DNA
tgaagaagaaagaataaaataat

SEQ ID NO: 295 - DNA, 25 bp, synthetic construct
  misc_feature 1..25: note = Synthetic polynucleotide
  source 1..25: mol_type = other DNA
ttttccagtcccattattttcacag

SEQ ID NO: 296 - DNA, 25 bp, synthetic construct
  misc_feature 1..25: note = Synthetic polynucleotide
  source 1..25: mol_type = other DNA
agtcccttctttcctttcagtctcc

SEQ ID NO: 297 - DNA, 25 bp, synthetic construct
  misc_feature 1..25: note = Synthetic polynucleotide
  source 1..25: mol_type = other DNA
tcaaggcaaagagaaaaagagtatt

SEQ ID NO: 298 - DNA, 25 bp, synthetic construct
  misc_feature 1..25: note = Synthetic polynucleotide
  source 1..25: mol_type = other DNA
agacaccaaagtaagaccacagatt

SEQ ID NO: 299 - DNA, 25 bp, synthetic construct
  misc_feature 1..25: note = Synthetic polynucleotide
  source 1..25: mol_type = other DNA
agacttggagacttcttcggagacc

SEQ ID NO: 300 - DNA, 25 bp, synthetic construct
  misc_feature 1..25: note = Synthetic polynucleotide
  source 1..25: mol_type = other DNA
taaaacacaggcatcaatcaaaata

SEQ ID NO: 301 - DNA, 21 bp, synthetic construct
  misc_feature 1..21: note = Synthetic polynucleotide
  source 1..21: mol_type = other DNA
gaactgggtgatgcggatgct

SEQ ID NO: 302 - DNA, 21 bp, synthetic construct
  misc_feature 1..21: note = Synthetic polynucleotide
  source 1..21: mol_type = other DNA
gctggaagaggaaggagagaa

SEQ ID NO: 303 - DNA, 27 bp, synthetic construct
  misc_feature 1..27: note = Synthetic polynucleotide
  source 1..27: mol_type = other DNA
acaaggtgaaaccccgtctctactaaa

SEQ ID NO: 304 - DNA, 27 bp, synthetic construct
  misc_feature 1..27: note = Synthetic polynucleotide
  source 1..27: mol_type = other DNA
ttcttccacctaagctttgccataaat

SEQ ID NO: 305 - DNA, 27 bp, synthetic construct
  misc_feature 1..27: note = Synthetic polynucleotide
  source 1..27: mol_type = other DNA
atcaaaaacatcatcatccaggactgt

SEQ ID NO: 306 - DNA, 27 bp, synthetic construct
  misc_feature 1..27: note = Synthetic polynucleotide
  source 1..27: mol_type = other DNA
tctcaggactctgacactgtccaactt

SEQ ID NO: 307 - DNA, 27 bp, synthetic construct
  misc_feature 1..27: note = Synthetic polynucleotide
  source 1..27: mol_type = other DNA
caggatggttcgtactaagacatggac

SEQ ID NO: 308 - DNA, 27 bp, synthetic construct
  misc_feature 1..27: note = Synthetic polynucleotide
  source 1..27: mol_type = other DNA
gctttcttcaaggtttcttccaaagac

SEQ ID NO: 309 - DNA, 27 bp, synthetic construct
  misc_feature 1..27: note = Synthetic polynucleotide
  source 1..27: mol_type = other DNA
catgcgttgtagaatgaatgaagacac

SEQ ID NO: 310 - DNA, 27 bp, synthetic construct
  misc_feature 1..27: note = Synthetic polynucleotide
  source 1..27: mol_type = other DNA
tgtactgagaaatttgaccacacagga

SEQ ID NO: 311 - DNA, 26 bp, synthetic construct
  misc_feature 1..26: note = Synthetic polynucleotide
  source 1..26: mol_type = other DNA
agcaggttaatggataatttcttgat

SEQ ID NO: 312 - DNA, 26 bp, synthetic construct
  misc_feature 1..26: note = Synthetic polynucleotide
  source 1..26: mol_type = other DNA
ggtcctttgtaaaaaccattacactt

SEQ ID NO: 313 - DNA, 27 bp, synthetic construct
  misc_feature 1..27: note = Synthetic polynucleotide
  source 1..27: mol_type = other DNA
cgtttccttttattttggagctaatgc

SEQ ID NO: 314 - DNA, 27 bp, synthetic construct
  misc_feature 1..27: note = Synthetic polynucleotide
  source 1..27: mol_type = other DNA
aaggaagtcactccaaggtacacactg

SEQ ID NO: 315 - DNA, 27 bp, synthetic construct
  misc_feature 1..27: note = Synthetic polynucleotide
  source 1..27: mol_type = other DNA
atgcaagaaatgtctggagaaattgaa

SEQ ID NO: 316 - DNA, 27 bp, synthetic construct
  misc_feature 1..27: note = Synthetic polynucleotide
  source 1..27: mol_type = other DNA
ccttgtattttctcagtacgagctcca

SEQ ID NO: 317 - DNA, 25 bp, synthetic construct
  misc_feature 1..25: note = Synthetic polynucleotide
  source 1..25: mol_type = other DNA
gcgacggcggctgcggctactggag

SEQ ID NO: 318 - DNA, 25 bp, synthetic construct
  misc_feature 1..25: note = Synthetic polynucleotide
  source 1..25: mol_type = other DNA
ttcttctaatgcttcttgactaccc

SEQ ID NO: 319 - DNA, 25 bp, synthetic construct
  misc_feature 1..25: note = Synthetic polynucleotide
  source 1..25: mol_type = other DNA
tgaaaggaaggagaaggagaaaaag

SEQ ID NO: 320 - DNA, 25 bp, synthetic construct
  misc_feature 1..25: note = Synthetic polynucleotide
  source 1..25: mol_type = other DNA
agaggcagtgagggcagcattagta

SEQ ID NO: 321 - DNA, 25 bp, synthetic construct
  misc_feature 1..25: note = Synthetic polynucleotide
  source 1..25: mol_type = other DNA
taaatggagaggaggtagaagaaac

SEQ ID NO: 322 - DNA, 25 bp, synthetic construct
  misc_feature 1..25: note = Synthetic polynucleotide
  source 1..25: mol_type = other DNA
ggtggtagaaagagaacgaaaagtc

SEQ ID NO: 323 - DNA, 23 bp, synthetic construct
  misc_feature 1..23: note = Synthetic polynucleotide
  source 1..23: mol_type = other DNA
gcgtgacattaaggagaagctgt

SEQ ID NO: 324 - DNA, 23 bp, synthetic construct
  misc_feature 1..23: note = Synthetic polynucleotide
  source 1..23: mol_type = other DNA
cttgttttctgcgcaagttaggt

SEQ ID NO: 325 - DNA, 18 bp, synthetic construct
  misc_feature 1..18: note = Synthetic polynucleotide
  source 1..18: mol_type = other DNA
aggagccaggagaggact

SEQ ID NO: 326 - DNA, 18 bp, synthetic construct
  misc_feature 1..18: note = Synthetic polynucleotide
  source 1..18: mol_type = other DNA
gcagggacgaaaggtatc

SEQ ID NO: 327 - DNA, 23 bp, synthetic construct
  misc_feature 1..23: note = Synthetic polynucleotide
  source 1..23: mol_type = other DNA
gtatcgtggaaggactcatgacc

SEQ ID NO: 328 - DNA, 23 bp, synthetic construct
  misc_feature 1..23: note = Synthetic polynucleotide
  source 1..23: mol_type = other DNA
tctcttcctcttgtgctcttgct

SEQ ID NO: 329 - DNA, 120 bp, synthetic construct
  misc_feature 1..120: note = Synthetic polynucleotide
  source 1..120: mol_type = other DNA
gcacacagaagcgctcagtaagggctttgaaacttaacagtttgggagccagatcctcaggccacatctctctcctcccacgacctccgcggtcctccagaaccatagagagttgtacag

SEQ ID NO: 330 - DNA, 120 bp, synthetic construct
  misc_feature 1..120: note = Synthetic polynucleotide
  source 1..120: mol_type = other DNA
agatcgccctgctctatgctctactctcctggggagcggggccagagaggccggaagtgctgcgagccctgggccacgctggccgtgctggcagtgggccgcctcgatccctctgcagtc

SEQ ID NO: 331 - DNA, 120 bp, synthetic construct
  misc_feature 1..120: note = Synthetic polynucleotide
  source 1..120: mol_type = other DNA
tttcccttgaggctccaagaccagcaggtgaggcctcgcggcgctgaaaccgtgaggcccggaccacaggtgcgggaggcggagactgcgggtggagattggcgccgcggaagccaatca

SEQ ID NO: 332 - DNA, 120 bp, synthetic construct
  misc_feature 1..120: note = Synthetic polynucleotide
  source 1..120: mol_type = other DNA
gcgatgggacttgtggacctgtaaggggcggggcgagccgaaggtggaggtcaaaggggcgtggcgttacagagcctctagcgctgggtgttggggacctgacgctatggagctctcgga

SEQ ID NO: 333 - DNA, 120 bp, synthetic construct
  misc_feature 1..120: note = Synthetic polynucleotide
  source 1..120: mol_type = other DNA
gttttgtgggggacggctgtgagtggggggttcctgctgcgggatgagaacgtagacgccagtggctcactcgctcctggcaccttccctttcaggctccagatggaccctgggaaggac

SEQ ID NO: 334 - DNA, 120 bp, synthetic construct
  misc_feature 1..120: note = Synthetic polynucleotide
  source 1..120: mol_type = other DNA
aaagagggggtgccccagccctcagggccgccagcaaggaagaaatttgtgatacccctcgacgaggatgaggtccctcctggagtggtaggacaaggagatgcggggcccctgggaggc

SEQ ID NO: 335 - DNA, 120 bp, synthetic construct
  misc_feature 1..120: note = Synthetic polynucleotide
  source 1..120: mol_type = other DNA
ttctccccacaggccaagcccttattccgatctacacagagccttcccactgtggacacctcggcccaggcggcccctcagacctacgccgaatatgccatctcacagcctctggaaggg

SEQ ID NO: 336 - DNA, 120 bp, synthetic construct
  misc_feature 1..120: note = Synthetic polynucleotide
  source 1..120: mol_type = other DNA
gctggggccacgtgccccacagggtcagagcccctggcaggagagacgcccaaccaggccctgaaacccggggcaaaatccaacagcatcattgtgagccctcggcaggtgaggagggag

SEQ ID NO: 337 - DNA, 120 bp, synthetic construct
  misc_feature 1..120: note = Synthetic polynucleotide
  source 1..120: mol_type = other DNA
ctccccagaggggcaatcccgtactgaagttcgtgcgcaatgtgccctgggaatttggcgacgtaattcccgactatgtgctgggccagagcacctgtgccctgttcctcaggtgagctc

SEQ ID NO: 338 - DNA, 120 bp, synthetic construct
  misc_feature 1..120: note = Synthetic polynucleotide
  source 1..120: mol_type = other DNA
cccccaccagcctccgctaccacaacctgcacccagactacatccatgggcggctgcagagcctggggaagaacttcgccttgcgggtcctgcttgtccaggtggatgtggtaagcaggg

SEQ ID NO: 339 - DNA, 120 bp, synthetic construct
  misc_feature 1..120: note = Synthetic polynucleotide
  source 1..120: mol_type = other DNA
aattctgattttctcctccagaaagatccccagcaggccctcaaggagctggctaagatgtgtatcctggccgactgcacattgatcctcgcctggaggtgagatgagggcttccctgcc

SEQ ID NO: 340 - DNA, 120 bp, synthetic construct
  misc_feature 1..120: note = Synthetic polynucleotide
  source 1..120: mol_type = other DNA
gttatcccagccccgaggaagctgggcggtacctggagacctacaaggcctatgagcagaaaccagcggacctcctgatggagaagctagagcaggacttcgtctcccgggtgaggccac

SEQ ID NO: 341 - DNA, 120 bp, synthetic construct
  misc_feature 1..120: note = Synthetic polynucleotide
  source 1..120: mol_type = other DNA
cactcctgcctccaccctttccaggtgactgaatgtctgaccaccgtgaagtcagtcaacaaaacggacagtcagaccctcctgaccacatttggagtaaggaatggctcccctgcccca

SEQ ID NO: 342 - DNA, 120 bp, synthetic construct
  misc_feature 1..120: note = Synthetic polynucleotide
  source 1..120: mol_type = other DNA
ggaggcttttgtgctcaactgccctgacccctcgctttcacctttcagtctctggaacagctcatcgccgcatcaagagaagatctggccttatgcccaggcctgggccctcagaaagta

SEQ ID NO: 343 - DNA, 119 bp, synthetic construct
  misc_feature 1..119: note = Synthetic polynucleotide
  source 1..119: mol_type = other DNA
agagctctgggaaagaacccaaggagttgggggaaggagagagccccaaataaacacaacctgagaccccaaagttttaaggtgaaaaaagaaccaaagaccagacacagtggcttccg

SEQ ID NO: 344 - DNA, 120 bp, synthetic construct
  misc_feature 1..120: note = Synthetic polynucleotide
  source 1..120: mol_type = other DNA
ggcgggtcgaccccgctgcacagtccggccggcgccatgaagtgagaagggggctgggggtcgcgctcgctagcgggcgcggggggtcttgaagatggggtcatcggtgggcgcgcctgg

SEQ ID NO: 345 - DNA, 120 bp, synthetic construct
  misc_feature 1..120: note = Synthetic polynucleotide
  source 1..120: mol_type = other DNA
gtccccaagggggcgaggggagggtgaaggggtgggacgggggcagccgcagggagcagcagtgatagcgaggagacactgagggggccccgaggctcctgaggacctgagggttaccgg

SEQ ID NO: 346 - DNA, 120 bp, synthetic construct
  misc_feature 1..120: note = Synthetic polynucleotide
  source 1..120: mol_type = other DNA
gggcgccgggcccgtcacccttctctgggctcgacgaccgggcactgtggaggcgggagaggggctgaggggacgggaactgacccagcagcccctgccgccaggctcaacgtggacggg

SEQ ID NO: 347 - DNA, 120 bp, synthetic construct
  misc_feature 1..120: note = Synthetic polynucleotide
  source 1..120: mol_type = other DNA
ctcctggtctacttcccgtacgactacatctaccccgagcagttctcctacatgcgggagctcaaacgcacgctggacgccaaggtgggtggccggtgggcccgacccgcccactcgacc

SEQ ID NO: 348 - DNA, 120 bp, synthetic construct
  misc_feature 1..120: note = Synthetic polynucleotide
  source 1..120: mol_type = other DNA
cctctggtccccaacatgcagggtcatggagtcctggagatgccctcaggcaccgggaagacagtatccctgttggccctgatcatggcataccagagagtgagtgatgcgctgaacccg

SEQ ID NO: 349 - DNA, 120 bp, synthetic construct
  misc_feature 1..120: note = Synthetic polynucleotide
  source 1..120: mol_type = other DNA
agggactgagtccgcttgttatcggcaggcatatccgctggaggtgaccaaactcatctactgctcaagaactgtgccagagattgagaaggtaagctgggactcatcctggtgctccag

SEQ ID NO: 350 - DNA, 120 bp, synthetic construct
  misc_feature 1..120: note = Synthetic polynucleotide
  source 1..120: mol_type = other DNA
agtaggtgattgaagagcttcgaaagttgctcaacttctatgagaagcaggagggcgagaagctgccgtttctgggactggctctgagctcccgcaaaaacttgtgtattcaccctgagg

SEQ ID NO: 351 - DNA, 120 bp, synthetic construct
  misc_feature 1..120: note = Synthetic polynucleotide
  source 1..120: mol_type = other DNA
aggtgacacccctgcgctttgggaaggacgtcgatgggaaatgccacagcctcacagcctcctatgtgcgggcgcagtaccagcatgacaccagcctgccccactgccgattctatgagg

SEQ ID NO: 352 - DNA, 120 bp, synthetic construct
  misc_feature 1..120: note = Synthetic polynucleotide
  source 1..120: mol_type = other DNA
aggaatttgatgcccatgggcgtgaggtgcccctccccgctggcatctacaacctggatgacctgaaggccctggggcggcgccagggctggtgcccatacttccttgctcgatactcag

SEQ ID NO: 353 - DNA, 120 bp, synthetic construct
  misc_feature 1..120: note = Synthetic polynucleotide
  source 1..120: mol_type = other DNA
ggtaggggtaggggttggggtggcagggccctggtaaccctgctccccggccccccagatcctgcatgccaatgtggtggtttatagctaccactacctcctggaccccaagattgcaga

SEQ ID NO: 354 - DNA, 120 bp, synthetic construct
  misc_feature 1..120: note = Synthetic polynucleotide
  source 1..120: mol_type = other DNA
cctggtgtccaaggaactggcccgcaaggccgtcgtggtcttcgacgaggcccacaacattggtgaggggggcgccaggggccaaagggatgccagcccctctgagtgaggcccctgcag

SEQ ID NO: 355 - DNA, 120 bp, synthetic construct
  misc_feature 1..120: note = Synthetic polynucleotide
  source 1..120: mol_type = other DNA
ccccttctccagacaacgtctgcatcgactccatgagcgtcaacctcacccgccggacccttgaccggtgccagggcaacctggagaccctgcagaagacggtgctcaggtggggccggg

SEQ ID NO: 356 - DNA, 120 bp, synthetic construct
  misc_feature 1..120: note = Synthetic polynucleotide
  source 1..120: mol_type = other DNA
acttgtccccagggttgccagccccctcccagccccagctcatctctccgcaggatcaaagagacagacgagcagcgcctgcgggacgagtaccggcgtctggtggaggggctgcgggag

SEQ ID NO: 357 - DNA, 120 bp, synthetic construct
  misc_feature 1..120: note = Synthetic polynucleotide
  source 1..120: mol_type = other DNA
gccagcgccgcccgggagacggacgcccacctggccaaccccgtgctgcccgacgaagtgctgcagggtgagccccgaccccccgctgccccccagtccctttcccgcctccccgtcgcc

SEQ ID NO: 358 - DNA, 120 bp, synthetic construct
  misc_feature 1..120: note = Synthetic polynucleotide
  source 1..120: mol_type = other DNA
cgcctccccgtcgccgctgatagcgtctcctcgcagaggcagtgcctggctccatccgcacggccgagcatttcctgggcttcctgaggcggctgctggagtacgtgaagtggcggctgc

SEQ ID NO: 359 - DNA, 120 bp, synthetic construct
  misc_feature 1..120: note = Synthetic polynucleotide
  source 1..120: mol_type = other DNA
gtgtgcagcatgtggtgcaggagagcccgcccgccttcctgagcggcctggcccagcgcgtgtgcatccagcgcaagcccctcaggtgcggccccagacagcgcgcggggtgggggcccg

SEQ ID NO: 360 - DNA, 120 bp, synthetic construct
  misc_feature 1..120: note = Synthetic polynucleotide
  source 1..120: mol_type = other DNA
attctgtgctgaacgcctccggtccctgctgcatactctggagatcaccgaccttgctgacttctccccgctcaccctccttgctaactttgccacccttgtcagcacctacgccaaagg

SEQ ID NO: 361 - DNA, 120 bp, synthetic construct
  misc_feature 1..120: note = Synthetic polynucleotide
  source 1..120: mol_type = other DNA
acccctccttgcctgccctctgcaggcttcaccatcatcatcgagccctttgacgacagaaccccgaccattgccaaccccatcctgcacttcaggtgggaccctgcccggtgagggtgt

SEQ ID NO: 362 - DNA, 120 bp, synthetic construct
  misc_feature 1..120: note = Synthetic polynucleotide
  source 1..120: mol_type = other DNA
cctcactgccctcatctctctccagctgcatggacgcctcgctggccatcaaacccgtatttgagcgtttccagtctgtcatcatcacatctggggtaaggacccttccccgtcccctcc

SEQ ID NO: 363 - DNA, 120 bp, synthetic construct
  misc_feature 1..120: note = Synthetic polynucleotide
  source 1..120: mol_type = other DNA
ccgccacagacactgtccccgctggacatctaccccaagatcctggacttccaccccgtcaccatggcaaccttcaccatgacgctggcacgggtctgcctctgccctatggtgagtggg

SEQ ID NO: 364 - DNA, 120 bp, synthetic construct
  misc_feature 1..120: note = Synthetic polynucleotide
  source 1..120: mol_type = other DNA
gcttatcgtgacctctgttgctctccagatcatcggccgtggcaatgaccaggtggccatcagctccaaatttgagacccgggaggatattggtatgctgccagtggggctggtttctgt

SEQ ID NO: 365 - DNA, 120 bp, synthetic construct
  misc_feature 1..120: note = Synthetic polynucleotide
  source 1..120: mol_type = other DNA
agacagagaagggaggaggacctgggccaggagagggcccaacctctgaccccttgcagctgtgatccggaactatgggaacctcctgctggagatgtccgctgtggtccctgatggcat

SEQ ID NO: 366 - DNA, 120 bp, synthetic construct
  misc_feature 1..120: note = Synthetic polynucleotide
  source 1..120: mol_type = other DNA
cgtggccttcttcaccagctaccagtacatggagagcaccgtggcctcctggtatgagcaggtacgcctggccaccccctccctgcacctgctctcctcagtccctggcactgcaccgct

SEQ ID NO: 367 - DNA, 120 bp, synthetic construct
  misc_feature 1..120: note = Synthetic polynucleotide
  source 1..120: mol_type = other DNA
gggtccttccccaggggatccttgagaacatccagaggaacaagctgctctttattgagacccaggatggtgccgaaaccagtgtcgccctggagaagtaccaggaggtgggtgtgcgag

SEQ ID NO: 368 - DNA, 120 bp, synthetic construct
  misc_feature 1..120: note = Synthetic polynucleotide
  source 1..120: mol_type = other DNA
tgcgtctcggtctccccacctcaggcctgcgagaatggccgcggggccatcctgctgtcagtggcccggggcaaagtgtccgagggaatcgactttggtgagtggactgggctcttcctc

SEQ ID NO: 369 - DNA, 120 bp, synthetic construct
  misc_feature 1..120: note = Synthetic polynucleotide
  source 1..120: mol_type = other DNA
cgccccctctcctctgcccaccagtgcaccactacgggcgggccgtcatcatgtttggcgtcccctacgtctacacacagagccgcattctcaaggtgagtagctctgtctcccagggag

SEQ ID NO: 370 - DNA, 120 bp, synthetic construct
  misc_feature 1..120: note = Synthetic polynucleotide
  source 1..120: mol_type = other DNA
aggagtggctcccccacctccccagcttctcatcctccgtatctgcaggcgcggctggaatacctgcgggaccagttccagattcgtgagaatgactttcttaccttcgatgccatgcgc

SEQ ID NO: 371 - DNA, 120 bp, synthetic construct
  misc_feature 1..120: note = Synthetic polynucleotide
  source 1..120: mol_type = other DNA
cacgcggcccagtgtgtgggtcgggccatcaggggcaagacggactacggcctcatggtctttgccgacaaggtgcagcttcaggggtgcccctgtgctcccatcccaggctccaagaac

SEQ ID NO: 372 - DNA, 120 bp, synthetic construct
  misc_feature 1..120: note = Synthetic polynucleotide
  source 1..120: mol_type = other DNA
aggctccccgaatgaccttctgtccctggcctgcgcttctgcccacagcggtttgcccgtggggacaagcgggggaagctgccccgctggatccaggagcacctcacagatgccaacctc

SEQ ID NO: 373 - DNA, 120 bp, synthetic construct
  misc_feature 1..120: note = Synthetic polynucleotide
  source 1..120: mol_type = other DNA
aacctgaccgtggacgagggtgtccaggtggccaagtacttcctgcggcagatggcacagcccttccaccgggtgaggcctgcgtccccctcccggcaccctcccaggctgagcttctcc

SEQ ID NO: 374 - DNA, 120 bp, synthetic construct
  misc_feature 1..120: note = Synthetic polynucleotide
  source 1..120: mol_type = other DNA
tctgttctctgcaggaggatcagctgggcctgtccctgctcagcctggagcagctagaatcagaggagacgctgaagaggatagagcagattgctcagcagctctgagtggggcgggtgg

SEQ ID NO: 375 - DNA, 120 bp, synthetic construct
  misc_feature 1..120: note = Synthetic polynucleotide
  source 1..120: mol_type = other DNA
gagcggggtcatcttctctctgctgctgtagctgccatgggcaaaagagaccgagcggaccgcggtgagacgttgcgcgggcacgctcagccacgactgcccttgccggccctgcccccc

SEQ ID NO: 376 - DNA, 120 bp, synthetic construct
  misc_feature 1..120: note = Synthetic polynucleotide
  source 1..120: mol_type = other DNA
gctctgcctcggagctgctccgggcctctgcgccggccgaccctgctggccctcccgcgcgcaccccgttgggacaggcctttgggcgggagagatgctggacctgggcgcagcccagcg

SEQ ID NO: 377 - DNA, 120 bp, synthetic construct
  misc_feature 1..120: note = Synthetic polynucleotide
  source 1..120: mol_type = other DNA
aactcggcctaggggagacgggtgaggggcgcaacgcctgcgggatgcaggtggctcttagctaggagtttgcggcggcgcaggtgaaatgctgccaagcggtgcggagggaaccctgaa

SEQ ID NO: 378 - DNA, 120 bp, synthetic construct
  misc_feature 1..120: note = Synthetic polynucleotide
  source 1..120: mol_type = other DNA
gtgttggtatcttgcagacaagaagaaatccaggaagcggcactatgaggatgaagaggatgatgaagaggacgccccggggaacgaccctcaggaagcggttccctcggcggcggggaa

SEQ ID NO: 379 - DNA, 120 bp, synthetic construct
  misc_feature 1..120: note = Synthetic polynucleotide
  source 1..120: mol_type = other DNA
gcaggtggatgagtcaggcaccaaagtggatgaatatggagccaaggactacaggctgcaaatgccgctgaaggacgaccacacctccaggcccctctgggtggtaagcatgcccttagc

SEQ ID NO: 380 - DNA, 120 bp, synthetic construct
  misc_feature 1..120: note = Synthetic polynucleotide
  source 1..120: mol_type = other DNA
aggctcccgatggccatatcttcttggaagccttctctccagtttacaaatatgcccaagacttcttggtggctattgcagagccagtgtgccgaccaacccatgtgcatgagtacaaac

SEQ ID NO: 381 - DNA, 120 bp, synthetic construct
  misc_feature 1..120: note = Synthetic polynucleotide
  source 1..120: mol_type = other DNA
taactgcctactccttgtatgcagctgtcagcgttgggctgcaaaccagtgacatcaccgagtacctcaggaagctcagcaagactggagtccctgatggaattatgcagtttattaagg

SEQ ID NO: 382 - DNA, 120 bp, synthetic construct
  misc_feature 1..120: note = Synthetic polynucleotide
  source 1..120: mol_type = other DNA
cacatttacctgttggatgttatctgtatttgcagttgtgtactgtcagctatggaaaagtcaagctggtcttgaagcacaacaggtaagagattccatgacaggcctgtcccaaggcac

SEQ ID NO: 383 - DNA, 120 bp, synthetic construct
  misc_feature 1..120: note = Synthetic polynucleotide
  source 1..120: mol_type = other DNA
tccaggaggtacctgctggtcagtagaccccttcttcctcccctgcttgcagatacttcgttgaaagttgccaccctgatgtaatccagcatcttctccaggaccccgtgatccgagaat

SEQ ID NO: 384 - DNA, 120 bp, synthetic construct
  misc_feature 1..120: note = Synthetic polynucleotide
  source 1..120: mol_type = other DNA
gccgcttaagaaactctgaaggggaggccactgagctcatcacagagactttcacaagcaaatctgccgtatgtggactcctgggccacccttgggtgggggcaggcatttaggatttca

SEQ ID NO: 385 - DNA, 120 bp, synthetic construct
  misc_feature 1..120: note = Synthetic polynucleotide
  source 1..120: mol_type = other DNA
aatgttgtgtgcacgttcagcacctacctctctcacagatttctaagactgctgaaagcagtggtgggccctccacttcccgagtgacagatccacagggtaaatctgacatccccatgg

SEQ ID NO: 386 - DNA, 120 bp, synthetic construct
  misc_feature 1..120: note = Synthetic polynucleotide
  source 1..120: mol_type = other DNA
acctgtttgacttctatgagcaaatggacaaggatgaagaagaagaagaagagacacagacagtgtcttttgaagtcaagcaggttagtgaatgtaccttcctcctggttccttcactga

SEQ ID NO: 387 - DNA, 120 bp, synthetic construct
  misc_feature 1..120: note = Synthetic polynucleotide
  source 1..120: mol_type = other DNA
tttgtgccctctttccaggaaatgattgaggaactccagaaacgttgcatccacctggagtaccctctgttggcagaatatgacttccggaatgattctgtcaaccctgatatcaacatt

SEQ ID NO: 388 - DNA, 120 bp, synthetic construct
  misc_feature 1..120: note = Synthetic polynucleotide
  source 1..120: mol_type = other DNA
gacctaaagcccacagctgtcctcagaccctatcaggagaagagcttgcgaaagatgtttggaaacgggcgtgcacgttcgggggtcattgttcttccctgcggtaagtggtaccagagt

SEQ ID NO: 389 - DNA, 120 bp, synthetic construct
  misc_feature 1..120: note = Synthetic polynucleotide
  source 1..120: mol_type = other DNA
attgcactcttgtttcttgtaggtgctggaaagtccctggttggtgtgactgctgcatgcactgtcagaaaacgctgtctggtgctgggcaactcagctgtttctgtggagcagtggaaa

SEQ ID NO: 390 - DNA, 120 bp, synthetic construct
  misc_feature 1..120: note = Synthetic polynucleotide
  source 1..120: mol_type = other DNA
gcccagttcaagatgtggtccaccattgacgacagccagatctgccggttcacctccgatgccaaggacaagcccatcggctgctccgttgccattagcacctactccatgctgggccac

SEQ ID NO: 391 - DNA, 120 bp, synthetic construct
  misc_feature 1..120: note = Synthetic polynucleotide
  source 1..120: mol_type = other DNA
accaccaaaaggtcctgggaggccgagcgagtcatggagtggctcaagacccaggagtggggcctcatgatcctggatgaagtgcacaccataccaggtaagcaggctggagctgagctg

SEQ ID NO: 392 - DNA, 120 bp, synthetic construct
  misc_feature 1..120: note = Synthetic polynucleotide
  source 1..120: mol_type = other DNA
agtgtgaatgctcccattgtttcctcagccaagatgttccgaagggtgctcaccatcgtgcaggcccactgtaagctgggtttgactgcgaccctcgtccgcgaagatgacaaaattgtg

SEQ ID NO: 393 - DNA, 120 bp, synthetic construct
  misc_feature 1..120: note = Synthetic polynucleotide
  source 1..120: mol_type = other DNA
gatttaaattttctgattgggcctaagctctacgaagccaactggatggagctgcagaataatggctacatcgccaaagtccagtgtgctgaggtagctgggcctgggctgggggcgtct

SEQ ID NO: 394 - DNA, 120 bp, synthetic construct
  misc_feature 1..120: note = Synthetic polynucleotide
  source 1..120: mol_type = other DNA
gttcttactgtattacaggtctggtgccctatgtctcctgaattttaccgggaatatgtggcaatcaaaaccaagaaacgaatcttgctgtacaccatgaaccccaacaaatttagagct

SEQ ID NO: 395 - DNA, 120 bp, synthetic construct
  misc_feature 1..120: note = Synthetic polynucleotide
  source 1..120: mol_type = other DNA
tgccagtttctgatcaagtttcatgaaaggaggaatgacaagattattgtctttgctgacaatgtgtttgccctaaaggaatatgccattcgactgaacaagtaagaattgaaaacttgg

SEQ ID NO: 396 - DNA, 120 bp, synthetic construct
  misc_feature 1..120: note = Synthetic polynucleotide
  source 1..120: mol_type = other DNA
attcttctctagaccctatatctacggacctacgtctcagggggaaaggatgcaaattctccagaatttcaagcacaaccccaaaattaacaccatcttcatatccaaggtttgtgtggc

SEQ ID NO: 397 - DNA, 120 bp, synthetic construct
  misc_feature 1..120: note = Synthetic polynucleotide
  source 1..120: mol_type = other DNA
ggtaggtgacacttcgtttgatctgccggaagcaaatgtcctcattcagatctcatcccatggtggctccaggcgtcaggaagcccaaaggctagggcgggtgcttcgagctaaaaaagg

SEQ ID NO: 398 - DNA, 120 bp, synthetic construct
  misc_feature 1..120: note = Synthetic polynucleotide
  source 1..120: mol_type = other DNA
ggatggttgcagaagagtacaatgcctttttctactcactggtatcccaggacacacaggaaatggcttactcaaccaagcggcagagattcttggtagatcaaggttatagcttcaagg

SEQ ID NO: 399 - DNA, 120 bp, synthetic construct
  misc_feature 1..120: note = Synthetic polynucleotide
  source 1..120: mol_type = other DNA
agagagagcagaaaggggcatgtctgctggcttgggctttgcaggtgatcacgaaactcgctggcatggaggaggaagacttggcgttttcgacaaaagaagagcaacagcagctcttac

SEQ ID NO: 400 - DNA, 120 bp, synthetic construct
  misc_feature 1..120: note = Synthetic polynucleotide
  source 1..120: mol_type = other DNA
agaaagtcctggcagccactgacctggatgccgaggaggaggtggtggctggggaatttggctccagatccagccaggtgagtaaatgggtgaggaggttccaggtgtcagtgctctcac

SEQ ID NO: 401 - DNA, 120 bp, synthetic construct
  misc_feature 1..120: note = Synthetic polynucleotide
  source 1..120: mol_type = other DNA
aggagtatcttttcttctggagactaacatgggctggttcccccttcccggcaggcatctcggcgctttggcaccatgagttctatgtctggggccgacgacactgtgtacatggagtac

SEQ ID NO: 402 - DNA, 120 bp, synthetic construct
  misc_feature 1..120: note = Synthetic polynucleotide
  source 1..120: mol_type = other DNA
cactcatcgcggagcaaggcgcccagcaaacatgtacacccgctcttcaagcgctttaggaaatgatgcttaggcagggtacttcgttcaagaccggcgcttggcacccttgttggaaag

SEQ ID NO: 403 - DNA, 120 bp, synthetic construct
  misc_feature 1..120: note = Synthetic polynucleotide
  source 1..120: mol_type = other DNA
acccggaagagcttccatggagtcagggcagccggctcgacggattgccatggcgccgctgctggagtacgagcgacagctggtgctggaactgctcgacactgacgggctagtagtgtg

SEQ ID NO: 404 - DNA, 120 bp, synthetic construct
  misc_feature 1..120: note = Synthetic polynucleotide
  source 1..120: mol_type = other DNA
cgcccgcgggctcggcgcggaccggctcctctaccactttctccagctgcactgccacccagcctgcctggtgctggtgctcaacacgcagccggccgaggaggtgcggccgcgctggcg

SEQ ID NO: 405 - DNA, 120 bp, synthetic construct
  misc_feature 1..120: note = Synthetic polynucleotide
  source 1..120: mol_type = other DNA
agcctactaatcaagtttgatttgatttaggagtattttatcaatcagctgaagatagaaggagttgaacacctccctcgccgtgtaacaaatgaaatcacaagcaacagtcgctatgaa

SEQ ID NO: 406 - DNA, 120 bp, synthetic construct
  misc_feature 1..120: note = Synthetic polynucleotide
  source 1..120: mol_type = other DNA
gtttacacacaaggtggtgttatatttgcgacaagtaggatacttgtggttgacttcttgactgatagaataccttcagatttaattactggtaagaatttgaaatcttattattagtat

SEQ ID NO: 407 - DNA, 120 bp, synthetic construct
  misc_feature 1..120: note = Synthetic polynucleotide
  source 1..120: mol_type = other DNA
aaattatgtttctccccctcaggcatcttggtgtatagagcccacagaataatcgagtcttgtcaagaagcattcatcttgcgcctctttcgccagaaaaacaaacgtggttttattaaa

SEQ ID NO: 408 - DNA, 120 bp, synthetic construct
  misc_feature 1..120: note = Synthetic polynucleotide
  source 1..120: mol_type = other DNA
gctttcacagacaatgctgttgcctttgatactggtttttgtcatgtggaaagagtgatgagaaatctttttgtgaggaaactgtatctgtggccaaggtaaagaacattatgtgacaaa

SEQ ID NO: 409 - DNA, 120 bp, synthetic construct
  misc_feature 1..120: note = Synthetic polynucleotide
  source 1..120: mol_type = other DNA
atatttgtctctttaggttccatgtagcagtaaactcatttttagaacagcacaaacctgaagttgtagaaatccatgtttctatgacacctaccatgcttgctatacagactgctatac

SEQ ID NO: 410 - DNA, 120 bp, synthetic construct
  misc_feature 1..120: note = Synthetic polynucleotide
  source 1..120: mol_type = other DNA
tggacattttaaatgcatgtctaaaggaactaaaatgccataacccatcgcttgaagtggaagatttatctttagaaaatgctattggaaaaccttttgacaaggtactctttttccttt

SEQ ID NO: 411 - DNA, 120 bp, synthetic construct
  misc_feature 1..120: note = Synthetic polynucleotide
  source 1..120: mol_type = other DNA
tagcaataccaaattttattcttgttttagacaatccgccattatctggatcctttgtggcaccagcttggagccaagactaaatccttagttcaggatttgaagatattacgaactttg

SEQ ID NO: 412 - DNA, 120 bp, synthetic construct
  misc_feature 1..120: note = Synthetic polynucleotide
  source 1..120: mol_type = other DNA
ctgcagtatctctctcagtatgattgtgtcacatttcttaatcttctggaatctctgagagcaacggaaaaagcttttggtcagaattcaggtgggagattaaaatactaataatattct

SEQ ID NO: 413 - DNA, 120 bp, synthetic construct
  misc_feature 1..120: note = Synthetic polynucleotide
  source 1..120: mol_type = other DNA
ctgttttaaattaaccataaattgatggcactttttcttttaacttttcgtattaggttggctgtttcttgactccagcacctcgatgtttataaatgctcgagcaagggtttatcatct

SEQ ID NO: 414 - DNA, 120 bp, synthetic construct
  misc_feature 1..120: note = Synthetic polynucleotide
  source 1..120: mol_type = other DNA
tccagatgccaaaatgagtaaaaaagaaaaaatatctgaaaaaatggaaattaaagaaggggaaggtatcttgtggggttaagtctttaaatgtgttttttatttcggtatttggtatgg

SEQ ID NO: 415 - DNA, 120 bp, synthetic construct
  misc_feature 1..120: note = Synthetic polynucleotide
  source 1..120: mol_type = other DNA
acagaaacaaaaaaggaactggtcctagaaagcaacccaaagtgggaggcactgactgaagtattaaaagaaattgaggcagaaaataaggagagtgaagctcttggtggtccaggtagg

SEQ ID NO: 416 - DNA, 120 bp, synthetic construct
  misc_feature 1..120: note = Synthetic polynucleotide
  source 1..120: mol_type = other DNA
ggtcaagtactgatttgtgcaagtgatgaccgaacatgttcccagctgagagactatatcactcttggagcggaggccttcttattgaggctctacaggaaaacctttgagaaggatagc

SEQ ID NO: 417 - DNA, 120 bp, synthetic construct
  misc_feature 1..120: note = Synthetic polynucleotide
  source 1..120: mol_type = other DNA
aaagctgaagaagtctggatgaaatttaggaaggaagacagttcaaagagaattaggaaatctcacaaaagacctaaagacccccaaaacaaagaacgggcttctaccaaagaaagaacc

SEQ ID NO: 418 - DNA, 120 bp, synthetic construct
  misc_feature 1..120: note = Synthetic polynucleotide
  source 1..120: mol_type = other DNA
ctcaaaaagaaaaaacggaagttgaccttaactcaaatggtaggaaaacctgaagaactggaagaggaaggagatgtcgaggaaggatatcgtcgagaaataagcagtagcccagaaagc

SEQ ID NO: 419 - DNA, 120 bp, synthetic construct
  misc_feature 1..120: note = Synthetic polynucleotide
  source 1..120: mol_type = other DNA
tgcccggaagaaattaagcatgaagaatttgatgtaaatttgtcatcggatgctgctttcggaatcctgaaagaacccctcactatcatccatccgcttctgggttgcagcgacccctat

SEQ ID NO: 420 - DNA, 120 bp, synthetic construct
  misc_feature 1..120: note = Synthetic polynucleotide
  source 1..120: mol_type = other DNA
gctctgacaagggtactacatgaagtggagccaagatacgtggttctttatgacgcagagctaacctttgttcggcagcttgaaatttacagggcgagtaggcctgggaaacctctgagg

SEQ ID NO: 421 - DNA, 120 bp, synthetic construct
  misc_feature 1..120: note = Synthetic polynucleotide
  source 1..120: mol_type = other DNA
gtcttaacatgcagggtttactttcttatatacggaggttcaactgaggaacaacgctatctcactgctttgcggaaagaaaaggaagcttttgaaaaactcataaggtaatacatagaa

SEQ ID NO: 422 - DNA, 120 bp, synthetic construct
  misc_feature 1..120: note = Synthetic polynucleotide
  source 1..120: mol_type = other DNA
acagggaaaaagcaagcatggttgtccctgaagaaagagaaggcagagatgaaacaaacttagacctagtaagaggcacagcatctgcagatgtttccactgacactcggaaagccggtg

SEQ ID NO: 423 - DNA, 120 bp, synthetic construct
  misc_feature 1..120: note = Synthetic polynucleotide
  source 1..120: mol_type = other DNA
attccttctttgagagttcttccccagtgacattacttactttttctctgtaggtggccaggaacagaatggtacacagcaaagcatagttgtggatatgcgtgaatttcgaagtgagct

SEQ ID NO: 424 - DNA, 120 bp, synthetic construct
  misc_feature 1..120: note = Synthetic polynucleotide
  source 1..120: mol_type = other DNA
tccatctctgatccatcgtcggggcattgacattgaacccgtgactttagaggttggagattacatcctcactccagaaatgtgcgtggagcgcaagagtatcagtgatttaatcggctc

SEQ ID NO: 425 - DNA, 120 bp, synthetic construct
  misc_feature 1..120: note = Synthetic polynucleotide
  source 1..120: mol_type = other DNA
tttaaataacggccgcctctacagccagtgcatctccatgtcccgctactacaagcgtcccgtgcttctgattgagtttgaccctagcaagcctttctctctcacttcccgaggtgcctt

SEQ ID NO: 426 - DNA, 120 bp, synthetic construct
  misc_feature 1..120: note = Synthetic polynucleotide
  source 1..120: mol_type = other DNA
gtttcaggagatctccagcaatgacattagttccaaactcactcttcttacacttcacttccccagactacggattctctggtgcccctctcctcatgcaacggcggagttgtttgagga

SEQ ID NO: 427 - DNA, 120 bp, synthetic construct
  misc_feature 1..120: note = Synthetic polynucleotide
  source 1..120: mol_type = other DNA
gctgaaacaaagcaagccacagcctgatgcggcgacagcactggccattacagcagattctgaaacccttcccgagtcagagaagtataatcctggtccccaagacttcttgttaaaaat

SEQ ID NO: 428 - DNA, 120 bp, synthetic construct
  misc_feature 1..120: note = Synthetic polynucleotide
  source 1..120: mol_type = other DNA
gccaggggtgaatgccaaaaactgccgctccttgatgcaccacgttaagaacatcgcagaattagcagccctgtcacaagacgagctcacgagtattctggggaatgctgcaaatgccaa

SEQ ID NO: 429 - DNA, 120 bp, synthetic construct
  misc_feature 1..120: note = Synthetic polynucleotide
  source 1..120: mol_type = other DNA
acagctttatgatttcattcacacctcttttgcagaagtcgtatcaaaaggaaaagggaaaaagtgaacagtgatggctgttttcttatcccatgcctgtacttttcagcggctccttgc

SEQ ID NO: 430 - DNA, 120 bp, synthetic construct
  misc_feature 1..120: note = Synthetic polynucleotide
  source 1..120: mol_type = other DNA
aggacgcagccgcctcatgggggtccaggggctctggaagctgctggagtgctccgggcggcaggtcagccccgaagcgctggaagggaagatcctggctgttggtatccttaacgccgc

SEQ ID NO: 431 - DNA, 120 bp, synthetic construct
  misc_feature 1..120: note = Synthetic polynucleotide
  source 1..120: mol_type = other DNA
cccggagttttttccattaacaattctcccagatattagcatttggttaaaccaagcacttaaaggagtccgggatcgccatgggaactcaatagaaaatcctcatcttctcactttgtt

SEQ ID NO: 432 - DNA, 120 bp, synthetic construct
  misc_feature 1..120: note = Synthetic polynucleotide
  source 1..120: mol_type = other DNA
tcatcggctctgcaaactcttattttttcgaattcgtcctatttttgtgtttgatggggatgctccactattgaagaaacagactttggtaagtgtcgtatagtttttagtaagtgtcaa

SEQ ID NO: 433 - DNA, 120 bp, synthetic construct
  misc_feature 1..120: note = Synthetic polynucleotide
  source 1..120: mol_type = other DNA
aggtgaagagaaggcagagaaaggacttagcgtccagtgactccaggaaaacgacagagaagcttctgaaaacatttttgaaaagacaagccatcaaaactgccttcagaagcaaaaggc

SEQ ID NO: 434 - DNA, 120 bp, synthetic construct
  misc_feature 1..120: note = Synthetic polynucleotide
  source 1..120: mol_type = other DNA
tttgtgtactttccagagatgaagcactacccagtcttacccaagttcgaagagaaaacgacctctatgttttgcctcctttacaagaggaagaaaaacacaggtaaatgtttaactatt

SEQ ID NO: 435 - DNA, 120 bp, synthetic construct
  misc_feature 1..120: note = Synthetic polynucleotide
  source 1..120: mol_type = other DNA
agatatcgtaaaagtatgtttgactttcagttcagaagaggaagatgaaaaagaatggcaagaaagaatgaatcaaaaacaagcattacaggtatttagatcatttttgaattcagaatg

SEQ ID NO: 436 - DNA, 120 bp, synthetic construct
  misc_feature 1..120: note = Synthetic polynucleotide
  source 1..120: mol_type = other DNA
aagtgtatgaaatgtaaatttcatggtgctgtgattttatctttacaggaagagttctttcataatcctcaagcgatagatattgagtctgaggacttcagcagcctgccccctgaagta

SEQ ID NO: 437 - DNA, 120 bp, synthetic construct
  misc_feature 1..120: note = Synthetic polynucleotide
  source 1..120: mol_type = other DNA
aagcatgaaatcttgactgatatgaaagagttcaccaagcgcagaagaacattatttgaagcaatgccagaggtgaaatatgcaacagtacattcatgcttagaattaagaacttcagca

SEQ ID NO: 438 - DNA, 120 bp, synthetic construct
  misc_feature 1..120: note = Synthetic polynucleotide
  source 1..120: mol_type = other DNA
tttattttgcctttaggagtctgatgacttttcacagtaccaactcaaaggcttgcttaaaaagaactatctgaaccagcatatagaacatgtccaaaaggaaatgaatcagcaacattc

SEQ ID NO: 439 - DNA, 120 bp, synthetic construct
  misc_feature 1..120: note = Synthetic polynucleotide
  source 1..120: mol_type = other DNA
aggacacatccgaaggcagtatgaagatgaagggggctttctgaaggaggtagagtcaaggagagtggtctctgaagacacttcacattacatcttgataaaaggtatcaggcaccatca

SEQ ID NO: 440 - DNA, 120 bp, synthetic construct
  misc_feature 1..120: note = Synthetic polynucleotide
  source 1..120: mol_type = other DNA
aaggtattcaagctaagacagttgcagaagtggattcagagtctcttccttcttccagcaaaatgcacggcatgtcttttgacgtgaagtcatctccatgtgaaaaactgaagacagaga

SEQ ID NO: 441-450 - skipped (10 entries)

SEQ ID NO: 451 - DNA, 120 bp, synthetic construct
  misc_feature 1..120: note = Synthetic polynucleotide
  source 1..120: mol_type = other DNA
aagagcctgatgctacccctccttctccaagaactttactagctatgcaagctgccctgctgggaagtagctcagaagaggagctggagagtgaaaatcgaaggcaggcccgtgggagga

SEQ ID NO: 452 - DNA, 120 bp, synthetic construct
  misc_feature 1..120: note = Synthetic polynucleotide
  source 1..120: mol_type = other DNA
acgcacctgctgctgtagacgaaggctccatatcaccccggactctttcagccattaagagagctcttgacgatgacgaagatgtaaaagtgtgtgctggggatgatgtgcagacgggag

SEQ ID NO: 453 - DNA, 120 bp, synthetic construct
  misc_feature 1..120: note = Synthetic polynucleotide
  source 1..120: mol_type = other DNA
ggccaggagcagaagaaatgcgtataaacagctccaccgagaacagtgatgaaggacttaaagtgagagatggaaaaggaataccgtttactgcaacacttgcgtcatctagtgtgaact

SEQ ID NO: 454 - DNA, 120 bp, synthetic construct
  misc_feature 1..120: note = Synthetic polynucleotide
  source 1..120: mol_type = other DNA
ctgcagaggagcacgtagccagcactaatgaggggagagagcccacagactcagttccaaaagaacaaatgtcacttgttcacgtggggactgaagcctttccgataagtgatgagtcta

SEQ ID NO: 455 - DNA, 120 bp, synthetic construct
  misc_feature 1..120: note = Synthetic polynucleotide
  source 1..120: mol_type = other DNA
tgattaaggacagaaaagatcggctgcctctggagagtgcagtggttagacatagtgacgcacctgggctcccgaatggaagggaactgacaccggcatctccaacttgtacaaattctg

SEQ ID NO: 456 - DNA, 120 bp, synthetic construct
  misc_feature 1..120: note = Synthetic polynucleotide
  source 1..120: mol_type = other DNA
tgtcaaagaatgaaacacatgctgaagtgcttgagcagcagaacgaactttgcccatatgagagtaaattcgattcttctcttctttcaagtgatgatgaaacaaaatgtaaaccgaatt

SEQ ID NO: 457 - DNA, 120 bp, synthetic construct
  misc_feature 1..120: note = Synthetic polynucleotide
  source 1..120: mol_type = other DNA
ctgcttctgaagtcattggccctgtcagtttgcaagaaacaagtagcatagtaagtgtcccttcagaggcagtagataatgtggaaaatgtggtgtcatttaatgctaaagagcatgaga

SEQ ID NO: 458 - DNA, 120 bp, synthetic construct
  misc_feature 1..120: note = Synthetic polynucleotide
  source 1..120: mol_type = other DNA
attttctggaaaccatccaagaacagcagaccactgaatctgcaggccaggatttaatttccattccaaaggccgtggaaccaatggaaattgactcggaagaaagtgaatctgatggta

SEQ ID NO: 459 - DNA, 120 bp, synthetic construct
  misc_feature 1..120: note = Synthetic polynucleotide
  source 1..120: mol_type = other DNA
tgcaggatcattttaatgttttgattgtagatgaagtgaccttttaattttggtacaggaagtttcattgaagtgcaaagtgtgattagtgatgaggaacttcaagcagaattccctgaa

SEQ ID NO: 460 - DNA, 120 bp, synthetic construct
  misc_feature 1..120: note = Synthetic polynucleotide
  source 1..120: mol_type = other DNA
acttccaaacctccctcagaacaaggcgaagaggaactggtaggaactagggagggagaagcccctgctgagtccgagagcctcctgagggacaactctgagagggacgacgtggatggt

SEQ ID NO: 461 - DNA, 120 bp, synthetic construct
  misc_feature 1..120: note = Synthetic polynucleotide
  source 1..120: mol_type = other DNA
gagccacaggaagctgagaaagatgcggaagattcgctccatgaatggcaagatattaatttggtaataccgtaacattgtgtttcgacttcttgctgaggaagccaggttaagtaggtt

SEQ ID NO: 462 - DNA, 120 bp, synthetic construct
  misc_feature 1..120: note = Synthetic polynucleotide
  source 1..120: mol_type = other DNA
tgtaaacaaaactcatttggattattaatataaatctataaatgaaaaaacattttataggaggagttggaaactctggagagcaacctcttagcacagcagaattcactgaaagctcaa

SEQ ID NO: 463 - DNA, 120 bp, synthetic construct
  misc_feature 1..120: note = Synthetic polynucleotide
  source 1..120: mol_type = other DNA
aaacagcagcaagaacggatcgctgctactgtcaccggacagatgttcctggaaagccaggtgggtgcaggcagcttgggtttcctttaccaccttcttcagacccctgggggaatgcac

SEQ ID NO: 464 - DNA, 120 bp, synthetic construct
  misc_feature 1..120: note = Synthetic polynucleotide
  source 1..120: mol_type = other DNA
cctcactctgcaggaactcctgcgcctgttcggcattccctacatccaggctcccatggaagcagaggcgcagtgcgccatcctggacctgactgatcagacttccggaaccatcactga

SEQ ID NO: 465 - DNA, 120 bp, synthetic construct
  misc_feature 1..120: note = Synthetic polynucleotide
  source 1..120: mol_type = other DNA
tgacagtgatatctggctgtttggagcgcggcatgtctatagaaacttttttaataaaaacaagtttgtagaatattatcaatatgtggactttcacaatcaattgggtaagacttcaga

SEQ ID NO: 466 - DNA, 120 bp, synthetic construct
  misc_feature 1..120: note = Synthetic polynucleotide
  source 1..120: mol_type = other DNA
tcatataagaaatcttgataaaaattaaaaaatattgttactctttaggattggaccggaataagttaataaatttggcttatttgcttggaagtgattataccgaaggaataccaactg

SEQ ID NO: 467 - DNA, 120 bp, synthetic construct
  misc_feature 1..120: note = Synthetic polynucleotide
  source 1..120: mol_type = other DNA
tgggttgtgtaaccgccatggaaattctcaatgaattccctgggcatggcctggaacctctcctaaaattctcgtaaggtcttttatttctttaatttggataattgtgtaaatacccaa

SEQ ID NO: 468 - DNA, 120 bp, synthetic construct
  misc_feature 1..120: note = Synthetic polynucleotide
  source 1..120: mol_type = other DNA
ttgtttcctttattttacagagaatggtggcatgaagctcaaaaaaatccaaagataagacctaatcctcatgacaccaaagtgaaaaaaaaattacggacattgcaactcacccctggc

SEQ ID NO: 469 - DNA, 120 bp, synthetic construct
  misc_feature 1..120: note = Synthetic polynucleotide
  source 1..120: mol_type = other DNA
tttcctaacccagctgttgccgaggcctacctcaaacccgtggtggatgactcgaagggatcctttctgtgggggaaacctgatctcgacaaaattagagaatatcctttgcttcttaaa

SEQ ID NO: 470 - DNA, 120 bp, synthetic construct
  misc_feature 1..120: note = Synthetic polynucleotide
  source 1..120: mol_type = other DNA
agtcttaactgcatgcatattttgtcagcggtatttcggctggaacagaacgaagacagatgaatctctgtttcctgtattaaagcaactcgatgcccagcaggtaatcatggtggaccc

SEQ ID NO: 471 - DNA, 120 bp, synthetic construct
  misc_feature 1..120: note = Synthetic polynucleotide
  source 1..120: mol_type = other DNA
agacacagctccgaattgattccttctttagattagcacaacaggagaaagaagatgctaaacgtattaagagccagagactaaacagagctgtgacatgtatgctaaggaaagagaaag

SEQ ID NO: 472 - DNA, 120 bp, synthetic construct
  misc_feature 1..120: note = Synthetic polynucleotide
  source 1..120: mol_type = other DNA
aagcagcagccagcgaaatagaagcagtttctgttgccatggagaaagaatttgagctacttgataaggcaaaaggaaaaacccagaagagaggcataacaaataccttagaagagtcat

SEQ ID NO: 473 - DNA, 120 bp, synthetic construct
  misc_feature 1..120: note = Synthetic polynucleotide
  source 1..120: mol_type = other DNA
caagcctgaaaagaaagaggctttcagattctaaaggaaagaatacatgcggtggatttttgggggagacctgcctctcagaatcatctgatggatcttcaagtgaagatgctgaaagtt

SEQ ID NO: 474 - DNA, 120 bp, synthetic construct
  misc_feature 1..120: note = Synthetic polynucleotide
  source 1..120: mol_type = other DNA
catctttaatgaatgtacaaaggagaacagctgcgaaagagccaaaaaccagtgcttcagattcgcagaactcagtgaaggaagctcccgtgaagaatggaggtgcgaccaccagcagct

SEQ ID NO: 475 - DNA, 120 bp, synthetic construct
  misc_feature 1..120: note = Synthetic polynucleotide
  source 1..120: mol_type = other DNA
ctagtgatagtgatgacgatggagggaaagagaagatggtcctcgtgaccgccagatctgtgtttgggaagaaaagaaggaaactaagacgtgcgaggggaagaaaaaggaaaacctaat

SEQ ID NO: 476 - DNA, 120 bp, synthetic construct
  misc_feature 1..120: note = Synthetic polynucleotide
  source 1..120: mol_type = other DNA
tttttttccttttaggtagtctgtagagaatgccaaatgagggaatcccccactcaagtcaaactcaggagcaagactgtttacagagtcaacctgtcagtaataatgaagaaatggcaa

SEQ ID NO: 477 - DNA, 120 bp, synthetic construct
  misc_feature 1..120: note = Synthetic polynucleotide
  source 1..120: mol_type = other DNA
tcaagcaagaaagtggtggtgatggggaggtggaggagtacctctcctttcgttctgtgggtgacgggctgtccacctctgctgtggggtgcgcatcagcagctccgaggagagggccag

SEQ ID NO: 478 - DNA, 120 bp, synthetic construct
  misc_feature 1..120: note = Synthetic polynucleotide
  source 1..120: mol_type = other DNA
ccctgctgcacatcgaccgacatcagatccaggcagtagagcctagcgcccaggcccttgagctgcagggtttgggtgtggacgtctatgaccaggacgtgctggaacagggagtgcttc

SEQ ID NO: 479 - DNA, 120 bp, synthetic construct
  misc_feature 1..120: note = Synthetic polynucleotide
  source 1..120: mol_type = other DNA
agcaggtggacaatgccatccatgaggccagccgtgcctcccagctcgttgacgtggagaaggagtatcggtcggtcctggatgacctcacgtgagtgcagcccatcttcttcctttcaa

SEQ ID NO: 480 - DNA, 120 bp, synthetic construct
  misc_feature 1..120: note = Synthetic polynucleotide
  source 1..120: mol_type = other DNA
tatattatgctataaaaatgttatcagagtgcaaaatagacaatttatcttatttttcaggtcatgtacgacatccctaaggcaaatcaataaaattattgaacagcttagccctcaagc

SEQ ID NO: 481 - DNA, 120 bp, synthetic construct
  misc_feature 1..120: note = Synthetic polynucleotide
  source 1..120: mol_type = other DNA
tgccaccagcagagacatcaacaggaaactagattctgtaaaacgacagaagtataataaggtgattcagaataacattcagtattgcatttttgaaaaagtcatcatttaggggcactt

SEQ ID NO: 482 - DNA, 120 bp, synthetic construct
  misc_feature 1..120: note = Synthetic polynucleotide
  source 1..120: mol_type = other DNA
ttcaaggaacaacagctaaaaaagatcactgcaaaacaaaagcatctccaggccatccttggaggagcagaggtgaaaattgaactagatcacgccagtctggaggaggatgcaggtgag

SEQ ID NO: 483 - DNA, 120 bp, synthetic construct
  misc_feature 1..120: note = Synthetic polynucleotide
  source 1..120: mol_type = other DNA
ggtggagggggcagaggcggacctgtctggagatggtactgactatgagctgaagcctctgcccaagggcgggaaacggcagaagaaagtgccagtgcaggagattgatgatgacttttt

SEQ ID NO: 484 - DNA, 120 bp, synthetic construct
  misc_feature 1..120: note = Synthetic polynucleotide
  source 1..120: mol_type = other DNA
cccaagttctggggaagaagctgaagctgcttctgtaggagaaggaggaggaggaggtcggaaagtgggaagataccgagatgatggagatgaagattattataagcagcggttaaggtc

SEQ ID NO: 485 - DNA, 120 bp, synthetic construct
  misc_feature 1..120: note = Synthetic polynucleotide
  source 1..120: mol_type = other DNA
atttgtagctgcctaatatttcttgaaagagtgagcatttcttttgtaccctcggaaagtttcatgctagtgcgaaacgcattgctattgttctttcagagccggggccatccagtcttg

SEQ ID NO: 486 - DNA, 120 bp, synthetic construct
  misc_feature 1..120: note = Synthetic polynucleotide
  source 1..120: mol_type = other DNA
gcagcatgctcatgcctgtccaggagactgcctgggaagagctcatccgcactggccagatgacaccttttggtacccagatccctcagaaacaggagaaaaagcccagaaaaatcatgc

SEQ ID NO: 487 - DNA, 120 bp, synthetic construct
  misc_feature 1..120: note = Synthetic polynucleotide
  source 1..120: mol_type = other DNA
ttaatgaagcatcaggcttcgaaaagtatttggcagatcaagcaaaactgtcttttgaaaggaagaagcaaggttgtaataaaagagcagctagaaaagctccagccccagtcacgcctc

SEQ ID NO: 488 - DNA, 120 bp, synthetic construct
  misc_feature 1..120: note = Synthetic polynucleotide
  source 1..120: mol_type = other DNA
cagccccagtgcaaaataaaaacaaaccaaacaagaaagccagagttctgtccaaaaaagaggagcgtttgaaaaagcacatcaagaaactccagaagagggctttgcagttccagggga

SEQ ID NO: 489 - DNA, 119 bp, synthetic construct
  misc_feature 1..119: note = Synthetic polynucleotide
  source 1..119: mol_type = other DNA
aagtgggattgccaaaggcaaggagaccttgggagtcagacatgaggccagaggcagagggagactctgagggtgaagagtctgagtatttccccacagaggaggaggaagaggaggaa

SEQ ID NO: 490 - DNA, 120 bp, synthetic construct
  misc_feature 1..120: note = Synthetic polynucleotide
  source 1..120: mol_type = other DNA
tggaacactaggcaaaaccactaccactaggactaataataccttttttccgttttagtcccaagatgcctcgaacactaagtttacatgaaataactgaccttttagagacagatgaca

SEQ ID NO: 491 - DNA, 120 bp, synthetic construct
  misc_feature 1..120: note = Synthetic polynucleotide
  source 1..120: mol_type = other DNA
ttacagaggcacaccctggacaataccattttgtattcaataactttttcaccagtattgcacttcttgataagctcagttcaatgggacatcaggcaacaggtacagtgagaaaggatc

SEQ ID NO: 492 - DNA, 120 bp, synthetic construct
  misc_feature 1..120: note = Synthetic polynucleotide
  source 1..120: mol_type = other DNA
acattgacagagttccactggaatcagatgtagctttaaagaaaaaagaaagaggcacatttgattatcgaattgatggcaaaggcaatattgtctgcagatggaatgataacagtgttg

SEQ ID NO: 493 - DNA, 120 bp, synthetic construct
  misc_feature 1..120: note = Synthetic polynucleotide
  source 1..120: mol_type = other DNA
tcactgttgcctcatctggtgctggtatccatcccctgtgtcttgtcagtcgttactcccagaaactgaaaaagaagatacaagttcagcagccaaacatgatcaaagtgtataaccagt

SEQ ID NO: 494 - DNA, 120 bp, synthetic construct
  misc_feature 1..120: note = Synthetic polynucleotide
  source 1..120: mol_type = other DNA
tcatgggaggcgtagacagagctgatgaaaacattgataagtatcgggcatcaatccgtggaaagaaatggtattcaagccctcttttgttctgtttcgaactggtcttacaaaatgctt

SEQ ID NO: 495 - DNA, 120 bp, synthetic construct
  misc_feature 1..120: note = Synthetic polynucleotide
  source 1..120: mol_type = other DNA
ggcaattgcataaaacatatgatgagaaaccagtggattttctggagtttcgtcgacgtgtggtatgccattatctggagacccatggtcatcctccagaacctggccaaaaaggaagac

SEQ ID NO: 496 - DNA, 120 bp, synthetic construct
  misc_feature 1..120: note = Synthetic polynucleotide
  source 1..120: mol_type = other DNA
ctcagaagcgtaacattgactcacgttatgatggcataaatcatgtgatagtcaaacagggaaagcaaacgcgatgcgctgaatgtcataagaacacaacttttcgatgtgaaaaatgtg

SEQ ID NO: 497 - DNA, 120 bp, synthetic construct
  misc_feature 1..120: note = Synthetic polynucleotide
  source 1..120: mol_type = other DNA
atgttgccttacatgtgaagtgttccgttgaatatcacactgaatagcaggtgtcaccacctcctgagataagaaacatagttttatacattatgtacagtgtagcagtggttttgccta

SEQ ID NO: 498 - DNA, 120 bp, synthetic construct
  misc_feature 1..120: note = Synthetic polynucleotide
  source 1..120: mol_type = other DNA
gtgttccaattttggaacgtcacataacaatggaacataataaatttttttttctcttcaaatttttgttccttgaatttttctaggtaacatatatgaatttcatgcaaaaattcaaaa

SEQ ID NO: 499 - DNA, 120 bp, synthetic construct
  misc_feature 1..120: note = Synthetic polynucleotide
  source 1..120: mol_type = other DNA
gcatagaagcaagtgctatagtgatacaaccacctgaaaatgctacagcacctgtttctgatgaggaatcaggagatgaagaaggtggaacaataaataatctgccaggttctttgttgc

SEQ ID NO: 500 - DNA, 120 bp, synthetic construct
  misc_feature 1..120: note = Synthetic polynucleotide
  source 1..120: mol_type = other DNA
acacagctgcgtatcttattcaagatggctctgatgctgagtctgactcagatgatccctcatacgcacctaaagatgactctcctgatgaagttccatctacgtttactgtgcagcaac

SEQ ID NO: 501 - DNA, 120 bp, synthetic construct
  misc_feature 1..120: note = Synthetic polynucleotide
  source 1..120: mol_type = other DNA
ctccaccatcaaggaggaggaaaatgacaaaaattctttgcaaatggaaaaaagccgacctaactgtacaacccgtagcaggtagagttacagcaccaccaaacgatttcttcaccgtaa

SEQ ID NO: 502 - DNA, 120 bp, synthetic construct
  misc_feature 1..120: note = Synthetic polynucleotide
  source 1..120: mol_type = other DNA
tgagaactcccacagaaattcttgaactttttcttgatgacgaggtcattgaactcattgtcaagtactccaacttatatgcttgcagtaaaggtgtacatcttggcttgactagctctg

SEQ ID NO: 503 - DNA, 120 bp, synthetic construct
  misc_feature 1..120: note = Synthetic polynucleotide
  source 1..120: mol_type = other DNA
aattcaaatgttttctgggaattatttttctgagtggttatgtctcagttcctagaaggcgtatgttttgggaacaaagaacagatgtgcataatgtactggttagtgctgccatgagac

SEQ ID NO: 504 - DNA, 120 bp, synthetic construct
  misc_feature 1..120: note = Synthetic polynucleotide
  source 1..120: mol_type = other DNA
gtgaccggtttgaaactatattttctaatttgcatgttgctgacaatgcaaatttggatccagtggacaaattttccaaattgcgacctctcataagcaaacttaatgagagatgcatga

SEQ ID NO: 505 - DNA, 120 bp, synthetic construct
  misc_feature 1..120: note = Synthetic polynucleotide
  source 1..120: mol_type = other DNA
aatttgttccaaatgaaacatatttcagctttgatgaattcatggttccttattttggtcgtcacgggtgcaaacaatttattcggggaaagcccattcggtttggctataagttttggt

SEQ ID NO: 506 - DNA, 120 bp, synthetic construct
  misc_feature 1..120: note = Synthetic polynucleotide
  source 1..120: mol_type = other DNA
gtggtgccacctgtctgggctacatttgctggtttcagccgtatcagggtaaaaacccaaatactaaacatgaggaatatggtgtcggtgcgtcacttgtccttcagtttagtgaggcac

SEQ ID NO: 507 - DNA, 120 bp, synthetic construct
  misc_feature 1..120: note = Synthetic polynucleotide
  source 1..120: mol_type = other DNA
tttaaatctgtcttgtgatcaaaataatggaaatgtgatttttattttcatggtaggagatggaataaactgagactgcaggacaaagagaaacgtctgaagctggaggacgattctgag

SEQ ID NO: 508 - DNA, 120 bp, synthetic construct
  misc_feature 1..120: note = Synthetic polynucleotide
  source 1..120: mol_type = other DNA
gaaagtgatgctgaatttgacgaaggttttaaagtgccaggttttctgttcaaaaagctttttaagtatgtaccatatgttctttcctttctatttgctatcaagccattatgtacaaat

SEQ ID NO: 509 - DNA, 120 bp, synthetic construct
  misc_feature 1..120: note = Synthetic polynucleotide
  source 1..120: mol_type = other DNA
ttgccattttctcttttcttgttggtgtttgttgtcataggtaccagcagacaggtgttaggtggctgtgggaattgcactgccagcaggcaggaggaattctgggagatgaaatgggat

SEQ ID NO: 510 - DNA, 120 bp, synthetic construct
  misc_feature 1..120: note = Synthetic polynucleotide
  source 1..120: mol_type = other DNA
tgggcaagaccatccagataattgccttcttggcaggtctgagctacagcaagatcaggactcgtggttcaaattacaggcaagtgctcctctgcagactgtcagtctgtggagctcaat

SEQ ID NO: 511 - DNA, 120 bp, synthetic construct
  misc_feature 1..120: note = Synthetic polynucleotide
  source 1..120: mol_type = other DNA
tgactgtatatgctctttcatggttgttttcttcttggacgtttggatgcaggtttgaggggttgggtccaactgtaattgtctgtccaacaacagtgatgcatcagtgggtgaaggaat

SEQ ID NO: 512 - DNA, 120 bp, synthetic construct
  misc_feature 1..120: note = Synthetic polynucleotide
  source 1..120: mol_type = other DNA
ttcacacgtggtggcctccgttcagagtggcaattctacatgaaaccggttcctatacccacaaaaaggtaacacaatatttcagtacctttttgttttgttttaatgccctccatttta

SEQ ID NO: 513 - DNA, 120 bp, synthetic construct
  misc_feature 1..120: note = Synthetic polynucleotide
  source 1..120: mol_type = other DNA
taaaactgactttaccattttattgtggtctcaggagaaactaattcgagatgttgctcattgtcatggaattttgatcacatcttactcctacattcgattgatgcaggatgacattag

SEQ ID NO: 514 - DNA, 120 bp, synthetic construct
  misc_feature 1..120: note = Synthetic polynucleotide
  source 1..120: mol_type = other DNA
caggtatgactggcactatgtgatcttggacgaaggacacaaaattcgaaatccaaatgctgctgtcacccttgcttgcaaacaggtatgacctcttttaacaagggagatttccaagtg

SEQ ID NO: 515 - DNA, 120 bp, synthetic construct
  misc_feature 1..120: note = Synthetic polynucleotide
  source 1..120: mol_type = other DNA
ttaataaaaaggtgcttcctttcctcaaatagtttcgcacccctcatcggatcattctgtctggctcaccgatgcaaaataacctccgagagctgtggtcgctctttgacttcatcttcc

SEQ ID NO: 516 - DNA, 120 bp, synthetic construct
  misc_feature 1..120: note = Synthetic polynucleotide
  source 1..120: mol_type = other DNA
cgggaaagttaggcacgttgcctgtgtttatggagcagttctccgtccccatcaccatggggggatattcaaatgcttccccagtacaggtaaaatattaggatgataatacttttggca

SEQ ID NO: 517 - DNA, 120 bp, synthetic construct
  misc_feature 1..120: note = Synthetic polynucleotide
  source 1..120: mol_type = other DNA
aggtcaaaactgcttacaagtgtgcatgtgtcttacgagataccataaatccatacctactgcggagaatgaagtcagatgtcaagatgagcctttctttgccagataaaaatgaacagg

SEQ ID NO: 518 - DNA, 120 bp, synthetic construct
  misc_feature 1..120: note = Synthetic polynucleotide
  source 1..120: mol_type = other DNA
tcccgtccgtaggtcttattttgccgtcttacagatgagcagcataaagtctaccaaaatttcgttgattccaaagaagtttacaggattctcaatggagagatgcaggtcagctaaaaa

SEQ ID NO: 519 - DNA, 120 bp, synthetic construct
  misc_feature 1..120: note = Synthetic polynucleotide
  source 1..120: mol_type = other DNA
ttcttaccacagattttctccggacttatagccctaagaaaaatttgcaaccaccctgatctcttttctggaggtcccaagaatctcaaaggtcttcctgatgatgaactagaagaagat

SEQ ID NO: 520 - DNA, 120 bp, synthetic construct
  misc_feature 1..120: note = Synthetic polynucleotide
  source 1..120: mol_type = other DNA
cagtttgggtactggaaacgttctgggaaaatgattgttgttgagtctttgttgaaaatatggcacaagcagggtcagcgagtattgctgttttctcagtcaaggcaggtgagtgcacag

SEQ ID NO: 521 - DNA, 120 bp, synthetic construct
  misc_feature 1..120: note = Synthetic polynucleotide
  source 1..120: mol_type = other DNA
acagatgctggacatacttgaagtattccttagagcccaaaagtatacctatctcaagatggatggtaccactacaatagcttcaagacagccactgattacgagatacaatgaggtaac

SEQ ID NO: 522 - DNA, 120 bp, synthetic construct
  misc_feature 1..120: note = Synthetic polynucleotide
  source 1..120: mol_type = other DNA
ggctgagaactgtaactggtcttaagtgtgtgtgctcagtgttgtgtgtcttacctctaggacacatccatatttgtgtttcttctgaccacgcgggtgggcggcttaggtgtcaacctg

SEQ ID NO: 523 - DNA, 120 bp, synthetic construct
  misc_feature 1..120: note = Synthetic polynucleotide
  source 1..120: mol_type = other DNA
acgggggcaaacagagttgtcatctatgacccagactggaacccaagcacggacacgcaggtttgtttttattttttttttaaaagaatgtattagtagaaatatagtttcatggttagc

SEQ ID NO: 524 - DNA, 120 bp, synthetic construct
  misc_feature 1..120: note = Synthetic polynucleotide
  source 1..120: mol_type = other DNA
tctctgttgcaggcccgggagcgagcatggagaataggccagaagaagcaagtgactgtgtacaggctcctgactgcgggcaccattgaagaaaagatctaccaccggtcagtgcacaca

SEQ ID NO: 525 - DNA, 120 bp, synthetic construct
  misc_feature 1..120: note = Synthetic polynucleotide
  source 1..120: mol_type = other DNA
gtagagctacacattgttttataccagcttatcttttatttttttagacaaatcttcaagcagtttttgacaaatagagtgctaaaagacccaaaacaaaggcggtttttcaaatccaat

SEQ ID NO: 526 - DNA, 120 bp, synthetic construct
  misc_feature 1..120: note = Synthetic polynucleotide
  source 1..120: mol_type = other DNA
gatctctatgagctatttactctgactagtcctgatgcatcccagagcactgaaacaagtgcaatttttgcaggtattacataaaatcatttaatttaaagtaatcaattgcacaagatg

SEQ ID NO: 527 - DNA, 120 bp, synthetic construct
  misc_feature 1..120: note = Synthetic polynucleotide
  source 1..120: mol_type = other DNA
ttacaggaactggatcagatgttcagacacccaaatgccatctaaaaagaaggattcaaccagcctttggagcagaccatgatgttccaaaacgcaagaagttccctgcttctaacatat

SEQ ID NO: 528 - DNA, 120 bp, synthetic construct
  misc_feature 1..120: note = Synthetic polynucleotide
  source 1..120: mol_type = other DNA
ctgtaaatgatgccacatcatctgaagagaaatctgaggctaaaggagctgaagtaaatgcagtaacttctaatcgaagtgatcctttgaaagatgaccctcacatgagtagtaatgtaa

SEQ ID NO: 529 - DNA, 120 bp, synthetic construct
  misc_feature 1..120: note = Synthetic polynucleotide
  source 1..120: mol_type = other DNA
ctagcaatgataggcttggagaagagacaaatgcagtatctggaccagaagagttgtcagtgattagtggaaatggggaatgttcaaattcttcaggaacaggcaaaacttctatgccat

SEQ ID NO: 530 - DNA, 120 bp, synthetic construct
  misc_feature 1..120: note = Synthetic polynucleotide
  source 1..120: mol_type = other DNA
ctggtgatgaaagcattgatgaaaagttaggtctttcttacaaaagagaaagacccagccaggctcaaacagaagctttttgggagaataaacaaatggaaaataatttttataagcaca

SEQ ID NO: 531 - DNA, 120 bp, synthetic construct
  misc_feature 1..120: note = Synthetic polynucleotide
  source 1..120: mol_type = other DNA
agtcaaaaacaaaacatcatagtgtggcagaagaagagaccctggagaaacatctgagaccaaagcaaaagcctaagaactctaagcattgcagagacgccaagtttgaaggaactcgaa

SEQ ID NO: 532 - DNA, 120 bp, synthetic construct
  misc_feature 1..120: note = Synthetic polynucleotide
  source 1..120: mol_type = other DNA
ttccacacctggtgaagaaaaggcgttaccagaagcaagacagtgaaaacaagagtgaggccaaggaacagagcaatgacgattatgttttggaaaagcttttcaaaaaatcaggtaatc

SEQ ID NO: 533 - DNA, 120 bp, synthetic construct
  misc_feature 1..120: note = Synthetic polynucleotide
  source 1..120: mol_type = other DNA
tatttcttttcttgctagttggcgtgcacagtgtcatgaagcacgatgccatcatggatggagccagcccagattatgtactggtggaggcagaagccaaccgagtggcccaggatgccc

SEQ ID NO: 534 - DNA, 120 bp, synthetic construct
  misc_feature 1..120: note = Synthetic polynucleotide
  source 1..120: mol_type = other DNA
tgaaagcactgaggctctctcgtcagcggtgtctgggagcagtgtctggtgttcccacctggactggccacagggggatttctggtgcaccagcaggaaaaaagtaagagattgctgcat

SEQ ID NO: 535 - DNA, 120 bp, synthetic construct
  misc_feature 1..120: note = Synthetic polynucleotide
  source 1..120: mol_type = other DNA
tgatgcttttccttttttaggagtagatttggtaagaaaaggaattctaacttctctgtgcagcatccttcatcaacatctccaacagagaagtgccaggtaatatagataacctttttg

SEQ ID NO: 536 - DNA, 120 bp, synthetic construct
  misc_feature 1..120: note = Synthetic polynucleotide
  source 1..120: mol_type = other DNA
tatatcagtatagtggtcttctttttataggatggcatcatgaaaaaggagggaaaagataatgtccctgagcattttagtggaagagcagaagatgcagactcttcatccgggcccctc

SEQ ID NO: 537 - DNA, 120 bp, synthetic construct
  misc_feature 1..120: note = Synthetic polynucleotide
  source 1..120: mol_type = other DNA
gcttcctcctcactcttggctaaaatgagagctagaaaccacctgattctgccagagcgtttagaaagtgaaagcgggcacctgcaggaagcttctgccctgctgcccaccacagaacac

SEQ ID NO: 538 - DNA, 120 bp, synthetic construct
  misc_feature 1..120: note = Synthetic polynucleotide
  source 1..120: mol_type = other DNA
gatgaccttctggtggagatgagaaacttcatcgctttccaggcccacactgatggccaggccagcaccagggagatactgcaggagtttgaatccaagttatctgcatcacagtcttgt

SEQ ID NO: 539 - DNA, 120 bp, synthetic construct
  misc_feature 1..120: note = Synthetic polynucleotide
  source 1..120: mol_type = other DNA
gtcttccgagaactattgagaaatctgtgcactttccatagaacttctggtggtgaaggaatttggaaactcaagccagaatactgctaaacaacattgcttcctaaactttcaagtccc

SEQ ID NO: 540 - DNA, 120 bp, synthetic construct
  misc_feature 1..120: note = Synthetic polynucleotide
  source 1..120: mol_type = other DNA
ggtaactggaacccaatccgagggtcatggaggcatcccgaaggtttccggaagccgaggccttgagcccagagcaggctgctcattacctaaggtatgtctggaccgctgggcgggtct

SEQ ID NO: 541 - DNA, 120 bp, synthetic construct
  misc_feature 1..120: note = Synthetic polynucleotide
  source 1..120: mol_type = other DNA
tctttttttttctatagatatgtgaaagaggccaaagaagcaactaagaatggagacctggaagaagcatttaaacttttcaatttggcaaaggacatttttcccaatgaaaaagtgctg

SEQ ID NO: 542 - DNA, 120 bp, synthetic construct
  misc_feature 1..120: note = Synthetic polynucleotide
  source 1..120: mol_type = other DNA
gaagaaatatacaggaaatttgtgtctttagatcatatcaaggagttgctaatggagacgcgctcacctttggctgagctaggtgtcttaaagaagctgtgtgatcatcctaggctgctg

SEQ ID NO: 543 - DNA, 120 bp, synthetic construct
  misc_feature 1..120: note = Synthetic polynucleotide
  source 1..120: mol_type = other DNA
tctgcacgggcttgttgtttgctaaatcttgggacattctctgctcaagatggaaatgagggggaagattccccagatgtggaccatattgatcaagtaactgatgacacattgatggaa

SEQ ID NO: 544 - DNA, 120 bp, synthetic construct
  misc_feature 1..120: note = Synthetic polynucleotide
  source 1..120: mol_type = other DNA
gaatctggaaaaatgatattcctaatggacctacttaagaggctgcgagatgagggacatcaaactctggtgttttctcaatcgaggcaaattctaaacatcattgaacgcctcttaaag

SEQ ID NO: 545 - DNA, 120 bp, synthetic construct
  misc_feature 1..120: note = Synthetic polynucleotide
  source 1..120: mol_type = other DNA
aataggcactttaagacattgcgaatcgatgggacagttactcatcttttggaacgagaaaaaagaattaacttattccagcaaaataaagattactctgtttttctgcttaccactcaa

SEQ ID NO: 546 - DNA, 120 bp, synthetic construct
  misc_feature 1..120: note = Synthetic polynucleotide
  source 1..120: mol_type = other DNA
gtaggtggtgtcggtttaacattaactgcagcaactagagtggtcatttttgaccctagctggaatcctgcaactgatgctcaagctgtggatagagtttaccgaattggacaaaaagag

SEQ ID NO: 547 - DNA, 120 bp, synthetic construct
  misc_feature 1..120: note = Synthetic polynucleotide
  source 1..120: mol_type = other DNA
aatgttgtggtttataggctaatcacttgtgggactgtagaggaaaaaatatacagaagacaggttttcaaggactcattaataagacaaactactggtgaaaaaaagaaccctttccga

SEQ ID NO: 548 - DNA, 120 bp, synthetic construct
  misc_feature 1..120: note = Synthetic polynucleotide
  source 1..120: mol_type = other DNA
tattttagtaaacaagaattaagagagctctttacaatcgaggatcttcagaactctgtaacccagctgcagcttcagtctttgcatgctgctcagaggaaatctgatataaaactagat

SEQ ID NO: 549 - DNA, 120 bp, synthetic construct
  misc_feature 1..120: note = Synthetic polynucleotide
  source 1..120: mol_type = other DNA
gaacatattgcctacctgcagtctttggggatagctggaatctcagaccatgatttgatgtacacatgtgatctgtctgttaaagaagagcttgatgtggtagaagaatctcactatatt

SEQ ID NO: 550 - DNA, 120 bp, synthetic construct
  misc_feature 1..120: note = Synthetic polynucleotide
  source 1..120: mol_type = other DNA
caacaaagggttcagaaagctcaattcctcgttgaattcgagtctcaaaataaagagttcctgatggaacaacaaagaactagaaatgagggggcctggctaagagaacctgtatttcct

SEQ ID NO: 551 - DNA, 120 bp, synthetic construct
  misc_feature 1..120: note = Synthetic polynucleotide
  source 1..120: mol_type = other DNA
tcttcaacaaagaagaaatgccctaaattgaataaaccacagcctcagccttcacctcttctaagtactcatcatactcaggaagaagatatcagttccaaaatggcaagtgtagtcatt

SEQ ID NO: 552 - DNA, 120 bp, synthetic construct
  misc_feature 1..120: note = Synthetic polynucleotide
  source 1..120: mol_type = other DNA
agcagaatccaaaaaatacaggaagccttggaggagttggcagaacagggagatgatgaatttacagatgtgtgcaactctggcttgctactttatcgagaactgcacaaccaactcttt

SEQ ID NO: 553 - DNA, 120 bp, synthetic construct
  misc_feature 1..120: note = Synthetic polynucleotide
  source 1..120: mol_type = other DNA
gatgatctgcccaaagagggtgagaaacaagatctctccagtataaaggtgaatgttaccaccttgcaagatggtaaaggtacaggtagtgctgactctatagctactttaccaaagggg

SEQ ID NO: 554 - DNA, 120 bp, synthetic construct
  misc_feature 1..120: note = Synthetic polynucleotide
  source 1..120: mol_type = other DNA
tttggaagtgtagaagaactttgtactaactcttcattgggaatggaaaaaagctttgcaactaaaaatgaagctgtacaaaaagagacattacaagaggggcctaagcaagaggcactg

SEQ ID NO: 555 - DNA, 120 bp, synthetic construct
  misc_feature 1..120: note = Synthetic polynucleotide
  source 1..120: mol_type = other DNA
caagaggatcctctggaaagttttaattatgtacttagcaaatcaaccaaagctgatattgggccaaatttagatcaactaaaggatgatgagattttacgtcattgcaatccttggccc

SEQ ID NO: 556 - DNA, 120 bp, synthetic construct
  misc_feature 1..120: note = Synthetic polynucleotide
  source 1..120: mol_type = other DNA
attatttccataacaaatgaaagtcaaaatgcagaatcaaatgtatccattattgaaatagctgatgacctttcagcatcccatagtgcactgcaggatgctcaagcaagtgaggccaag

SEQ ID NO: 557 - DNA, 120 bp, synthetic construct
  misc_feature 1..120: note = Synthetic polynucleotide
  source 1..120: mol_type = other DNA
ttggaagaggaaccttcagcatcttcaccacagtatgcatgtgatttcaatcttttcttggaagactcagcagacaacagacaaaatttttccagtcagtctttagagcatgttgagaaa

SEQ ID NO: 558 - DNA, 120 bp, synthetic construct
  misc_feature 1..120: note = Synthetic polynucleotide
  source 1..120: mol_type = other DNA
gaaaatagcttgtgtggctctgcacctaattccagagcagggtttgtgcatagcaaaacatgtctcagttgggagttttctgagaaagacgatgaaccagaagaagtagtagttaaagca

SEQ ID NO: 559 - DNA, 120 bp, synthetic construct
  misc_feature 1..120: note = Synthetic polynucleotide
  source 1..120: mol_type = other DNA
aaaatcagaagtaaagctagaaggattgtttcagatggcgaagatgaagatgattcttttaaagatacctcaagcataaatccattcaacacatctctctttcaattctcatctgtgaaa

SEQ ID NO: 560 - DNA, 120 bp, synthetic construct
  misc_feature 1..120: note = Synthetic polynucleotide
  source 1..120: mol_type = other DNA
caatttgatgcttcaactcccaaaaatgacatcagtccaccaggaaggttcttttcatctcaaatacccagtagtgtaaataagtctatgaactctagaagatctctggcttctaggagg

SEQ ID NO: 561 - DNA, 120 bp, synthetic construct
  misc_feature 1..120: note = Synthetic polynucleotide
  source 1..120: mol_type = other DNA
tctcttattaatatggttttagaccacgtggaggacatggaggaaagacttgacgacagcagtgaagcaaagggtcctgaagattatccagaagaaggggtggaggaaagcagtggcgaa

SEQ ID NO: 562 - DNA, 120 bp, synthetic construct
  misc_feature 1..120: note = Synthetic polynucleotide
  source 1..120: mol_type = other DNA
gcctccaagtatacagaagaggatccttccggagaaacactgtcttcagaaaacaagtccagctggttaatgacgtctaagcctagtgctctagctcaagagacctctcttggtgcccct

SEQ ID NO: 563 - DNA, 120 bp, synthetic construct
  misc_feature 1..120: note = Synthetic polynucleotide
  source 1..120: mol_type = other DNA
gagcaccagaaggaaggcatagctttcctctatagcctgtatagggatggaagaaaaggtggtatattggctgatgatatgggattagggaagactgttcaaatcattgctttcctttcc

SEQ ID NO: 564 - DNA, 120 bp, synthetic construct
  misc_feature 1..120: note = Synthetic polynucleotide
  source 1..120: mol_type = other DNA
gagcctttgtctggtgaacagttggttggttctccccaggataaggcggcagaggctacaaatgactatgagactcttgtaaagcgtggaaaagaactaaaagagtgtggaaaaatccag

SEQ ID NO: 565 - DNA, 120 bp, synthetic construct
  misc_feature 1..120: note = Synthetic polynucleotide
  source 1..120: mol_type = other DNA
gaggccctaaactgcttagttaaagcgcttgacataaaaagtgcagatcctgaagttatgctcttgactttaagtttgtataagcaacttaataacaattgagaatgtaacctgtttatt

SEQ ID NO: 566 - DNA, 120 bp, synthetic construct
  misc_feature 1..120: note = Synthetic polynucleotide
  source 1..120: mol_type = other DNA
ggtatgtttgatgcatcacttgtgaatcatgtgctgctgatcatgccaaccaatcttattaacacatgggtaaaagaattcatcaagtggactccaggaatgagagtcaaaacctttcat

SEQ ID NO: 567 - DNA, 120 bp, synthetic construct
  misc_feature 1..120: note = Synthetic polynucleotide
  source 1..120: mol_type = other DNA
ggtcctagcaaggatgaacggaccagaaacctcaatcggattcagcaaaggaatggtgttattatcactacataccaaatgttaatcaataactggcagcaactttcaagctttaggggc

SEQ ID NO: 568 - DNA, 120 bp, synthetic construct
  misc_feature 1..120: note = Synthetic polynucleotide
  source 1..120: mol_type = other DNA
caagagtttgtgtgggactatgtcatcctcgatgaagcacataaaataaaaacctcatctactaagtcagcaatatgtgctcgtgctattcctgcaagtaatcgcctcctcctcacagga

SEQ ID NO: 569 - DNA, 120 bp, synthetic construct
  misc_feature 1..120: note = Synthetic polynucleotide
  source 1..120: mol_type = other DNA
accccaatccagaataatttacaagaactatggtccctatttgattttgcttgtcaagggtccctgctgggaacattaaaaacttttaagatggagtatgaaaatcctattactagagca

SEQ ID NO: 570 - DNA, 120 bp, synthetic construct
  misc_feature 1..120: note = Synthetic polynucleotide
  source 1..120: mol_type = other DNA
agagagaaggatgctaccccaggagaaaaagccttgggatttaaaatatctgaaaacttaatggcaatcataaaaccctattttctcaggaggactaaagaagacgtacagaagaaaaag

SEQ ID NO: 571 - DNA, 120 bp, synthetic construct
  misc_feature 1..120: note = Synthetic polynucleotide
  source 1..120: mol_type = other DNA
tcaagcaacccagaggccagacttaatgaaaagaatccagatgttgatgccatttgtgaaatgccttccctttccaggaaaaatgatttaattatttggatacgacttgtgcctttacaa

SEQ ID NO: 572 - DNA, 120 bp, synthetic construct
  misc_feature 1..120: note = Synthetic polynucleotide
  source 1..120: mol_type = other DNA
cgccgccttccgggtgttacatgcagccgggctcggcccctccccctggccggatggatccgtcggcgccacagccccgcgcggaaacctcaggcaaaggtaccagctccgcgctcgccc

SEQ ID NO: 573 - DNA, 120 bp, synthetic construct
  misc_feature 1..120: note = Synthetic polynucleotide
  source 1..120: mol_type = other DNA
ggaaactttattttctttattcttgcagacatatggcatccaggagaaagatgtcttgccccttctccagataatggaaaactttgtgaagcaagcataaaatctatcacagtggatgaa

SEQ ID NO: 574 - DNA, 120 bp, synthetic construct
  misc_feature 1..120: note = Synthetic polynucleotide
  source 1..120: mol_type = other DNA
aatggcaagtcatttgcagtcgtcttatatgcagattttcaagaaaggaaaatacctcttaaacagcttcaagaagtgaaatttgttaaagattgccctaggaatcttatatttgatgat

SEQ ID NO: 575 - DNA, 120 bp, synthetic construct
  misc_feature 1..120: note = Synthetic polynucleotide
  source 1..120: mol_type = other DNA
gaagatttagaaaaaccttatttcccaaaccgaaaatttccatcatcttctgttgcttttaaattatctgacaatggagactctattccttataccatcaataggtatttgagagactac

SEQ ID NO: 576 - DNA, 120 bp, synthetic construct
  misc_feature 1..120: note = Synthetic polynucleotide
  source 1..120: mol_type = other DNA
caaagagaaggaacccggtttctttatggacactacatccatggaggagggtgcattctgggtgatgacatgggacttggaaaaacagtacaggtatttaattatgttataacagtaaag

SEQ ID NO: 577 - DNA, 120 bp, synthetic construct
  misc_feature 1..120: note = Synthetic polynucleotide
  source 1..120: mol_type = other DNA
aaattttgtacgtcatgatacattatacacagtgattgaaaaagtcttttttccccaggttatttcatttctggctgcagttttgcataaaaagggaactcgtgaggatattgaaaataa

SEQ ID NO: 578 - DNA, 120 bp, synthetic construct
  misc_feature 1..120: note = Synthetic polynucleotide
  source 1..120: mol_type = other DNA
catgccagagtttttactaagaagtatgaaaaaggaacccctttcttctacagcaaaaaaggtaaaatctctagacaatgtatattctactcatgatgctggtattacaaaagcatatag

SEQ ID NO: 579 - DNA, 120 bp, synthetic construct
  misc_feature 1..120: note = Synthetic polynucleotide
  source 1..120: mol_type = other DNA
aaatttttttattcttgttttagatgttcttaatagttgctcctctttctgtcctctacaactggaaggatgaattggacacctggggatatttcagagtcactgttttacatggaaaca

SEQ ID NO: 580 - DNA, 120 bp, synthetic construct
  misc_feature 1..120: note = Synthetic polynucleotide
  source 1..120: mol_type = other DNA
gaaaagataatgaattaattcgtgtaaagcagaggaaatgtgaaattgctctaacaacttatgaaacactacgcttatgcctggatgaacttaacaggtaatgggaataataggaatagg

SEQ ID NO: 581 - DNA, 120 bp, synthetic construct
  misc_feature 1..120: note = Synthetic polynucleotide
  source 1..120: mol_type = other DNA
gttttctcaacctcacccttcttttttcttactttatagtttggaatggtcagctgtcattgtggatgaagctcatagaatcaagaatccaaaagctagagtaacagaagttatgaaagc

SEQ ID NO: 582 - DNA, 120 bp, synthetic construct
  misc_feature 1..120: note = Synthetic polynucleotide
  source 1..120: mol_type = other DNA
tttgaaatgtaatgtccgcattggcctcactggaaccatccttcagaacaacatgaaggaactgtggtgtgttatggactggtgagagaaaacactttttaaaaaattgtttaatagttc

SEQ ID NO: 583 - DNA, 120 bp, synthetic construct
  misc_feature 1..120: note = Synthetic polynucleotide
  source 1..120: mol_type = other DNA
tattgtctttataaagggctgtgccaggccttttagggagtgggacctacttcaagaagcagttttctgacccagtagaacatggtcagagacacacggcaacaaagagagaactagcca

SEQ ID NO: 584 - DNA, 120 bp, synthetic construct
  misc_feature 1..120: note = Synthetic polynucleotide
  source 1..120: mol_type = other DNA
ctggccgaaaggccatgcaaagacttgccaaaaagatgtctggctggtttctcaggcgcaccaagactcttatcaaggatcagttgcctaagaaggaagaccgggtaagaaccgcatttg

SEQ ID NO: 585 - DNA, 120 bp, synthetic construct
  misc_feature 1..120: note = Synthetic polynucleotide
  source 1..120: mol_type = other DNA
tattgttataaacaaaatactaataactaccttgtattttatcttggcagatggtgtattgttctttgacagatttccagaaagctgtctatcaaacagtgttagaaacagaggacgtga

SEQ ID NO: 586 - DNA, 120 bp, synthetic construct
  misc_feature 1..120: note = Synthetic polynucleotide
  source 1..120: mol_type = other DNA
ctttgatacttcaatcttctgagccttgtacctgtaggagtggccaaaaaaggagaaattgttgttataaggcaagcatttcaatatatctttataatcatgcttttgattacatagtac

SEQ ID NO: 587 - DNA, 120 bp, synthetic construct
  misc_feature 1..120: note = Synthetic polynucleotide
  source 1..120: mol_type = other DNA
cagaccaattctcatggtgaaacagtgaaaaccttgtatctcagttaccttacagtccttcagaaggtagctaaccatgtcgcgctactgcaagctgctagtacttccaaacaacaggtt

SEQ ID NO: 588 - DNA, 120 bp, synthetic construct
  misc_feature 1..120: note = Synthetic polynucleotide
  source 1..120: mol_type = other DNA
attatttcaatttatacaggtttgaatgtgttaagtggaaatatcttttcttctgccttttcccttcaaggaaacacttatcaaaaggatatgtgatcaggtattttccagattcccaga

SEQ ID NO: 589 - DNA, 119 bp, synthetic construct
  misc_feature 1..119: note = Synthetic polynucleotide
  source 1..119: mol_type = other DNA
ttttgtgcagaaaagcaaagatgcagcctttgaaacactttctgaccctaaatacagtggaaaaatgaaggtaagtgctcctctttcaggttgcatacagacatgatacacaaaatatt

SEQ ID NO: 590 - DNA, 120 bp, synthetic construct
  misc_feature 1..120: note = Synthetic polynucleotide
  source 1..120: mol_type = other DNA
tgattttctgtggttcattttcaggtccttcagcagcttttaaatcattgcaggaaaaacagagataaagttcttctcttttctttttccaccaaggtgagttcatctaaagtatatcct

SEQ ID NO: 591 - DNA, 120 bp, synthetic construct
  misc_feature 1..120: note = Synthetic polynucleotide
  source 1..120: mol_type = other DNA
tacttaaccaagattcatgtttgcccatcttcatacctctcgtctagttgcttgacgtgctacagcagtactgtatggcgtctgggcttgattaccgacgacttgatggaagtacaaaat

SEQ ID NO: 592 - DNA, 120 bp, synthetic construct
  misc_feature 1..120: note = Synthetic polynucleotide
  source 1..120: mol_type = other DNA
cagaggaaagactcaagattgtaaaagagttcaacagtacacaagatgttaacatttgccttgtctctacaatgtaagaaaattaaatttaataactagatttttatccaattgtttttg

SEQ ID NO: 593 - DNA, 120 bp, synthetic construct
  misc_feature 1..120: note = Synthetic polynucleotide
  source 1..120: mol_type = other DNA
tctttcctccagggctggtggactaggcctcaattttgtcggtgccaatgttgttgtattatttgatcctacttggaatccagccaatgatcttcaagccattgacaggtataatactga

SEQ ID NO: 594 - DNA, 120 bp, synthetic construct
  misc_feature 1..120: note = Synthetic polynucleotide
  source 1..120: mol_type = other DNA
ccttttacagagcatataggattggacaatgtagagatgtcaaagtgcttaggctgatatccttgggaactgtggaggaaatcatgtatttacgacagatatacaagcaggtaaatatgt

SEQ ID NO: 595 - DNA, 120 bp, synthetic construct
  misc_feature 1..120: note = Synthetic polynucleotide
  source 1..120: mol_type = other DNA
gcaaacacttcaaaaatgtcttgtgttttttctgttttagcaacttcactgtgtggtggttggaagtgaaaatgccaaacgatattttgaagcagttcaaggatctaaagagcatcaagg

SEQ ID NO: 596 - DNA, 120 bp, synthetic construct
  misc_feature 1..120: note = Synthetic polynucleotide
  source 1..120: mol_type = other DNA
agagctttttgggatccataacctcttcaaatttaggtcccaagggtcttgtcttacgaaggacatcctggaggtgtgaacttcttctctgaccttttcaataatattttaaatacagtt

SEQ ID NO: 597 - DNA, 120 bp, synthetic construct
  misc_feature 1..120: note = Synthetic polynucleotide
  source 1..120: mol_type = other DNA
ttttcttcatcttttttttctctcttagcaccttctagccaccatggcaacctcatctgaagaagttttgctgattgtaaagaaagtgcgtcaaaagaagcaggatggagctctgtacct

SEQ ID NO: 598 - DNA, 120 bp, synthetic construct
  misc_feature 1..120: note = Synthetic polynucleotide
  source 1..120: mol_type = other DNA
catggcagaaagaattgcttgggcacctgaaggcaaagatagatttacaatcagccatatgtatgcagatattaaatgtaagtcagctatactaagttctgatgtatttgtatgtcatag

SEQ ID NO: 599 - DNA, 120 bp, synthetic construct
  misc_feature 1..120: note = Synthetic polynucleotide
  source 1..120: mol_type = other DNA
aactgccctcatgcttctttttaggccagaaaattagtccagaaggaaaagctaaaattcagcttcagctggtcctacatgcaggggacacaactaacttccatttttccaatgaaagca

SEQ ID NO: 600 - DNA, 120 bp, synthetic construct
  misc_feature 1..120: note = Synthetic polynucleotide
  source 1..120: mol_type = other DNA
cagcagtgaaagagcgagatgcagtaaaagaccttcttcagcagctgctgcccaaattcaagaggaaagcaaataaagaactggaagagaagaacaggtgggaggaaaagaatagccttt

SEQ ID NO: 601 - DNA, 120 bp, synthetic construct
  misc_feature 1..120: note = Synthetic polynucleotide
  source 1..120: mol_type = other DNA
agaagttcagttatattcagtatattctgctttacagaatgctgcaagaagatcctgttttgtttcagctttataaagaccttgttgtgagtcaagtgatcagtgctgaggaattctggg

SEQ ID NO: 602 - DNA, 120 bp, synthetic construct
  misc_feature 1..120: note = Synthetic polynucleotide
  source 1..120: mol_type = other DNA
ccaatcgtttaaatgtgaatgcaacagatagttcttccacatccaatcataagcaggatgttggcatttctgctgcatttctggtatgtgagccttctagatttctgaagaaaataaaaa

SEQ ID NO: 603 - DNA, 120 bp, synthetic construct
  misc_feature 1..120: note = Synthetic polynucleotide
  source 1..120: mol_type = other DNA
ggctttgttttaggctgatgtccggccccaaactgatggctgtaacggtctaagatataatttaacttctgatatcattgagtccatatttaggacctatccagcaggtaagaagaatca

SEQ ID NO: 604 - DNA, 120 bp, synthetic construct
  misc_feature 1..120: note = Synthetic polynucleotide
  source 1..120: mol_type = other DNA
aaaatggaaattcagtatataatatttgtgggtttttttccacagtaaaaatgaaatatgcagaaaatgttccccacaacatgacagagaaggaattctggacacgttttttccagtccc

SEQ ID NO: 605 - DNA, 120 bp, synthetic construct
  misc_feature 1..120: note = Synthetic polynucleotide
  source 1..120: mol_type = other DNA
attattttcacagggatcggctgaatacagggtcaaaggatctctttgcagaatgtgccaaaatagatgaaaaaggtaactgtttatctctgatagacactggtatttaacttgccaccc

SEQ ID NO: 606 - DNA, 120 bp, synthetic construct
  misc_feature 1..120: note = Synthetic polynucleotide
  source 1..120: mol_type = other DNA
ttattttgttgattttctaggcctaaaaacaatggtttcattaggagtgaaaaacccactactagatttaacagctttggaagataaaccattagatgaggtaagaagcaataaaagaag

SEQ ID NO: 607 - DNA, 120 bp, synthetic construct
  misc_feature 1..120: note = Synthetic polynucleotide
  source 1..120: mol_type = other DNA
tataataagaaaagcagaacctttttaaaaaatttttaatctattatttctcacagggctatggcatttcctctgtgccatctgcttccaattctaaatccataaaagagaatagtaatg

SEQ ID NO: 608 - DNA, 120 bp, synthetic construct
  misc_feature 1..120: note = Synthetic polynucleotide
  source 1..120: mol_type = other DNA
ctgccatcatcaagagatttaaccatcacagtgccatggtcctggcagctggactcagaaaacagttaagtataaatgcagaggtgcagtaactgggcttttcaggatatccagatggag

SEQ ID NO: 609 - DNA, 120 bp, synthetic construct
  misc_feature 1..120: note = Synthetic polynucleotide
  source 1..120: mol_type = other DNA
tcccctttattttaagagaagcacaaaatgaacaaactagtgagcccagcaacatggatggaaattccggagatgcagactgctttcagccagcagtcaaaagggtatgggcaaaaaaat

SEQ ID NO: 610 - DNA, 120 bp, synthetic construct
  misc_feature 1..120: note = Synthetic polynucleotide
  source 1..120: mol_type = other DNA
gtgttaataattataggcgaaattacaagagtccattgaatatgaagacttggggaaaaataattctgtaaaaacgattgcactaaacctcaagaagtcagataggtaagtttggtcaat

SEQ ID NO: 611 - DNA, 120 bp, synthetic construct
  misc_feature 1..120: note = Synthetic polynucleotide
  source 1..120: mol_type = other DNA
ggtattatcatggtccaactccaatccagtcactacagtatgcaacaagtcaggacattattaattcttttcaaagtattagacaagaaatggaagcttatacacccaagttaactcagg

SEQ ID NO: 612 - DNA, 120 bp, synthetic construct
  misc_feature 1..120: note = Synthetic polynucleotide
  source 1..120: mol_type = other DNA
ttcatttttttcaggttctctcaagtagtgctgccagtagtaccatcacagcactgtcacctggaggggcacttatgcagggaggaacacagcaagccataaaccgtatgtgccgggcca

SEQ ID NO: 613 - DNA, 120 bp, synthetic construct
  misc_feature 1..120: note = Synthetic polynucleotide
  source 1..120: mol_type = other DNA
agagatggtgccaaatgatattcaatctgaattgaaacacttatatgtagctgttggagaacttctacgacatttctggtcctgctttcctgttaatacgccattcctagaagaaaaggt

SEQ ID NO: 614 - DNA, 120 bp, synthetic construct
  misc_feature 1..120: note = Synthetic polynucleotide
  source 1..120: mol_type = other DNA
ctcatcttttttaggtagtgaaaatgaaaagtaatttggaacgattccaagttacgaagctctgtccattccaagaaaagattcggagacagtatttaagcacaaatgtaaggcagcaat

SEQ ID NO: 615 - DNA, 120 bp, synthetic construct
  misc_feature 1..120: note = Synthetic polynucleotide
  source 1..120: mol_type = other DNA
ttgctgtgtttttcagttggtaagtcacatagaagagatgctccagacagcctacaacaagctccacacatggcagtcacggcgtctgatgaagaaaacgtgaggtggccatgatgctta

SEQ ID NO: 616 - DNA, 120 bp, synthetic construct
  misc_feature 1..120: note = Synthetic polynucleotide
  source 1..120: mol_type = other DNA
ttttacagattattgaataataaaatacagttttgaaaaaaatggatgaagaacctgaaagaactaagcgatgggaaggaggctatgaaagaacatggtaaggagagctttattgccctg

SEQ ID NO: 617 - DNA, 120 bp, synthetic construct
  misc_feature 1..120: note = Synthetic polynucleotide
  source 1..120: mol_type = other DNA
aacaaataattatgacttatagggagattcttaaagaagatgaatctggatcacttaaagctacaatagaagacattctattcaaggcaaagagaaaaaggtatgtaaccttcctatgta

SEQ ID NO: 618 - DNA, 120 bp, synthetic construct
  misc_feature 1..120: note = Synthetic polynucleotide
  source 1..120: mol_type = other DNA
agtatatggaattacattaaaatgtttgtataattttaacagagtatttgagcaccatggacaagttcgacttggaatggtatgtcattattttttcttttactagtacagaactagttt

SEQ ID NO: 619 - DNA, 120 bp, synthetic construct
  misc_feature 1..120: note = Synthetic polynucleotide
  source 1..120: mol_type = other DNA
ttcttttttccgatagatgcgccacctttatgtggtagtagatggatcaagaacaatggaagaccaagatttaaagcctaatagactgacgtgtactttaaaggtaaaatttaagtttat

SEQ ID NO: 620 - DNA, 120 bp, synthetic construct
  misc_feature 1..120: note = Synthetic polynucleotide
  source 1..120: mol_type = other DNA
cctttatctagtatgtctttttttgtttaaacagttgttggaatactttgtagaggaatattttgatcaaaatcctattagtcaggtacgtatctaagtgatagaattcagaattagatt

SEQ ID NO: 621 - DNA, 120 bp, synthetic construct
  misc_feature 1..120: note = Synthetic polynucleotide
  source 1..120: mol_type = other DNA
atattaataataatttttgtttttatttcaagattggaataattgtaactaagagtaaaagagctgaaaaattgactgaactttcaggtatgcataaaattacctttacatgactcaagg

SEQ ID NO: 622 - DNA, 120 bp, synthetic construct
  misc_feature 1..120: note = Synthetic polynucleotide
  source 1..120: mol_type = other DNA
attttaggaaacccaagaaaacatataacgtctttgaagaaagctgtggatatgacctgccatggagagccatctctttataattccctaagcatagctatgcagactctaaagttagta

SEQ ID NO: 623 - DNA, 120 bp, synthetic construct
  misc_feature 1..120: note = Synthetic polynucleotide
  source 1..120: mol_type = other DNA
atctttcttttaagacacatgcctggacatacaagtcgagaagtactaatcatctttagcagccttacaacttgcgatccatctaatatttatgatctaatcaaggtagaccaaaaaatc

SEQ ID NO: 624 - DNA, 120 bp, synthetic construct
  misc_feature 1..120: note = Synthetic polynucleotide
  source 1..120: mol_type = other DNA
cttggattttatgaagaccctaaaggcagctaaaattagagtatctgttattggattgtctgcagaagttcgcgtttgcactgtacttgctcgtgaaactggtggtatatatataattta

SEQ ID NO: 625 - DNA, 120 bp, synthetic construct
  misc_feature 1..120: note = Synthetic polynucleotide
  source 1..120: mol_type = other DNA
tgttaggcacgtaccatgttattttagatgaaagccattacaaagagttgctcacacatcatgttagtcctcctcctgctagctcaagttctgaatgctcacttattcgtatgggtaagt

SEQ ID NO: 626 - DNA, 120 bp, synthetic construct
  misc_feature 1..120: note = Synthetic polynucleotide
  source 1..120: mol_type = other DNA
agaaagtaatacattttcttctatgaaggatttcctcagcacaccattgcttctttatctgaccaggatgcaaaaccctctttcagcatggcgtaagtaaagaccttgaaaatatcagtg

SEQ ID NO: 627 - DNA, 120 bp, synthetic construct
  misc_feature 1..120: note = Synthetic polynucleotide
  source 1..120: mol_type = other DNA
ccctacaggcatttggatggcaatactgagccagggcttacattaggaggctatttctgcccacagtgtcgggcaaagtactgtgagctacctgttgagtgtaaaatctgtggtaagaaa

SEQ ID NO: 628 - DNA, 120 bp, synthetic construct
  misc_feature 1..120: note = Synthetic polynucleotide
  source 1..120: mol_type = other DNA
tgtgttaggtcttactttggtgtctgctccccacttggcacggtcttaccatcatttgtttcctttggatgcttttcaagaaattcccctagaagaatataatggagaaaggtatttcag

SEQ ID NO: 629 - DNA, 120 bp, synthetic construct
  misc_feature 1..120: note = Synthetic polynucleotide
  source 1..120: mol_type = other DNA
gtaaattctaagaaaaaacattgttaaacttttttttcagattttgttatggatgtcagggggaattgaaagaccaacatgtaagttctttggctttctaaatattaagtaatgtacaag

SEQ ID NO: 630 - DNA, 120 bp, synthetic construct
  misc_feature 1..120: note = Synthetic polynucleotide
  source 1..120: mol_type = other DNA
tagctgtgctgtaattaacttttggaaactagtttatcacttttcttcttttcactacaggtttatgtttgtgctgtgtgccaaaatgttttctgtgtggactgtgatgtttttgttcat

SEQ ID NO: 631 - DNA, 120 bp, synthetic construct
  misc_feature 1..120: note = Synthetic polynucleotide
  source 1..120: mol_type = other DNA
gattctctacactgttgccctggctgtattcataagattccagctccttcaggtgtttgattccagcatgtagtatacattgtatgtgttaaaaagaaatttgcaactgtgaataaaagg

SEQ ID NO: 632 - DNA, 120 bp, synthetic construct
  misc_feature 1..120: note = Synthetic polynucleotide
  source 1..120: mol_type = other DNA
tttgagacgctcccctgaccaccacttgctctgcgctgaggtgctgggacagccatggtttcagacggtgaggaccctgcagggcgggacttcgactccggggctcggctgtctcggtcc

SEQ ID NO: 633 - DNA, 120 bp, synthetic construct
  misc_feature 1..120: note = Synthetic polynucleotide
  source 1..120: mol_type = other DNA
tttcttgttaatattttcagaagatgaattgaatcttctggttattgtagttgatgccaacccaatttggtggggaaagcaagcattaaaggaatctcaggtaagactgcttgaggaggc

SEQ ID NO: 634 - DNA, 120 bp, synthetic construct
  misc_feature 1..120: note = Synthetic polynucleotide
  source 1..120: mol_type = other DNA
caacagttcactttatccaaatgcatagatgccgtgatggtgctgggaaattcgcatttattcatgaatcgttccaacaaacttgctgtgatagcaagtcacattcaagaaaggtatgac

SEQ ID NO: 635 - DNA, 120 bp, synthetic construct
  misc_feature 1..120: note = Synthetic polynucleotide
  source 1..120: mol_type = other DNA
ggcctggtgtaaccaggttttttcccctgctgtttcagccgattcttatatcctggaaagaatggcagacttggagacttcttcggagaccctggcaaccctcctgaatttaatccctct

SEQ ID NO: 636 - DNA, 120 bp, synthetic construct
  misc_feature 1..120: note = Synthetic polynucleotide
  source 1..120: mol_type = other DNA
gggagtaaagatggaaaatacgaacttttaacctcagcaaatgaagttattgttgaagagattaaagatctaatgaccaaaagtaacaacttttaaacattgttattttgcaaatagtgt

SEQ ID NO: 637 - DNA, 120 bp, synthetic construct
  misc_feature 1..120: note = Synthetic polynucleotide
  source 1..120: mol_type = other DNA
cccttttaatgttttctgttatttgcaggtgacataaagggtcaacatacagaaactttgctggcaggatccctggccaaagccctttgctgtatccttggtgtctgaatcatttagaag

SEQ ID NO: 638 - DNA, 120 bp, synthetic construct
  misc_feature 1..120: note = Synthetic polynucleotide
  source 1..120: mol_type = other DNA
ttatgaagcaaaataatatttttgtattccttaatgttaagaaattaagacattcatagaatgaacaaggaagttaaaggtaagagcctacgttttcctatgagaactgtaatcctactt

SEQ ID NO: 639 - DNA, 120 bp, synthetic construct
  misc_feature 1..120: note = Synthetic polynucleotide
  source 1..120: mol_type = other DNA
ttagtaagatttagttaaatttttttctgcttttttctctttgtagacaatcaggaaatgaaatcaaggatattggtaagataaaaaaatcattacttttttatatgacaactataatta

SEQ ID NO: 640 - DNA, 120 bp, synthetic construct
  misc_feature 1..120: note = Synthetic polynucleotide
  source 1..120: mol_type = other DNA
ttaacatgactgtgatttttaggtgattaaggctgcagaagacagtgcgttgcagtatatgaacttcatgaatgtcatctttgcagcacagaaacaggtgaactgagagcctgccgttta

SEQ ID NO: 641 - DNA, 120 bp, synthetic construct
  misc_feature 1..120: note = Synthetic polynucleotide
  source 1..120: mol_type = other DNA
attcctgtaatattttcaaaatgtttcttttagaatattttgattgatgcctgtgttttagactccgactcagggctcctccaacaggtatgttttaaaaccatgctttgtgtcggtggt

SEQ ID NO: 642 - DNA, 120 bp, synthetic construct
  misc_feature 1..120: note = Synthetic polynucleotide
  source 1..120: mol_type = other DNA
ggcagcaagccgcctgtgtcttgcaggcttgtgacatcacgggaggactgtacctgaaggtgcctcagatgccttctcttctgcagtatttgctggtaaggagacagcagcggcgaccct

SEQ ID NO: 643 - DNA, 120 bp, synthetic construct
  misc_feature 1..120: note = Synthetic polynucleotide
  source 1..120: mol_type = other DNA
atttttttcccatgttttaaataaaagtttcttttgtttgtttgttttacagtgggtgtttcttcccgatcaagatcagagatctcagttaatcctcccacccccagttcatgttgacta

SEQ ID NO: 644 - DNA, 120 bp, synthetic construct
  misc_feature 1..120: note = Synthetic polynucleotide
  source 1..120: mol_type = other DNA
cagggctgcttgcttctgtcatcgaaatctcattgaaattggttatgtctgttctgtgtgtttgtcaagtaagttaatgtacctagtttttcttttttttctatgttggggggcaggaaa

SEQ ID NO: 645 - DNA, 120 bp, synthetic construct
  misc_feature 1..120: note = Synthetic polynucleotide
  source 1..120: mol_type = other DNA
ttttcagccaccctattgtttctttttttttttaatttacagtattctgcaatttcagccccatttgtactacgtgcgagtaagtatctttgagattgtgtgggtggctaatacttcaca

SEQ ID NO: 646 - DNA, 120 bp, synthetic construct
  misc_feature 1..120: note = Synthetic polynucleotide
  source 1..120: mol_type = other DNA
aatgttttcctctctgtaatttcaggacagcctttaaaatttctctgcctccagtgctgaaagccaagaaaaagaaactgaaagtgtctgcctgaggataaaatattttccccatctttt

SEQ ID NO: 647 - DNA, 120 bp, synthetic construct
  misc_feature 1..120: note = Synthetic polynucleotide
  source 1..120: mol_type = other DNA
acactggcatggatggtgaggttgcacttctgacgtttgcattcctcaggtgatggagagcaccccttcaaggggactgaaccgagtacacctacaatgcaggaatctgcaggaattctt

SEQ ID NO: 648 - DNA, 120 bp, synthetic construct
  misc_feature 1..120: note = Synthetic polynucleotide
  source 1..120: mol_type = other DNA
agggggcctgagccctggggtattggaccgattgtatgggcaccctgccacatgtctggctgtcttcaggtgagaagccccttcatggcagggaaatgtaatggggtctgcggagtggaa

SEQ ID NO: 649 - DNA, 120 bp, synthetic construct
  misc_feature 1..120: note = Synthetic polynucleotide
  source 1..120: mol_type = other DNA
ctgtacagggagctcccatccttggctaagaactgggtgatgcggatgctctttctggagcagcctttgccacaggctgctgtagctctgtgggtaaagaaggaattcagcaagtaagtc

SEQ ID NO: 650 - DNA, 120 bp, synthetic construct
  misc_feature 1..120: note = Synthetic polynucleotide
  source 1..120: mol_type = other DNA
gggcctcctttttgttttccaaataccctactcacctctctgcttctgttccagggctcaggaggaaagtacagggctgctgagcggcctccggatctggcacacacagctgctcccagg

SEQ ID NO: 651 - DNA, 120 bp, synthetic construct
  misc_feature 1..120: note = Synthetic polynucleotide
  source 1..120: mol_type = other DNA
cgggctccagggcctcatcctcaaccccattttccgccagaacctccgcattgcccttctgggtgggtatgtcacttctctctcttcctaagctagggcaggggaactgctgcttattaa

SEQ ID NO: 652 - DNA, 120 bp, synthetic construct
  misc_feature 1..120: note = Synthetic polynucleotide
  source 1..120: mol_type = other DNA
cctggccgtagggggaaggcctggtctgatgacacaagtcagctgggaccagacaagcatgcccgggacgttccctcccttgacaagtacgccgaggagcgatgggaggtaagcacttgg

SEQ ID NO: 653 - DNA, 120 bp, synthetic construct
  misc_feature 1..120: note = Synthetic polynucleotide
  source 1..120: mol_type = other DNA
ctcttctgttcttcaggtggtcttgcacttcatggtgggctcccccagtgcagctgtcagccaggacttggctcagctcctcagccaggctgggctcatgaagaggtgaggaagccggag

SEQ ID NO: 654 - DNA, 120 bp, synthetic construct
  misc_feature 1..120: note = Synthetic polynucleotide
  source 1..120: mol_type = other DNA
ctagtactgaacctggagagccgccctgcattacttccgctggcttccagttcctgttgctggacaccccggctcagctctggtactttatgttgcagtatttgcagacagcccaggtga

SEQ ID NO: 655 - DNA, 120 bp, synthetic construct
  misc_feature 1..120: note = Synthetic polynucleotide
  source 1..120: mol_type = other DNA
tttgtctctgcctctttctccctagagccggggcatggacctggtagagattctctccttcctcttccagctcagcttctctactctgggcaaggtaagcagggggctgaaaggtataga

SEQ ID NO: 656 - DNA, 120 bp, synthetic construct
  misc_feature 1..120: note = Synthetic polynucleotide
  source 1..120: mol_type = other DNA
gaaacctctgttcctcaggattactctgtggaaggtatgagtgattctctgttgaacttcctgcaacatctgcgtgagtttgggcttgttttccagaggaaggtatgagcgcctagataa

SEQ ID NO: 657 - DNA, 120 bp, synthetic construct
  misc_feature 1..120: note = Synthetic polynucleotide
  source 1..120: mol_type = other DNA
agatgtaaggcagtgacttctgagacaaggcatctgcctttctattcttttcagaggaaatctcggcgttactaccccacacgcctggccatcaatctctcatcaggtgtctctggagct

SEQ ID NO: 658 - DNA, 120 bp, synthetic construct
  misc_feature 1..120: note = Synthetic polynucleotide
  source 1..120: mol_type = other DNA
gggggcactgtgcatcagccaggtttcattgtcgtggaaaccaattaccgactgtatgcctacacgggtgaggcgggacagagggcccctggaagaggaggttgggggtgagggaatgcc

SEQ ID NO: 659 - DNA, 120 bp, synthetic construct
  misc_feature 1..120: note = Synthetic polynucleotide
  source 1..120: mol_type = other DNA
agggagtctgggtgtgggggtggcctcctcatcctctttctatccctggctcagagtcggagctgcagattgccctcattgccctcttctctgagatgctctatcggttccccaacatgg

SEQ ID NO: 660 - DNA, 120 bp, synthetic construct
  misc_feature 1..120: note = Synthetic polynucleotide
  source 1..120: mol_type = other DNA
tggtggcgcaggtgacccgggagagtgtgcagcaggcaatcgccagtggcatcacagcccagcaggtattcccacttgggagaggtggagcaggaagacaggctgcacttgggctgcggg

SEQ ID NO: 661 - DNA, 120 bp, synthetic construct
  misc_feature 1..120: note = Synthetic polynucleotide
  source 1..120: mol_type = other DNA
tcttgctggagccctcatgccattcttgtctgttttcctagataatccatttcctaaggacaagagcccacccagtgatgctcaaacaggtatagacaggctccaagatgtcagaggctg

SEQ ID NO: 662 - DNA, 120 bp, synthetic construct
  misc_feature 1..120: note = Synthetic polynucleotide
  source 1..120: mol_type = other DNA
gcagctggtgatgacatgatggaaaagaaaaaggggcatccaaatctggggaagaaacagagggccgggttgtctggggcagtattctgagtccctacagtcaacccttgctccttgcag

SEQ ID NO: 663 - DNA, 120 bp, synthetic construct
  misc_feature 1..120: note = Synthetic polynucleotide
  source 1..120: mol_type = other DNA
acacctgtgctgccccccaccatcaccgaccagatccggctctgggagctggaaagggacagactccggttcactgagggtgagtagcttctggtggccaagtcttggtcattggccaga

SEQ ID NO: 664 - DNA, 120 bp, synthetic construct
  misc_feature 1..120: note = Synthetic polynucleotide
  source 1..120: mol_type = other DNA
tctccccgcgcccctcccgtcctgccgaccccaggtgtcctgtataaccagttcctgtcgcaagtggactttgagctgctgctggcccacgcgcgggagctgggcgtgctcgtgttcgag

SEQ ID NO: 665 - DNA, 120 bp, synthetic construct
  misc_feature 1..120: note = Synthetic polynucleotide
  source 1..120: mol_type = other DNA
aactcggccaagcggctcatggtggtgaccccggccgggcacagcgacgtcaagcgcttttggaagcggcagaaacatagctcctgagagcgcgggacttggacacggacctcggcgggc

SEQ ID NO: 666 - DNA, 120 bp, synthetic construct
  misc_feature 1..120: note = Synthetic polynucleotide
  source 1..120: mol_type = other DNA
tttattagcattcttcaggtcatctgaaccttctgagaaaacatggtcaacgtcttgaaaggagtgcttatagaatggttagtagttttgatactgcatgagttttaagtttaaatattg

SEQ ID NO: 667 - DNA, 120 bp, synthetic construct
  misc_feature 1..120: note = Synthetic polynucleotide
  source 1..120: mol_type = other DNA
tgtcttacaatcatgtgtttgtctttacagtgatcctgccatgaagcagtttctgctgtacttggatgagtccaatgccctggggaagaagttcatcattcaagacattgatgacactca

SEQ ID NO: 668 - DNA, 120 bp, synthetic construct
  misc_feature 1..120: note = Synthetic polynucleotide
  source 1..120: mol_type = other DNA
cgtctttgtaatagcagaattggttaatgtcctccaggagcgagtgggtgaattaatggaccaaaatgctttttcccttacccagaaatgaaaatactcaatatggaccatttaggaatt

SEQ ID NO: 669 - DNA, 120 bp, synthetic construct
  misc_feature 1..120: note = Synthetic polynucleotide
  source 1..120: mol_type = other DNA
caggacccgcttctctgaaaggctctccttgcagctgcttagacgctggatttttttcgggtagtggaaaaccaggtaagcaccgaagtccacttgccttttaatttatttttttatcac

SEQ ID NO: 670 - DNA, 120 bp, synthetic construct
  misc_feature 1..120: note = Synthetic polynucleotide
  source 1..120: mol_type = other DNA
aagactgcctcccgctttgtgtgccccgctccagcagcctcccgcgacgatgcccctcaacgttagcttcaccaacaggaactatgacctcgactacgactcggtgcagccgtatttcta

SEQ ID NO: 671 - DNA, 120 bp, synthetic construct
  misc_feature 1..120: note = Synthetic polynucleotide
  source 1..120: mol_type = other DNA
ctgcgacgaggaggagaacttctaccagcagcagcagcagagcgagctgcagcccccggcgcccagcgaggatatctggaagaaattcgagctgctgcccaccccgcccctgtcccctag

SEQ ID NO: 672 - DNA, 120 bp, synthetic construct
  misc_feature 1..120: note = Synthetic polynucleotide
  source 1..120: mol_type = other DNA
ccgccgctccgggctctgctcgccctcctacgttgcggtcacacccttctcccttcggggagacaacgacggcggtggcgggagcttctccacggccgaccagctggagatggtgaccga

SEQ ID NO: 673 - DNA, 120 bp, synthetic construct
  misc_feature 1..120: note = Synthetic polynucleotide
  source 1..120: mol_type = other DNA
gctgctgggaggagacatggtgaaccagagtttcatctgcgacccggacgacgagaccttcatcaaaaacatcatcatccaggactgtatgtggagcggcttctcggccgccgccaagct

SEQ ID NO: 674 - DNA, 120 bp, synthetic construct
  misc_feature 1..120: note = Synthetic polynucleotide
  source 1..120: mol_type = other DNA
cgtctcagagaagctggcctcctaccaggctgcgcgcaaagacagcggcagcccgaaccccgcccgcggccacagcgtctgctccacctccagcttgtacctgcaggatctgagcgccgc

SEQ ID NO: 675 - DNA, 120 bp, synthetic construct
  misc_feature 1..120: note = Synthetic polynucleotide
  source 1..120: mol_type = other DNA
cgcctcagagtgcatcgacccctcggtggtcttcccctaccctctcaacgacagcagctcgcccaagtcctgcgcctcgcaagactccagcgccttctctccgtcctcggattctctgct

SEQ ID NO: 676 - DNA, 120 bp, synthetic construct
  misc_feature 1..120: note = Synthetic polynucleotide
  source 1..120: mol_type = other DNA
ctcctcgacggagtcctccccgcagggcagccccgagcccctggtgctccatgaggagacaccgcccaccaccagcagcgactctggtaagcgaagcccgcccaggcctgtcaaaagtgg

SEQ ID NO: 677 - DNA, 120 bp, synthetic construct
  misc_feature 1..120: note = Synthetic polynucleotide
  source 1..120: mol_type = other DNA
ctatttcctttcttaaagaggaggaacaagaagatgaggaagaaatcgatgttgtttctgtggaaaagaggcaggctcctggcaaaaggtcagagtctggatcaccttctgctggaggcc

SEQ ID NO: 678 - DNA, 120 bp, synthetic construct
  misc_feature 1..120: note = Synthetic polynucleotide
  source 1..120: mol_type = other DNA
acagcaaacctcctcacagcccactggtcctcaagaggtgccacgtctccacacatcagcacaactacgcagcgcctccctccactcggaaggactatcctgctgccaagagggtcaagt

SEQ ID NO: 679 - DNA, 120 bp, synthetic construct
  misc_feature 1..120: note = Synthetic polynucleotide
  source 1..120: mol_type = other DNA
tggacagtgtcagagtcctgagacagatcagcaacaaccgaaaatgcaccagccccaggtcctcggacaccgaggagaatgtcaagaggcgaacacacaacgtcttggagcgccagagga

SEQ ID NO: 680 - DNA, 120 bp, synthetic construct
  misc_feature 1..120: note = Synthetic polynucleotide
  source 1..120: mol_type = other DNA
ggaacgagctaaaacggagcttttttgccctgcgtgaccagatcccggagttggaaaacaatgaaaaggcccccaaggtagttatccttaaaaaagccacagcatacatcctgtccgtcc

SEQ ID NO: 681 - DNA, 120 bp, synthetic construct
  misc_feature 1..120: note = Synthetic polynucleotide
  source 1..120: mol_type = other DNA
aagcagaggagcaaaagctcatttctgaagaggacttgttgcggaaacgacgagaacagttgaaacacaaacttgaacagctacggaactcttgtgcgtaaggaaaagtaaggaaaacga

SEQ ID NO: 682 - DNA, 120 bp, synthetic construct
  misc_feature 1..120: note = Synthetic polynucleotide
  source 1..120: mol_type = other DNA
cttcaggatggttcgtactaagacatggaccctgaagaagcactttgttggctatcctactaatagtgactttgagttgaagacagctgagctcccacccttaaaaaatggaggtaagtc

SEQ ID NO: 683 - DNA, 120 bp, synthetic construct
  misc_feature 1..120: note = Synthetic polynucleotide
  source 1..120: mol_type = other DNA
gagtttctaattgcctatggaatgctttattttgtagaggtcctgcttgaagctttgttcctcaccgtggatccctacatgaggtattatttatttcgcccctcaaaatgctaatgctgt

SEQ ID NO: 684 - DNA, 120 bp, synthetic construct
  misc_feature 1..120: note = Synthetic polynucleotide
  source 1..120: mol_type = other DNA
tttttttttttttttttttttgcagagtggcagccaaaagattgaaggaaggtgatacaatgatggggcagcaagtggccaagtaattatttccatttgtcccagtgcttggaaaatatg

SEQ ID NO: 685 - DNA, 119 bp, synthetic construct
  misc_feature 1..119: note = Synthetic polynucleotide
  source 1..119: mol_type = other DNA
tctcttaaaaaaaccaaacatctcattctttatttttttaaattttgtgtctcaatgactagccagttattataataccattggtagaaatcgtctatggatctttactggaatttcag

SEQ ID NO: 686 - DNA, 120 bp, synthetic construct
  misc_feature 1..120: note = Synthetic polynucleotide
  source 1..120: mol_type = other DNA
acatgccttaatgccttaatgtgctttatctttcagagttgtggaaagtaaaaatgtagccctaccaaaaggaactattgtactggcttctccaggctggacaacgcactccatttctga

SEQ ID NO: 687 - DNA, 120 bp, synthetic construct
  misc_feature 1..120: note = Synthetic polynucleotide
  source 1..120: mol_type = other DNA
tgggaaagatctggaaaagctgctgacagagtggccagacacaataccactgtctttggctctggggacagttggcatgccagggtgagtttcatggatatattccatttgtttgaatgt

SEQ ID NO: 688 - DNA, 120 bp, synthetic construct
  misc_feature 1..120: note = Synthetic polynucleotide
  source 1..120: mol_type = other DNA
gcctgactgcctactttggcctacttgaaatctgtggtgtgaagggtggagaaacagtgatggttaatgcagcagctggagctgtgggctcagtcgtggggcagattgcaaagctcaagg

SEQ ID NO: 689 - DNA, 120 bp, synthetic construct
  misc_feature 1..120: note = Synthetic polynucleotide
  source 1..120: mol_type = other DNA
tgacttattcatggtttcctttttttttttttttctacttagggctgcaaagttgttggagcagtagggtctgatgaaaaggttgcctaccttcaaaagcttggatttgatgtcgtcttt

SEQ ID NO: 690 - DNA, 120 bp, synthetic construct
  misc_feature 1..120: note = Synthetic polynucleotide
  source 1..120: mol_type = other DNA
aactacaagacggtagagtctttggaagaaaccttgaagaaagcgtctcctgatggttatgattgttattttgataatgtaagtacaaactggacttaatatctaatttattatggaagt

SEQ ID NO: 691 - DNA, 120 bp, synthetic construct
  misc_feature 1..120: note = Synthetic polynucleotide
  source 1..120: mol_type = other DNA
gtttaggtaggtggagagttttcaaacactgttatcggccagatgaagaaatttggaaggattgccatatgtggagccatctctacatataacagaaccggcccacttcccccaggtaat

SEQ ID NO: 692 - DNA, 120 bp, synthetic construct
  misc_feature 1..120: note = Synthetic polynucleotide
  source 1..120: mol_type = other DNA
gcccacccccagagattgttatctatcaggagcttcgcatggaagcttttgtcgtctaccgctggcaaggagatgcccgccaaaaagctctgaaggacttgctgaaatgggtcttagagg

SEQ ID NO: 693 - DNA, 120 bp, synthetic construct
  misc_feature 1..120: note = Synthetic polynucleotide
  source 1..120: mol_type = other DNA
ttgagaatattaaattaagttttaaaatttgttgttgctttgatacagggtaaaatccagtacaaggaatatatcattgaaggatttgaaaacatgccagctgcatttatgggaatgctg

SEQ ID NO: 694 - DNA, 120 bp, synthetic construct
  misc_feature 1..120: note = Synthetic polynucleotide
  source 1..120: mol_type = other DNA
aaaggagataatttggggaagacaatagtgaaagcatgaaaaagaggacacatggaatctggaggccatttagatgattagttaatttgtttttcaccatttagcaaaaatgtatactac

SEQ ID NO: 695 - DNA, 120 bp, synthetic construct
  misc_feature 1..120: note = Synthetic polynucleotide
  source 1..120: mol_type = other DNA
cttaaatgtcttaagaaatagtactcataatgagtttgagctacttaataaaatacatttaagtggtatgtaattagtgatggaggatggaagtttcaaagtcaacaacaaccagtcacc

SEQ ID NO: 696 - DNA, 120 bp, synthetic construct
  misc_feature 1..120: note = Synthetic polynucleotide
  source 1..120: mol_type = other DNA
gatcaaaagagaaaatgaagaagattgaagcttcaaagcagaaaaatgaagggaatatgtatcattcaccattaccttagaaggcaagggtccactttacaaaaggactacaaacattta

SEQ ID NO: 697 - DNA, 120 bp, synthetic construct
  misc_feature 1..120: note = Synthetic polynucleotide
  source 1..120: mol_type = other DNA
gggtggctgacccccagcatcctcgggagcgaccatggactccctggccgagtctcggtggcctccgggcctggcagtcatgaaggtgagtgcttcggggagtttgggagctcccgggtg

SEQ ID NO: 698 - DNA, 120 bp, synthetic construct
  misc_feature 1..120: note = Synthetic polynucleotide
  source 1..120: mol_type = other DNA
atgcatatttgcatttcagacaatagatgatttgctgcggtgtggaatttgcttcgagtatttcaacattgcaatgataatacctcagtgttcacataactgtaagtatgttttgttcct

SEQ ID NO: 699 - DNA, 120 bp, synthetic construct
  misc_feature 1..120: note = Synthetic polynucleotide
  source 1..120: mol_type = other DNA
aacattatgttgcactttgtgttttgcagactgctctctctgtataagaaaatttctgtcctataaaactcagtgtccaacttgctgtgtggtgagtttttgtttcctttttttaaaact

SEQ ID NO: 700 - DNA, 120 bp, synthetic construct
  misc_feature 1..120: note = Synthetic polynucleotide
  source 1..120: mol_type = other DNA
ccatctgttgtttgttttttctagactgtcacagagccggatctgaaaaataaccgcatattagatgaactggtaaaaagcttgaattttgcacggtatgatttaattttgtgactaacc

SEQ ID NO: 701 - DNA, 120 bp, synthetic construct
  misc_feature 1..120: note = Synthetic polynucleotide
  source 1..120: mol_type = other DNA
tgtgcttcaaggaatcatctgctgcagtttgctttagagtcaccagccaaatctcctgcttcttcctcttcaaagaatcttgctgtcaaagtatatactcctgtagcctccagacagtct

SEQ ID NO: 702 - DNA, 120 bp, synthetic construct
  misc_feature 1..120: note = Synthetic polynucleotide
  source 1..120: mol_type = other DNA
ttaaagcaggggagcaggttaatggataatttcttgatcagagaaatgagtggttctacatcagagttgttgataaaagaaaataaaagcaaattcagccctcaaaaagaggcgagccct

SEQ ID NO: 703 - DNA, 120 bp, synthetic construct
  misc_feature 1..120: note = Synthetic polynucleotide
  source 1..120: mol_type = other DNA
gctgcaaagaccaaagagacacgttctgtagaagagatcgctccagatccctcagaggctaagcgtcctgagccaccctcgacatccactttgaaacaagttactaaaggtaggaagtta

SEQ ID NO: 704 - DNA, 120 bp, synthetic construct
  misc_feature 1..120: note = Synthetic polynucleotide
  source 1..120: mol_type = other DNA
aaaaatacagtggattgtcctgtttgcggggttaacattccagaaagtcacattaataagcatttagacagctgtttatcacgcgaagagaagaaggaaagcctcagaaggtaaggaagt

SEQ ID NO: 705 - DNA, 120 bp, synthetic construct
  misc_feature 1..120: note = Synthetic polynucleotide
  source 1..120: mol_type = other DNA
atcatcaggtatcttccccccttttcagttctgttcacaaaaggaagccgctgcccaaaactgtatataatttgctctctgatcgtgatttaaagaaaaagctaaaagagcatggattat

SEQ ID NO: 706 - DNA, 120 bp, synthetic construct
  misc_feature 1..120: note = Synthetic polynucleotide
  source 1..120: mol_type = other DNA
ctattcaaggaaataaacaacagctcattaaaaggcaccaagaatttgtacacatgtacaatgcccaatgcgatgctttgcatcctaaatcaggtaaagtaataaaacagtgagttatgc

SEQ ID NO: 707 - DNA, 120 bp, synthetic construct
  misc_feature 1..120: note = Synthetic polynucleotide
  source 1..120: mol_type = other DNA
gttattttcttattttaacagctgctgaaatagttcgagaaatcgaaaatatagagaagactaggatgcgtcttgaagctagtaaactcaatgaaagtgtatgttaatgggctatttcag

SEQ ID NO: 708 - DNA, 120 bp, synthetic construct
  misc_feature 1..120: note = Synthetic polynucleotide
  source 1..120: mol_type = other DNA
tttattaaggtctttttttgattattttaggtaatggtttttacaaaggaccaaacagaaaaggaaatagatgaaatccacagtaaatatcgtaagttgcacgactttatttaatttgta

SEQ ID NO: 709 - DNA, 120 bp, synthetic construct
  misc_feature 1..120: note = Synthetic polynucleotide
  source 1..120: mol_type = other DNA
ggttaactttcatagtcctagctttactgagaagatcttttgaatttcaggtaaaaaacataagagtgaatttcagcttctggtggatcaggctagaaaaggatacaagaaaattgctgg

SEQ ID NO: 710 - DNA, 120 bp, synthetic construct
  misc_feature 1..120: note = Synthetic polynucleotide
  source 1..120: mol_type = other DNA
aatgtcacaaaaaacagtaacaataacaaaagaagatgaatctacagaaaagctatcttctgtatgcatgggtaagagactataattaaaaacagacatatgtcgaatttcattatttac

SEQ ID NO: 711 - DNA, 120 bp, synthetic construct
  misc_feature 1..120: note = Synthetic polynucleotide
  source 1..120: mol_type = other DNA
aagttgtagattttaaggtactcttaaatttattttttcctaggacaggaagataatatgacctcagtaacaaaccacttttctcaatcaaagctggactccccagaggaattggaacct

SEQ ID NO: 712 - DNA, 120 bp, synthetic construct
  misc_feature 1..120: note = Synthetic polynucleotide
  source 1..120: mol_type = other DNA
gacagagaagaggattcttctagctgtattgatattcaagaagttctttcttcatcagaatcagattcatgcaataggtaagaaacatgcagtagttgattattttaaatatctacttca

SEQ ID NO: 713 - DNA, 120 bp, synthetic construct
  misc_feature 1..120: note = Synthetic polynucleotide
  source 1..120: mol_type = other DNA
tgtcaatactgatgtacatatccttttagttccagttcagacatcataagagatcttttagaagaagaggaagcctgggaagcatcacataagtgagttctcataatcatgaaataagca

SEQ ID NO: 714 - DNA, 120 bp, synthetic construct
  misc_feature 1..120: note = Synthetic polynucleotide
  source 1..120: mol_type = other DNA
gtcttcagaaacgatcttcaagacacagaaataagtccaagacagaatcgccgcacaagagccgctgaaagtgctgagattgaaccaagaaacaagcgtaataggaattaatgtgggctt

SEQ ID NO: 715 - DNA, 120 bp, synthetic construct
  misc_feature 1..120: note = Synthetic polynucleotide
  source 1..120: mol_type = other DNA
ttttatacagaaatcttgtgaaaccactgcccaaccagagcaatgattgttcaaagagtggtattgaattctcgacctggtatgtatttgcgatgaatgaacaactctgatctttgataa

SEQ ID NO: 716 - DNA, 120 bp, synthetic construct
  misc_feature 1..120: note = Synthetic polynucleotide
  source 1..120: mol_type = other DNA
gaaaaaatggtaatccagtggcagagaatttccgaatggaagaagtctatttaccagataatattaatgaaggacaagtacaagttagaactctttatctttctgtggatccttacatgg

SEQ ID NO: 717 - DNA, 120 bp, synthetic construct
  misc_feature 1..120: note = Synthetic polynucleotide
  source 1..120: mol_type = other DNA
tatcttaatgttttcttttttcagcgttgtagaatgaatgaagacactggcactgattatataacaccttggcagctatctcaagtcgttgatggaggaggtattggaattatagaagaa

SEQ ID NO: 718 - DNA, 120 bp, synthetic construct
  misc_feature 1..120: note = Synthetic polynucleotide
  source 1..120: mol_type = other DNA
agcaaacacacaaatttgactaaaggcgattttgtgacttctttctattggccctggcaaaccaaggttattctggatggaaatagccttgaaaaggtgatatatatatgaacatatctg

SEQ ID NO: 719 - DNA, 120 bp, synthetic construct
  misc_feature 1..120: note = Synthetic polynucleotide
  source 1..120: mol_type = other DNA
cctagtatttttaaagggtatatattttattttaggtagacccacaacttgtggatggacacctttcatattttcttggagctataggtatgcctggtttgacttccttgattgggatac

SEQ ID NO: 720 - DNA, 120 bp, synthetic construct
  misc_feature 1..120: note = Synthetic polynucleotide
  source 1..120: mol_type = other DNA
aggaaaaaggtcatataactgctggatctaataagacaatggttgtcagtggggccgcaggtgcctgtggatctgtggctgggcaggtaaactttctgagaattatttgattttctgatt

SEQ ID NO: 721 - DNA, 120 bp, synthetic construct
  misc_feature 1..120: note = Synthetic polynucleotide
  source 1..120: mol_type = other DNA
aaatttcccccctagattggccatttcttaggttgttccagagtggtgggaatttgtggaacacatgagaaatgcatcctcttgacctcagaactgggctttgatgctgcaattaattat

SEQ ID NO: 722 - DNA, 120 bp, synthetic construct
  misc_feature 1..120: note = Synthetic polynucleotide
  source 1..120: mol_type = other DNA
aaaaaagacaatgtggcagaacagctccgtgaatcatgcccagctggagtggatgtttattttgacaatgttggtggtaacatcagtgatacagtgataagtcaggttgtttgctgattt

SEQ ID NO: 723 - DNA, 120 bp, synthetic construct
  misc_feature 1..120: note = Synthetic polynucleotide
  source 1..120: mol_type = other DNA
agccataaaatcagtaaacataggaaagggatattttatcattatttttctctgtgcagatgaatgagaacagccacatcatcctgtgtggtcaaatttctcagtacaacaaagatgtgc

SEQ ID NO: 724 - DNA, 120 bp, synthetic construct
  misc_feature 1..120: note = Synthetic polynucleotide
  source 1..120: mol_type = other DNA
cttatcctcccccgctatcccctgctatagaggcaatccagaaagaaagaaacatcacaaggtgtgttcttcctctttgcccttattaccaggttcatggggttttaaatatcatagatg

SEQ ID NO: 725 - DNA, 120 bp, synthetic construct
  misc_feature 1..120: note = Synthetic polynucleotide
  source 1..120: mol_type = other DNA
ttcttcctcactgcagggaaagatttctggtattaaattataaagacaaatttgagcctggcattctacagctgagtcagtggtttaaagaaggaaagctaaaggtagaacttctattct

SEQ ID NO: 726 - DNA, 120 bp, synthetic construct
  misc_feature 1..120: note = Synthetic polynucleotide
  source 1..120: mol_type = other DNA
aagtttaaagactattaaatctagctattttgatttacagattaaagagacggtaataaatgggttggaaaacatgggaggtaagatgaatgtagacttattatatacacatgctcagca

SEQ ID NO: 727 - DNA, 120 bp, synthetic construct
  misc_feature 1..120: note = Synthetic polynucleotide
  source 1..120: mol_type = other DNA
aacctactttctctttttccagctgcattccagtccatgatgacaggaggtaacattggaaagcagatagtttgcatttcagaagaaatctctttgtaattgctgtaaatgtcatcaagg

SEQ ID NO: 728 - DNA, 120 bp, synthetic construct
  misc_feature 1..120: note = Synthetic polynucleotide
  source 1..120: mol_type = other DNA
gagctgggagctaggtcctcggagtgggccagagatggcggcggccgacggggctttgccggaggcggcggctttagagcaacccgcggagctgcctgcctcggtgcgggcgagtatcga

SEQ ID NO: 729 - DNA, 120 bp, synthetic construct
  misc_feature 1..120: note = Synthetic polynucleotide
  source 1..120: mol_type = other DNA
gcggaagcggcagcgggcactgatgctgcgccaggcccggctggctgcccggccctactcggcgacggcggctgcggctactggaggtttgggccgcgtccgcgctttccccttccctct

SEQ ID NO: 730 - DNA, 120 bp, synthetic construct
  misc_feature 1..120: note = Synthetic polynucleotide
  source 1..120: mol_type = other DNA
gtaggcatggctaatgtaaaagcagccccaaagataattgacacaggaggaggcttcattttagaagaggaagaagaagaagaacagaaaattggaaaagttgttcatcaaccaggtaaa

SEQ ID NO: 731 - DNA, 120 bp, synthetic construct
  misc_feature 1..120: note = Synthetic polynucleotide
  source 1..120: mol_type = other DNA
ttcttaggacctgttatggaatttgattatgtaatatgcgaagaatgtgggaaagaatttatggattcttatcttatgaaccactttgatttgccaacttgtgataactgcaggtactta

SEQ ID NO: 732 - DNA, 120 bp, synthetic construct
  misc_feature 1..120: note = Synthetic polynucleotide
  source 1..120: mol_type = other DNA
ataactaatttatataatggacttaatctgttttcagagatgctgatgataaacacaagcttataaccaaaacagaggcaaaacaagaatatcttctgaaagactgtgatttagaaaaaa

SEQ ID NO: 733 - DNA, 120 bp, synthetic construct
  misc_feature 1..120: note = Synthetic polynucleotide
  source 1..120: mol_type = other DNA
gagagccacctcttaaatttattgtgaagaagaatccacatcattcacaatggggtgatatgaaactctacttaaagttacaggtctctaataagttgtatttattatttttcactctgg

SEQ ID NO: 734 - DNA, 120 bp, synthetic construct
  misc_feature 1..120: note = Synthetic polynucleotide
  source 1..120: mol_type = other DNA
gattgtgaagaggtctcttgaagtttggggtagtcaagaagcattagaagaagcaaaggaagtccgacaggaaaaccgagaaaaaatgaaacagaagaaatttgataaaaaagtaaaagg

SEQ ID NO: 735 - DNA, 120 bp, synthetic construct
  misc_feature 1..120: note = Synthetic polynucleotide
  source 1..120: mol_type = other DNA
cctgaatagcaccactgaaaagatgactttaaaatttttttttcagaattgcggcgagcagtaagaagcagcgtgtggaaaagggagacgattgttcatcaacatgagtatggaccagaa

SEQ ID NO: 736 - DNA, 120 bp, synthetic construct
  misc_feature 1..120: note = Synthetic polynucleotide
  source 1..120: mol_type = other DNA
gaaaacctagaagatgacatgtaccgtaagacttgtactatgtgtggccatgaactgacatatgaaaaaatgtgattttttagttcagtgacctgttttatagaattttatatttaaata

SEQ ID NO: 737 - DNA, 120 bp, synthetic construct
  misc_feature 1..120: note = Synthetic polynucleotide
  source 1..120: mol_type = other DNA
caagcaacatggctcggaaacgcgcggccggcggggagccgcggggacgcgaactgcgcagccagaaatccaaggccaagagcaaggcccggcgtgaggaggaggaggagggtgagagcg

SEQ ID NO: 738 - DNA, 120 bp, synthetic construct
  misc_feature 1..120: note = Synthetic polynucleotide
  source 1..120: mol_type = other DNA
tatgttctgtgttgtcacctagatgcctttgaagatgagaaacccccaaagaagagccttctctccaaagtttcacaaggaaagaggaaaagaggctgcagtcatcctgggggttcagca

SEQ ID NO: 739 - DNA, 120 bp, synthetic construct
  misc_feature 1..120: note = Synthetic polynucleotide
  source 1..120: mol_type = other DNA
gatggtccagcaaaaaagaaagtggccaaggtgactgttaaatctgaaaacctcaaggttataaaggatgaagccctcagcgatggggatgacctcaggtgagatgtctgcaaagctttg

SEQ ID NO: 740 - DNA, 120 bp, synthetic construct
  misc_feature 1..120: note = Synthetic polynucleotide
  source 1..120: mol_type = other DNA
acagggactttccaagtgacctcaagaaggcacaccatctgaagagaggggctaccatgaatgaagacagcaatgaagaagaggaagaaagtgaaaatgattgggaagaggttgaaggtg

SEQ ID NO: 741 - DNA, 120 bp, synthetic construct
  misc_feature 1..120: note = Synthetic polynucleotide
  source 1..120: mol_type = other DNA
acagtagctattattattgttattactattactgatttttaaaaatgcttgttgatagaacttagtgagcctgtgctgggtgacgtgagagaaagtacagccttctctcgatctcttctg

SEQ ID NO: 742 - DNA, 120 bp, synthetic construct
  misc_feature 1..120: note = Synthetic polynucleotide
  source 1..120: mol_type = other DNA
cctgtgaagccagtggagatagagattgaaacgccagagcaggcgaagacaagagaaagaaggtaagcttaggcccttgcttctaggctcctgtcacatgcagtaggcaacagctgctgc

SEQ ID NO: 743 - DNA, 120 bp, synthetic construct
  misc_feature 1..120: note = Synthetic polynucleotide
  source 1..120: mol_type = other DNA
tttcactatttgttgcagtgaaaagataaaactggagtttgagacatatcttcggagggcgatgaaacgtttcaataaaggggtccatgaggacacacacaaggtaagggcaaggaatga

SEQ ID NO: 744 - DNA, 120 bp, synthetic construct
  misc_feature 1..120: note = Synthetic polynucleotide
  source 1..120: mol_type = other DNA
ccttccttccatgctgccccttcctcctttcctcttcacaggttcaccttctctgcctgctagcaaatggcttctatcgaaataacatctgcagccagccagatctgcatgctattggcc

SEQ ID NO: 745 - DNA, 120 bp, synthetic construct
  misc_feature 1..120: note = Synthetic polynucleotide
  source 1..120: mol_type = other DNA
tgtccatcatcccagcccgctttaccagagtgctgcctcgagatgtggacacctactacctctcaaacctggtgaagtggtaaggccctccgcttgtcctgcagagctggggagtgtagg

SEQ ID NO: 746 - DNA, 120 bp, synthetic construct
  misc_feature 1..120: note = Synthetic polynucleotide
  source 1..120: mol_type = other DNA
gaaatgaaaattcccctttgccctgacctctgacacaaggaatgcctgctttctccccaggttcattggaacatttacagttaatgcagaactttcagccagtgaacaagataacctgca

SEQ ID NO: 747 - DNA, 120 bp, synthetic construct
  misc_feature 1..120: note = Synthetic polynucleotide
  source 1..120: mol_type = other DNA
gactacattggaaaggagatttgctatttactctgctcgagatgatgaggaattggtccatgtaagtgatcctcccggatcactgttttttatcagtactgttaactaatgataatggca

SEQ ID NO: 748 - DNA, 120 bp, synthetic construct
  misc_feature 1..120: note = Synthetic polynucleotide
  source 1..120: mol_type = other DNA
tccttttcatcatagatattcttactgattctccgggctctgcagctcttgacccggctggtattgtctctacagccaattcctctgaagtcagcaacagcaaaggtgaggtgcgcaggg

SEQ ID NO: 749 - DNA, 120 bp, synthetic construct
  misc_feature 1..120: note = Synthetic polynucleotide
  source 1..120: mol_type = other DNA
gggggagaaaaaaaagcaaaatttcttattctgtttaagggaaagaaaccttccaaggaaagattgactgcggatccaggaggctcctcagaaacttccagccaagttctagaaaaccac

SEQ ID NO: 750 - DNA, 120 bp, synthetic construct
  misc_feature 1..120: note = Synthetic polynucleotide
  source 1..120: mol_type = other DNA
accaaaccaaagaccagcaaaggaaccaaacaagaggaaacctttgctaagggcacctgcaggccaagtgccaaagggaagaggaacaagggaggcagaaagaaacggagcaagccctcc

SEQ ID NO: 751 - DNA, 120 bp, synthetic construct
  misc_feature 1..120: note = Synthetic polynucleotide
  source 1..120: mol_type = other DNA
tccagcgaggaagatgagggcccaggagacaagcaggagaaggcaacccagcgacgtccgcatggccgggagcggcgggtggcctccagggtgtcttataaagaggagagtgggagtgat

SEQ ID NO: 752 - DNA, 120 bp, synthetic construct
  misc_feature 1..120: note = Synthetic polynucleotide
  source 1..120: mol_type = other DNA
gaggctggcagcggctctgattttgagctctccagtggagaagcctctgatccctctgatgaggattccgaacctggccctccaaagcagaggaaagcccccgctcctcagaggacaaag

SEQ ID NO: 753 - DNA, 120 bp, synthetic construct
  misc_feature 1..120: note = Synthetic polynucleotide
  source 1..120: mol_type = other DNA
gctgggtccaagagtgcctccaggacccatcgtgggagccatcgtaaggacccaagcttgccagcggcatcctcaagctcttcaagcagtaaaagaggcaagaaaatgtgcagcgatggt

SEQ ID NO: 754 - DNA, 120 bp, synthetic construct
  misc_feature 1..120: note = Synthetic polynucleotide
  source 1..120: mol_type = other DNA
gagaaggcagaaaaaagaagcatagctggtatagaccagtggctagaggtgttctgtgagcaggaggaaaagtgggtatgtgtagactgtgtgcacggtgtggtgggccagcctctgacc

SEQ ID NO: 755 - DNA, 120 bp, synthetic construct
  misc_feature 1..120: note = Synthetic polynucleotide
  source 1..120: mol_type = other DNA
tgttacaagtacgccaccaagcccatgacctatgtggtgggcattgacagtgacggctgggtccgagatgtcacacagaggtacgacccagtctggatgacagtgacccgcaagtgccgg

SEQ ID NO: 756 - DNA, 120 bp, synthetic construct
  misc_feature 1..120: note = Synthetic polynucleotide
  source 1..120: mol_type = other DNA
gttgatgctgagtggtgggccgagaccttgagaccataccagagcccatttatggacagggagaagaaagaagacttggaggtaaggccttggctgccaggggctccaagacacagtcag

SEQ ID NO: 757 - DNA, 120 bp, synthetic construct
  misc_feature 1..120: note = Synthetic polynucleotide
  source 1..120: mol_type = other DNA
ctaaataacatgctcaaccttgtctgtctggcctttgcagtttcaggctaaacacatggaccagcctttgcccactgccattggcttatataagaaccaccctctgtatgccctgaagcg

SEQ ID NO: 758 - DNA, 120 bp, synthetic construct
  misc_feature 1..120: note = Synthetic polynucleotide
  source 1..120: mol_type = other DNA
gcatctcctgaaatatgaggccatctatcccgagacagctgccatccttgggtattgtcgtggagaagcggtctactccaggtgcgtgaggcagcctggttggcctcaggggcttcctga

SEQ ID NO: 759 - DNA, 120 bp, synthetic construct
  misc_feature 1..120: note = Synthetic polynucleotide
  source 1..120: mol_type = other DNA
tcctgtgttggttccacagggattgtgtgcacactctgcattccagggacacgtggctgaagaaagcaagagtggtgaggcttggagaagtaccctacaaggtaactggagctggggggt

SEQ ID NO: 760 - DNA, 120 bp, synthetic construct
  misc_feature 1..120: note = Synthetic polynucleotide
  source 1..120: mol_type = other DNA
ctgaggaactggatgcctttgttgtaaacggtggccgtgtcctctggtgcagatggtgaaaggcttttctaaccgtgctcggaaagcccgacttgctgagccccagctgcgggaagaaaa

SEQ ID NO: 761 - DNA, 120 bp, synthetic construct
  misc_feature 1..120: note = Synthetic polynucleotide
  source 1..120: mol_type = other DNA
tgacctgggcctgtttggctactggcagacagaggagtatcagcccccagtggccgtggacgggaaggtaagggcagcatcagaagggctcaggaccaggccgccttgttcccctgctgg

SEQ ID NO: 762 - DNA, 120 bp, synthetic construct
  misc_feature 1..120: note = Synthetic polynucleotide
  source 1..120: mol_type = other DNA
tgctggccaaatgctgacttgctcacccgacacaggtgccccggaacgagtttgggaatgtgtacctcttcctgcccagcatgatgcctattggctgtgtccagctgaacctgcccaatc

SEQ ID NO: 763 - DNA, 120 bp, synthetic construct
  misc_feature 1..120: note = Synthetic polynucleotide
  source 1..120: mol_type = other DNA
tacaccgcgtggcccgcaagctggacatcgactgtgtccaggccatcactggctttgatttccatggcggctactcccatcccgtgtgcgtgaggggccttcgatggaggctaaacacag

SEQ ID NO: 764 - DNA, 120 bp, synthetic construct
  misc_feature 1..120: note = Synthetic polynucleotide
  source 1..120: mol_type = other DNA
tgtgtccccacaggactgatggatacatcgtctgcgaggaattcaaagacgtgctcctgactgcctgggaaaatgagcaggcagtcattgaaaggaaggagaaggaggtaagcgcatatg

SEQ ID NO: 765 - DNA, 120 bp, synthetic construct
  misc_feature 1..120: note = Synthetic polynucleotide
  source 1..120: mol_type = other DNA
catttttgcctgcagaaaaaggagaagcgggctctagggaactggaagttgctggccaaaggtctgctcatcagggagaggctgaagcgtcgctacgggcccaaggtcagtgcaggttct

SEQ ID NO: 766 - DNA, 120 bp, synthetic construct
  misc_feature 1..120: note = Synthetic polynucleotide
  source 1..120: mol_type = other DNA
acctgtccagagtgaggcagcagctccccacacagatgcaggaggtggactctcttctgatgaagaggaggggaccagctctcaagcagaagcggccaggatactggctgcctcctggcc

SEQ ID NO: 767 - DNA, 120 bp, synthetic construct
  misc_feature 1..120: note = Synthetic polynucleotide
  source 1..120: mol_type = other DNA
tcaaaaccgagaagatgaagaaaagcagaagctgaagggtgggcccaagaagaccaaaagggaaaagaaagcagcagcttcccacctgttcccatttgagcagctgtgagctgagcgccc

SEQ ID NO: 768 - DNA, 120 bp, synthetic construct
  misc_feature 1..120: note = Synthetic polynucleotide
  source 1..120: mol_type = other DNA
cagaaagcgggcgagcagcagttgagcgagcccgaggacatggagatggaaggtgaggcccgagggccggccgcggcggggccggggggcgcctttcattgtggccgggggagccgcggg

SEQ ID NO: 769 - DNA, 120 bp, synthetic construct
  misc_feature 1..120: note = Synthetic polynucleotide
  source 1..120: mol_type = other DNA
tgaatgtttctggtcctaaagacaaggcttctgtgggatgctttaaaaaataaaaaagaatgctgaggtagagctcctagagattctatgcagtcactctgatgtgggtgagagaacctc

SEQ ID NO: 770 - DNA, 120 bp, synthetic construct
  misc_feature 1..120: note = Synthetic polynucleotide
  source 1..120: mol_type = other DNA
ccccatcccccagcagaggccactgcagagccatgcagagatggctgggaaccacaggcttgggctggaaggtgagtcaggttttcaggttgagctccttatgctggtgatggatggaga

SEQ ID NO: 771 - DNA, 120 bp, synthetic construct
  misc_feature 1..120: note = Synthetic polynucleotide
  source 1..120: mol_type = other DNA
ctttaaagcgggagatacagatgacccaccaagaattactcagaaccctgtgatcaatgggaatgtggccctgagtgatggacacaacaccgcggaggaggacatggaggatggtaagtg

SEQ ID NO: 772 - DNA, 120 bp, synthetic construct
  misc_feature 1..120: note = Synthetic polynucleotide
  source 1..120: mol_type = other DNA
atgatgtgttttttttgcagacaccagttggcgctccgaggcaacctttcagttcactgtggagcgcttcagcagactgagtgagtcggtccttagccctccgtgttttgtgcgaaatct

SEQ ID NO: 773 - DNA, 120 bp, synthetic construct
  misc_feature 1..120: note = Synthetic polynucleotide
  source 1..120: mol_type = other DNA
gccatggaagattatggtgatgccacgcttttatccagacagaccacaccaaaaaagcgtaggattctttctccagtgcaatgctgaatctgattccacgtaagacagtatttcattact

SEQ ID NO: 774 - DNA, 120 bp, synthetic construct
  misc_feature 1..120: note = Synthetic polynucleotide
  source 1..120: mol_type = other DNA
aacatctggtaaataatgtcaactaaggctcagattattcttttaaacaggtcatggtcttgccatgcacaagcagtgctgaagataataaattacagagatgatgaaaagtcgttcagt

SEQ ID NO: 775 - DNA, 120 bp, synthetic construct
  misc_feature 1..120: note = Synthetic polynucleotide
  source 1..120: mol_type = other DNA
cgtcgtattagtcatttgttcttccataaagaaaatgattggggattttccaattttatggcctggagtgtaagtaacagtgtatttagtctgaacaattggtgcattactgcttgttgt

SEQ ID NO: 776 - DNA, 120 bp, synthetic construct
  misc_feature 1..120: note = Synthetic polynucleotide
  source 1..120: mol_type = other DNA
tatttatgttttataggaagtgaccgatcctgagaaaggatttatagatgatgacaaagttacctttgaagtctttgtacaggcggatgctccccatggagttgcgtaagtttttcttta

SEQ ID NO: 777 - DNA, 120 bp, synthetic construct
  misc_feature 1..120: note = Synthetic polynucleotide
  source 1..120: mol_type = other DNA
tttcaggtgggattcaaagaagcacacaggctacgtcggcttaaagaatcagggagcgacttgttacatgaacagcctgctacagacgttatttttcacgaatcagctacgaaaggtaaa

SEQ ID NO: 778 - DNA, 120 bp, synthetic construct
  misc_feature 1..120: note = Synthetic polynucleotide
  source 1..120: mol_type = other DNA
taaattcccctgcatcttctcagagtaaaaattcttatttcttaattgtttcaggctgtgtacatgatgccaaccgagggggatgattcgtctaaaagcgtccctttagcattacaaaga

SEQ ID NO: 779 - DNA, 120 bp, synthetic construct
  misc_feature 1..120: note = Synthetic polynucleotide
  source 1..120: mol_type = other DNA
gtgttctatgaattacagcatagtgataaacctgtaggaacaaaaaagttaacaaagtcatttgggtatgtattagacatctgcctttgccattctgcttcctctttttttttttttttt

SEQ ID NO: 780 - DNA, 120 bp, synthetic construct
  misc_feature 1..120: note = Synthetic polynucleotide
  source 1..120: mol_type = other DNA
ttaaatggagttgtttgttttatctttgaaaggtgggaaactttagatagcttcatgcaacatgatgttcaggagctttgtcgagtggtaagtttcaagcaatacttgtatatttcttac

SEQ ID NO: 781 - DNA, 120 bp, synthetic construct
  misc_feature 1..120: note = Synthetic polynucleotide
  source 1..120: mol_type = other DNA
gccaaaacattctttttcagttgctcgataatgtggaaaataagatgaaaggcacctgtgtagagggcaccatacccaaattattccgcggcaaaatggtggtatgtggctaccagttct

SEQ ID NO: 782 - DNA, 120 bp, synthetic construct
  misc_feature 1..120: note = Synthetic polynucleotide
  source 1..120: mol_type = other DNA
ttccttatttgcagtcctatatccagtgtaaagaagtagactatcggtctgatagaagagaagattattatgatatccagctaagtatcaaaggaaagaaaaatagtaagtgttgtgtgt

SEQ ID NO: 783 - DNA, 120 bp, synthetic construct
  misc_feature 1..120: note = Synthetic polynucleotide
  source 1..120: mol_type = other DNA
ctctctctctctttaaagtatttgaatcatttgtggattatgtggcagtagaacagctcgatggggacaataaatacgacgctggggaacatggcttacaggtaaattgagtgttttgtg

SEQ ID NO: 784 - DNA, 120 bp, synthetic construct
  misc_feature 1..120: note = Synthetic polynucleotide
  source 1..120: mol_type = other DNA
tttaggaagcagagaaaggtgtgaaattcctaacattgccaccagtgttacatctacaactgatgagatttatgtatgaccctcagacggaccaaaatatcaagatcaatgataggtaat

SEQ ID NO: 785 - DNA, 120 bp, synthetic construct
  misc_feature 1..120: note = Synthetic polynucleotide
  source 1..120: mol_type = other DNA
tgtaaattaatactgaatttcttttaaaatgttttctaataggtttgaattcccagagcagttaccacttgatgaatttttgcaaaaaacagatcctaaggaccctgcaaattatattct

SEQ ID NO: 786 - DNA, 120 bp, synthetic construct
  misc_feature 1..120: note = Synthetic polynucleotide
  source 1..120: mol_type = other DNA
tcatgcagtcctggttcatagtggagataatcatggtggacattatgtggtttatctaaaccccaaaggggatggcaaagtaagtggtgggacccagggctgaatgcaagctcatcagaa

SEQ ID NO: 787 - DNA, 120 bp, synthetic construct
  misc_feature 1..120: note = Synthetic polynucleotide
  source 1..120: mol_type = other DNA
cttgtcacagtgcttgatcgctgctggtgtgtgttgttttccttgcagtggtgtaaatttgatgacgacgtggtgtcaaggtgtactaaagaggaagcaattgagcacaattatgggggt

SEQ ID NO: 788 - DNA, 120 bp, synthetic construct
  misc_feature 1..120: note = Synthetic polynucleotide
  source 1..120: mol_type = other DNA
cacgatgacgacctgtctgttcgacactgcactaatgcttacatgttagtctacatcagggaatcaaaactgagtgagtagtgttcacttttgttctgttctttactgtggtggacttgg

SEQ ID NO: 789 - DNA, 120 bp, synthetic construct
  misc_feature 1..120: note = Synthetic polynucleotide
  source 1..120: mol_type = other DNA
ctaagaggggaaagtccatctgcaggggtcaaagaggatgcctttgtcctgcaggtgaagttttacaggcggtcaccgaccatgatattcctcagcagttggtggagcgattacaagaag

SEQ ID NO: 790 - DNA, 120 bp, synthetic construct
  misc_feature 1..120: note = Synthetic polynucleotide
  source 1..120: mol_type = other DNA
agaaaaggatcgaggctcagaagcggaaggagcggcaggaagcccatctctatatgcaagtgcaggtcagcccccgcccttctgggactccgggtcaccatgcagccgggcacccatctc

SEQ ID NO: 791 - DNA, 120 bp, synthetic construct
  misc_feature 1..120: note = Synthetic polynucleotide
  source 1..120: mol_type = other DNA
tcattttttgtcagttgtcaagtcttttctgtgctttcatacatattttcagatagtcgcagaggaccagttttgtggccaccaagggaatgacatgtacgatgaagaaaaagtgaaata

SEQ ID NO: 792 - DNA, 120 bp, synthetic construct
  misc_feature 1..120: note = Synthetic polynucleotide
  source 1..120: mol_type = other DNA
cactgtgttcaaagtattgaagaactcctcgcttgctgagtttgttcagagcctctctcagaccatggtgcgtaccggtcccgtggatataccccaccgtggctcccaacgtccaccttc

SEQ ID NO: 793 - DNA, 120 bp, synthetic construct
  misc_feature 1..120: note = Synthetic polynucleotide
  source 1..120: mol_type = other DNA
ttttttcagggatttccacaagatcaaattcgattgtggcccatgcaagcaaggagtaatggaacaaaacgaccagcaatgttagataatgaagccgacggcaataaaacagtaaatatt

SEQ ID NO: 794 - DNA, 120 bp, synthetic construct
  misc_feature 1..120: note = Synthetic polynucleotide
  source 1..120: mol_type = other DNA
gaaatagatgattgagctcagtgataatgaaaacccttggacaatattcctggaaacagttgatcccgagctggctgctagtggagcgaccttacccaagtttgataaagatcgtaagtg

SEQ ID NO: 795 - DNA, 120 bp, synthetic construct
  misc_feature 1..120: note = Synthetic polynucleotide
  source 1..120: mol_type = other DNA
tattaaccttgtagatgatgtaatgttatttttgaagatgtatgatcccaaaacgcggagcttgaattactgtgggcatatctacacaccaatatcctgtaaaatacgtaagtccttcgt

SEQ ID NO: 796 - DNA, 120 bp, synthetic construct
  misc_feature 1..120: note = Synthetic polynucleotide
  source 1..120: mol_type = other DNA
catttaaaatatcttctttctcctaggtgacttgctcccagttatgtgtgacagagcaggatttattcaagatactagccttatcctctatgaggtttggatggtttatttttccataat

SEQ ID NO: 797 - DNA, 120 bp, synthetic construct
  misc_feature 1..120: note = Synthetic polynucleotide
  source 1..120: mol_type = other DNA
ttcactgtaggaagttaaaccgaatttaacagagagaattcaggactatgacgtgtctcttgataaagcccttgatgaactaatggatggtgacatcatagtatttcagaagtatgtact

SEQ ID NO: 798 - DNA, 120 bp, synthetic construct
  misc_feature 1..120: note = Synthetic polynucleotide
  source 1..120: mol_type = other DNA
ccatggactttgattcagactgcaaactttctccctccaccagggatgaccctgaaaatgataacagtgaattacccaccgcaaaggagtatttccgagatctctaccaccgcgttgatg

SEQ ID NO: 799 - DNA, 120 bp, synthetic construct
  misc_feature 1..120: note = Synthetic polynucleotide
  source 1..120: mol_type = other DNA
tcattttctgtgataaaacaatccctaatgatcctggatttgtggttacgttatcaaatagaatgaattattttcaggtatttaataaatgctcctttcctcttcagatcccactccaga

SEQ ID NO: 800 - DNA, 120 bp, synthetic construct
  misc_feature 1..120: note = Synthetic polynucleotide
  source 1..120: mol_type = other DNA
tgtgaaccttcctttctgtgtcttaggttgcaaagacagttgcacagaggctcaacacagatccaatgttgctgcagtttttcaagtctcaagggtaggtcacatgtgtggacacttgct

SEQ ID NO: 801 - DNA, 120 bp, synthetic construct
  misc_feature 1..120: note = Synthetic polynucleotide
  source 1..120: mol_type = other DNA
gtgtagttatagggatggcccaggtaatcctcttagacataattatgaaggtactttaagagatcttctacagttcttcaagcctagacaacctaagaaactttactatcagcaggtatg

SEQ ID NO: 802 - DNA, 120 bp, synthetic construct
  misc_feature 1..120: note = Synthetic polynucleotide
  source 1..120: mol_type = other DNA
gaatctttcttttcttaatagcttaagatgaaaatcacagactttgagaacaggcgaagttttaaatgtatatggttaaacagccaatttagggaagaggtaagtttttttaatactatg

SEQ ID NO: 803 - DNA, 120 bp, synthetic construct
  misc_feature 1..120: note = Synthetic polynucleotide
  source 1..120: mol_type = other DNA
cattacttaggaaataacactatatccagacaagcatgggtgtgtccgggacctgttagaagaatgtaaaaaggccgtggagcttggggagaaagcatcagggaaacttaggcaagtatt

SEQ ID NO: 804 - DNA, 120 bp, synthetic construct
  misc_feature 1..120: note = Synthetic polynucleotide
  source 1..120: mol_type = other DNA
ctctgttcaggctgctagaaattgtaagctacaaaatcattggtgttcatcaagaagatgaactattagaatgtttatctcctgcaacgagccggacgtttcgaatagaggtatctgtcc

SEQ ID NO: 805 - DNA, 120 bp, synthetic construct
  misc_feature 1..120: note = Synthetic polynucleotide
  source 1..120: mol_type = other DNA
tggacactttgaccacatcacttgcacttacagaactgtgtctgttgtccgtgtccccaggaaatccctttggaccaggtggacatagacaaagagaatgagatgcttgtcacagtggcg

SEQ ID NO: 806 - DNA, 120 bp, synthetic construct
  misc_feature 1..120: note = Synthetic polynucleotide
  source 1..120: mol_type = other DNA
catttccacaaagaggtcttcggaacgttcggaatcccgtttttgctgaggatacaccaggtatgctgttgtggttggccgagtggctcacgtcaaaacctggagcctttgagtggccgc

SEQ ID NO: 807 - DNA, 120 bp, synthetic construct
  misc_feature 1..120: note = Synthetic polynucleotide
  source 1..120: mol_type = other DNA
agcacatgtcctttgccattctagggcgagcattttcgagaagtgatgaagcgaatccagagcctgctggacatccaggagaaggagtttgagaaggtgtgcagctggggcctcctgggg

SEQ ID NO: 808 - DNA, 120 bp, synthetic construct
  misc_feature 1..120: note = Synthetic polynucleotide
  source 1..120: mol_type = other DNA
gtttaaatttgcaattgtaatgatgggccgacaccagtacataaatgaagacgagtatgaagtaaatttgaaagactttgagccacagcccggtaagggttctcccccccccgggggctg

SEQ ID NO: 809 - DNA, 120 bp, synthetic construct
  misc_feature 1..120: note = Synthetic polynucleotide
  source 1..120: mol_type = other DNA
caccaggtaatatgtctcatcctcggccttggctagggctcgaccacttcaacaaagccccaaagaggagtcgctacacttaccttgaaaaggccattaaaatccataactgatttccaa

SEQ ID NO: 810 - DNA, 120 bp, synthetic construct
  misc_feature 1..120: note = Synthetic polynucleotide
  source 1..120: mol_type = other DNA
tatttctagatatggatcagaaactttcgaagttggtagaagagctcacaacttcaggagaaccccgactaaatcctgagaaaatgaaggaactgaagaaaatttgcaagtatgtcttag

SEQ ID NO: 811 - DNA, 120 bp, synthetic construct
  misc_feature 1..120: note = Synthetic polynucleotide
  source 1..120: mol_type = other DNA
gctcgcatccccaggtcttcagaggagcagctgagccgcgcctaccgcctgctgatagcacagctgacccaggagcacgccgagatccgtctctcagccttccagattgtggaggaactc

SEQ ID NO: 812 - DNA, 120 bp, synthetic construct
  misc_feature 1..120: note = Synthetic polynucleotide
  source 1..120: mol_type = other DNA
ttcgtcaggtctcaccagttccggatgctggttgtttccaacttccaggagttcctggagctcacgctgggcacagaccccgcacagcctctgccgccccccagggaggcggcacagagg

SEQ ID NO: 813 - DNA, 120 bp, synthetic construct
  misc_feature 1..120: note = Synthetic polynucleotide
  source 1..120: mol_type = other DNA
ctgaggcaggcgaccacccgggccgtggaagggtggaatgagaagtttggggaggcctacaagaagcttgccttgggctaccacttcttaagacacaacaaaaaggtaggtgggcctggc

SEQ ID NO: 814 - DNA, 120 bp, synthetic construct
  misc_feature 1..120: note = Synthetic polynucleotide
  source 1..120: mol_type = other DNA
gtgagccaccgcgcctgtcccctagtctttattttcaattgctcaggtggattttcaagacacgaatgctcggagtctggcagaaaggaagagagaagaggagaagcagaagcacttgga

SEQ ID NO: 815 - DNA, 120 bp, synthetic construct
  misc_feature 1..120: note = Synthetic polynucleotide
  source 1..120: mol_type = other DNA
taaaatttatcaagaaagagccagccaggcggagagggagatgcaaggcaagtgtccaggacggagggaggggcccacgcctctgagggttgcccgtggtccagcagttcatcctcctgg

SEQ ID NO: 816 - DNA, 120 bp, synthetic construct
  misc_feature 1..120: note = Synthetic polynucleotide
  source 1..120: mol_type = other DNA
cttttgctccacatagcgcagcaaacaggtgtcttgatcttccggcagaaatgtctggagaaattgaatcctgcttgacggaggtagagagctgctttaggctgctggtgccttttgact

SEQ ID NO: 817 - DNA, 120 bp, synthetic construct
  misc_feature 1..120: note = Synthetic polynucleotide
  source 1..120: mol_type = other DNA
ttgacccgaacccggagacggaatcccttggcatggcttctggcatgtccgatgcccttcgctcctcctgcgcgggccaggtgggcccctgccggtctggcacccctgacccccgggacg

SEQ ID NO: 818 - DNA, 120 bp, synthetic construct
  misc_feature 1..120: note = Synthetic polynucleotide
  source 1..120: mol_type = other DNA
gggagcagccctgctgcagtagagacctgcctgcctctgcaggccaccccagagcgggcggcggggcacagccatcccagacagccacaggtgacccctcagatgaggacgaggacagcg

SEQ ID NO: 819 - DNA, 120 bp, synthetic construct
  misc_feature 1..120: note = Synthetic polynucleotide
  source 1..120: mol_type = other DNA
acctcgaggagtttgtgcggagccacgggctgggctcgcacaagtacacgctggatgtggagctctgctcaggtaactgccttcgcggggtctctgtggcgccaccctgccccggctccc

SEQ ID NO: 820 - DNA, 120 bp, synthetic construct
  misc_feature 1..120: note = Synthetic polynucleotide
  source 1..120: mol_type = other DNA
ccagagggcctgaaggtgcaggagaacgaggacaaccttgctctcatccacgccgcccgcgacacactcaagctcatccggaacaagttcctgccggctgtgtgctcgtggatccaggtg

SEQ ID NO: 821 - DNA, 120 bp, synthetic construct
  misc_feature 1..120: note = Synthetic polynucleotide
  source 1..120: mol_type = other DNA
gtgtgccagggaccccccgggccagtggacctggcatgtgcctcccagcaggacagcttcccaggtcctgcccggccggcccctgagctgttcgcacccccgtttcgcagcgcttcaccc

SEQ ID NO: 822 - DNA, 120 bp, synthetic construct
  misc_feature 1..120: note = Synthetic polynucleotide
  source 1..120: mol_type = other DNA
gcgtcgggacccacggtggatgtttaaagcgtgccattgacctgaaggctgaattggagctcgtactgagaaaatacaaggagctggacatcgagcctgagggaggggaaaggcgcaggg

SEQ ID NO: 823 - DNA, 120 bp, synthetic construct
  misc_feature 1..120: note = Synthetic polynucleotide
  source 1..120: mol_type = other DNA
acagacagaagccctgggggatgcggaggaagatgaggacgatgaggactttgtggaggtccctgagaaggaggggtatgagccacacatccccgaccacttgcggcctgagtatggtga

SEQ ID NO: 824 - DNA, 120 bp, synthetic construct
  misc_feature 1..120: note = Synthetic polynucleotide
  source 1..120: mol_type = other DNA
ggcgggttgtgggagtggtggcagggagtggacacgtgtctgtttcagggctggaggcagcaccagagaaagacacagttgtgcggtgcttgcggacgaggacgaggatggacgaggagg

SEQ ID NO: 825 - DNA, 120 bp, synthetic construct
  misc_feature 1..120: note = Synthetic polynucleotide
  source 1..120: mol_type = other DNA
tgtcggaccccacctctgcggctgctcagctgcggcagctccgggaccacttgcctccaccctcatctgccaggtgactcccagtgtcctgtgtgctgagccccctgcccggcgctgcca

SEQ ID NO: 826 - DNA, 120 bp, synthetic construct
  misc_feature 1..120: note = Synthetic polynucleotide
  source 1..120: mol_type = other DNA
gctgtgggtggcaccagcagcaccgtcaggctgtcccactctgctcctgtagcccctccagagcgttgccagagccacaggaggcccagaagctggcagcagagcgggcccgggcgcctg

SEQ ID NO: 827 - DNA, 120 bp, synthetic construct
  misc_feature 1..120: note = Synthetic polynucleotide
  source 1..120: mol_type = other DNA
tggtgccctacggcgtggacctgcactactggggccaggagctccccacagccgggaagattgtcaagtgagtccccatgtgtctgaagtcggccagggcacacaaccagggtcccagcc

SEQ ID NO: 828 - DNA, 120 bp, synthetic construct
  misc_feature 1..120: note = Synthetic polynucleotide
  source 1..120: mol_type = other DNA
ctatgagggcctctggctgtgtctgcaggtctgactcccagcaccgcttctggaagcccagcgaggtggaggaggaagtggtcaatgccgacatctccgagatgctccggagccgccaca

SEQ ID NO: 829 - DNA, 120 bp, synthetic construct
  misc_feature 1..120: note = Synthetic polynucleotide
  source 1..120: mol_type = other DNA
tcacttttgccgggaagtttgagcctgtgcagcactggtgccgtgccccgaggccagacggccggctctgtgagcgccaagaccggctgaaggtgaggccgtggcccgagggcgggggtg

SEQ ID NO: 830 - DNA, 120 bp, synthetic construct
  misc_feature 1..120: note = Synthetic polynucleotide
  source 1..120: mol_type = other DNA
gactttccacagctgcccaagaccttccggctccagaggagggacggagccatccccacgtccctgtggctctcaggacgccctcctctgaggggcgcgtgattccaggttgtgtacact

SEQ ID NO: 831 - DNA, 120 bp, synthetic construct
  misc_feature 1..120: note = Synthetic polynucleotide
  source 1..120: mol_type = other DNA
ttggctctgtgataccacccaccctggtcgctgtttgtgcccaagtcgtggtggtggggggaggtggtcaaggcaagcggacccctccccgccatcagccaccgtgtcctcgctgtgcag

SEQ ID NO: 832 - DNA, 120 bp, synthetic construct
  misc_feature 1..120: note = Synthetic polynucleotide
  source 1..120: mol_type = other DNA
tgccctttccatgggaagattgttccacgggacgacgaaggacggccgctcgacccggaagacagggctcgtgagcagcggcggcagctgcagaagcaggagcgcccgggtaggtctggg

SEQ ID NO: 833 - DNA, 120 bp, synthetic construct
  misc_feature 1..120: note = Synthetic polynucleotide
  source 1..120: mol_type = other DNA
cgtctcctgaagtgcttgagttttgtttgcagaatggcaggaccctgagttgatgagagacgtggaagcagccacagggcaggatctcggctcatccaggtacagcgggaaaggcagggg

SEQ ID NO: 834 - DNA, 120 bp, synthetic construct
  misc_feature 1..120: note = Synthetic polynucleotide
  source 1..120: mol_type = other DNA
gaagaagaggaggtaccccagcctcaccaacctgaaggctcaggctgataccgcccgcgctcgcattgggagaaaagtcttcgccaagtaagagtggctgctgggtcacctcccaccgcg

SEQ ID NO: 835 - DNA, 120 bp, synthetic construct
  misc_feature 1..120: note = Synthetic polynucleotide
  source 1..120: mol_type = other DNA
tgtttccccacagggcagctgtgcggagggtagtggcagccatgaaccggatggaccagaagaagcacgagaagttttcaaaccagtttaactacgcactgaactagagagcggggccca

SEQ ID NO: 836 - DNA, 120 bp, synthetic construct
  misc_feature 1..120: note = Synthetic polynucleotide
  source 1..120: mol_type = other DNA
tttttctttatcttttttttttttagactacaacaaaatcacaattaaatccggcctttgaagatgcctggagcagcagtgatgaagaaggatgaatatcatctttagtacctttttgtc

SEQ ID NO: 837 - DNA, 120 bp, synthetic construct
  misc_feature 1..120: note = Synthetic polynucleotide
  source 1..120: mol_type = other DNA
ctttgccttctatgttataggaactttatagtggtagcagagactgcaacattctggcttgggttccatccttatatgaaccagttcctgatgatgatgaggtaaatattattttcacaa

SEQ ID NO: 838 - DNA, 120 bp, synthetic construct
  misc_feature 1..120: note = Synthetic polynucleotide
  source 1..120: mol_type = other DNA
accattgctgtttatacagtttactcaggagaacagataactatgcttaagggacattataaaactgttgactgctgtgtatttcagtcaaatttccaggtaagagtgatacaggaattt

SEQ ID NO: 839 - DNA, 120 bp, synthetic construct
  misc_feature 1..120: note = Synthetic polynucleotide
  source 1..120: mol_type = other DNA
cttattattttaatcctacaggtgaactatggaaaagtttgtaataacagtaaaaaaggattgaaattcactgtctcctgtggctgcagttcagaatttgtttttgtaccatatggtagc

SEQ ID NO: 840 - DNA, 120 bp, synthetic construct
  misc_feature 1..120: note = Synthetic polynucleotide
  source 1..120: mol_type = other DNA
ctcactgttggtacagataatcgaatgaggctctggaatagttccaatggagaaaacacacttgtaagagatttttaagtatatatttgggtataaattaagaaatggatctgtgacata

SEQ ID NO: 841 - DNA, 120 bp, synthetic construct
  misc_feature 1..120: note = Synthetic polynucleotide
  source 1..120: mol_type = other DNA
acattaaatatgtcctgaatacaagaacagataaactgaactatgtttttcattgcagcaaacactgctcataatgggaaagttaatggcttatgttttacaagtgatggacttcacctc

SEQ ID NO: 842 - DNA, 120 bp, synthetic construct
  misc_feature 1..120: note = Synthetic polynucleotide
  source 1..120: mol_type = other DNA
ttcttctcagtgctgacagtagagtaaaattatgggatgtgagaagagcatcaggatgtttgattactcttgatcaacataatgggaaaaagtcacaagctgttgaatcaggtaaggaag

SEQ ID NO: 843 - DNA, 120 bp, synthetic construct
  misc_feature 1..120: note = Synthetic polynucleotide
  source 1..120: mol_type = other DNA
gacttttcatatagttgtatttgcaggtcacagacaagaaatattagcagtttcctggtctccacgttatgactatatcttggcaacagcaaggtaaaatttaacttatttgcttttgaa

SEQ ID NO: 844 - DNA, 120 bp, synthetic construct
  misc_feature 1..120: note = Synthetic polynucleotide
  source 1..120: mol_type = other DNA
aatgtcttttgattaccagactgcagatgtatttaattttgaggaaacagtttatagtcatcatatgtctccagtctccaccaagcactgtttggtagcaggtttgtaagtgtattcttt

SEQ ID NO: 845 - DNA, 120 bp, synthetic construct
  misc_feature 1..120: note = Synthetic polynucleotide
  source 1..120: mol_type = other DNA
tttatttttaagagttttgggactggaattaaataaagacagagatgttgaaagaatccacggcggtggaattaacacccttgacattgaacctgttgaagggagatagtaagtttatta

SEQ ID NO: 846 - DNA, 120 bp, synthetic construct
  misc_feature 1..120: note = Synthetic polynucleotide
  source 1..120: mol_type = other DNA
ccagccggtgtgaggacacgatatgctggggtttttgtccgcacgccaaacgggtttggaggaccctcttcgccttcggagagcagagtcaacacggaggtaaagaaaaccttacttttt

SEQ ID NO: 847 - DNA, 120 bp, synthetic construct
  misc_feature 1..120: note = Synthetic polynucleotide
  source 1..120: mol_type = other DNA
gcatgttcacatcaagctcatttgataaaactctgaaagtatgggatacaaatacattacaagtaagtacattaaaacatttgcagattgctttgtaggataaaggagaatttaatccta

SEQ ID NO: 848 - DNA, 120 bp, synthetic construct
  misc_feature 1..120: note = Synthetic polynucleotide
  source 1..120: mol_type = other DNA
taccagttttgaaactcacataagtcagttaatgtaaattttgattgctgttttgtagagatcatcctgatgttcacagatacagtgtggagactgtacagtggtatcctcatgacactg

SEQ ID NO: 849 - DNA, 120 bp, synthetic construct
  misc_feature 1..120: note = Synthetic polynucleotide
  source 1..120: mol_type = other DNA
tattatcagcatgttatcaggtggttcagatggtgtgattgtactttatgaccttgagaactccagcagacaatcttattacacatgtaaagcagtgtgttccattggcaggtatgtatt

SEQ ID NO: 850 - DNA, 120 bp, synthetic construct
  misc_feature 1..120: note = Synthetic polynucleotide
  source 1..120: mol_type = other DNA
ggtatttttattttatttcaaacggcaactactttgagtaaacgattcaataaaaagaaacgttactaacagtgtattctttgtaagtgacatgactaatgtactttgtgctggttgttg

SEQ ID NO: 851 - DNA, 120 bp, synthetic construct
  misc_feature 1..120: note = Synthetic polynucleotide
  source 1..120: mol_type = other DNA
ttttgatgatgacttatgataaaattatcttaaaccggttctgtttttacagttggtactagaggacccaaagtacaactttgtgacttgaagtctggatcctgttctcacattctacag

SEQ ID NO: 852 - DNA, 21 bp, synthetic construct
  misc_feature 1..21: note = Synthetic polynucleotide
  source 1..21: mol_type = other DNA
aatttgtgatacccctcgacg

SEQ ID NO: 853 - DNA, 24 bp, synthetic construct
  misc_feature 1..24: note = Synthetic polynucleotide
  source 1..24: mol_type = other DNA
tgggaaggctctgtgtagatcgga

SEQ ID NO: 854 - DNA, 20 bp, synthetic construct
  misc_feature 1..20: note = Synthetic polynucleotide
  source 1..20: mol_type = other DNA
tgtgagatggcatattcggc

SEQ ID NO: 855 - DNA, 20 bp, synthetic construct
  misc_feature 1..20: note = Synthetic polynucleotide
  source 1..20: mol_type = other DNA
tccttgctaactttgccacc

SEQ ID NO: 856 - DNA, 21 bp, synthetic construct
  misc_feature 1..21: note = Synthetic polynucleotide
  source 1..21: mol_type = other DNA
tgcttccacagtgctgagcct

SEQ ID NO: 857 - DNA, 22 bp, synthetic construct
  misc_feature 1..22: note = Synthetic polynucleotide
  source 1..22: mol_type = other DNA
catctttgctactggttttccc

SEQ ID NO: 858 - DNA, 22 bp, synthetic construct
  misc_feature 1..22: note = Synthetic polynucleotide
  source 1..22: mol_type = other DNA
gtccgcgaagatgacaaaattg

SEQ ID NO: 859 - DNA, 24 bp, synthetic construct
  misc_feature 1..24: note = Synthetic polynucleotide
  source 1..24: mol_type = other DNA
agcacactggactttggcgatgta

SEQ ID NO: 860 - DNA, 21 bp, synthetic construct
  misc_feature 1..21: note = Synthetic polynucleotide
  source 1..21: mol_type = other DNA
aattcaggagacatagggcac

SEQ ID NO: 861 - DNA, 22 bp, synthetic construct
  misc_feature 1..22: note = Synthetic polynucleotide
  source 1..22: mol_type = other DNA
tctttatgacgcagagctaacc

SEQ ID NO: 862 - DNA, 25 bp, synthetic construct
  misc_feature 1..25: note = Synthetic polynucleotide
  source 1..25: mol_type = other DNA
ttcggcagcttgaaatttacagggc

SEQ ID NO: 863 - DNA, 20 bp, synthetic construct
  misc_feature 1..20: note = Synthetic polynucleotide
  source 1..20: mol_type = other DNA
tccgcaaagcagtgagatag

SEQ ID NO: 864 - DNA, 21 bp, synthetic construct
  misc_feature 1..21: note = Synthetic polynucleotide
  source 1..21: mol_type = other DNA
gactccaggaaaacgacagag

SEQ ID NO: 865 - DNA, 28 bp, synthetic construct
  misc_feature 1..28: note = Synthetic polynucleotide
  source 1..28: mol_type = other DNA
agcaaaagagatgaagcactacccagtc

SEQ ID NO: 866 - DNA, 21 bp, synthetic construct
  misc_feature 1..21: note = Synthetic polynucleotide
  source 1..21: mol_type = other DNA
ggaggcaaaacatagaggtcg

SEQ ID NO: 867 - DNA, 21 bp, synthetic construct
  misc_feature 1..21: note = Synthetic polynucleotide
  source 1..21: mol_type = other DNA
ggaaagtgggaagataccgag

SEQ ID NO: 868 - DNA, 27 bp, synthetic construct
  misc_feature 1..27: note = Synthetic polynucleotide
  source 1..27: mol_type = other DNA
tgcaggacaaagagaaacgtctgaagc

SEQ ID NO: 869 - DNA, 22 bp, synthetic construct
  misc_feature 1..22: note = Synthetic polynucleotide
  source 1..22: mol_type = other DNA
ccttcgtcaaattcagcatcac

SEQ ID NO: 870 - DNA, 20 bp, synthetic construct
  misc_feature 1..20: note = Synthetic polynucleotide
  source 1..20: mol_type = other DNA
agtagaaatacggctgcacc

SEQ ID NO: 871 - DNA, 20 bp, synthetic construct
  misc_feature 1..20: note = Synthetic polynucleotide
  source 1..20: mol_type = other DNA
tccggaaggccgaggccttg

SEQ ID NO: 872 - DNA, 22 bp, synthetic construct
  misc_feature 1..22: note = Synthetic polynucleotide
  source 1..22: mol_type = other DNA
ccaggtctccattcttagttgc

SEQ ID NO: 873 - DNA, 22 bp, synthetic construct
  misc_feature 1..22: note = Synthetic polynucleotide
  source 1..22: mol_type = other DNA
agccattgacagagcatatagg

SEQ ID NO: 874 - DNA, 26 bp, synthetic construct
  misc_feature 1..26: note = Synthetic polynucleotide
  source 1..26: mol_type = other DNA
cctccacagttcccaaggatatcagc

SEQ ID NO: 875 - DNA, 21 bp, synthetic construct
  misc_feature 1..21: note = Synthetic polynucleotide
  source 1..21: mol_type = other DNA
ggcattttcacttccaaccac

SEQ ID NO: 876 - DNA, 20 bp, synthetic construct
  misc_feature 1..20: note = Synthetic polynucleotide
  source 1..20: mol_type = other DNA
gtgaggacacgatatgctgg

SEQ ID NO: 877 - DNA, 26 bp, synthetic construct
  misc_feature 1..26: note = Synthetic polynucleotide
  source 1..26: mol_type = other DNA
aacacggagagttttgggactggaat

SEQ ID NO: 878 - DNA, 21 bp, synthetic construct
  misc_feature 1..21: note = Synthetic polynucleotide
  source 1..21: mol_type = other DNA
gccgtggattctttcaacatc

SEQ ID NO: 879 - DNA, 22 bp, synthetic construct
  misc_feature 1..22: note = Synthetic polynucleotide
  source 1..22: mol_type = other DNA
tccacatccaatcataagcagg

SEQ ID NO: 880 - DNA, 23 bp, synthetic construct
  misc_feature 1..23: note = Synthetic polynucleotide
  source 1..23: mol_type = other DNA
cggacatcagccagaaatgcagc

SEQ ID NO: 881 - DNA, 22 bp, synthetic construct
  misc_feature 1..22: note = Synthetic polynucleotide
  source 1..22: mol_type = other DNA
cttagaccgttacagccatcag

SEQ ID NO: 882 - DNA, 21 bp, synthetic construct
  misc_feature 1..21: note = Synthetic polynucleotide
  source 1..21: mol_type = other DNA
caaaaccctctttcagcatgg

SEQ ID NO: 883 - DNA, 25 bp, synthetic construct
  misc_feature 1..25: note = Synthetic polynucleotide
  source 1..25: mol_type = other DNA
tggctcagtgttgctatccagatgc

SEQ ID NO: 884 - DNA, 21 bp, synthetic construct
  misc_feature 1..21: note = Synthetic polynucleotide
  source 1..21: mol_type = other DNA
agcagacaccaaagtaagacc

SEQ ID NO: 885 - DNA, 20 bp, synthetic construct
  misc_feature 1..20: note = Synthetic polynucleotide
  source 1..20: mol_type = other DNA
agccatggtttcagacgaag

SEQ ID NO: 886 - DNA, 26 bp, synthetic construct
  misc_feature 1..26: note = Synthetic polynucleotide
  source 1..26: mol_type = other DNA
ttcctttaatgcttgctttccccacc

SEQ ID NO: 887 - DNA, 18 bp, synthetic construct
  misc_feature 1..18: note = Synthetic polynucleotide
  source 1..18: mol_type = other DNA
tttcccagcaccatcacg

SEQ ID NO: 888 - DNA, 22 bp, synthetic construct
  misc_feature 1..22: note = Synthetic polynucleotide
  source 1..22: mol_type = other DNA
ccatctttccctcaatctccag

SEQ ID NO: 889 - DNA, 25 bp, synthetic construct
  misc_feature 1..25: note = Synthetic polynucleotide
  source 1..25: mol_type = other DNA
cgtcgcttaatcggagccaaagtct

SEQ ID NO: 890 - DNA, 21 bp, synthetic construct
  misc_feature 1..21: note = Synthetic polynucleotide
  source 1..21: mol_type = other DNA
ctgcattgtaggtgtactcgg

SEQ ID NO: 891 - DNA, 21 bp, synthetic construct
  misc_feature 1..21: note = Synthetic polynucleotide
  source 1..21: mol_type = other DNA
tggtcaacgtcttgaaaggag

SEQ ID NO: 892 - DNA, 26 bp, synthetic construct
  misc_feature 1..26: note = Synthetic polynucleotide
  source 1..26: mol_type = other DNA
tgaatgatgaacttcttccccagggc

SEQ ID NO: 893 - DNA, 25 bp, synthetic construct
  misc_feature 1..25: note = Synthetic polynucleotide
  source 1..25: mol_type = other DNA
aaccaattctgctattacaaagacg

SEQ ID NO: 894 - DNA, 20 bp, synthetic construct
  misc_feature 1..20: note = Synthetic polynucleotide
  source 1..20: mol_type = other DNA
ttcgggtagtggaaaaccag

SEQ ID NO: 895 - DNA, 25 bp, synthetic construct
  misc_feature 1..25: note = Synthetic polynucleotide
  source 1..25: mol_type = other DNA
cccctcaacgttagcttcaccaaca

SEQ ID NO: 896 - DNA, 20 bp, synthetic construct
  misc_feature 1..20: note = Synthetic polynucleotide
  source 1..20: mol_type = other DNA
agtagaaatacggctgcacc

SEQ ID NO: 897 - DNA, 20 bp, synthetic construct
  misc_feature 1..20: note = Synthetic polynucleotide
  source 1..20: mol_type = other DNA
atataacagaaccggcccac

SEQ ID NO: 898 - DNA, 23 bp, synthetic construct
  misc_feature 1..23: note = Synthetic polynucleotide
  source 1..23: mol_type = other DNA
catctccttgccagcggtagacg

SEQ ID NO: 899 - DNA, 21 bp, synthetic construct
  misc_feature 1..21: note = Synthetic polynucleotide
  source 1..21: mol_type = other DNA
ccctctaagacccatttcagc

SEQ ID NO: 900 - DNA, 21 bp, synthetic construct
  misc_feature 1..21: note = Synthetic polynucleotide
  source 1..21: mol_type = other DNA
ccctctaagacccatttcagc

SEQ ID NO: 901 - DNA, 24 bp, synthetic construct
  misc_feature 1..24: note = Synthetic polynucleotide
  source 1..24: mol_type = other DNA
tcttgtgaaaccactgcccaacca

SEQ ID NO: 902 - DNA, 21 bp, synthetic construct
  misc_feature 1..21: note = Synthetic polynucleotide
  source 1..21: mol_type = other DNA
attctctgccactggattacc

SEQ ID NO: 903 - DNA, 20 bp, synthetic construct
  misc_feature 1..20: note = Synthetic polynucleotide
  source 1..20: mol_type = other DNA
gcaaagaccaaagagacacg

SEQ ID NO: 904 - DNA, 23 bp, synthetic construct
  misc_feature 1..23: note = Synthetic polynucleotide
  source 1..23: mol_type = other DNA
tcaaagtggatgtcgagggtggc

SEQ ID NO: 905 - DNA, 20 bp, synthetic construct
  misc_feature 1..20: note = Synthetic polynucleotide
  source 1..20: mol_type = other DNA
ggaatgttaaccccgcaaac

SEQ ID NO: 906 - DNA, 22 bp, synthetic construct
  misc_feature 1..22: note = Synthetic polynucleotide
  source 1..22: mol_type = other DNA
ccaggagaaggagtttgagaag

SEQ ID NO: 907 - DNA, 25 bp, synthetic construct
  misc_feature 1..25: note = Synthetic polynucleotide
  source 1..25: mol_type = other DNA
tttgaaagactttgagccacagccc

SEQ ID NO: 908 - DNA, 22 bp, synthetic construct
  misc_feature 1..22: note = Synthetic polynucleotide
  source 1..22: mol_type = other DNA
ggccgaggatgagacatattac

SEQ ID NO: 909 - DNA, 22 bp, synthetic construct
  misc_feature 1..22: note = Synthetic polynucleotide
  source 1..22: mol_type = other DNA
gatgaggacgatgaggactttg

SEQ ID NO: 910 - DNA, 23 bp, synthetic construct
  misc_feature 1..23: note = Synthetic polynucleotide
  source 1..23: mol_type = other DNA
cgaccacttgcggcctgagtatg

SEQ ID NO: 911 - DNA, 20 bp, synthetic construct
  misc_feature 1..20: note = Synthetic polynucleotide
  source 1..20: mol_type = other DNA
tgtgtctttctctggtgctg

SEQ ID NO: 912 - DNA, 18 bp, synthetic construct
  misc_feature 1..18: note = Synthetic polynucleotide
  source 1..18: mol_type = other DNA
gagtatcgagcggaagcg

SEQ ID NO: 913 - DNA, 23 bp, synthetic construct
  misc_feature 1..23: note = Synthetic polynucleotide
  source 1..23: mol_type = other DNA
attagccatgcctccagtagccg

SEQ ID NO: 914 - DNA, 23 bp, synthetic construct
  misc_feature 1..23: note = Synthetic polynucleotide
  source 1..23: mol_type = other DNA
ctcctgtgtcaattatctttggg

SEQ ID NO: 915 - DNA, 22 bp, synthetic construct
  misc_feature 1..22: note = Synthetic polynucleotide
  source 1..22: mol_type = other DNA
gtctctacagccaattcctctg

SEQ ID NO: 916 - DNA, 26 bp, synthetic construct
  misc_feature 1..26: note = Synthetic polynucleotide
  source 1..26: mol_type = other DNA
tggatccgcagtcaatctttccttgg

SEQ ID NO: 917 - DNA, 21 bp, synthetic construct
  misc_feature 1..21: note = Synthetic polynucleotide
  source 1..21: mol_type = other DNA
cctttgctggtctttggtttg

SEQ ID NO: 918 - DNA, 20 bp, synthetic construct
  misc_feature 1..20: note = Synthetic polynucleotide
  source 1..20: mol_type = other DNA
accttctacaatgagctgcg

SEQ ID NO: 919 - DNA, 24 bp, synthetic construct
  misc_feature 1..24: note = Synthetic polynucleotide
  source 1..24: mol_type = other DNA
atctgggtcatcttctcgcggttg

SEQ ID NO: 920 - DNA, 21 bp, synthetic construct
  misc_feature 1..21: note = Synthetic polynucleotide
  source 1..21: mol_type = other DNA
cctggatagcaacgtacatgg

SEQ ID NO: 921 - DNA, 20 bp, synthetic construct
  misc_feature 1..20: note = Synthetic polynucleotide
  source 1..20: mol_type = other DNA
acttgcccagatgttctcag

SEQ ID NO: 922 - DNA, 24 bp, synthetic construct
  misc_feature 1..24: note = Synthetic polynucleotide
  source 1..24: mol_type = other DNA
accaaccgatccaaagtctccagc

SEQ ID NO: 923 - DNA, 22 bp, synthetic construct
  misc_feature 1..22: note = Synthetic polynucleotide
  source 1..22: mol_type = other DNA
gtatccctctagccattcagtg

SEQ ID NO: 924 - DNA, 19 bp, synthetic construct
  misc_feature 1..19: note = Synthetic polynucleotide
  source 1..19: mol_type = other DNA
acatcgctcagacaccatg

SEQ ID NO: 925 - DNA, 25 bp, synthetic construct
  misc_feature 1..25: note = Synthetic polynucleotide
  source 1..25: mol_type = other DNA
aaggtcggagtcaacggatttggtc

SEQ ID NO: 926 - DNA, 22 bp, synthetic construct
  misc_feature 1..22: note = Synthetic polynucleotide
  source 1..22: mol_type = other DNA
tgtagttgaggtcaatgaaggg